(19) United States
(12) Patent Application Publication
(10) Pub. No.: US 2024/0408235 A1
(43) Pub. Date: Dec. 12, 2024

(54) GENE EDITING METHODS FOR TREATING ALPHA-1 ANTITRYPSIN (AAT) DEFICIENCY

(71) Applicant: Precision BioSciences, Inc., Durham, NC (US)

(72) Inventors: Cassandra Gorsuch, Hillsborough, NC (US); Paige Scarlett Nemec, Durham, NC (US); Jason Richard Harris, Durham, NC (US); Wendy Shoop, Durham, NC (US)

(21) Appl. No.: 18/702,688

(22) PCT Filed: Oct. 19, 2022
(86) PCT No.: PCT/US2022/078388

§ 371 Date: Apr. 18, 2024

Related U.S. Application Data
(60) Provisional application No. 63/257,528, filed on Oct. 19, 2021.

(51) Int. Cl. A61K 48/00; C07K 14/81; C12N 9/22; C12N 15/86; C12N 15/88

(52) U.S. Cl. CPC A61K 48/005; C07K 14/8125; C12N 9/22; C12N 15/86; C12N 15/88; C12N 2750/14143

(57) ABSTRACT

Disclosed are engineered meganucleases that bind and cleave a recognition sequence within a serine peptidase inhibitor, Clade A, Member 1 (SERPINA1) gene, which encodes alpha-1 antitrypsin (AAT). Further disclosed are donor polynucleotides that encode functional AAT proteins. The present disclosure also encompasses methods of using such engineered meganucleases and donor polynucleotides to make genetically-modified cells and use of such compositions for treatment of AAT deficiency.

### CROSS-REFERENCE TO RELATED APPLICATION

This application claims the benefit under 35 U.S.C. § 119 (e) of the earlier filing dates of U.S. Provisional Application No. 63/257,528, filed Oct. 19, 2021, the contents of which are incorporated by reference herein in their entirety.

### REFERENCE TO AN ELECTRONIC SEQUENCE LISTING

The contents of the electronic sequence listing (P109070065WO00-SEQ-NTJ.xml; Size: 215,042 bytes; and Date of Creation: Oct. 19, 2022) are herein incorporated by reference in their entirety.

### FIELD OF THE INVENTION

The invention relates to the field of molecular biology and recombinant nucleic acid technology. In particular, the embodiments of the present disclosure described herein relate to engineered meganucleases having specificity for a recognition sequence within a serpin family A member 1 (SERPINA1) gene encoding alpha-1 antitrypsin (AAT). Such engineered meganucleases are useful in methods for treating AAT deficiency by gene editing to restore AAT function.

### BACKGROUND OF THE INVENTION

AAT deficiency is an autosomal codominant disorder caused by a mutation in the SERPINA1 gene, which encodes the AAT protein. Mutations in the coding sequence of the SERPINA1 gene result in expression of a mutant AAT protein with reduced or abrogated function. AAT is produced in the liver, and transported to the lungs, where it inhibits the activities of serine proteases (serpins), such as neutrophil elastase. Uncontrolled neutrophil elastase activity degrades connective tissue in the lungs. Additionally, mutated AAT is less able to be exported from the liver, causing buildup of AAT aggregates in the liver and subsequent liver toxicity. Current therapies for AAT deficiency are limited to liver transplant and/or regular injections of a plasma containing elevated amounts of functional AAT.

AAT deficiency may be caused by one of multiple mutations in the SERPINA1 gene. The most common mutation in severe disease is the Pi*Z mutation, a single nucleotide polymorphism that results in the substitution of glutamate with lysine at residue 342 (Glu342Lys or E342K). A second common mutation is the Pi*S mutation, which results in the substitution of valine with glutamic acid at residue 264 (V264E).

### SUMMARY OF THE INVENTION

The present disclosure provides compositions and methods that allow for the simultaneous silencing of a mutant SERPINA1 gene, and production of a modified SERPINA1 gene comprising an inserted template nucleic acid. Transcription of the modified SERPINA1 gene, which includes an inserted template nucleic acid sequence, results in a pre-mRNA that is spliced during processing to form an mRNA encoding a full-length, functional (e.g., wild-type) AAT protein, while excluding mutations such as the Pi*Z an Pi*S mutations. By reducing expression of the mutant, dysfunctional AAT protein and promoting the expression of a functional (e.g., wild-type) AAT protein, this gene editing approach alleviates the progression of AAT deficiency. Furthermore, the approach described herein allows for a one-step knockout of endogenous mutant AAT protein expression and knock-in of a donor template that allows for expression of a functional (e.g., wild-type) AAT protein. Accordingly, the present disclosure fulfills a need in the art for gene therapy approaches to treat AAT deficiency.

Thus, in one aspect, the present disclosure provides a polynucleotide comprising a template nucleic acid, wherein the template nucleic acid comprises, from 5′ to 3′: (a) a splicing sequence comprising a splice acceptor sequence capable of pairing with an endogenous splice donor sequence in a SERPINA1 gene; (b) a donor nucleic acid sequence encoding an alpha-1 antitrypsin (AAT) protein, or a portion thereof; and (c) a termination sequence.

In some embodiments, the polynucleotide comprises a 5′ homology arm and a 3′ homology arm flanking the template nucleic acid.

In some embodiments, the polynucleotide does not comprise an exogenous promoter.

In some embodiments, the splicing sequence comprises a branch point. In some embodiments, the splicing sequence is a naturally-occurring splicing sequence (e.g., a naturally occurring intron). In some embodiments, the splicing sequence comprises an SV40 splicing sequence (e.g., intron), a CMV splicing sequence (e.g., intron), or a transferrin gene splicing sequence (e.g., intron). In some embodiments, the splicing sequence is a synthetic splicing sequence (e.g., a synthetic intron).

In some embodiments, the termination sequence comprises a stop codon. In some embodiments, the termination sequence comprises a polyA sequence. In some embodiments, the termination sequence comprises a stop codon and a polyA sequence.

In some embodiments, the AAT protein, or portion thereof, encoded by the donor nucleic acid sequence is a wild-type AAT protein, or a portion thereof. In some embodiments, the donor nucleic acid sequence comprises one or more exons of a wild-type SERPINA1 gene. In some embodiments, the donor nucleic acid sequence comprises one or more exons of a SERPINA1 gene that have been codon-modified but encode a wild-type AAT protein, or a portion thereof.

In some embodiments, the donor nucleic acid sequence encodes an AAT protein encoded by exons 2, 3, 4, and 5 of a SERPINA1 gene, and the splice acceptor sequence is capable of pairing with an endogenous splice donor sequence that is positioned 3′ downstream and adjacent to exon 1a in a SERPINA1 gene. In some such embodiments, the donor nucleic acid sequence comprises exons 1b, 1c, 2, 3, 4, and 5 of a SERPINA1 gene, or codon-modified variants of one or more of exons 1b, 1c, 2, 3, 4, and 5 of a SERPINA1 gene. In some such embodiments, the donor nucleic acid sequence does not comprise one or more of introns 1b, 1c, 2, 3, and 4 of a SERPINA1 gene. In some such embodiments, the donor nucleic acid sequence comprises one or more of introns 1b, 1c, 2, 3, and 4 of a SERPINA1 gene. In some such embodiments, the donor nucleic acid sequence comprises introns 1b, 1c, 2, 3, and 4 of a SERPINA1 gene. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 3. In some such embodiments, the donor nucleic acid sequence comprises a nucleic acid sequence set forth in SEQ ID NO: 3. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 4. In some such embodiments, the donor nucleic acid sequence comprises a nucleic acid sequence set forth in SEQ ID NO: 4.

In some embodiments, the donor nucleic acid sequence encodes an AAT protein encoded by exons 2, 3, 4, and 5 of a SERPINA1 gene, and the splice acceptor sequence is capable of pairing with an endogenous splice donor sequence that is positioned 3′ downstream and adjacent to exon 1b in a SERPINA1 gene. In some such embodiments, the donor nucleic acid sequence comprises exons 1c, 2, 3, 4, and 5 of a SERPINA1 gene, or codon-modified variants of one or more of exons 1c, 2, 3, 4, and 5 of a SERPINA1 gene. In some such embodiments, the donor nucleic acid sequence does not comprise one or more of introns 1c, 2, 3, and 4 of a SERPINA1 gene. In some such embodiments, the donor nucleic acid sequence comprises one or more of introns 1c, 2, 3, and 4 of a SERPINA1 gene. In some such embodiments, the donor nucleic acid sequence comprises introns 1c, 2, 3, and 4 of a SERPINA1 gene. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 5. In some such embodiments, the donor nucleic acid sequence comprises a nucleic acid sequence set forth in SEQ ID NO: 5. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 6. In some such embodiments, the donor nucleic acid sequence comprises a nucleic acid sequence set forth in SEQ ID NO: 6.

In some embodiments, the donor nucleic acid sequence encodes an AAT protein encoded by exons 2, 3, 4, and 5 of a SERPINA1 gene, and wherein the splice acceptor sequence is capable of pairing with an endogenous splice donor sequence that is positioned 3′ downstream and adjacent to exon 1c in a SERPINA1 gene. In some such embodiments, the donor nucleic acid sequence comprises exons 2, 3, 4, and 5 of a SERPINA1 gene, or codon-modified variants of one or more of exons 2, 3, 4, and 5 of a SERPINA1 gene. In some such embodiments, the donor nucleic acid sequence does not comprise one or more of introns 2, 3, and 4 of a SERPINA1 gene. In some such embodiments, the donor nucleic acid sequence comprises one or more of introns 2, 3, and 4 of a SERPINA1 gene. In some such embodiments, the donor nucleic acid sequence comprises introns 2, 3, and 4 of a SERPINA1 gene. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 7. In some such embodiments, the donor nucleic acid sequence comprises a nucleic acid sequence set forth in SEQ ID NO: 7. In some such embodiments, the donor nucleic acid sequence comprises a nucleic acid sequence having at least 80%, at least 85%, at least 90%, at least 91%, at least 99%, or more, sequence identity to a sequence set forth in SEQ ID NO: 8. In some such embodiments, the donor nucleic acid sequence comprises a nucleic acid sequence set forth in SEQ ID NO: 8.

In some embodiments, the donor nucleic acid sequence encodes a portion of an AAT protein encoded by exons 3, 4, and 5 of a SERPINA1 gene, and the splice acceptor sequence is capable of pairing with an endogenous splice donor sequence that is positioned 3′ downstream and adjacent to exon 2 in a SERPINA1 gene. In some such embodiments, the donor nucleic acid sequence comprises exons 3, 4, and 5 of a SERPINA1 gene, or codon-modified variants of one or more of exons 3, 4, and 5 of a SERPINA1 gene. In some such embodiments, the donor nucleic acid sequence does not comprise one or more of introns 3 and 4 of a SERPINA1 gene. In some such embodiments, the donor nucleic acid sequence comprises one or more of introns 3 and 4 of a SERPINA1 gene. In some such embodiments, the donor nucleic acid sequence comprises introns 3 and 4 of a SERPINA1 gene. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 9. In some such embodiments, the donor nucleic acid sequence comprises a nucleic acid sequence set forth in SEQ ID NO: 9. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 10. In some such embodiments, the donor nucleic acid sequence comprises a nucleic acid sequence set forth in SEQ ID NO: 10.

In some embodiments, the donor nucleic acid sequence encodes a portion of an AAT protein encoded by exons 4 and 5 of a SERPINA1 gene, and wherein the splice acceptor sequence is capable of pairing with an endogenous splice donor sequence that is positioned 3′ downstream and adjacent to exon 3 in a SERPINA1 gene. In some such embodiments, the donor nucleic acid sequence comprises exons 4 and 5 of a SERPINA1 gene, or codon-modified variants of one or more of exons 4 and 5 of a SERPINA1 gene. In some such embodiments, the donor nucleic acid sequence does not comprise intron 4 of a SERPINA1 gene. In some such embodiments, the donor nucleic acid sequence comprises intron 4 of a SERPINA1 gene. In some such embodiments, the donor nucleic acid sequence comprises a nucleic acid sequence having at least 80%, at least 85%, at least 90%, at least 91%, at least 99%, or more, sequence identity to a sequence set forth in SEQ ID NO: 11. In some such embodiments, the donor nucleic acid sequence comprises a nucleic acid sequence set forth in SEQ ID NO: 11. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 12. In some such embodiments, the donor nucleic acid sequence comprises a nucleic acid sequence set forth in SEQ ID NO: 12.

In some embodiments, the donor nucleic acid sequence encodes a portion of an AAT protein encoded by exon 5 of a SERPINA1 gene, and wherein the splice acceptor sequence is capable of pairing with an endogenous splice donor sequence that is positioned 3′ downstream and adjacent to exon 4 in a SERPINA1 gene. In some such embodiments, the donor nucleic acid sequence comprises exon 5 of a SERPINA1 gene, or a codon-modified variant of exon 5 of a SERPINA1 gene. In some such embodiments, the donor nucleic acid sequence comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 13. In some such embodiments, the donor nucleic acid sequence comprises a nucleic acid sequence set forth in SEQ ID NO: 13.

In some embodiments, the template nucleic acid is a bidirectional template nucleic acid. In some such embodiments, the donor nucleic acid sequence further comprises a reverse segment that is 3′ downstream of the termination sequence, wherein the reverse segment comprises, from 5′ to 3′: (a) a reverse complement of a second termination sequence; (b) a reverse complement of a second donor nucleic acid sequence encoding an AAT protein, or a portion thereof; and (c) a reverse complement of a second splicing sequence comprising a splice acceptor sequence capable of pairing with an endogenous splice donor sequence in a SERPINA1 gene. In some such embodiments, the second termination sequence is identical to the first termination sequence described herein. In some such embodiments, the second termination sequence differs from the first termination sequence described herein. In some such embodiments, the second donor nucleic acid sequence is identical to the first donor nucleic acid sequence described herein. In some such embodiments, the second donor nucleic acid sequence differs from the first donor nucleic acid sequence described herein, but encodes the same AAT protein, or portion thereof. In some such embodiments, the second splicing sequence is identical to the first splicing sequence described herein. In some such embodiments, the second splicing sequence differs from the splicing sequence described herein, but is still capable of pairing with the same endogenous splice donor sequence in a SERPINA1 gene.

In another aspect, the present disclosure provides a recombinant DNA construct comprising a polynucleotide comprising a template nucleic acid described herein.

In some embodiments, the recombinant DNA construct encodes a recombinant virus comprising the polynucleotide. In some embodiments, the recombinant virus is a recombinant adenovirus, a recombinant lentivirus, a recombinant retrovirus, or a recombinant adeno-associated virus (AAV). In some embodiments, the recombinant virus is a recombinant AAV. In some embodiments, the recombinant AAV has an AAV8 capsid.

In another aspect, the present disclosure provides a recombinant virus comprising a polynucleotide comprising a template nucleic acid described herein.

In some embodiments, the recombinant virus is a recombinant adenovirus, a recombinant lentivirus, a recombinant retrovirus, or a recombinant AAV. In some embodiments, the recombinant virus is a recombinant AAV. In some embodiments, the recombinant AAV has an AAV8 capsid. In some embodiments, the polynucleotide is flanked by inverted terminal repeat (ITR) sequences.

In another aspect, the present disclosure provides a lipid nanoparticle composition comprising lipid nanoparticles comprising a polynucleotide comprising a template nucleic acid described herein.

In another aspect, the present disclosure provides a lipid nanoparticle composition comprising lipid nanoparticles comprising a recombinant DNA construct described herein (i.e., comprising a polynucleotide comprising a template nucleic acid described herein).

In another aspect, the present disclosure provides a pharmaceutical composition comprising a pharmaceutically acceptable carrier and a polynucleotide comprising a template nucleic acid described herein.

In another aspect, the present disclosure provides a pharmaceutical composition comprising a pharmaceutically acceptable carrier and a recombinant DNA construct described herein (i.e., comprising a polynucleotide comprising a template nucleic acid described herein).

In another aspect, the present disclosure provides a pharmaceutical composition comprising a pharmaceutically acceptable carrier and a recombinant virus described herein (i.e., comprising a polynucleotide comprising a template nucleic acid described herein).

In another aspect, the present disclosure provides a pharmaceutical composition comprising a pharmaceutically acceptable carrier and a lipid nanoparticle composition described herein (i.e., comprising a polynucleotide comprising a template nucleic acid described herein).

In another aspect, the present disclosure provides a method for producing a genetically-modified eukaryotic cell comprising a modified SERPINA1 gene, the method comprising introducing into a eukaryotic cell: (a) a polynucleotide comprising a template nucleic acid described herein; and (b) an engineered nuclease, or a second polynucleotide comprising a nucleic acid sequence encoding an engineered nuclease that is expressed in the eukaryotic cell; wherein the engineered nuclease binds and cleaves a recognition sequence within an endogenous SERPINA1 gene to generate a cleavage site, and wherein the template nucleic acid is inserted into the cleavage site to generate the modified SERPINA1 gene.

In some embodiments, the endogenous SERPINA1 gene comprises at least one mutation relative to a wild-type SERPINA1 gene and encodes a mutant AAT protein. In some embodiments, the endogenous SERPINA1 gene comprises a Z allele mutation in exon 5. In some embodiments, the endogenous SERPINA1 gene comprises an S allele mutation in exon 3.

In some embodiments, insertion of the template nucleic acid into the cleavage site disrupts expression of an endogenous AAT protein encoded by the endogenous SERPINA1 gene. In some embodiments, the genetically-modified cell expresses less of a mutant AAT protein, relative to an unmodified cell.

In some embodiments, the template nucleic acid is inserted in-frame in the SERPINA1 gene.

In some embodiments, the donor nucleic acid sequence of the template nucleic acid is operably linked to an endogenous SERPINA1 promoter following insertion of the template nucleic acid into the cleavage site.

In some embodiments, the template nucleic acid does not comprise an exogenous promoter.

In some embodiments, the modified SERPINA1 gene encodes a full-length AAT protein that does not comprise a Z allele mutation or an S allele mutation. In some embodiments, the modified SERPINA1 gene encodes a full-length wild-type AAT protein. In some embodiments, the modified SERPINA1 gene comprises a nucleic acid sequence of a wild-type SERPINA1 gene. In some embodiments, the modified SERPINA1 gene comprises one or more codon-modified exons and/or introns and encodes a wild-type AAT protein. In some embodiments, the modified SERPINA1 gene comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 16. In some embodiments, the modified SERPINA1 gene comprises a nucleic acid sequence set forth in SEQ ID NO: 16.

In some embodiments, the engineered nuclease is an engineered meganuclease, a CRISPR system nuclease, a TALEN, a compact TALEN, a zinc finger nuclease, or a megaTAL. In some embodiments, the engineered nuclease is an engineered meganuclease.

In some embodiments, the second polynucleotide comprises a promoter that is operably linked to the nucleic acid sequence encoding the engineered nuclease. In some embodiments, the promoter is a liver-specific promoter. In some embodiments, the liver-specific promoter is a TBG promoter, alpha-1 antitrypsin promoter, hybrid liver-specific promoter comprising a hepatic locus control region from an ApoE gene and an alpha-1 antitrypsin promoter, or apolipoprotein A-II promoter. In some embodiments, the liver-specific promoter is a TBG promoter.

In some embodiments, the polynucleotide comprises a 5′ homology arm and a 3′ homology arm flanking the template nucleic acid that are homologous to sequences flanking the cleavage site. In some embodiments, the template nucleic acid is inserted into the cleavage site by homologous recombination.

In some embodiments, the recognition sequence is positioned within intron 1a of the SERPINA1 gene. In some such embodiments, the recognition sequence comprises SEQ ID NO: 22. In some such embodiments, the polynucleotide comprising a template nucleic acid is a polynucleotide described herein which comprises a donor nucleic acid sequence that encodes an AAT protein encoded by exons 2, 3, 4, and 5 of a SERPINA1 gene, and a splice acceptor sequence capable of pairing with an endogenous splice donor sequence that is positioned 3′ downstream and adjacent to exon 1a in a SERPINA1 gene.

In some embodiments, the recognition sequence is positioned within intron 1b of the SERPINA1 gene. In some such embodiments, the polynucleotide comprising a template nucleic acid is a described herein which comprises a donor nucleic acid sequence that encodes an AAT protein encoded by exons 2, 3, 4, and 5 of a SERPINA1 gene, and comprises a splice acceptor sequence capable of pairing with an endogenous splice donor sequence that is positioned 3′ downstream and adjacent to exon 1b in a SERPINA1 gene.

In some embodiments, the recognition sequence is positioned within intron 1c of the SERPINA1 gene. In some such embodiments, the recognition sequence comprises SEQ ID NO: 24. In some such embodiments, the recognition sequence comprises SEQ ID NO: 26. In some such embodiments, the recognition sequence comprises SEQ ID NO: 28. In some such embodiments, the recognition sequence comprises SEQ ID NO: 30. In some such embodiments, the recognition sequence comprises SEQ ID NO: 32. In some such embodiments, the polynucleotide comprising a template nucleic acid is a polynucleotide described herein that comprises a donor nucleic acid sequence encoding an AAT protein encoded by exons 2, 3, 4, and 5 of a SERPINA1 gene, and comprising a splice acceptor sequence capable of pairing with an endogenous splice donor sequence that is positioned 3′ downstream and adjacent to exon 1c in a SERPINA1 gene.

In some embodiments, the recognition sequence is positioned within intron 2 of the SERPINA1 gene. In some such embodiments, the polynucleotide comprising a template nucleic acid is a polynucleotide described herein comprising a donor nucleic acid sequence encoding a portion of an AAT protein encoded by exons 3, 4, and 5 of a SERPINA1 gene, and comprising a splice acceptor sequence capable of pairing with an endogenous splice donor sequence that is positioned 3′ downstream and adjacent to exon 2 in a SERPINA1 gene.

In some embodiments, the recognition sequence is positioned within intron 3 of the SERPINA1 gene. In some such embodiments, the recognition sequence comprises SEQ ID NO: 18. In some such embodiments, the polynucleotide comprising a template nucleic acid is a polynucleotide described herein comprising a donor nucleic acid sequence encoding a portion of an AAT protein encoded by exons 4 and 5 of a SERPINA1 gene, and comprising a splice acceptor sequence capable of pairing with an endogenous splice donor sequence that is positioned 3′ downstream and adjacent to exon 3 in a SERPINA1 gene.

In some embodiments, the recognition sequence is positioned within intron 4 of the SERPINA1 gene. In some such embodiments, the polynucleotide comprising a template nucleic acid is a polynucleotide described herein comprising a donor nucleic acid sequence encoding a portion of an AAT protein encoded by exon 5 of a SERPINA1 gene, and comprising a splice acceptor sequence capable of pairing with an endogenous splice donor sequence that is positioned 3′ downstream and adjacent to exon 4 in a SERPINA1 gene.

In some embodiments, the polynucleotide comprising a template nucleic acid is introduced into the eukaryotic cell by a first recombinant virus and the second polynucleotide is introduced into the eukaryotic cell by a second recombinant virus. In some such embodiments, the first recombinant virus and/or the second recombinant virus is a recombinant AAV. In some such embodiments, the first recombinant AAV and/or the second recombinant AAV has a capsid of serotype AAV8. In some such embodiments, the polynucleotide comprising a template nucleic acid and the second polynucleotide are flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is introduced into the eukaryotic cell by a recombinant virus, and wherein the engineered nuclease or the second polynucleotide is introduced into the eukaryotic cell by a lipid nanoparticle. In some such embodiments, the recombinant virus is a recombinant AAV. In some such embodiments, the recombinant AAV has a capsid of serotype AAV8. In some such embodiments, the polynucleotide comprising a template nucleic acid is flanked by ITR sequences. In some such embodiments, the second polynucleotide is an mRNA. In some such embodiments, the second polynucleotide is a double-stranded DNA encapsulated by the lipid nanoparticle.

In some embodiments, the polynucleotide comprising a template nucleic acid is introduced into the eukaryotic cell by a lipid nanoparticle, and wherein the second polynucleotide is introduced into the eukaryotic cell by a recombinant virus. In some such embodiments, the polynucleotide comprising a template nucleic acid is a double-stranded DNA encapsulated by the lipid nanoparticle. In some such embodiments, the recombinant virus is a recombinant AAV. In some such embodiments, the recombinant AAV has a capsid of serotype AAV8. In some such embodiments, the second polynucleotide is flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is introduced into the eukaryotic cell by a first lipid nanoparticle, and the engineered nuclease or the second polynucleotide is introduced into the eukaryotic cell by a second lipid nanoparticle. In some such embodiments, the polynucleotide comprising a template nucleic acid is a double-stranded DNA encapsulated by the first lipid nanoparticle. In some such embodiments, the second polynucleotide is an mRNA encapsulated by the second lipid nanoparticle. In some such embodiments, the second polynucleotide is a double-stranded DNA encapsulated by the second lipid nanoparticle.

In some embodiments, the eukaryotic cell is a mammalian cell. In some embodiments, the mammalian cell is a human cell. In some embodiments, the mammalian cell is a liver cell (e.g., a hepatocyte). In some embodiments, the mammalian cell is a liver progenitor cell or stem cell.

In another aspect, the present disclosure provides a method for modifying a SERPINA1 gene in a target cell in a subject, the method comprising delivering to the target cell: (a) a polynucleotide comprising a template nucleic acid described herein; and (b) an engineered nuclease, or a second polynucleotide comprising a nucleic acid sequence encoding an engineered nuclease that is expressed in the target cell; wherein the engineered nuclease binds and cleaves a recognition sequence within an endogenous SERPINA1 gene in the target cell to generate a cleavage site, and wherein the template nucleic acid is inserted into the cleavage site to generate the modified SERPINA1 gene.

In some embodiments, the endogenous SERPINA1 gene comprises at least one mutation relative to a wild-type SERPINA1 gene and encodes a mutant AAT protein. In some embodiments, the endogenous SERPINA1 gene comprises a Z allele mutation in exon 5. In some embodiments, the endogenous SERPINA1 gene comprises an S allele mutation in exon 3.

In some embodiments, insertion of the template nucleic acid into the cleavage site disrupts expression of an endogenous AAT protein encoded by the endogenous SERPINA1 gene. In some embodiments, the target cell expresses less of a mutant AAT protein after insertion of the template nucleic acid, relative to before insertion.

In some embodiments, the template nucleic acid is inserted in-frame in the SERPINA1 gene.

In some embodiments, the donor nucleic acid sequence of the template nucleic acid is operably linked to an endogenous SERPINA1 promoter following insertion of the template nucleic acid into the cleavage site.

In some embodiments, the template nucleic acid does not comprise an exogenous promoter.

In some embodiments, the modified SERPINA1 gene encodes a full-length AAT protein that does not comprise a Z allele mutation or an S allele mutation. In some embodiments, the modified SERPINA1 gene encodes a full-length wild-type AAT protein. In some embodiments, the modified SERPINA1 gene comprises a nucleic acid sequence of a wild-type SERPINA1 gene. In some embodiments, the modified SERPINA1 gene comprises one or more codon-modified exons and/or introns and encodes a wild-type AAT protein. In some embodiments, the modified SERPINA1 gene comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 16. In some embodiments, the modified SERPINA1 gene comprises a nucleic acid sequence set forth in SEQ ID NO: 16.

In some embodiments, the engineered nuclease is an engineered meganuclease, a CRISPR system nuclease, a TALEN, a compact TALEN, a zinc finger nuclease, or a megaTAL. In some embodiments, the engineered nuclease is an engineered meganuclease.

In some embodiments, the second polynucleotide comprises a promoter that is operably linked to the nucleic acid sequence encoding the engineered nuclease. In some embodiments, the promoter is a liver-specific promoter. In some embodiments, the liver-specific promoter is a TBG promoter, alpha-1 antitrypsin promoter, hybrid liver-specific promoter comprising a hepatic locus control region from an ApoE gene and an alpha-1 antitrypsin promoter, or apolipoprotein A-II promoter. In some embodiments, the liver-specific promoter is a TBG promoter.

In some embodiments, the polynucleotide comprises a 5′ homology arm and a 3′ homology arm flanking the template nucleic acid that are homologous to sequences flanking the cleavage site. In some embodiments, the template nucleic acid is inserted into the cleavage site by homologous recombination.

In some embodiments, the recognition sequence is positioned within intron 1a of the SERPINA1 gene. In some such embodiments, the recognition sequence comprises SEQ ID NO: 22. In some such embodiments, the polynucleotide comprising a template nucleic acid is a polynucleotide described herein which comprises a donor nucleic acid sequence that encodes an AAT protein encoded by exons 2, 3, 4, and 5 of a SERPINA1 gene, and a splice acceptor sequence capable of pairing with an endogenous splice donor sequence that is positioned 3′ downstream and adjacent to exon 1a in a SERPINA1 gene.

In some embodiments, the recognition sequence is positioned within intron 1b of the SERPINA1 gene. In some such embodiments, the polynucleotide comprising a template nucleic acid is a described herein which comprises a donor nucleic acid sequence that encodes an AAT protein encoded by exons 2, 3, 4, and 5 of a SERPINA1 gene, and comprises a splice acceptor sequence capable of pairing with an endogenous splice donor sequence that is positioned 3′ downstream and adjacent to exon 1b in a SERPINA1 gene.

In some embodiments, the recognition sequence is positioned within intron 1c of the SERPINA1 gene. In some such embodiments, the recognition sequence comprises SEQ ID NO: 24. In some such embodiments, the recognition sequence comprises SEQ ID NO: 26. In some such embodiments, the recognition sequence comprises SEQ ID NO: 28. In some such embodiments, the recognition sequence comprises SEQ ID NO: 30. In some such embodiments, the recognition sequence comprises SEQ ID NO: 32. In some such embodiments, the polynucleotide comprising a template nucleic acid is a polynucleotide described herein that comprises a donor nucleic acid sequence encoding an AAT protein encoded by exons 2, 3, 4, and 5 of a SERPINA1 gene, and comprising a splice acceptor sequence capable of pairing with an endogenous splice donor sequence that is positioned 3′ downstream and adjacent to exon 1c in a SERPINA1 gene.

In some embodiments, the recognition sequence is positioned within intron 2 of the SERPINA1 gene. In some such embodiments, the polynucleotide comprising a template nucleic acid is a polynucleotide described herein comprising a donor nucleic acid sequence encoding a portion of an AAT protein encoded by exons 3, 4, and 5 of a SERPINA1 gene, and comprising a splice acceptor sequence capable of pairing with an endogenous splice donor sequence that is positioned 3′ downstream and adjacent to exon 2 in a SERPINA1 gene.

In some embodiments, the recognition sequence is positioned within intron 3 of the SERPINA1 gene. In some such embodiments, the recognition sequence comprises SEQ ID NO: 18. In some such embodiments, the polynucleotide comprising a template nucleic acid is a polynucleotide described herein comprising a donor nucleic acid sequence encoding a portion of an AAT protein encoded by exons 4 and 5 of a SERPINA1 gene, and comprising a splice acceptor sequence capable of pairing with an endogenous splice donor sequence that is positioned 3′ downstream and adjacent to exon 3 in a SERPINA1 gene.

In some embodiments, the recognition sequence is positioned within intron 4 of the SERPINA1 gene. In some such embodiments, the polynucleotide comprising a template nucleic acid is a polynucleotide described herein comprising a donor nucleic acid sequence encoding a portion of an AAT protein encoded by exon 5 of a SERPINA1 gene, and comprising a splice acceptor sequence capable of pairing with an endogenous splice donor sequence that is positioned 3′ downstream and adjacent to exon 4 in a SERPINA1 gene.

In some embodiments, the polynucleotide comprising a template nucleic acid is delivered to the target cell by a first recombinant virus and the second polynucleotide is delivered to the eukaryotic cell by a second recombinant virus. In some such embodiments, the first recombinant virus and/or the second recombinant virus is a recombinant AAV. In some such embodiments, the first recombinant AAV and/or the second recombinant AAV has a capsid of serotype AAV8. In some such embodiments, the polynucleotide comprising a template nucleic acid and the second polynucleotide are flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is delivered to the target cell by a recombinant virus, and the engineered nuclease or the second polynucleotide is delivered to the target cell by a lipid nanoparticle. In some such embodiments, the recombinant virus is a recombinant AAV. In some such embodiments, the recombinant AAV has a capsid of serotype AAV8. In some such embodiments, the polynucleotide comprising a template nucleic acid is flanked by ITR sequences. In some such embodiments, the second polynucleotide is an mRNA. In some such embodiments, the second polynucleotide is a double-stranded DNA encapsulated by the lipid nanoparticle.

In some embodiments, the polynucleotide comprising a template nucleic acid is delivered to the target cell by a lipid nanoparticle, and the second polynucleotide is delivered to the target cell by a recombinant virus. In some such embodiments, the polynucleotide comprising a template nucleic acid is a double-stranded DNA encapsulated by the lipid nanoparticle. In some such embodiments, the recombinant virus is a recombinant AAV. In some such embodiments, the recombinant AAV has a capsid of serotype AAV8. In some such embodiments, the second polynucleotide is flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is delivered to the target cell by a first lipid nanoparticle, and the engineered nuclease or the second polynucleotide is delivered to the target cell by a second lipid nanoparticle. In some such embodiments, the polynucleotide comprising a template nucleic acid is a double-stranded DNA encapsulated by the first lipid nanoparticle. In some such embodiments, the second polynucleotide is an mRNA encapsulated by the second lipid nanoparticle. In some such embodiments, the second polynucleotide is a double-stranded DNA encapsulated by the second lipid nanoparticle.

In some embodiments, the target cell is a mammalian cell. In some embodiments, the mammalian cell is a human cell. In some embodiments, the mammalian cell is a liver cell (e.g., a hepatocyte). In some embodiments, the mammalian cell is a liver progenitor cell or stem cell.

In another aspect, the present disclosure provides a method for treating AAT deficiency in a subject in need thereof, the method comprising administering to the subject: (a) a pharmaceutical composition comprising an effective amount of a polynucleotide comprising a template nucleic acid described herein; and (b) a pharmaceutical composition comprising an effective amount of an engineered nuclease or a second polynucleotide comprising a nucleic acid sequence encoding an engineered nuclease; wherein the polynucleotide comprising a template nucleic acid, and the engineered nuclease or the second polynucleotide, are delivered to a target cell in the subject, and wherein the engineered nuclease is expressed in the target cell if encoded by the second polynucleotide, wherein the engineered nuclease binds and cleaves a recognition sequence within an endogenous SERPINA1 gene in the target cell to generate a cleavage site, and wherein the template nucleic acid is inserted into the cleavage site to generate the modified SERPINA1 gene.

In some embodiments, the endogenous SERPINA1 gene comprises at least one mutation relative to a wild-type SERPINA1 gene and encodes a mutant AAT protein. In some embodiments, the endogenous SERPINA1 gene comprises a Z allele mutation in exon 5. In some embodiments, the endogenous SERPINA1 gene comprises an S allele mutation in exon 3.

In some embodiments, insertion of the template nucleic acid into the cleavage site disrupts expression of an endogenous AAT protein encoded by the endogenous SERPINA1 gene. In some embodiments, the target cell expresses less of a mutant AAT protein after insertion of the template nucleic acid, relative to before insertion.

In some embodiments, the template nucleic acid is inserted in-frame in the SERPINA1 gene.

In some embodiments, the donor nucleic acid sequence of the template nucleic acid is operably linked to an endogenous SERPINA1 promoter following insertion of the template nucleic acid into the cleavage site.

In some embodiments, the template nucleic acid does not comprise an exogenous promoter.

In some embodiments, the modified SERPINA1 gene encodes a full-length AAT protein that does not comprise a Z allele mutation or an S allele mutation. In some embodiments, the modified SERPINA1 gene encodes a full-length wild-type AAT protein. In some embodiments, the modified SERPINA1 gene comprises a nucleic acid sequence of a wild-type SERPINA1 gene. In some embodiments, the modified SERPINA1 gene comprises one or more codon-modified exons and/or introns and encodes a wild-type AAT protein. In some embodiments, the modified SERPINA1 gene comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 16. In some embodiments, the modified SERPINA1 gene comprises a nucleic acid sequence set forth in SEQ ID NO: 16.

In some embodiments, the engineered nuclease is an engineered meganuclease, a CRISPR system nuclease, a TALEN, a compact TALEN, a zinc finger nuclease, or a megaTAL. In some embodiments, the engineered nuclease is an engineered meganuclease.

In some embodiments, the second polynucleotide comprises a promoter that is operably linked to the nucleic acid sequence encoding the engineered nuclease. In some embodiments, the promoter is a liver-specific promoter. In some embodiments, the liver-specific promoter is a TBG promoter, alpha-1 antitrypsin promoter, hybrid liver-specific promoter comprising a hepatic locus control region from an ApoE gene and an alpha-1 antitrypsin promoter, or apolipoprotein A-II promoter. In some embodiments, the liver-specific promoter is a TBG promoter.

In some embodiments, the polynucleotide comprises a 5′ homology arm and a 3′ homology arm flanking the template nucleic acid that are homologous to sequences flanking the cleavage site. In some embodiments, the template nucleic acid is inserted into the cleavage site by homologous recombination.

In some embodiments, the recognition sequence is positioned within intron 1a of the SERPINA1 gene. In some such embodiments, the recognition sequence comprises SEQ ID NO: 22. In some such embodiments, the polynucleotide comprising a template nucleic acid is a polynucleotide described herein which comprises a donor nucleic acid sequence that encodes an AAT protein encoded by exons 2, 3, 4, and 5 of a SERPINA1 gene, and a splice acceptor sequence capable of pairing with an endogenous splice donor sequence that is positioned 3′ downstream and adjacent to exon 1a in a SERPINA1 gene.

In some embodiments, the recognition sequence is positioned within intron 1b of the SERPINA1 gene. In some such embodiments, the polynucleotide comprising a template nucleic acid is a described herein which comprises a donor nucleic acid sequence that encodes an AAT protein encoded by exons 2, 3, 4, and 5 of a SERPINA1 gene, and comprises a splice acceptor sequence capable of pairing with an endogenous splice donor sequence that is positioned 3′ downstream and adjacent to exon 1b in a SERPINA1 gene.

In some embodiments, the recognition sequence is positioned within intron 1c of the SERPINA1 gene. In some such embodiments, the recognition sequence comprises SEQ ID NO: 24. In some such embodiments, the recognition sequence comprises SEQ ID NO: 26. In some such embodiments, the recognition sequence comprises SEQ ID NO: 28. In some such embodiments, the recognition sequence comprises SEQ ID NO: 30. In some such embodiments, the recognition sequence comprises SEQ ID NO: 32. In some such embodiments, the polynucleotide comprising a template nucleic acid is a polynucleotide described herein that comprises a donor nucleic acid sequence encoding an AAT protein encoded by exons 2, 3, 4, and 5 of a SERPINA1 gene, and comprising a splice acceptor sequence capable of pairing with an endogenous splice donor sequence that is positioned 3′ downstream and adjacent to exon 1c in a SERPINA1 gene.

In some embodiments, the recognition sequence is positioned within intron 2 of the SERPINA1 gene. In some such embodiments, the polynucleotide comprising a template nucleic acid is a polynucleotide described herein comprising a donor nucleic acid sequence encoding a portion of an AAT protein encoded by exons 3, 4, and 5 of a SERPINA1 gene, and comprising a splice acceptor sequence capable of pairing with an endogenous splice donor sequence that is positioned 3′ downstream and adjacent to exon 2 in a SERPINA1 gene.

In some embodiments, the recognition sequence is positioned within intron 3 of the SERPINA1 gene. In some such embodiments, the recognition sequence comprises SEQ ID NO: 18. In some such embodiments, the polynucleotide comprising a template nucleic acid is a polynucleotide described herein comprising a donor nucleic acid sequence encoding a portion of an AAT protein encoded by exons 4 and 5 of a SERPINA1 gene, and comprising a splice acceptor sequence capable of pairing with an endogenous splice donor sequence that is positioned 3′ downstream and adjacent to exon 3 in a SERPINA1 gene.

In some embodiments, the recognition sequence is positioned within intron 4 of the SERPINA1 gene. In some such embodiments, the polynucleotide comprising a template nucleic acid is a polynucleotide described herein comprising a donor nucleic acid sequence encoding a portion of an AAT protein encoded by exon 5 of a SERPINA1 gene, and comprising a splice acceptor sequence capable of pairing with an endogenous splice donor sequence that is positioned 3′ downstream and adjacent to exon 4 in a SERPINA1 gene.

In some embodiments, the polynucleotide comprising a template nucleic acid is administered to the subject in a first recombinant virus and the second polynucleotide is administered to the subject in a second recombinant virus. In some such embodiments, the first recombinant virus and/or the second recombinant virus is a recombinant AAV. In some such embodiments, the first recombinant AAV and/or the second recombinant AAV has a capsid of serotype AAV8. In some such embodiments, the polynucleotide comprising a template nucleic acid and the second polynucleotide are flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is administered to the subject in a recombinant virus, and the engineered nuclease or the second polynucleotide is administered to the subject in a lipid nanoparticle. In some such embodiments, the recombinant virus is a recombinant AAV. In some such embodiments, the recombinant AAV has a capsid of serotype AAV8. In some such embodiments, the polynucleotide comprising a template nucleic acid is flanked by ITR sequences. In some such embodiments, the second polynucleotide is an mRNA. In some such embodiments, the second polynucleotide is a double-stranded DNA encapsulated by the lipid nanoparticle.

In some embodiments, the polynucleotide comprising a template nucleic acid is administered to the subject using a lipid nanoparticle, and wherein the second polynucleotide is administered to the subject using a recombinant virus. In some such embodiments, the polynucleotide comprising a template nucleic acid is a double-stranded DNA encapsulated by the lipid nanoparticle. In some such embodiments, the recombinant virus is a recombinant AAV. In some such embodiments, the recombinant AAV has a capsid of serotype AAV8. In some such embodiments, the second polynucleotide is flanked by ITR sequences. In some such embodiments, the second polynucleotide comprises a promoter that is operably linked to the nucleic acid sequence encoding the engineered nuclease. In some such embodiments, the promoter is a liver-specific promoter. In some such embodiments, the liver-specific promoter is a TBG promoter.

In some embodiments, the polynucleotide comprising a template nucleic acid is administered to the subject using a first lipid nanoparticle, and the engineered nuclease or the second polynucleotide is administered to the subject using a second lipid nanoparticle. In some such embodiments, the polynucleotide comprising a template nucleic acid is a double-stranded DNA encapsulated by the first lipid nanoparticle. In some such embodiments, the second polynucleotide is an mRNA encapsulated by the second lipid nanoparticle. In some such embodiments, the second polynucleotide is a double-stranded DNA encapsulated by the second lipid nanoparticle.

In some embodiments, the subject is a human. In some embodiments, the target cell is a liver cell (e.g., a hepatocyte). In some embodiments, the target cell is a liver progenitor cell or stem cell.

### BRIEF DESCRIPTION OF THE FIGURES

FIG. 1 illustrates engineered meganuclease recognition sequences in the human AAT gene for the indicated recognition sequence locations. Each AAT recognition sequence targeted by engineered meganucleases disclosed herein comprises two recognition half-sites. Each recognition half-site comprises 9 base pairs, separated by a 4 base pair central sequence.

FIG. 2 illustrates engineered meganuclease recognition sequences in the human AAT gene for the indicated recognition sequence locations. Each AAT recognition sequence targeted by engineered meganucleases disclosed herein comprises two recognition half-sites. Each recognition half-site comprises 9 base pairs, separated by a 4 base pair central sequence.

FIG. 3 illustrates orientations of engineered meganucleases described herein. The engineered meganucleases described herein comprise two subunits, wherein the first subunit comprising the HVR1 region binds to a first recognition half-site and the second subunit comprising the HVR2 region binds to a second recognition half-site. In embodiments where the engineered meganuclease is a single-chain meganuclease, the first subunit comprising the HVR1 region can be positioned as either the N-terminal or C-terminal subunit. Likewise, the second subunit comprising the HVR2 region can be positioned as either the N-terminal or C-terminal subunit.

FIG. 4 provides a schematic of a reporter assay in CHO cells for evaluating engineered meganucleases targeting recognition sequences found in the AAT gene. For the engineered meganucleases described herein, a CHO cell line was produced in which a reporter cassette was integrated stably into the genome of the cell. The reporter cassette comprised, in 5′ to 3′ order: an SV40 Early Promoter; the 5′ 2/3 of the GFP gene; the recognition sequence for an engineered meganuclease described herein (e.g., the AAT 35-36 sequence; SEQ ID NO: 26); the recognition sequence for the CHO-23/24 meganuclease (WO/2012/167192); and the 3′ 2/3 of the GFP gene. Cells stably transfected with this cassette did not express GFP in the absence of a DNA break-inducing agent. Meganucleases were introduced by transduction of an mRNA encoding each meganuclease. When a DNA break was induced at either of the meganuclease recognition sequences, the duplicated regions of the GFP gene recombined with one another to produce a functional GFP gene. The percentage of GFP-expressing cells could then be determined by flow cytometry as an indirect measure of the frequency of genome cleavage by the meganucleases.

FIGS. 5A-5E provide data showing the efficiency of engineered AAT meganucleases described herein for recognizing and cleaving recognition sequences in a CHO cell reporter assay. The activity index represents % GFP positive cells for each cell line expressing the test meganucleases normalized to the cell line expressing the CHO-23/24 meganuclease accounting for the toxicity of the meganuclease. FIG. 5A shows results of the indicated engineered meganucleases for cleaving the AAT 33-34 recognition sequence. FIG. 5B shows results of the indicated engineered meganucleases for cleaving the AAT 35-36 recognition sequence. FIG. 5C shows results of the indicated engineered meganucleases for cleaving the AAT 37-38 recognition sequence. FIG. 5D shows results of the indicated engineered meganucleases for cleaving the AAT 41-42 recognition sequence. FIG. 5E shows results of the indicated engineered meganucleases for cleaving the AAT 43-44 recognition sequence.

FIGS. 6A-6K provide data showing the efficiency of engineered AAT meganucleases described herein for recognizing and cleaving recognition sequences in a CHO cell reporter assay indicated as % GFP positive cells. FIG. 6A-FIG. 6C shows results of the indicated engineered meganucleases for cleaving the AAT 35-36 recognition sequence. FIG. 6D and FIG. 6E shows results of the indicated engineered meganucleases for cleaving the AAT 37-38 recognition sequence. FIGS. 6F-FIG. 6H shows results of the indicated engineered meganucleases for cleaving the AAT 41-42 recognition sequence. FIG. 6I-FIG. 6K shows results of the indicated engineered meganucleases for cleaving the AAT 43-44 recognition sequence.

FIG. 7 provides a bar graph indicating the percentage (%) of insertions and deletions (indels) for each of the indicated engineered meganucleases in Hep3B cells at Day 2 (black bars) and Day 6 (gray bars) post transfection. A total of 50 ng or 5 ng of each meganuclease was transfected.

FIG. 8 provides a schematic of the gene editing approach used in the PiZ AAT murine model of Example 3. As shown, the AAT gene comprises seven exons (exons 1a, 1b, 1c and exons 2, 3, 4, and 5). The gene contains the ‘Z’ mutation in exon 5. The strategy used in this mouse model utilizes an engineered nuclease that cleaves an AAT recognition sequence in the intron between exon 3 and 4 (AAT 9-10). The repair WT AAT sequence shown in the middle of the figure contains exons 4 and 5, a stop codon, and poly A signal preventing the endogenous Z-AAT allele from being expressed. In addition, a flag tag was added for histological detection of WT AAT expressed off of the repair construct. The schematic at the bottom of the figure shows the edited and repaired AAT gene containing the exogenously inserted WT exons 4 and 5 with the stop codon and poly A signal to terminate translation and transcription of the allele prior to expression of the Z-AAT portion of the endogenous exon 5.

FIG. 9 provides schematics of AAT donor polynucleotide constructs used in the PiZ AAT murine model of Example 3.

FIG. 10A provides a bar graph showing the number of hAAT transgene copies inserted for each group of mice corresponding to the PiZ AAT murine model of Example 3. FIG. 10B provides a bar graph showing the percentage (%) of diploid mouse genomes with gene insertion for each group of mice corresponding to the PiZ AAT murine model of Example 3. FIG. 10C provides a bar graph showing the percentage (%) of hAAT alleles with gene insertion for each group of mice corresponding to the PiZ AAT murine model of Example 3. FIG. 10D provides a bar graph showing the percentage (%) of indels for each group of mice corresponding to the PiZ AAT murine model of Example 3.

FIG. 11A-FIG. 11D provide line graphs showing ratios of mutant Z-AAT to total AAT until day 40 of the study of Example 3 for study groups 1, 2, 3, and 4, respectively (solid symbols represent animals treated with a nuclease and hollow symbols represent animals treated with mock PBS).

FIG. 12 provides a line graph showing the total amount of WT AAT in μM secreted into the plasma of tested animals of the study of Example 3 for groups 1, 2, 3, and 4.

FIG. 13 provides a gene schematic of the hAAT gene indicating approximately where in the genome each of the engineered meganuclease recognition sequences are located for engineered meganucleases being tested in the study of Example 4.

FIG. 14 provides schematics for two AAT donor polynucleotide constructs that will be used in the in the PiZ AAT murine model of Example 4.

FIG. 15A and FIG. 15B provide bar graphs indicating the total human AAT Tg copy number in the blood at week 1 and week 6 and in the blood and liver at week 6 of PiZ mice treated with the indicated meganucleases, respectively.

FIG. 16A and FIG. 16B provide bar graphs showing the percentage of insertions at each recognition sequence after treatment with the AAT 33-34x.13, AAT 35-36x.70, AAT 37-38x.50, and AAT 43-44x.58 meganucleases in either a self-complementary (sc) AAV vector or single stranded (ss) AAV vector. FIG. 16A provides the percentage of productive insertions (transgene inserted in the correct orientation). FIG. 16B provides the total insertion for each site and the mechanism of action for that insertion as being homology directed repair (HDR) or non-homologous end joining (NHEJ).

FIG. 17. Provides a bar graph quantifying the percentage of PAS-D positive cells in the PiZ mouse liver of the indicated treatment groups of Example 4.

FIG. 18. Provides representative a representative histological image of PAS-D stained liver sections from PiZ mouse from Example 4.

FIG. 19A and FIG. 19B. Provide graphs indicating the amount of WT AAT protein in PiZ mice treated with the AAT 33-34x.13, AAT 35-36x.70, AAT 37-38x.50, and AAT 43-44x.58 meganucleases in either an SC AAV or SS AAV from the study of Example 4.

FIG. 20. Provides a bar graph indicating the amount of Flag tagged AAT detected in the liver of PiZ mice from the indicated treatment groups of Example 4.

FIG. 21. Provides a graph indicating the body weight in grams of PiZ mice from the indicated treatment groups corresponding to Example 5.

FIG. 22. Provides a bar graph showing the percentage of insertions of AAT (indicated as SERPINA1) template after 1 and 2 doses for each of the indicated treatment groups of Example 5.

FIG. 23. Provides a bar graph showing the copy number of AAT (indicated as SERPINA1) after 1 and 2 doses for each of the indicated treatment groups of Example 5.

FIG. 24. Provides a graph of the amount of WT AAT after one and two doses of the indicated treatment in the treatment groups of Example 5.

FIG. 25. Provides a schematic of the oligocapture assay utilized to determine off-target effects of an engineered nuclease (e.g., an engineered meganuclease described herein). As shown, the integration cassette or oligo anneals with a double stranded break in the genome that may be due to engineered nuclease cleavage. The DNA is then sheared by sonication, adapters are ligated and PCR amplified followed by sequence analysis to determine location of the double strand break.

FIG. 26A-FIG. 26H. Provides a bar graph indicating the percentage (%) of insertions and deletions (indels) for each of the indicated engineered meganucleases in Hep3B cells at Day 2 (dark gray bars) and Day 6 (light gray bars) post transfection, respectively.

FIG. 27A and FIG. 27B. Provide bar graphs indicating the percentage of insertions for each of the indicated AAT 35-36 meganucleases,

FIG. 28. Provides a graph depicting results from an oligo capture assay to identify off-target cutting induced by the indicated AAT 35-36 meganucleases transfected in HEK 293 cells. The circled dots indicate the on-target site and the non-circled dots indicate off-target sites with the X axis representing the number of sequencing reads for each detected off-target site. The shade of the dot indicates the number of base-pair mismatches between the on target site and each of the detected off-target sites.

FIG. 29A and FIG. 29B. Provide bar graphs indicating the percentage of insertions for each of the indicated AAT 37-38 meganucleases.

FIG. 30. Provides a graph depicting results from an oligo capture assay to identify off-target cutting induced by the indicated AAT 37-38 meganucleases transfected in HEK 293 cells. The circled dots indicate the on-target site and the non-circled dots indicate off-target sites with the X axis representing the number of sequencing reads for each detected off-target site. The shade of the dot indicates the number of base-pair mismatches between the on target site and each of the detected off-target sites.

FIG. 31A and FIG. 31B. Provide bar graphs indicating the percentage of insertions for each of the indicated AAT 41-42 meganucleases.

FIG. 32. Provides a graph depicting results from an oligo capture assay to identify off-target cutting induced by the indicated AAT 41-42 meganucleases transfected in HEK 293 cells. The circled dots indicate the on-target site and the non-circled dots indicate off-target sites with the X axis representing the number of sequencing reads for each detected off-target site. The shade of the dot indicates the number of base-pair mismatches between the on target site and each of the detected off-target sites.

FIG. 33A-FIG. 33C. Provide bar graphs indicating the percentage of insertions for each of the indicated AAT 43-44 meganucleases.

FIG. 34. Provides a graph depicting results from an oligo capture assay to identify off-target cutting induced by the indicated AAT 43-44 meganucleases transfected in HEK 293 cells. The circled dots indicate the on-target site and the non-circled dots indicate off-target sites with the X axis representing the number of sequencing reads for each detected off-target site. The shade of the dot indicates the number of base-pair mismatches between the on target site and each of the detected off-target sites.

FIG. 35. Provides a bar graph showing the copy number/diploid cell of AAT (indicated as hSERPINA1) after administration of PBS, an AAV8 containing repair construct, or the AAV8 repair construct and the indicated AAT 37-38 meganucleases. Asterisks indicate statistically significant difference between the treatment groups.

FIG. 36. Provides a bar graph showing the copy number/diploid cell of AAT (indicated as hSERPINA1) after administration of PBS, an AAV8 containing repair construct, or the AAV8 repair construct and the indicated AAT 41-42 meganucleases (ns indicates no statistical significance).

FIG. 37. Provides a bar graph showing the copy number/diploid cell of AAT (indicated as hSERPINA1) after administration of PBS, an AAV8 containing repair construct, or the AAV8 repair construct and the indicated AAT 43-44 meganucleases. Asterisks indicate a statistically significant difference between the treatment groups.

FIG. 38. Provides a bar graph showing percentage of repair construct insertion after administration of PBS, an AAV8 containing repair construct, or the AAV8 repair construct and the indicated AAT 37-38 meganucleases.

FIG. 39. Provides a bar graph showing percentage of repair construct insertion after administration of PBS, an AAV8 containing repair construct, or the AAV8 repair construct and the indicated AAT 41-42 meganucleases.

FIG. 40. Provides a bar graph showing percentage of repair construct insertion after administration of PBS, an AAV8 containing repair construct, or the AAV8 repair construct and the indicated AAT 43-44 meganucleases.

FIG. 41. Provides a graph showing concentration of WT AAT protein (μg/mL) in the plasma from PiZ mice after administration of PBS, an AAV8 containing repair construct, or the AAV8 repair construct and the indicated AAT 37-38 meganucleases at 7, 9, 10, 11, and 12 weeks.

FIG. 42. Provides a graph showing concentration of WT AAT protein (μg/mL) in the plasma from PiZ mice after administration of PBS, an AAV8 containing repair construct, or the AAV8 repair construct and the indicated AAT 41-42 meganucleases at 7, 9, 10, 11, and 12 weeks.

FIG. 43. Provides a graph showing concentration of WT AAT protein (μg/mL) in the plasma from PiZ mice after administration of PBS, an AAV8 containing repair construct, or the AAV8 repair construct and the indicated AAT 43-44 meganucleases at 7, 9, 10, 11, and 12 weeks.

FIG. 44. Provides a bar graph showing the percentage of PAS-D positive staining in the livers of PiZ mice after administration of PBS, an AAV8 containing repair construct, or the AAV8 repair construct and the indicated AAT 37-38, AAT 41-42, and AAT 43-44 meganucleases.

FIG. 45. Provides a bar graph showing the percentage of Flag positive staining for detection of Flag tagged WT-AAT insert in the livers of PiZ mice after administration of PBS, an AAV8 containing repair construct, or the AAV8 repair construct and the indicated AAT 37-38, AAT 41-42, and AAT 43-44 meganucleases.

FIG. 46. Provides a bar graph showing the detection of WT AAT RNA transcript in the livers of PiZ mice after administration of PBS, an AAV8 containing repair construct, or the AAV8 repair construct and the indicated AAT 37-38, AAT 41-42, and AAT 43-44 meganucleases.

FIG. 47. Provides a bar graph showing the detection of AAT Z RNA transcript in the livers of PiZ mice after administration of PBS, an AAV8 containing repair construct, or the AAV8 repair construct and the indicated AAT 37-38, AAT 41-42, and AAT 43-44 meganucleases.

FIG. 48. Provides graphs showing the levels of liver enzymes ALT, AST, ALP, and TBIL for PiZ mice treated with PBS, an AAV8 repair template alone (3e13 vg/kg), or an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 49. Provides a graph showing the body weight of PiZ mice treated with PBS, an AAV8 repair template alone (3e13 vg/kg), or an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 50. Provides a bar graph showing the percentage of productive insertions (insert oriented in the functional orientation) per hAAT from the livers of PiZ mice treated with an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 51. Provides a bar graph showing the percentage of indels at the AAT 37-38 recognition sequence from the livers of PiZ mice treated with an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 52. Provides a graph showing the WT AAT protein concentration in μg/mL from the livers of PiZ mice at two weeks, three weeks, and four weeks after treatment with an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 53. Provides a graph showing the Z AAT protein concentration in μg/mL from the livers of PiZ mice at two weeks, three weeks, and four weeks after treatment with an AAV8 repair template (3e13 vg/kg) and an LNP-A containing the AAT 37-38L.262 meganuclease at 0.5 mg/kg, 0.3 mg/kg, 0.1 mg/kg, or 0.05 mg/kg.

FIG. 54. Provides graphs showing the levels of liver enzymes ALT, AST, ALP, and TBIL from the livers of rats treated with an LNP-A containing the AAT 37-38L.262 meganuclease at 3.5 mg/kg, 3 mg/kg, and 1 mg/kg.

FIG. 55. Provides graphs showing the concentration of various cytokines (pg/mL) IFN-y, TNF-a, IL-6, IL-1β, IL-10 and KC/GRO from the livers of rats treated with an LNP-A containing the AAT 37-38L.262 meganuclease at 3.5 mg/kg, 3 mg/kg, and 1 mg/kg.

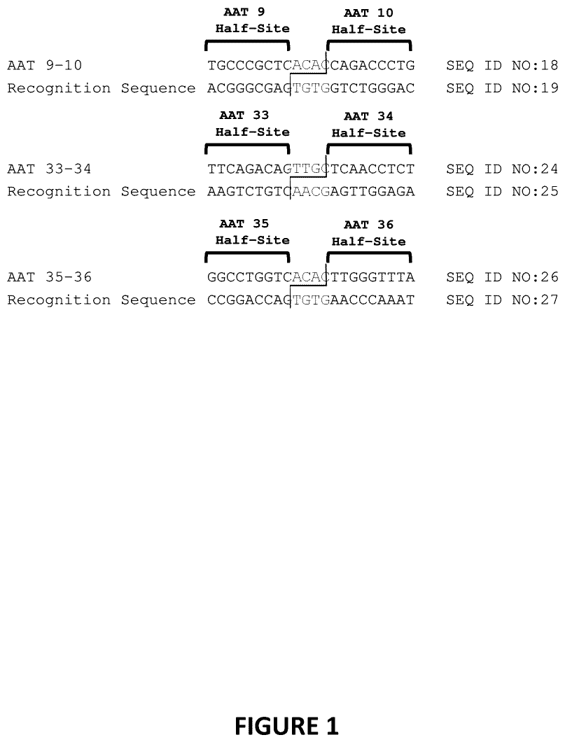

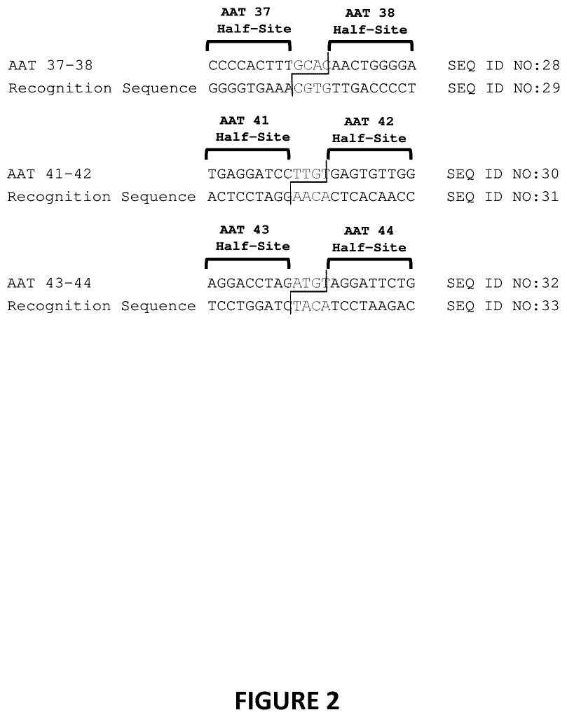

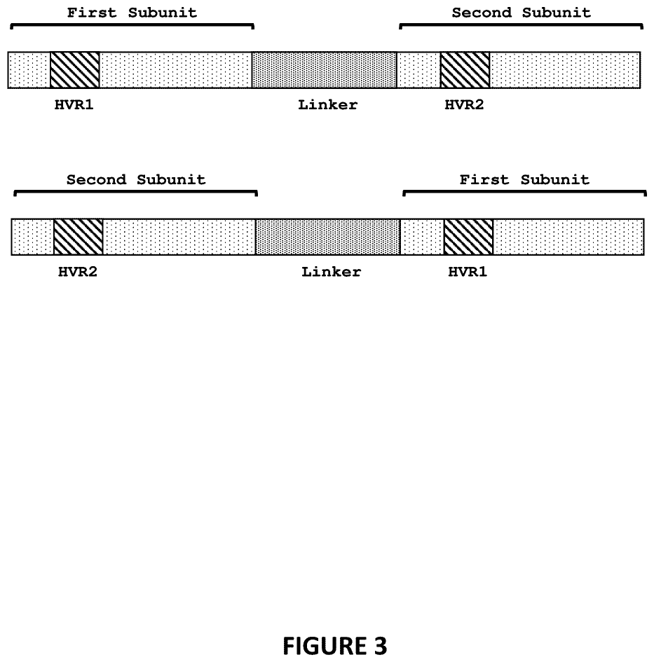

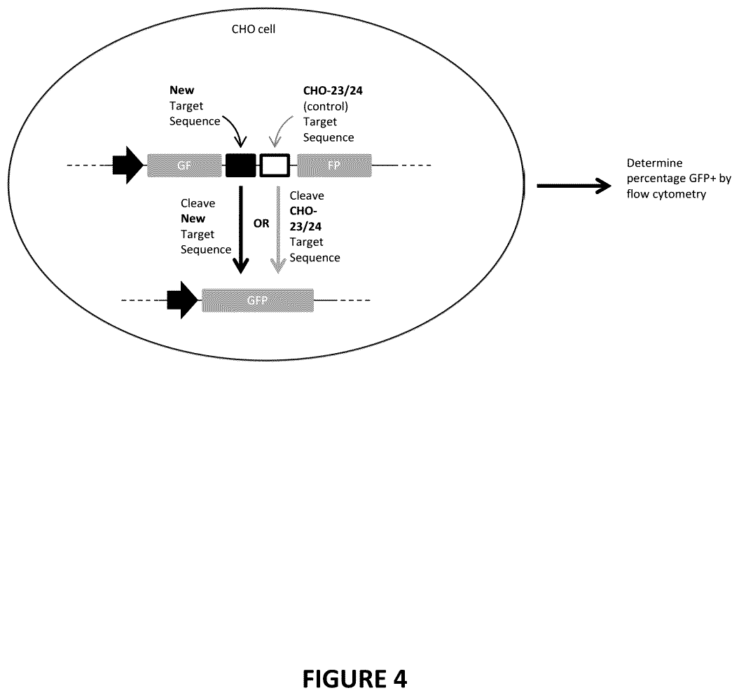

### BRIEF DESCRIPTION OF THE SEQUENCES

SEQ ID NO: 1 sets forth the amino acid sequence of a wild-type I-CreI meganuclease.

SEQ ID NO: 2 sets forth the amino acid sequence of a LAGLIDADG motif.

SEQ ID NO: 3 sets forth the nucleic acid sequence of a donor nucleic acid sequence comprising wild-type SERPINA1 exons 1b, 1c, 2, 3, 4, and 5, excluding introns 1b, 1c, 2, 3, and 4.

SEQ ID NO: 4 sets forth the nucleic acid sequence of a donor nucleic acid sequence comprising wild-type SERPINA1 exons 1b, 1c, 2, 3, 4, and 5, including introns 1b, 1c, 2, 3, and 4.

SEQ ID NO: 5 sets forth the nucleic acid sequence of a donor nucleic acid sequence comprising wild-type SERPINA1 exons 1c, 2, 3, 4, and 5, excluding introns 1c, 2, 3, and 4.

SEQ ID NO: 6 sets forth the nucleic acid sequence of a donor nucleic acid sequence comprising wild-type SERPINA1 exons 1c, 2, 3, 4, and 5, including introns 1c, 2, 3, and 4.

SEQ ID NO: 7 sets forth the nucleic acid sequence of a donor nucleic acid sequence comprising wild-type SERPINA1 exons 2, 3, 4, and 5, excluding introns 2, 3, and 4.

SEQ ID NO: 8 sets forth the nucleic acid sequence of a donor nucleic acid sequence comprising wild-type SERPINA1 exons 2, 3, 4, and 5, including introns 2, 3, and 4.

SEQ ID NO: 9 sets forth the nucleic acid sequence of a donor nucleic acid sequence comprising wild-type SERPINA1 exons 3, 4, and 5, excluding introns 3 and 4.

SEQ ID NO: 10 sets forth the nucleic acid sequence of a donor nucleic acid sequence comprising wild-type SERPINA1 exons 3, 4, and 5, including introns 3 and 4.

SEQ ID NO: 11 sets forth the nucleic acid sequence of a donor nucleic acid sequence comprising wild-type SERPINA1 exons 4 and 5, excluding intron 4.

SEQ ID NO: 12 sets forth the nucleic acid sequence of a donor nucleic acid sequence comprising wild-type SERPINA1 exons 4 and 5, including intron 4.

SEQ ID NO: 13 sets forth the nucleic acid sequence of a donor nucleic acid sequence comprising wild-type SERPINA1 exon 5.

SEQ ID NO: 14 sets forth the amino acid sequence of a wild-type AAT protein.

SEQ ID NO: 15 sets forth the nucleic acid sequence of a modified SERPINA1 gene that includes wild-type exon 1a, intron 1a, exon 1b, intron 1b, exon 1c, intron 1c, exon 2, intron 2, exon 3, intron 3, exon 4, intron 4, and exon 5.

SEQ ID NO: 16 sets forth the nucleic acid sequence of a modified SERPINA1 gene that includes exon 1a, intron 1a, exon 1b, intron 1b, exon 1c, intron 1c, and exons 2-5 without introns 2, 3, or 4.

SEQ ID NO: 17 sets forth the amino acid sequence of an SV40 nuclear localization sequence.

SEQ ID NO: 18 sets forth the nucleic acid sequence of an AAT 9-10 recognition sequence (sense).

SEQ ID NO: 19 sets forth the nucleic acid sequence of an AAT 9-10 recognition sequence (antisense).

SEQ ID NO: 20 sets forth the nucleic acid sequence of an AAT 13-14 recognition sequence (sense).

SEQ ID NO: 21 sets forth the nucleic acid sequence of an AAT 13-14 recognition sequence (antisense).

SEQ ID NO: 22 sets forth the nucleic acid sequence of an AAT 31-32 recognition sequence (sense).

SEQ ID NO: 23 sets forth the nucleic acid sequence of an AAT 31-32 recognition sequence (antisense).

SEQ ID NO: 24 sets forth the nucleic acid sequence of an AAT 33-34 recognition sequence (sense).

SEQ ID NO: 25 sets forth the nucleic acid sequence of an AAT 33-34 recognition sequence (antisense).

SEQ ID NO: 26 sets forth the nucleic acid sequence of an AAT 35-36 recognition sequence (sense).

SEQ ID NO: 27 sets forth the nucleic acid sequence of an AAT 35-36 recognition sequence (antisense).

SEQ ID NO: 28 sets forth the nucleic acid sequence of an AAT 37-38 recognition sequence (sense).

SEQ ID NO: 29 sets forth the nucleic acid sequence of an AAT 37-38 recognition sequence (antisense).

SEQ ID NO: 30 sets forth the nucleic acid sequence of an AAT 41-42 recognition sequence (sense).

SEQ ID NO: 31 sets forth the nucleic acid sequence of an AAT 41-42 recognition sequence (antisense).

SEQ ID NO: 32 sets forth the nucleic acid sequence of an AAT 43-44 recognition sequence (sense).

SEQ ID NO: 33 sets forth the nucleic acid sequence of an AAT 43-44 recognition sequence (antisense).

SEQ ID NO: 34 sets forth the nucleic acid sequence of a TTR 5-6 recognition sequence (sense).

SEQ ID NO: 35 sets forth the nucleic acid sequence of a TTR 5-6 recognition sequence (antisense).

SEQ ID NO: 36 sets forth the nucleic acid sequence of an AAT 31-32 Fwd primer.

SEQ ID NO: 37 sets forth the nucleic acid sequence of an AAT 31-32 Fwd primer.

SEQ ID NO: 38 sets forth the nucleic acid sequence of an AAT 31-32 Fwd primer.

SEQ ID NO: 39 sets forth the nucleic acid sequence of an AAT 31-32 Fwd primer.

SEQ ID NO: 40 sets forth the nucleic acid sequence of an AAT 31-32 Rvs primer.

SEQ ID NO: 41 sets forth the nucleic acid sequence of an AAT 31-32 Rvs primer.

SEQ ID NO: 42 sets forth the nucleic acid sequence of an AAT 31-32 Rvs primer.

SEQ ID NO: 43 sets forth the nucleic acid sequence of an AAT 31-32 Rvs primer.

SEQ ID NO: 44 sets forth the nucleic acid sequence of an AAT 31-32 Rvs primer.

SEQ ID NO: 45 sets forth the nucleic acid sequence of an AAT 31-32 Rvs primer.

SEQ ID NO: 46 sets forth the nucleic acid sequence of an AAT 31-32 Rvs primer.

SEQ ID NO: 47 sets forth the nucleic acid sequence of an AAT 31-32 Rvs primer.

SEQ ID NO: 48 sets forth the nucleic acid sequence of an AAT 33-34 Fwd primer.

SEQ ID NO: 49 sets forth the nucleic acid sequence of an AAT 33-34 Fwd primer.

SEQ ID NO: 50 sets forth the nucleic acid sequence of an AAT 33-34 Fwd primer.

SEQ ID NO: 51 sets forth the nucleic acid sequence of an AAT 33-34 Fwd primer.

SEQ ID NO: 52 sets forth the nucleic acid sequence of an AAT 33-34 Rvs primer.

SEQ ID NO: 53 sets forth the nucleic acid sequence of an AAT 33-34 Rvs primer.

SEQ ID NO: 54 sets forth the nucleic acid sequence of an AAT 33-34 Rvs primer.

SEQ ID NO: 55 sets forth the nucleic acid sequence of an AAT 33-34 Rvs primer.

SEQ ID NO: 56 sets forth the nucleic acid sequence of an AAT 33-34 Rvs primer.

SEQ ID NO: 57 sets forth the nucleic acid sequence of an AAT 33-34 Rvs primer.

SEQ ID NO: 58 sets forth the nucleic acid sequence of an AAT 33-34 Rvs primer.

SEQ ID NO: 59 sets forth the nucleic acid sequence of an AAT 33-34 Rvs primer.

SEQ ID NO: 60 sets forth the nucleic acid sequence of an AAT 35-36 Fwd primer.

SEQ ID NO: 61 sets forth the nucleic acid sequence of an AAT 35-36 Fwd primer.

SEQ ID NO: 62 sets forth the nucleic acid sequence of an AAT 35-36 Fwd primer.

SEQ ID NO: 63 sets forth the nucleic acid sequence of an AAT 35-36 Fwd primer.

SEQ ID NO: 64 sets forth the nucleic acid sequence of an AAT 35-36 Rvs primer.

SEQ ID NO: 65 sets forth the nucleic acid sequence of an AAT 35-36 Rvs primer.

SEQ ID NO: 66 sets forth the nucleic acid sequence of an AAT 35-36 Rvs primer.

SEQ ID NO: 67 sets forth the nucleic acid sequence of an AAT 35-36 Rvs primer.

SEQ ID NO: 68 sets forth the nucleic acid sequence of an AAT 35-36 Rvs primer.

SEQ ID NO: 69 sets forth the nucleic acid sequence of an AAT 35-36 Rvs primer.

SEQ ID NO: 70 sets forth the nucleic acid sequence of an AAT 35-36 Rvs primer.

SEQ ID NO: 71 sets forth the nucleic acid sequence of an AAT 35-36 Rvs primer.

SEQ ID NO: 72 sets forth the nucleic acid sequence of an AAT 37-38 Fwd primer.

SEQ ID NO: 73 sets forth the nucleic acid sequence of an AAT 37-38 Fwd primer.

SEQ ID NO: 74 sets forth the nucleic acid sequence of an AAT 37-38 Fwd primer.

SEQ ID NO: 75 sets forth the nucleic acid sequence of an AAT 37-38 Fwd primer.

SEQ ID NO: 76 sets forth the nucleic acid sequence of an AAT 37-38 Fwd primer.

SEQ ID NO: 77 sets forth the nucleic acid sequence of an AAT 37-38 Fwd primer.

SEQ ID NO: 78 sets forth the nucleic acid sequence of an AAT 37-38 Fwd primer.

SEQ ID NO: 79 sets forth the nucleic acid sequence of an AAT 37-38 Fwd primer.

SEQ ID NO: 80 sets forth the nucleic acid sequence of an AAT 37-38 Fwd primer.

SEQ ID NO: 81 sets forth the nucleic acid sequence of an AAT 37-38 Rvs primer.

SEQ ID NO: 82 sets forth the nucleic acid sequence of an AAT 37-38 Rvs primer.

SEQ ID NO: 83 sets forth the nucleic acid sequence of an AAT 37-38 Rvs primer.

SEQ ID NO: 84 sets forth the nucleic acid sequence of an AAT 37-38 Rvs primer.

SEQ ID NO: 85 sets forth the nucleic acid sequence of an AAT 37-38 Rvs primer.

SEQ ID NO: 86 sets forth the nucleic acid sequence of an AAT 37-38 Rvs primer.

SEQ ID NO: 87 sets forth the nucleic acid sequence of an AAT 37-38 Rvs primer.

SEQ ID NO: 88 sets forth the nucleic acid sequence of an AAT 37-38 Rvs primer.

SEQ ID NO: 89 sets forth the nucleic acid sequence of an AAT 41-42 Fwd primer.

SEQ ID NO: 90 sets forth the nucleic acid sequence of an AAT 41-42 Rvs primer.

SEQ ID NO: 91 sets forth the nucleic acid sequence of an AAT 41-42 Rvs primer.

SEQ ID NO: 92 sets forth the nucleic acid sequence of an AAT 41-42 Rvs primer.

SEQ ID NO: 93 sets forth the nucleic acid sequence of an AAT 41-42 Rvs primer.

SEQ ID NO: 94 sets forth the nucleic acid sequence of an AAT 41-42 Rvs primer.

SEQ ID NO: 95 sets forth the nucleic acid sequence of an AAT 41-42 Rvs primer.

SEQ ID NO: 96 sets forth the nucleic acid sequence of an AAT 41-42 Rvs primer.

SEQ ID NO: 97 sets forth the nucleic acid sequence of an AAT 41-42 Rvs primer.

SEQ ID NO: 98 sets forth the nucleic acid sequence of an AAT 43-44 Fwd primer.

SEQ ID NO: 99 sets forth the nucleic acid sequence of an AAT 43-44 Fwd primer.

SEQ ID NO: 100 sets forth the nucleic acid sequence of an AAT 43-44 Fwd primer.

SEQ ID NO: 101 sets forth the nucleic acid sequence of an AAT 43-44 Fwd primer.

SEQ ID NO: 102 sets forth the nucleic acid sequence of an AAT 43-44 Rvs primer.

SEQ ID NO: 103 sets forth the nucleic acid sequence of an AAT 43-44 Rvs primer.

SEQ ID NO: 104 sets forth the nucleic acid sequence of an AAT 43-44 Rvs primer.

SEQ ID NO: 105 sets forth the nucleic acid sequence of an AAT 43-44 Rvs primer.

SEQ ID NO: 106 sets forth the nucleic acid sequence of an AAT 43-44 Rvs primer.

SEQ ID NO: 107 sets forth the nucleic acid sequence of an AAT 43-44 Rvs primer.

SEQ ID NO: 108 sets forth the nucleic acid sequence of an AAT 43-44 Rvs primer.

SEQ ID NO: 109 sets forth the nucleic acid sequence of an AAT 43-44 Rvs primer.

SEQ ID NO: 110 sets forth the nucleic acid sequence of a P1 primer.

SEQ ID NO: 111 sets forth the nucleic acid sequence of a F1 primer.

SEQ ID NO: 112 sets forth the nucleic acid sequence of a R1 primer.

SEQ ID NO: 113 sets forth the nucleic acid sequence of a P2 primer.

SEQ ID NO: 114 sets forth the nucleic acid sequence of a F2 primer.

SEQ ID NO: 115 sets forth the nucleic acid sequence of a R2 primer.

SEQ ID NO: 116 sets forth the nucleic acid sequence of a P3 primer.

SEQ ID NO: 117 sets forth the nucleic acid sequence of a F3 primer.

SEQ ID NO: 118 sets forth the nucleic acid sequence of a R3 primer.

SEQ ID NO: 119 sets forth the nucleic acid sequence of a P4 primer.

SEQ ID NO: 120 sets forth the nucleic acid sequence of a F4 primer.

SEQ ID NO: 121 sets forth the nucleic acid sequence of a R4 primer.

SEQ ID NO: 122 sets forth the nucleic acid sequence of an AAT33-34 Fwd primer.

SEQ ID NO: 123 sets forth the nucleic acid sequence of an AAT33-34 Rvs primer.

SEQ ID NO: 124 sets forth the nucleic acid sequence of an AAT33-34 Probe.

SEQ ID NO: 125 sets forth the nucleic acid sequence of an AAT35-36 Fwd primer.

SEQ ID NO: 126 sets forth the nucleic acid sequence of an AAT35-36 Rvs primer.

SEQ ID NO: 127 sets forth the nucleic acid sequence of an AAT35-36 Probe.

SEQ ID NO: 128 sets forth the nucleic acid sequence of an AAT37-38 Fwd primer.

SEQ ID NO: 129 sets forth the nucleic acid sequence of an AAT37-38 Rvs primer.

SEQ ID NO: 130 sets forth the nucleic acid sequence of an AAT37-38 Probe.

SEQ ID NO: 131 sets forth the nucleic acid sequence of an AAT41-42 Fwd primer.

SEQ ID NO: 132 sets forth the nucleic acid sequence of an AAT41-42 Rvs primer.

SEQ ID NO: 133 sets forth the nucleic acid sequence of an AAT41-42 Probe.

SEQ ID NO: 134 sets forth the nucleic acid sequence of an AAT43-44 Fwd primer.

SEQ ID NO: 135 sets forth the nucleic acid sequence of an AAT43-44 Rvs primer.

SEQ ID NO: 136 sets forth the nucleic acid sequence of an AAT43-44 Probe.

SEQ ID NO: 137 sets forth the nucleic acid sequence of an AAT33-34 Fwd primer.

SEQ ID NO: 138 sets forth the nucleic acid sequence of an AAT33-34 Rvs primer.

SEQ ID NO: 139 sets forth the nucleic acid sequence of an AAT33-34 Probe.

SEQ ID NO: 140 sets forth the nucleic acid sequence of an AAT35-36 Fwd primer.

SEQ ID NO: 141 sets forth the nucleic acid sequence of an AAT35-36 Rvs primer.

SEQ ID NO: 142 sets forth the nucleic acid sequence of an AAT35-36 Probe.

SEQ ID NO: 143 sets forth the nucleic acid sequence of an AAT37-38 Fwd primer.

SEQ ID NO: 144 sets forth the nucleic acid sequence of an AAT37-38 Rvs primer.

SEQ ID NO: 145 sets forth the nucleic acid sequence of an AAT37-38 Probe.

SEQ ID NO: 146 sets forth the nucleic acid sequence of an AAT41-42 Fwd primer.

SEQ ID NO: 147 sets forth the nucleic acid sequence of an AAT41-42 Rvs primer.

SEQ ID NO: 148 sets forth the nucleic acid sequence of an AAT41-42 Probe.

SEQ ID NO: 149 sets forth the nucleic acid sequence of an AAT43-44 Fwd primer.

SEQ ID NO: 150 sets forth the nucleic acid sequence of an AAT43-44 Rvs primer.

SEQ ID NO: 151 sets forth the nucleic acid sequence of an AAT43-44 Probe.

SEQ ID NO: 152 sets forth the nucleic acid sequence of an AAT33-34 Fwd primer for the indel assay of Example 6.

SEQ ID NO: 153 sets forth the nucleic acid sequence of an AAT33-34 Rev primer for the indel assay of example 6.

SEQ ID NO: 154 sets forth the nucleic acid sequence of an AAT33-34 Probe for the indel assay of Example 6.

SEQ ID NO: 155 sets forth the nucleic acid sequence of an AAT35-36 Fwd primer for the indel assay of example 6.

SEQ ID NO: 156 sets forth the nucleic acid sequence of an AAT35-36 Rev primer for the indel assay of example 6.

SEQ ID NO: 157 sets forth the nucleic acid sequence of an AAT35-36 Probe for the indel assay of Example 6.

SEQ ID NO: 158 sets forth the nucleic acid sequence of an AAT37-38 Fwd primer for the indel assay of example 6.

SEQ ID NO: 159 sets forth the nucleic acid sequence of an AAT37-38 Rev primer for the indel assay of example 6.

SEQ ID NO: 160 sets forth the nucleic acid sequence of an AAT37-38 Probe for the indel assay of Example 6.

SEQ ID NO: 161 sets forth the nucleic acid sequence of an AAT41-42 Fwd primer for the indel assay of example 6.

SEQ ID NO: 162 sets forth the nucleic acid sequence of an AAT41-42 Rev primer for the indel assay of example 6.

SEQ ID NO: 163 sets forth the nucleic acid sequence of an AAT41-42 Probe for the indel assay of Example 6.

SEQ ID NO: 164 sets forth the nucleic acid sequence of an AAT43-44 Fwd primer for the indel assay of example 6.

SEQ ID NO: 165 sets forth the nucleic acid sequence of an AAT43-44 Rev primer for the indel assay of example 6.

SEQ ID NO: 166 sets forth the nucleic acid sequence of an AAT43-44 Probe for the indel assay of Example 6.

SEQ ID NO: 167 sets forth the nucleic acid sequence of an AAT Transcript fwd primer for the AAT transcript assay of Example 7.

SEQ ID NO: 168 sets forth the nucleic acid sequence of an AAT Transcript rev primer for the AAT transcript assay of Example 7.

SEQ ID NO: 169 sets forth the nucleic acid sequence of an WT AAT Transcript Probe for the AAT transcript assay of Example 7.

SEQ ID NO: 170 sets forth the nucleic acid sequence of an Z AAT Transcript Probe for the AAT transcript assay of Example 7.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the terms “nuclease” and “endonuclease” are used interchangeably to refer to naturally-occurring or engineered enzymes, which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. application No. 20130117869 (which is incorporated by reference in its entirety), including but not limited to MmeI, EndA, End1, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9 or Cas12a, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi: 10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, SI nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326 (5959): 1509-1512 and Moscou and Bogdanove (2009) Science 326 (5959): 1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi: 10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, SI nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue): W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered. Exemplary transfection techniques of the disclosure include, but are not limited to, electroporation and lipofection using Lipofectamine (e.g., Lipofectamine® MessengerMax (ThermoFisher)).

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term with respect to recombinant proteins, the term “modification” means any insertion, deletion, or substitution of an amino acid residue in the recombinant sequence relative to a reference sequence (e.g., a wild-type or a native sequence).

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template into a gene can result in no expression of an encoded protein, expression of a truncated protein, and/or expression of a protein that does not retain its wild-type function.

As used herein, the term “intron” refers to a nucleotide sequence within a gene that is removed from an RNA by RNA splicing prior to translation of the RNA. An intron in a DNA sequence refers to a nucleotide sequence that is transcribed during transcription and thus present in pre-mRNA, but removed from the pre-mRNA by splicing in the production of mature mRNA.

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a nonspecific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease. This term embraces chromosomal DNA duplexes as well as single-stranded chromosomal DNA.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “specificity” refers to the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art, such as unbiased identification of DSBs enabled by sequencing (GUIDE-seq), oligonucleotide (oligo) capture assay, whole genome sequencing, and long-range next generation sequencing of the recognition sequence. In some embodiments, specificity is measured using GUIDE-seq. As used herein, “specificity” is synonymous with a low incidence of cleavage of sequences different from the target sequences (non-target sequences), i.e., off-target cutting. A low incidence of off-target cutting may comprise an incidence of cleavage of non-target sequences of less than 25%, less than 20%, less than 18%, less than 15%, less than 12.5%, less than 10%, less than 9%, less than 8%, less than 7%, less than 6%, less than 5%, less than 4%, less than 3%, less than 2.5%, less than 2%, less than 1.5%, less than 1%, less than 0.75%, less than 0.5%, or less than 0.25%. Off-target cleavage by a nuclease can be measured using any method known in the art, including for example, oligo capture analysis as described here, a T7 endonuclease (T7E) assay as described herein, digital PCR as described herein, targeted sequencing of particular off-target sites, exome sequencing, whole genome sequencing, direct in situ breaks labeling enrichment on streptavidin and next-generation sequencing (BLESS), genome-wide, GUIDE-seq, and linear amplification-mediated high-throughput genome-wide translocation sequencing (LAM-HTGTS) (see, e.g., Zischewski et al. (2017), Biotechnology Advances 35 (1): 95-104, which is incorporated by reference in its entirety).

As used herein, a nuclease has “altered” specificity if it binds to and cleaves a recognition sequence which is not bound to and cleaved by a reference nuclease (e.g., a wild-type) under physiological conditions, or if the rate of cleavage of a recognition sequence is increased or decreased by a biologically significant amount (e.g., at least 2×, or 2×-10×) relative to a reference nuclease.

As used herein, the term “efficiency of cleavage” refers to the incidence by which a nuclease cleaves a recognition sequence in a double-stranded DNA molecule relative to the incidence of all cleavage events by the nuclease on the DNA molecule. “Efficiency of cleavage” is synonymous with DNA editing efficiency or on-target editing. Efficiency of cleavage and/or indel formation by a nuclease can be measured using any method known in the art, including T7E assay, digital PCR (ddPCR), mismatch detection assays, mismatch cleavage assay, high-resolution melting analysis (HRMA), heteroduplex mobility assay, sequencing, and fluorescent PCR capillary gel electrophoresis (see, e.g., Zischewski et al. (2017) Biotechnology Advances 35 (1): 95-104, which is incorporated by reference in its entirety). In some embodiments, efficiency of cleavage is measured by ddPCR. In some embodiments, the disclosed nucleases generate efficiencies of cleavage of at least about 35%, 40%, 50%, 60%, 70%, 80%, 85%, 90%, 95%, 98%, or 99% at the recognition sequence.

As used herein, “SERPINA1 gene” refers to a gene encoding a polypeptide having antitrypsin activity, or a variant thereof, particularly the AAT polypeptide, which is also referred to as the serpin peptidase inhibitor, member 1. A SERPINA1 gene can include a human SERPINA1 gene (NCBI Accession No.: NM_000295.5; Gene ID: 5265); cynomolgus monkey (Macaca fascicularis) SERPINA1 (NCBI Accession No.: XM_005562106.2); and mouse (Mus musculus) SERPINA1, (NM_009243.4). Additional examples of SERPINA1 mRNA sequences are readily available using publicly available databases, e.g., GenBank, UniProt, OMIM, and the Macaca genome project web site. The term SERPINA1 also refers to naturally occurring DNA sequence variations of the SERPINA1 gene, such as a single nucleotide polymorphism (SNP) in the SERPINA1 gene. Exemplary SNPs may be found through the publicly accessible National Center for Biotechnology Information dbSNP Short Genetic Variations database.

As used herein, the term “AAT polypeptide” refers to a polypeptide encoded by a SERPINA1 gene. The AAT polypeptide is also known as alpha-1-antitrypsin.

As used herein, the term “AAT deficiency” refers to an autosomal codominant disorder caused by a mutation in the SERPINA1 gene encoding AAT, a serine protease inhibitor, in which the mutation results in the expression of a mutant AAT protein with reduced ability to inhibit serine protease activity, and consequently results in increased serine protease activity.

An “indel”, as used herein, refers to the insertion or deletion of a nucleobase within a nucleic acid, such as DNA. In some embodiments, it is desirable to generate one or more insertions or deletions (i.e., indels) in the nucleic acid, e.g., in a foreign nucleic acid such as viral DNA. Accordingly, as used herein, “efficiency of indel formation” refers to the incidence by which a nuclease generates one or more indels through cleavage of a recognition sequence relative to the incidence of all cleavage events by the nuclease on the DNA molecule. In some embodiments, efficiency of indel formation is measured by ddPCR. In some embodiments, the disclosed nucleases generate efficiencies of indel formation of at least about 35%, 40%, 50%, 60%, 70%, 80%, 85%, 90%, 95%, 98%, or 99% at the recognition sequence. The disclosed nucleases may generate efficiencies of cleavage and/or efficiencies of indel formation of at least about 60%, 61%, 62%, 63%, 64%, 65%, 66%, 67%, 68%, 69%, 70%, 71%, 72%, 73%, 74%, or 75% at the recognition sequence.

As used herein, the term “homologous recombination” or “HR” refers to the natural, cellular process in which a double-stranded DNA-break is repaired using a homologous DNA sequence as the repair template (see, e.g., Cahill et al. (2006), Front. Biosci. 11:1958-1976). The homologous DNA sequence may be an endogenous chromosomal sequence or an exogenous nucleic acid that was delivered to the cell.

As used herein, a “template nucleic acid,” “donor nucleic acid,” “donor template,” or “donor polynucleotide” refers to a nucleic acid that is desired to be inserted into a cleavage site within a cell's genome. Such template nucleic acids or donor templates can comprise, for example, a transgene, such as an exogenous transgene, which encodes a protein of interest. The template nucleic acid or donor template can comprise 5′ and 3′ homology arms having homology to 5′ and 3′ sequences, respectively, that flank a cleavage site in the genome where insertion of the template is desired. Insertion can be accomplished, for example, by homology-directed repair (HDR).

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “homology arms” or “sequences homologous to sequences flanking a nuclease cleavage site” refer to sequences flanking the 5′ and 3′ ends of a nucleic acid molecule, which promote insertion of the nucleic acid molecule into a cleavage site generated by a nuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome. In some embodiments, the homology arms are about 500 base pairs.

As used herein, the term with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7 (1-2): 203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein, the term “corresponding to” with respect to modifications of two proteins or amino acid sequences is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “recognition half-site,” “recognition sequence half-site,” or simply “half-site” means a nucleic acid sequence in a double-stranded DNA molecule that is recognized and bound by a monomer of a homodimeric or heterodimeric meganuclease or by one subunit of a single-chain meganuclease or by one subunit of a single-chain meganuclease, or by a monomer of a TALEN or zinc finger nuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 of I-CreI (SEQ ID NO: 1). A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the disclosure, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of SEQ ID NO: 1.

As used herein, the term “reference level” in the context of AAT protein or mRNA levels refers to a level of AAT protein or mRNA as measured in, for example, a control cell, control cell population or a control subject, at a previous time point in the control cell, the control cell population or the subject undergoing treatment (e.g., a pre-dose baseline level obtained from the control cell, control cell population or subject), or a pre-defined threshold level of AAT protein or mRNA (e.g., a threshold level identified through previous experimentation).

As used herein, the term “a control” or “a control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype. A control subject may comprise, for example: a wild-type subject, i.e., of the same genotype as the starting subject for the genetic alteration which resulted in the genetically-modified subject (e.g., a subject having the same mutation in a SERPINA1 gene), which is not exposed to conditions or stimuli or further genetic modifications that would induce expression of altered genotype or phenotype in the subject.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “vector” or “recombinant DNA vector” may be a construct that includes a replication system and sequences that are capable of transcription and translation of a polypeptide-encoding sequence in a given host cell. If a vector is used then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the disclosure.

As used herein, a “vector” can also refer to a viral vector. Viral vectors can include, without limitation, retroviral vectors, lentiviral vectors, adenoviral vectors, and adeno-associated viral vectors (AAV).

As used herein, the term “operably linked” is intended to mean a functional linkage between two or more elements. For example, an operable linkage between a nucleic acid sequence encoding a nuclease as disclosed herein and a regulatory sequence (e.g., a promoter) is a functional link that allows for expression of the nucleic acid sequence encoding the nuclease. Operably linked elements may be contiguous or non-contiguous. When used to refer to the joining of two protein coding regions, by operably linked is intended that the coding regions are in the same reading frame.

As used herein, the terms “treatment” or “treating a subject” refers to the administration of an engineered nuclease described herein, or a polynucleotide encoding an engineered nuclease described herein, in combination with a donor polynucleotide comprising a template nucleic acid described herein, to a subject having AAT deficiency for the purpose of increasing levels of wild-type AAT in the blood of the subject. In some embodiments, expression of a full-length and/or functional version of the AAT protein results from cleavage by one or more of the disclosed nucleases, followed by insertion of the donor polynucleotide encoding functional AAT or a portion thereof into the SERPINA1 locus. In some embodiments, cleavage by one or more of the disclosed nucleases generates a nonsense mutation (e.g., introduction of a stop codon) upstream from the nucleic acid sequence encoding the non-functional portion of AAT, such that translation of the non-functional portion of AAT is prevented.

As used herein, the term “gc/kg” or “gene copies/kilogram” refers to the number of copies of a nucleic acid sequence encoding an engineered nuclease described herein, or the number of copies of a template nucleic acid described herein, per weight in kilograms of a subject that is administered a polynucleotide comprising the nucleic acid sequence or a polynucleotide comprising a template nucleic acid.

As used herein, the term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of an engineered nuclease described herein, or a polynucleotide encoding the same, or pharmaceutical compositions disclosed herein, in combination with a donor polynucleotide encoding functional AAT or a portion thereof, increases the level of expression of a functional AAT protein (e.g., a full-length AAT protein) and ameliorates at least one symptom associated with AAT deficiency.

As used herein, the term “lipid nanoparticle” refers to a lipid composition having a typically spherical structure with an average diameter between 10 and 1000 nanometers. In some formulations, lipid nanoparticles can comprise at least one cationic lipid, at least one non-cationic lipid, and at least one conjugated lipid. Lipid nanoparticles known in the art that are suitable for encapsulating nucleic acids, such as mRNA, are contemplated for use in the invention.

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

The present invention is based, in part, on the hypothesis that engineered nucleases can be designed to bind and cleave recognition sequences found within a SERPINA1 gene (e.g., the human SERPINA1 gene), and that a donor polynucleotide can provide a template nucleic acid encoding an AAT protein, or a portion thereof, that can be inserted into the cleavage site to generate a modified SERPINA1 gene that encodes a full-length, functional (e.g., wild-type) AAT protein that does not include any mutations found in the mutant endogenous gene. As a result, it is expected that trypsin protease activity in the liver and lungs will be reduced due to inhibition by the encoded functional AAT protein, relative to protease activity prior to editing by nuclease-mediated cleavage and insertion of the template nucleic acid. Effectiveness of treatment may be evaluated by measurement of lung and liver function inflammation, which may be measured by changes in levels of inflammatory cytokines, such as IL-1B and TNF-α, levels of fluid and swelling in the lungs, and signs of cirrhosis in the liver, all of which are characteristic of AAT deficiency.

Thus, the present invention encompasses engineered nucleases that bind and cleave a recognition sequence within the SERPINA1 gene. The present invention further provides methods comprising the delivery of an engineered protein, or nucleic acids encoding an engineered nuclease, to a eukaryotic cell in order to produce a genetically-modified eukaryotic cell. Further, the present invention provides pharmaceutical compositions, methods for treatment of AAT deficiency, and methods for increasing levels of wild-type AAT in the liver, blood, and lungs of a subject, which utilize an engineered nuclease having specificity for a recognition sequence positioned within the SERPINA1 gene, and a donor polynucleotide comprising a template nucleic acid encoding a functional AAT protein or portion thereof, such that nuclease-mediated cleavage and insertion of the template nucleic acid produces a SERPINA1 gene encoding a functional (e.g., wild-type) AAT protein.

### 2.2 Donor Polynucleotides and Template Nucleic Acids Encoding Functional AAT Proteins

In different aspects, the disclosure provides a polynucleotide, also referred to herein as a donor polynucleotide, that comprises a template nucleic acid encoding a functional AAT protein, or a portion thereof. Insertion of such a template nucleic acid into the SERPINA1 gene, such that RNA encoding a functional AAT protein is transcribed and translated, allows for the restoration of AAT activity in a cell or subject having one or more mutations that prevent expression of functional AAT from an endogenous SERPINA1 gene.

In some embodiments, the template nucleic acid comprises, in 5′ to 3′ order: (a) a splicing sequence comprising a splice acceptor sequence capable of pairing with an endogenous splice donor sequence in a SERPINA1 gene; (b) a donor nucleic acid sequence encoding an AAT protein or a portion thereof; and (c) a termination sequence.

In some embodiments, the polynucleotide comprising the template nucleic acid further comprises a 5′ homology arm and a 3′ homology arm flanking the template nucleic acid, wherein the 5′ homology arm and the 3′ homology arm share homology to sequences flanking a nuclease recognition sequence. Inclusion of the homology arms is meant to promote insertion of the template nucleic acid by HDR into the nuclease cleavage site.

Insertion of the template nucleic acid into the endogenous SERPINA1 gene produces a modified SERPINA1 gene. Transcription of this modified SERPINA1 gene produces an RNA with multiple exons separated by introns, which must be removed by splicing to produce messenger RNA (mRNA) containing an open reading frame encoding the AAT protein. Splicing occurs through interactions between a splice donor sequence at the 5′ end of an intron, and a splice acceptor sequence at the 3′ end of the intron. Pairing of the splice donor and splice acceptor results in the formation of a covalent bond between the nucleotide immediately 5′ to splice donor sequence (last nucleotide of a first exon) and the nucleotide immediately 3′ to the splice acceptor sequence (first nucleotide of a second exon), resulting in the excision of the intron from the RNA sequence, and joining of two exons into one nucleic acid sequence. Following transcription, the inserted splice acceptor sequence of (a) in the modified SERPINA1 gene allows the inserted AAT coding sequence of (b) to be joined to the upstream exon in the modified SERPINA1 gene, thereby forming an open reading frame encoding a functional AAT protein (e.g., a wild-type AAT protein). The termination sequence downstream of the AAT coding sequence causes translation to stop, preventing the addition of any additional amino acids to a polypeptide beyond those encoded by the AAT coding sequence of (b). Thus, the AAT protein expressed from the modified SERPINA1 gene does not comprise amino acids encoded by nucleic acid sequences downstream of the template nucleic acid in the modified SERPINA1 gene. Thus, the termination sequence prevents translation of mutant AAT proteins, even if such exons containing missense or frameshift mutations are incorporated into mRNA produced by transcription of the modified SERPINA1 gene.

In some embodiments of the template nucleic acid described herein, the termination sequence comprises a stop codon. In some embodiments, the stop codon comprises the nucleotide sequence TAG. In some embodiments, the stop codon comprises the nucleotide sequence TAA. In some embodiments, the stop codon comprises the nucleotide sequence TGA. In some embodiments, the termination sequence comprises a poly A sequence. In some embodiments, the termination sequence comprises a stop codon and a polyA sequence. Transcription of a DNA polynucleotide comprising a polyA sequence produces an RNA comprising a poly A tail. A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments of the donor polynucleotides and methods of using the donor polynucleotides provided herein, the polynucleotide comprises a 5′ homology arm and a 3′ homology arm flanking the template nucleic acid. In some embodiments, the donor polynucleotide and the template nucleic acid do not comprise a promoter (i.e., an exogenous promoter). Rather, the donor nucleic acid sequence encoding the AAT protein becomes operably linked to the endogenous SERPINA1 promoter after insertion into the nuclease cleavage site.

In some embodiments of the template nucleic acid described herein, the splicing sequence comprises a branch point. A branch point refers to a site in an intron that forms an intermediate structure during splicing. Typically, a branch point nucleotide forms a covalent bond with the first nucleotide of an intron via nucleophilic attack, forming a lariat intermediate. Then, the 3′ OH of the released first exon forms a covalent bond with the first nucleotide downstream from the intron, which is the first nucleotide of the following exon, resulting in the formation of a nucleic acid sequence containing the two exons. In some embodiments, the splicing sequence is a naturally-occurring splicing sequence. In some embodiments, the splicing sequence comprises an SV40 splicing sequence (e.g., intron), a CMV splicing sequence (e.g., intron), or a transferrin gene splicing sequence (e.g., intron). In some embodiments, the splicing sequence is a synthetic splicing sequence (e.g., intron).

In some embodiments, the AAT protein, or portion thereof encoded by the donor nucleic acid sequence is a wild-type AAT protein, or portion thereof. A wild-type AAT protein refers to an AAT protein comprising the amino acid sequence of the common form of human AAT, defined by UniProt Accession No. P01009.

In some embodiments, the donor nucleic acid sequence comprises one or more exons of a wild-type SERPINA1 gene. In some embodiments, the donor nucleic acid sequence comprises only wild-type exons of a SERPINA1 gene. In some embodiments, the donor nucleic acid sequence comprises one or more exons of a SERPINA1 gene that have been codon-modified but still encode a wild-type AAT protein, or a portion thereof. Codon modification, or codon optimization, refers to modifications in a DNA or RNA sequence that do not affect the amino acids encoded by the DNA or RNA sequence. Codon optimization may, however, improve expression of an encoded protein due to changes in the relative number and frequency of tRNAs used in translation. Replacing codons for a given amino acid with codons corresponding to tRNAs that are more abundant in human cells, for example, allows for faster translation of an RNA sequence into the same amino acid sequence.

The endogenous SERPINA1 gene comprises seven exons, referred to as exons 1a, 1b, 1c, 2, 3, 4, and 5, and six introns, referred to as introns 1a, 1b, 1c, 2, 3, and 4. The endogenous SERPINA1 gene comprises, in 5′ to 3′ order: exon 1a, intron 1a, exon 1b, intron 1b, exon 1c, intron 1b, exon 2, intron 2, exon 3, intron 3, exon 4, intron 4, and exon 5. The open reading frame encoding AAT comprises exons 2, 3, 4, and 5, while exons 1a, 1b, and 1c are comprised in the 5′ UTR of the RNA produced by transcription of the SERPINA1 gene. Thus, exons 1a, 1b, and 1c do not encode amino acids of AAT. Accordingly, the same coding sequence encoding functional AAT, comprising exons 2, 3, 4, and 5 of a wild-type SERPINA1 gene, may be inserted downstream of exon 1a, 1b, or 1c, and still encode wild-type AAT. Additionally, a donor nucleic acid sequence may encode some, but not all, of exons 2, 3, 4, and 5, depending on where the template nucleic acid is inserted in the SERPINA1 gene, and still encode a functional AAT protein. For example, if the template nucleic acid is inserted within intron 2, the donor nucleic acid sequence can comprise exons 3, 4, and 5. If the template nucleic acid is inserted within intron 3, the donor nucleic acid sequence can comprise exons 4 and 5. If the template nucleic acid is inserted within intron 4, the template nucleic acid can comprise only exon 5. Furthermore, the position of donor polynucleotide insertion, and which splice donor sequence the introduced splice acceptor sequence interacts with, will cause mRNAs with different 5′ UTRs containing one or more of exons 1a, 1b, and 1c to be produced during pre-mRNA processing.

Insertion into Intron 1a

In some embodiments described herein, the donor nucleic acid sequence of the template nucleic acid encodes an AAT protein encoded by exons 2, 3, 4, and 5 of a SERPINA1 gene, and the splice acceptor sequence is capable of pairing with an endogenous splice donor sequence that is positioned 3′ downstream and adjacent to exon 1a in a SERPINA1 gene. Accordingly, the elements of the template nucleic acid are designed for insertion into intron 1a of the SERPINA1 gene. Splicing of RNA transcribed from the modified SERPINA1 gene formed by insertion of the template nucleic acid into intron 1a thus produces an open reading frame comprising in 5′ to 3′ order: exon 1a of the endogenous SERPINA1 gene, and exons 2, 3, 4, and 5 of the donor nucleic acid sequence. In some embodiments, the donor nucleic acid sequence comprises exons 1b, 1c, 2, 3, 4, and 5 of a SERPINA1 gene, or codon-modified variants of one or more of exons 1b, 1c, 2, 3, 4, and 5 of a SERPINA1 gene. In some embodiments, the donor nucleic acid sequence does not comprise one or more of introns 1b, 1c, 2, 3, and 4 of a SERPINA1 gene. Thus, the coding sequence of the donor nucleic acid sequence is joined to an upstream exon of the SERPINA1 gene during pre-mRNA processing, but less splicing is required to produce an mRNA encoding a full-length, functional AAT protein.

In some embodiments, the donor nucleic acid sequence does not comprise any of introns 1b, 1c, 2, 3, and 4 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 3. In some embodiments, the donor nucleic acid sequence comprises a nucleic acid sequence set forth in SEQ ID NO: 3.

In some embodiments, the donor nucleic acid sequence comprises one or more of introns 1b, 1c, 2, 3, and 4 of a SERPINA1 gene, which are appropriately positioned adjacent to and downstream of their respective exons. In some embodiments, the donor nucleic acid sequence comprises introns 1b, 1c, 2, 3, and 4 of a SERPINA1 gene, which are appropriately positioned adjacent to and downstream of their respective exons. Following transcription of the modified SERPINA1 gene produced by insertion of the donor polynucleotide, any of the introns present in the AAT coding sequence of the donor nucleic acid are excised by splicing during pre-mRNA processing. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 4. In some embodiments, the donor nucleic acid sequence comprises a nucleic acid sequence set forth in SEQ ID NO: 4.

Insertion into Intron 1b

In some embodiments described herein, the donor nucleic acid sequence of the template nucleic acid encodes an AAT protein, or a fragment thereof, encoded by exons 2, 3, 4, and 5 of a SERPINA1 gene, and the splice acceptor sequence is capable of pairing with an endogenous splice donor sequence that is positioned 3′ downstream and adjacent to exon 1b in a SERPINA1 gene. Accordingly, the elements of the template nucleic acid are designed for insertion into intron 1b of the SERPINA1 gene. Splicing of RNA transcribed from the modified SERPINA1 gene formed by insertion of the template nucleic acid into intron 1b thus produces an open reading frame comprising in 5′ to 3′ order: exons 1a and 1b of the endogenous SERPINA1 gene, and exons 2, 3, 4, and 5 of the donor nucleic acid sequence. In some embodiments, the donor nucleic acid sequence comprises exons 1c, 2, 3, 4, and 5 of a SERPINA1 gene, or codon-modified variants of one or more of exons 1c, 2, 3, 4, and 5 of a SERPINA1 gene.

In some embodiments, the donor nucleic acid sequence does not comprise one or more of introns 1c, 2, 3, and 4 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 5. In some embodiments, the donor nucleic acid sequence comprises a nucleic acid sequence set forth in SEQ ID NO: 5.

In some embodiments, the donor nucleic acid sequence comprises one or more of introns 1c, 2, 3, and 4 of a SERPINA1 gene, which are appropriately positioned adjacent to and downstream of their respective exons. In some embodiments, the donor nucleic acid sequence comprises introns 1c, 2, 3, and 4 of a SERPINA1 gene, which are appropriately positioned adjacent to and downstream of their respective exons. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 6. In some embodiments, the donor nucleic acid sequence comprises a nucleic acid sequence set forth in SEQ ID NO: 6.

Insertion into Intron 1c

In some embodiments described herein, the donor nucleic acid sequence of the template nucleic acid encodes an AAT protein encoded by exons 2, 3, 4, and 5 of a SERPINA1 gene, and the splice acceptor sequence is capable of pairing with an endogenous splice donor sequence that is positioned 3′ downstream and adjacent to exon 1c in a SERPINA1 gene. Accordingly, the elements of the template nucleic acid are designed for insertion into intron 1c of the SERPINA1 gene. Splicing of RNA transcribed from the modified SERPINA1 gene formed by insertion of the template nucleic acid into intron 1c thus produces an open reading frame comprising in 5′ to 3′ order: exons 1a, 1b, and 1c of the endogenous SERPINA1 gene, and exons 2, 3, 4, and 5 of the donor nucleic acid sequence. In some embodiments, the donor nucleic acid sequence comprises exons 2, 3, 4, and 5 of a SERPINA1 gene, or codon-modified variants of one or more of exons 2, 3, 4, and 5 of a SERPINA1 gene.

In some embodiments, the donor nucleic acid sequence does not comprise one or more of introns 2, 3, and 4 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 7. In some embodiments, the donor nucleic acid sequence comprises a nucleic acid sequence set forth in SEQ ID NO: 7.

In some embodiments, the donor nucleic acid sequence comprises one or more of introns 2, 3, and 4 of a SERPINA1 gene, which are appropriately positioned adjacent to and downstream of their respective exons. In some embodiments, the donor nucleic acid sequence comprises introns 2, 3, and 4 of a SERPINA1 gene, which are appropriately positioned adjacent to and downstream of their respective exons. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 8. In some embodiments, the donor nucleic acid sequence comprises a nucleic acid sequence set forth in SEQ ID NO: 8.

Insertion into Intron 2

In some embodiments described herein, the donor nucleic acid sequence of the template nucleic acid encodes a portion of an AAT protein encoded by exons 3, 4, and 5 of a SERPINA1 gene, and the splice acceptor sequence is capable of pairing with an endogenous splice donor sequence that is positioned 3′ downstream and adjacent to exon 2 in a SERPINA1 gene. Accordingly, the elements of the template nucleic acid are designed for insertion into intron 2 of the SERPINA1 gene. Splicing of RNA transcribed from the modified SERPINA1 gene formed by insertion of the template nucleic acid into intron 2 thus produces an open reading frame comprising in 5′ to 3′ order: exons 1a, 1b, 1c, and 2 of the endogenous SERPINA1 gene, and exons 3, 4, and 5 of the donor nucleic acid sequence. In some embodiments, the donor nucleic acid sequence comprises exons 3, 4, and 5 of a SERPINA1 gene, or codon-modified variants of one or more of exons 3, 4, and 5 of a SERPINA1 gene.

In some embodiments, the donor nucleic acid sequence does not comprise one or more of introns 3 and 4 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 9. In some embodiments, the donor nucleic acid sequence comprises a nucleic acid sequence set forth in SEQ ID NO: 9.

In some embodiments, the donor nucleic acid sequence comprises one or more of introns 3 and 4 of a SERPINA1 gene, which are appropriately positioned adjacent to and downstream of their respective exons. In some embodiments, the donor nucleic acid sequence comprises introns 3 and 4 of a SERPINA1 gene, which are appropriately positioned adjacent to and downstream of their respective exons. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 10. In some embodiments, the donor nucleic acid sequence comprises a nucleic acid sequence set forth in SEQ ID NO: 10.

Insertion into Intron 3

In some embodiments described herein, the donor nucleic acid sequence of the template nucleic acid encodes a portion of an AAT protein encoded by exons 4 and 5 of a SERPINA1 gene, and the splice acceptor sequence is capable of pairing with an endogenous splice donor sequence that is positioned 3′ downstream and adjacent to exon 3 in a SERPINA1 gene. Accordingly, the elements of the template nucleic acid are designed for insertion into intron 3 of the SERPINA1 gene. Splicing of RNA transcribed from the modified SERPINA1 gene formed by insertion of the template nucleic acid into intron 3 thus produces an open reading frame comprising in 5′ to 3′ order: exons 1a, 1b, 1c, 2, and 3 of the endogenous SERPINA1 gene, and exons 4 and 5 of the donor nucleic acid sequence. In some embodiments, the donor nucleic acid sequence comprises exons 4 and 5 of a SERPINA1 gene, or codon-modified variants of one or more of exons 4 and 5 of a SERPINA1 gene.

In some embodiments, the donor nucleic acid sequence does not comprise intron 4 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 11. In some embodiments, the donor nucleic acid sequence comprises a nucleic acid sequence set forth in SEQ ID NO: 11.

In some embodiments, the donor nucleic acid sequence comprises intron 4 of a SERPINA1 gene, which is appropriately positioned adjacent to and downstream of exon 4. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity a sequence set forth in SEQ ID NO: 12. In some embodiments, the donor nucleic acid sequence comprises a nucleic acid sequence set forth in SEQ ID NO: 12.

Insertion into Intron 4

In some embodiments described herein, the donor nucleic acid sequence of the template nucleic acid encodes a portion of an AAT protein encoded by exon 5 of a SERPINA1 gene, and the splice acceptor sequence is capable of pairing with an endogenous splice donor sequence that is positioned 3′ downstream and adjacent to exon 4 in a SERPINA1 gene. Accordingly, the elements of the template nucleic acid are designed for insertion into intron 4 of the SERPINA1 gene. Splicing of RNA transcribed from the modified SERPINA1 gene formed by insertion of the template nucleic acid into intron 4 thus produces an open reading frame comprising in 5′ to 3′ order: exons 1a, 1b, 1c, 2, 3, and 4 of the endogenous SERPINA1 gene, and exon 5 of the donor nucleic acid sequence. In some embodiments, the donor nucleic acid sequence comprises exon 5 of a SERPINA1 gene, or a codon-modified variant of exon 5 of a SERPINA1 gene. In some embodiments, the donor nucleic acid sequence comprises a nucleic acid sequence having at least 80%, at least 85%, at least 90%, at least 95%, at least 96%, at least 97%, at least 98%, at least 99%, or up to 100% sequence identity to a sequence set forth in SEQ ID NO: 13. In some embodiments, the donor nucleic acid sequence comprises a nucleic acid sequence set forth in SEQ ID NO: 13.

### Bidirectional Template Nucleic Acids

In some embodiments, the template nucleic acid is a bidirectional template nucleic acid. A bidirectional template nucleic acid refers to a template nucleic acid comprising a first nucleic acid sequence and a second nucleic sequence that is downstream of the first nucleic acid sequence, wherein the first nucleic acid sequence comprises one or more elements in 5′ to 3′ order, and the second nucleic acid sequence comprises reverse complements of the one or more elements of the first nucleic acid sequence (or variants thereof), wherein the reverse complements are arranged in reverse order relative to their arrangement in the first nucleic acid sequence. For example, in a bidirectional template nucleic acid comprising a first nucleic acid sequence comprising, in 5′ to 3′ order: an A element, a B element, and a C element, the second nucleic acid sequence comprises, in 5′ to 3′ order: a reverse complement of a C element, a reverse complement of a B element, and a reverse complement of an A element. Thus, regardless of the orientation in which the bidirectional template nucleic acid is introduced into the genome, the modified SERPINA1 gene produced by homologous recombination will contain, in 5′ to 3′ order: (a) a splice acceptor sequence capable of pairing with an endogenous splice donor sequence that is positioned 3′ downstream and adjacent to exon 1c; (b) a sequence encoding an AAT protein encoded by exons 2, 3, 4, and 5 of a SERPINA1 gene; and (c) a termination sequence.

An element in a bidirectional template nucleic acid may refer to a class of sequences, such as termination sequences (stop codons and/or poly A tails), or to a specific nucleic acid sequence (a stop codon having the nucleic acid sequence TAA, or a polyA tail consisting of exactly 50 adenosine monophosphate nucleotides). In some embodiments, the first nucleic acid sequence (forward segment) and second nucleic acid sequence (reverse segment) comprise identical elements arranged in reverse order, with the reverse element being a reverse complement of the forward element. In some embodiments, the forward segment and reverse segment are separated by one or more intervening nucleotides. In some embodiments, the forward segment and reverse segment are flanked by nucleic acid sequences that do not have a corresponding reverse complement in the donor polynucleotide.

In particular embodiments, the donor nucleic acid sequence further comprises a reverse segment that is 3′ downstream of the termination sequence, wherein the reverse segment comprises, from 5′ to 3′: (a) a reverse complement of a second termination sequence; (b) a reverse complement of a second donor nucleic acid sequence encoding an AAT protein, or a portion thereof; and (c) a reverse complement of a second splicing sequence comprising a splice acceptor sequence capable of pairing with an endogenous splice donor sequence in a SERPINA1 gene.

In some embodiments, the reverse segment comprises reverse complements of one or more elements of the forward segment that are not identical to their corresponding elements on the forward segment. For example, a bidirectional template nucleic acid may comprise identical SERPINA1 coding sequences, but the forward segment may comprise a CMV intron, while the reverse segment comprises a reverse complement of an SV40 intron. In some embodiments, the second termination sequence is identical to the first termination sequence. In some embodiments, the second termination sequence differs from the first termination sequence. Non-limiting examples of manners in which the second termination sequence may differ from the termination sequence include comprising a different stop codon, comprising multiple stop codons in sequence, comprising a poly A tail of a different length, presence of a poly A tail, and absence of a polyA tail.

In some embodiments, the second donor nucleic acid sequence is identical to the first donor nucleic acid sequence. In some embodiments, the second donor nucleic acid sequence differs from the first donor nucleic acid sequence, but encodes the same AAT protein. The first and second nucleic acid sequences encoding the AAT protein may be identical, or differ by one or more nucleotide substitutions, such as for codon modification, codon optimization, or the introduction of a barcode sequence that allows a primer or probe having a complementary sequence to bind specifically to the barcode sequence.

In some embodiments, the second splicing sequence is identical to the first splicing sequence. In some embodiments, the second splicing sequence differs from the first splicing sequence, but is still capable of pairing with the same endogenous splice donor sequence in a SERPINA1 gene.

2.3 Methods for Producing Modified SERPINA1 Genes and/or Treating AAT Deficiency

In another aspect, the present disclosure provides methods of editing one or more SERPINA1 genes in a cell by introducing into the cell (i) an engineered nuclease, or a polynucleotide comprising a nucleic acid sequence encoding an engineered nuclease; and (ii) a donor polynucleotide comprising a template nucleic acid described herein, comprising (a) a splicing sequence comprising a splice acceptor sequence capable of pairing with an endogenous splice donor sequence in a SERPINA1 gene; (b) a donor nucleic acid sequence encoding an AAT protein or a portion thereof; and (c) a termination sequence, wherein the engineered nuclease binds and cleaves a recognition sequence within an endogenous SERPINA1 gene in the cell to generate a cleavage site, and wherein the template nucleic acid is inserted into the cleavage site to generate the modified SERPINA1 gene.

In other aspects, disclosed herein is a method for modifying a SERPINA1 gene in a target cell in a subject, the method comprising delivering to the target cell: (a) a polynucleotide (i.e., a donor polynucleotide) comprising a template nucleic acid described herein; and (b) an engineered nuclease or a second polynucleotide comprising a nucleic acid sequence encoding an engineered nuclease, wherein the engineered nuclease is expressed in the target cell, wherein the engineered nuclease binds and cleaves a recognition sequence within an endogenous SERPINA1 gene in the target cell to generate a cleavage site, and wherein the template nucleic acid is inserted into the cleavage site to generate the modified SERPINA1 gene.

In other aspects, disclosed herein is a method for treating AAT deficiency in a subject in need thereof, the method comprising administering to the subject: (a) a pharmaceutical composition comprising an effective amount of a polynucleotide comprising a template nucleic described herein. and (b) a pharmaceutical composition comprising an effective amount of an engineered nuclease or a second polynucleotide comprising a nucleic acid sequence encoding an engineered nuclease, wherein the polynucleotide comprising the template nucleic acid, and the engineered nuclease or second polynucleotide, are delivered to a target cell in the subject, wherein the engineered nuclease is expressed in the target cell, wherein the engineered nuclease binds and cleaves a recognition sequence within an endogenous SERPINA1 gene in the target cell to generate a cleavage site, and wherein the template nucleic acid is inserted into the cleavage site to generate the modified SERPINA1 gene.

In some embodiments of the methods described herein, the polynucleotide comprising the template nucleic acid sequence further comprises a 5′ homology and a 3′ homology arm that flank the template nucleic acid, with one homology arm comprising a nucleic acid sequence with homology to a sequence 5′ upstream of the AAT 33-34 recognition sequence, and the other homology arm comprising a nucleic acid sequence with homology to a sequence 3′ downstream of a nuclease recognition sequence. The double-stranded break created by nuclease-mediated cleavage can stimulate a cell's endogenous DNA-repair pathways (e.g., homology-dependent repair (HDR)). HDR, which is also known as homologous recombination (HR), can occur when a homologous repair template (e.g., a donor polynucleotide) is available. In the absence of a donor polynucleotide, the sister chromatid is generally used by the cell as the repair template. However, for the purposes of genome editing, the repair template is often supplied as an exogenous nucleic acid, such as a plasmid, duplex oligonucleotide, single strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. Thus, in some embodiments, homologous recombination is used to insert an exogenous polynucleotide sequence into the target nucleic acid cleavage site.

As an alternative to homologous recombination, when an exogenous DNA molecule is supplied in sufficient concentration inside the nucleus of a cell in which the double-strand break occurs, the exogenous DNA can be inserted at the double-stranded break during the NHEJ repair process and thus become a permanent addition to the genome. If a template nucleic acid contains a coding sequence for a gene of interest such as an AAT gene, as well as a splice acceptor sequence and termination sequence, the gene of interest can be expressed from the integrated copy in the genome, resulting in permanent expression for the life of the cell. Moreover, the integrated copy of the template nucleic acid can be transmitted to the daughter cells when the cell divides.

In some embodiments of the methods described herein, the endogenous SERPINA1 gene comprises at least one mutation relative to a wild-type SERPINA1 gene and encodes a mutant AAT protein. In some embodiments, the endogenous SERPINA1 gene comprises a Z allele mutation in exon 5. The “Z allele” refers to an allele comprising a mutation that changes a glutamate-encoding codon to a lysine-encoding one at position 342 of the amino acid sequence of AAT. Thus, an AAT protein encoded by a SERPINA1 gene comprising a Z allele mutation has a lysine (K) in place of a glutamate (E) amino acid at position 342. In some embodiments, some embodiments, the endogenous SERPINA1 gene comprises an S allele mutation in exon 3. The “S allele” refers to an allele comprising a mutation that changes a glutamate-encoding codon to a valine-encoding one at position 264 of the amino acid sequence of AAT. Thus, an AAT protein encoded by a SERPINA1 gene comprising an S allele mutation has a valine (V) in place of a glutamate (E) amino acid at position 264. In some embodiments, the genetically-modified cell expresses less of the mutant AAT protein, relative to an unmodified cell.

In some embodiments, the template nucleic acid is inserted in-frame in the SERPINA1 gene. In some embodiments, the donor nucleic acid sequence of the template nucleic acid is operably linked to an endogenous SERPINA1 promoter following insertion of the template nucleic acid into the cleavage site. A promoter is said to be operably linked to a nucleic acid sequence if the promoter regulates transcription of the operably linked nucleic acid sequence. Thus, in such embodiments, the template nucleic acid does not comprise an exogenous promoter, and the endogenous SERPINA1 promoter governs transcription of the modified SERPINA1 gene encoding the functional AAT protein.

In some embodiments, the modified SERPINA1 gene encodes a full-length wild-type AAT protein that does not comprise a Z allele mutation or an S allele mutation. While the Z allele and/or S allele mutations may be present downstream of the inserted template nucleic acid, and possibly transcribed during transcription of the modified SERPINA1 gene, the termination sequence of the template nucleic acid prevents such mutant AAT proteins from being translated. In some embodiments, the modified SERPINA1 gene comprises a nucleic acid sequence of a wild-type SERPINA1 gene. In some embodiments, the modified SERPINA1 gene comprises one or more codon-modified exons and/or introns and encodes a wild-type AAT protein. In some embodiments, the modified SERPINA1 gene comprises a nucleic acid sequence having at least 80%, at least 85%, at least 90%, at least 91%, at least 92%, at least 93%, at least 94%, at least 95%, at least 96%, at least 97%, at least 98%, at least 99%, or more, sequence identity to a sequence set forth in SEQ ID NO: 16. In some embodiments, the modified SERPINA1 gene comprises a nucleic acid sequence set forth in SEQ ID NO: 16.

In some embodiments, the eukaryotic cell modified according to the present disclosure is a mammalian cell. Non-limiting examples of mammals include mice, rats, rabbits, hamsters, guinea pigs, swine, cattle, alpacas, llamas, and humans. In some embodiments, the mammalian cell is a human cell. In some embodiments, the mammalian cell is a liver cell (e.g., a hepatocyte). In some embodiments, the mammalian cell is a liver progenitor cell or stem cell. In some embodiments, the eukaryotic cell is a pluripotent stem cell. In some embodiments, the eukaryotic cell is an induced pluripotent stem cell. In specific embodiments, the eukaryotic cell is a human liver cell (e.g., a human hepatocyte).

In some embodiments of the methods of treating AAT deficiency described herein, the subject is a human. In some embodiments, the target cell in the subject is a liver cell (e.g., a hepatocyte). In some embodiments, the target cell in the subject is a liver progenitor cell or stem cell.

2.4 Nucleases that Bind and Cleave Recognition Sequences within a SERPINA1 Gene

The methods described herein include the use of engineered nucleases in combination with donor polynucleotides described herein. Such engineered nucleases bind and cleave a recognition sequence within the SERPINA1 gene to generate a cleavage site into which a template nucleic acid described herein (i.e., provided by the donor polynucleotide) is inserted. In particular examples, such engineered nucleases can bind and cleave a recognition sequence within an intron of the SERPINA1 gene, such as intron 1a, 1b, 1c, 2, 3, or 4. Non-limiting examples of engineered nucleases useful in the present disclosure include, among others, engineered meganucleases, CRISPR system nucleases, zinc finger nucleases, TALENS, compact TALENs, and megaTALs.

An engineered meganuclease can be, for example, an endonuclease that is derived from I-CreI and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker.

In particular embodiments, the meganucleases used to practice the embodiments of the present disclosure are single-chain meganucleases. A single-chain meganuclease comprises an N-terminal subunit and a C-terminal subunit joined by a linker peptide. Each of the two subunits recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. The first subunit of a single-chain meganuclease comprises a first hypervariable (HVR1) region, and the second subunit comprises a second hypervariable (HVR2) region. Further, the first subunit binds to a first recognition half-site in the recognition sequence, and the second subunit binds to a second recognition half-site in the recognition sequence.

In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit.

Zinc finger nucleases (ZFNs) can be engineered to recognize and cut pre-determined sites in a genome. ZFNs are chimeric proteins comprising a zinc finger DNA-binding domain fused to a nuclease domain from an endonuclease or exonuclease (e.g., Type IIs restriction endonuclease, such as the FokI restriction enzyme). The zinc finger domain can be a native sequence or can be redesigned through rational or experimental means to produce a protein which binds to a pre-determined DNA sequence ˜18 basepairs in length. By fusing this engineered protein domain to the nuclease domain, it is possible to target DNA breaks with genome-level specificity. ZFNs have been used extensively to target gene addition, removal, and substitution in a wide range of eukaryotic organisms (reviewed in S. Durai et al., Nucleic Acids Res 33, 5978 (2005)).

Likewise, TAL-effector nucleases (TALENs) can be generated to cleave specific sites in genomic DNA. Like a ZFN, a TALEN comprises an engineered, site-specific DNA-binding domain fused to an endonuclease or exonuclease (e.g., Type IIs restriction endonuclease, such as the FokI restriction enzyme) (reviewed in Mak, et al. (2013) Curr Opin Struct Biol. 23:93-9). In this case, however, the DNA binding domain comprises a tandem array of TAL-effector domains, each of which specifically recognizes a single DNA basepair.

Compact TALENs are an alternative endonuclease architecture that avoids the need for dimerization (Beurdeley, et al. (2013) Nat Commun. 4:1762). A Compact TALEN comprises an engineered, site-specific TAL-effector DNA-binding domain fused to the nuclease domain from the I-TevI homing endonuclease or any of the endonucleases listed in Table 2 in U.S. application No. 20130117869. Compact TALENs do not require dimerization for DNA processing activity, so a Compact TALEN is functional as a monomer.

Engineered endonucleases based on the CRISPR/Cas system are also known in the art (Ran, et al. (2013) Nat Protoc. 8:2281-2308; Mali et al. (2013) Nat Methods. 10:957-63). In those embodiments wherein a CRISPR system is used for insertion of a donor nucleic acid sequence into a heterologous polynucleotide or genomic locus, the CRISPR system comprises two components: (1) a CRISPR nuclease; and (2) a short “guide RNA” comprising a ˜20 nucleotide targeting sequence that directs the nuclease to a location of interest in the genome or on a polynucleotide. The CRISPR system may also comprise a tracrRNA. By expressing multiple guide RNAs in the same cell, each having a different targeting sequence, it is possible to target DNA breaks simultaneously to multiple sites in the genome. The presently disclosed compositions and methods utilizing a CRISPR system may comprise a CRISPR nuclease and the guide RNA(s) or nucleic acids encoding the CRISPR nuclease and/or the guide RNA(s).

Nucleases referred to as megaTALs are single-chain endonucleases comprising a transcription activator-like effector (TALE) DNA binding domain with an engineered, sequence-specific homing endonuclease.

The presently disclosed compositions and methods can utilize purified nuclease proteins, or nucleic acids encoding nucleases. These can be delivered into cells to cleave genomic DNA or a polynucleotide by a variety of different mechanisms known in the art, including those further detailed elsewhere herein. In some embodiments wherein a CRISPR system nuclease is utilized, a ribonucleoprotein complex comprising the CRISPR nuclease and guide RNA(s) can be introduced into a cell.

### 2.5 Methods for Delivering and Donor Polynucleotides and Engineered Nucleases

In different aspects, the present disclosure provides engineered nucleases that are useful for binding and cleaving recognition sequences within a SERPINA1 gene of a cell (e.g., the human SERPINA1 gene). The present disclosure further provides donor polynucleotides comprising a template nucleic acid that encodes a functional (e.g., wild-type) AAT protein, which are meant to be inserted into the cleavage site generated by the engineered nuclease in the SERPINA1 gene. The present disclosure provides various methods for modifying a SERPINA1 gene in cells using the engineered nucleases and donor polynucleotides described herein, methods for making genetically-modified cells comprising a modified SERPINA1 gene, and methods of modifying a SERPINA1 gene in a target cell in a subject. In further aspects, the present disclosure provides methods for treating AAT deficiency in a subject by administering the engineered nucleases (or polynucleotides encoding the same) and donor polynucleotides described herein to a subject, in some cases as part of a pharmaceutical composition. In each case, it is envisioned that the engineered nucleases (or polynucleotides encoding the same) and the donor polynucleotides are introduced into cells, such as liver cells (e.g., hepatocytes), liver progenitor cells, or stem cells that express an AAT protein.

Disruption of mutant AAT protein expression, either by gene knockout or by insertion of a template nucleic acid provided by a donor polynucleotide, can reduce the accumulation of mutant AAT proteins in the blood. Such reductions can be determined, for example, by measuring the amount of mutant AAT protein produced by the genetically-modified cell or the amount of mutant AAT protein present in a subject relative to a control (e.g., a control cell, a control subject, or a sample taken prior to treatment with the engineered meganuclease cell), using well-known protein measurement techniques known in the art including immunofluorescence, western blotting, and enzyme-linked immunosorbent assays (ELISA), which use antibodies that specifically bind mutant, but not functional, AAT protein. In specific embodiments, the expression or presence of a mutant AAT protein can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the expression or presence of a mutant AAT protein can be reduced by 1%-5%, 5%-10%, 10%-20%, 20%-30%, 30%-40%, 40%-50%, 50%-60%, 70%-80%, 90%-95%, 95%-98%, or up 100% relative to the control.

Modification of the SERPINA1 gene to introduce a coding sequence encoding a functional (e.g., wild-type) AAT protein can be determined, for example, by sequencing the SERPINA1 gene in a genetically-modified cell, by measuring the abundance of the RNA encoding functional AAT, or by measuring the protein level of the functional AAT protein by protein measurement techniques (immunofluorescence, western blotting, and ELISA) using antibodies that specifically bind functional, but not mutant, AAT protein.

Levels of functional AAT (e.g., wild-type AAT) can be increased in a genetically-modified eukaryotic cell relative to a control (e.g., a control cell, such as a eukaryotic cell treated with a meganuclease that does not target the SERPINA1 gene), and can be increased in the blood or serum of a subject relative to a control (e.g., a sample taken prior to treatment with the engineered nuclease). In some embodiments, the production of functional AAT, or functional AAT level, can be increased by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or up to 100% relative to the control. In some embodiments, the production of functional AAT can be increased by 1%-5%, 5%-10%, 10%-20%, 20%-30%, 30%-40%, 40%-50%, 50%-60%, 70%-80%, 90%-95%, 95%-98%, or up to 100% relative to the control. In various aspects, the methods described herein can increase protein levels of a functional (e.g., wild-type) AAT protein in a genetically-modified cell, target cell, or subject (e.g., as measured in a cell, a tissue, an organ, or a biological sample obtained from the subject), to at least 1%, 2%, 3%, 4%, 5%, 10%, 15%, 20%, 25%, 30%, 40%, 50%, 60%, 70%, 80%, 90%, 95%, 100%, 200%, 300%, 400%, 500%, or more, of a reference level (i.e., expression level of AAT in a wild-type cell or subject). Functional and/or wild-type AAT levels can be measured in a cell, tissue, organ, or blood, as described elsewhere herein.

The methods disclosed herein can be effective to decrease the risk of lung disease in the subject relative to a control subject having AAT deficiency. The control subject may be a subject having AAT deficiency treated with a nuclease that does not target the SERPINA1 gene or treated with a nuclease targeting the SERPINA1 gene but not a donor polynucleotide comprising a template nucleic acid encoding a functional AAT protein.

In some embodiments, the risk of lung disease can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the reference level. In some embodiments, the risk of lung disease can be reduced by 1%-5%, 5%-10%, 10%-20%, 20%-30%, 30%-40%, 40%-50%, 50%-60%, 70%-80%, 90%-95%, 95%-98%, or up to 100% relative to the reference level.

In some embodiments, the risk of liver disease can be reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, or 100% relative to the reference level. In some embodiments, the risk of liver disease can be reduced by 1%-5%, 5%-10%, 10%-20%, 20%-30%, 30%-40%, 40%-50%, 50%-60%, 70%-80%, 90%-95%, 95%-98%, or up to 100% relative to the reference level.

### Detection and Expression

Expression of a functional AAT protein (e.g., a wild-type AAT protein) in a genetically-modified cell or subject can be detected using standard methods in the art. For example, levels of functional AAT protein may be assessed based on the level of any variable associated with AAT gene expression, e.g., SERPINA1 mRNA levels or AAT protein levels. Increased levels or expression of functional AAT protein may be assessed by an increase in an absolute or relative level of one or more of these variables compared with a reference level. Such functional AAT protein levels may be measured in a biological sample isolated from a subject, such as a tissue biopsy or a bodily fluid including blood, serum, plasma, cerebrospinal fluid, or urine. Optionally, such functional AAT protein levels are normalized to a standard protein or substance in the sample. Further, such functional AAT protein levels can be assessed any time before, during, or after treatment in accordance with the methods herein.

Introduction of Engineered Nucleases and Donor Polynucleotides into Cells

Engineered nuclease proteins disclosed herein, polynucleotides encoding engineered nucleases described herein, and donor polynucleotides comprising a template nucleic acid described herein, can be delivered into cells by a variety of different mechanisms known in the art, including those further detailed herein below.

Engineered nucleases disclosed herein can be delivered into a cell in the form of protein or, preferably, as a polynucleotide comprising a nucleic acid sequence encoding the engineered nuclease. Such polynucleotides can be, for example, DNA (e.g., circular or linearized plasmid DNA, PCR products, or viral genomes) or RNA (e.g., mRNA).

For embodiments in which the engineered nuclease coding sequence is delivered in DNA form, it should be operably linked to a promoter to facilitate transcription of the nuclease gene. Mammalian promoters suitable for the invention include constitutive promoters such as the cytomegalovirus early (CMV) promoter (Thomsen et al. (1984), Proc Natl Acad Sci USA. 81 (3): 659-63) or the SV40 early promoter (Benoist and Chambon (1981), Nature. 290 (5804): 304-10) as well as inducible promoters such as the tetracycline-inducible promoter (Dingermann et al. (1992), Mol Cell Biol. 12 (9): 4038-45). An engineered nuclease of the present disclosure can also be operably linked to a synthetic promoter. Synthetic promoters can include, without limitation, the JeT promoter (WO 2002/012514).

In specific embodiments, a nucleic acid sequence encoding an engineered nuclease described herein is operably linked to a tissue-specific promoter, such as a liver-specific promoter. Examples of liver-specific promoters include, without limitation, a human thyroxine binding globulin (TBG) promoter, human alpha-1 antitrypsin promoter, hybrid liver-specific promoter (hepatic locus control region from ApoE gene (ApoE-HCR) and a liver-specific alpha1-antitrypsin promoter), and apolipoprotein A-II promoter. In particular embodiments, the liver-specific promoter is a TBG promoter.

In specific embodiments, a polynucleotide comprising a nucleic acid sequence encoding an engineered nuclease described herein is delivered on a recombinant DNA construct or expression cassette. For example, the recombinant DNA construct can comprise an expression cassette (i.e., “cassette”) comprising a promoter and a nucleic acid sequence encoding an engineered nuclease described herein.

In another particular embodiment, a polynucleotide comprising a nucleic acid sequence encoding an engineered nuclease described herein is introduced into the cell using a single-stranded DNA template. The single-stranded DNA can further comprise a 5′ and/or a 3′ AAV ITR upstream and/or downstream of the sequence encoding the engineered nuclease. The single-stranded DNA can further comprise a 5′ and/or a 3′ homology arm upstream and/or downstream of the sequence encoding the engineered nuclease.

In another particular embodiment, a polynucleotide comprising a nucleic acid sequence encoding an engineered nuclease described herein can be introduced into a cell using a linearized DNA template. Such linearized DNA templates can be produced by methods known in the art. For example, a plasmid DNA encoding a nuclease can be digested by one or more restriction enzymes such that the circular plasmid DNA is linearized prior to being introduced into a cell.

In some embodiments, mRNA encoding an engineered nuclease described herein is delivered to a cell because this reduces the likelihood that the gene encoding the engineered nuclease will integrate into the genome of the cell. Such mRNA can be produced using methods known in the art such as in vitro transcription. In some embodiments, the mRNA is 5′ capped using 7-methyl-guanosine, anti-reverse cap analogs (ARCA) (U.S. Pat. No. 7,074,596), CleanCap® analogs such as Cap 1 analogs (Trilink, San Diego, CA), or enzymatically capped using vaccinia capping enzyme or similar. In some embodiments, the mRNA may be polyadenylated. The mRNA may contain various 5′ and 3′ untranslated sequence elements to enhance expression the encoded engineered nuclease and/or stability of the mRNA itself. Such elements can include, for example, posttranslational regulatory elements such as a woodchuck hepatitis virus posttranslational regulatory element (WPRE). The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

In some embodiments, the nuclease proteins, or DNA/mRNA encoding the nuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25:679-736), MPG (Simeoni, et al. (2003) Nucleic Acids Res. 31:2717-2724), Pep-1 (Deshayes et al. (2004) Biochemistry 43:7698-7706, and HSV-1 VP-22 (Deshayes et al. (2005) Cell Mol Life Sci. 62:1839-49. In an alternative embodiment, engineered nucleases, or DNA/mRNA encoding nucleases, are coupled covalently or non-covalently to an antibody that recognizes a specific cell-surface receptor expressed on target cells such that the nuclease protein/DNA/mRNA binds to and is internalized by the target cells. Alternatively, engineered nuclease protein/DNA/mRNA can be coupled covalently or non-covalently to the natural ligand (or a portion of the natural ligand) for such a cell-surface receptor. (McCall, et al. (2014) Tissue Barriers. 2 (4): e944449; Dinda, et al. (2013) Curr Pharm Biotechnol. 14:1264-74; Kang, et al. (2014) Curr Pharm Biotechnol. 15 (3): 220-30; Qian et al. (2014) Expert Opin Drug Metab Toxicol. 10 (11): 1491-508).

In some embodiments, nuclease proteins, or DNA/mRNA encoding nucleases, are encapsulated within biodegradable hydrogels for injection or implantation within the desired region of the liver (e.g., in proximity to hepatic sinusoidal endothelial cells or hematopoietic endothelial cells, or progenitor cells which differentiate into the same). Hydrogels can provide sustained and tunable release of the therapeutic payload to the desired region of the target tissue without the need for frequent injections, and stimuli-responsive materials (e.g., temperature- and pH-responsive hydrogels) can be designed to release the payload in response to environmental or externally applied cues (Kang Derwent et al. (2008) Trans Am Ophthalmol Soc. 106:206-214).

In some embodiments, nuclease proteins, or DNA/mRNA encoding nucleases, are coupled covalently or, preferably, non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the nuclease proteins, mRNA, or DNA can be attached to or encapsulated with the nanoparticle core. This increases the copy number of the protein/mRNA/DNA that is delivered to each cell and, so, increases the intracellular expression of each nuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33 (30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell-surface receptors and the natural ligands (or portions of the natural ligands) for cell surface receptors.

In some embodiments, the nuclease proteins, or DNA/mRNA encoding nucleases, are encapsulated within liposomes or complexed using cationic lipids (see, e.g., LIPOFECTAMINE™, Life Technologies Corp., Carlsbad, CA; Zuris et al. (2015) Nat Biotechnol. 33:73-80; Mishra et al. (2011) J Drug Deliv. 2011:863734). In some embodiments, the nuclease proteins, or DNA/mRNA encoding nucleases, are encapsulated within Lipofectamine® MessengerMax cationic lipid. The liposome and lipoplex formulations can protect the payload from degradation, enhance accumulation and retention at the target site, and facilitate cellular uptake and delivery efficiency through fusion with and/or disruption of the cellular membranes of the target cells.

In some embodiments, nuclease proteins, or DNA/mRNA encoding nucleases, are encapsulated within polymeric scaffolds (e.g., PLGA) or complexed using cationic polymers (e.g., PEI, PLL) (Tamboli et al. (2011) Ther Deliv. 2 (4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, nuclease proteins, or DNA/mRNA encoding nucleases, are combined with amphiphilic molecules that self-assemble into micelles (Tong et al. (2007). J Gene Med. 9 (11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, nuclease proteins, or DNA/mRNA encoding nucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in U.S. Pat. Nos. 6,015,832, 6,506,803, 6,635,676, 6,559,189, and 7,767,216, each of which is incorporated herein by reference in its entirety.

In some embodiments, nuclease proteins, or DNA/mRNA encoding nucleases, are covalently attached to, or non-covalently associated with, multifunctional polymer conjugates, DNA dendrimers, and polymeric dendrimers (Mastorakos et al. (2015) Nanoscale. 7 (9): 3845-56; Cheng et al. (2008) J Pharm Sci. 97 (1): 123-43). The dendrimer generation can control the payload capacity and size and can provide a high payload capacity. Moreover, display of multiple surface groups can be leveraged to improve stability, reduce nonspecific interactions, and enhance cell-specific targeting and drug release.

In some embodiments, polynucleotides comprising a nucleic acid sequence encoding an engineered nuclease described herein are introduced into a cell using a recombinant virus (i.e., a recombinant viral vector). Such recombinant viruses are known in the art and include recombinant retroviruses, recombinant lentiviruses, recombinant adenoviruses, and recombinant AAVs (reviewed in Vannucci, et al. (2013 New Microbiol. 36:1-22). Recombinant AA Vs useful in the invention can have any serotype that allows for transduction of the virus into a target cell type and expression of the nuclease gene in the target cell. For example, in some embodiments, recombinant AAVs have a serotype (i.e., a capsid) of AAV1, AAV2, AAV5 AAV6, AAV7, AAV8, AAV9, or AAV12. It is known in the art that different AAVs tend to localize to different tissues (Wang et al., Expert Opin Drug Deliv 11 (3). 2014). In some particular embodiments, the AAV serotype is AAV8. AAVs can also be self-complementary such that they do not require second-strand DNA synthesis in the host cell (McCarty, et al. (2001) Gene Ther. 8:1248-54). Polynucleotides delivered by recombinant AAVs can include left (5′) and right (3′) ITRs as part of the viral genome. In some embodiments, the recombinant viruses are injected directly into target tissues. In alternative embodiments, the recombinant viruses are delivered systemically via the circulatory system.

The AAV vectors and viral particles of the disclosure may exhibit transduction and/or activity in a multitude of tissue types, including but not limited to liver tissue, spleen tissue, adrenal tissue, lung tissue and heart tissue. In certain embodiments, the AAV vectors and viral particles of the disclosure may exhibit high, efficient transduction and/or activity in liver tissues. In some embodiments, the AAV8 capsid is used in combination with the TBG liver-specific promoter. The AAV8 serotype exhibits preferential tropism for liver tissues, and the specificity of the liver TBG promoter may control, mediate or limit editing to liver tissues to the exclusion of non-liver tissues.

In one embodiment, a recombinant virus used for nuclease gene delivery is a self-limiting recombinant virus. A self-limiting virus can have limited persistence time in a cell or organism due to the presence of a recognition sequence for an engineered nuclease within the viral genome. Thus, a self-limiting recombinant virus can be engineered to provide a coding sequence for a promoter, an engineered nuclease described herein, and a nuclease recognition site within the ITRs. The self-limiting recombinant virus delivers the nuclease gene to a cell, tissue, or organism, such that the nuclease is expressed and able to cut the genome of the cell at an endogenous recognition sequence within the genome. The delivered nuclease will also find its target site within the self-limiting recombinant viral genome, and cut the recombinant viral genome at this target site. Once cut, the 5′ and 3′ ends of the viral genome will be exposed and degraded by exonucleases, thus killing the virus and ceasing production of the nuclease.

If a polynucleotide comprising a nucleic acid sequence encoding an engineered nuclease described herein is delivered to a cell by a recombinant virus (e.g., an AAV), the nucleic acid sequence encoding the engineered nuclease can be operably linked to a promoter. In some embodiments, this can be a viral promoter such as endogenous promoters from the recombinant virus (e.g., the LTR of a lentivirus) or the well-known cytomegalovirus- or SV40 virus-early promoters. In particular embodiments, a nucleic acid sequence encoding an engineered nuclease is operably linked to a promoter that drives gene expression preferentially in the target cells (e.g., liver cells). Examples of liver-specific tissue promoters include but are not limited to those liver-specific promoters previously described, including the TBG promoter.

In some embodiments, the methods include delivering an engineered nuclease described herein, or a polynucleotide encoding the same, to a cell in combination with a donor polynucleotide comprising a template nucleic acid described herein encoding a sequence of interest (i.e., a sequence encoding a functional AAT protein), wherein the engineered nuclease is expressed in the cells, binds, and cleaves a recognition sequence described herein within a SERPINA1 gene of the cell, and generates a cleavage site, wherein the template nucleic acid and sequence of interest are inserted into the genome at the cleavage site (e.g., by homologous recombination).

Such donor polynucleotides comprising a template nucleic acid can be introduced into a cell and/or delivered to a target cell in a subject by any of the means previously discussed for delivery of a polynucleotide. In particular embodiments, such donor polynucleotides comprising a template nucleic acid molecule are introduced by way of a recombinant virus (i.e., a viral vector), such as a recombinant lentivirus, recombinant retrovirus, recombinant adenovirus, or a recombinant AAV. Recombinant AAVs useful for introducing a donor polynucleotide comprising a template nucleic acid can have any serotype (i.e., capsid) that allows for transduction of the virus into the cell and insertion of the template nucleic acid molecule sequence into the cell genome. In some embodiments, recombinant AAVs have a serotype of AAV1, AAV2, AAV5 AAV6, AAV7, AAV8, AAV9, or AAV12. In some particular embodiments, the AAV serotype is AAV8. The recombinant AAV can also be self-complementary such that it does not require second-strand DNA synthesis in the host cell. Template nucleic acids introduced using a recombinant AAV can be flanked by a 5′ (left) and 3′ (right) ITR in the viral genome.

In another particular embodiment, a donor polynucleotide comprising a template nucleic acid can be introduced into a cell and/or delivered to a target cell in a subject by way of a lipid nanoparticle. Examples of lipid nanoparticles useful for delivery of a donor polynucleotide are known in the art, and certain examples are described herein. When a template nucleic acid molecule is introduced or delivered by a lipid nanoparticle, the template nucleic acid molecule can be, for example, in the form of a double-stranded DNA template. In other embodiments, the donor polynucleotide can be in the form of a single-stranded DNA template. The single-stranded DNA can comprise, for example, the template nucleic acid molecule and, in particular embodiments, 5′ and 3′ homology arms to promote insertion of the template nucleic acid sequence into the nuclease cleavage site by homologous recombination. The single-stranded DNA can, in some cases, further comprise a 5′ AAV ITR sequence 5′ upstream of the 5′ homology arm, and a 3′ AAV ITR sequence 3′ downstream of the 3′ homology arm. In yet further examples, a donor polynucleotide of the present disclosure can be in the form of a linearized DNA template. In some such examples, a plasmid DNA comprising an exogenous nucleic acid sequence can be digested by one or more restriction enzymes such that the circular plasmid DNA is linearized.

In particular examples, the donor polynucleotide does not comprise an exogenous promoter that is operably linked to the template nucleic acid. In such examples, the template nucleic acid is operably linked to an endogenous promoter (e.g., the endogenous SERPINA1 promoter) following insertion into the engineered nuclease cleavage site in the SERPINA1 gene.

In some examples, the donor polynucleotide does comprise an exogenous promoter, and the template nucleic acid described herein is operably linked to the exogenous promoter suitable which is suitable for expression of the encoded AAT protein in the cell. Such promoters can include, for example, those mammalian, inducible, and tissue-specific promoters previously discussed.

### Administration

The target tissue(s) or target cell(s) include, without limitation, liver cells (e.g., hepatocytes), such as human liver cells. In some embodiments, the target cell is a liver progenitor cell.

In some embodiments, engineered nucleases described herein, polynucleotides encoding the same, and/or donor polynucleotides comprising a template nucleic acid described herein are delivered to a cell in vitro. In some embodiments, engineered nucleases described herein, polynucleotides encoding the same, and/or donor polynucleotides comprising a template nucleic acid described herein are delivered to a target cell in a subject in vivo. In some embodiments, engineered nucleases described herein, polynucleotides encoding the same, and/or donor polynucleotides comprising a template nucleic acid described herein are supplied to target cells (e.g., a liver cell or liver progenitor cell) via injection directly to the target tissue. Alternatively, engineered nucleases described herein, polynucleotides encoding the same, and/or donor polynucleotides comprising a template nucleic acid described herein can be delivered systemically via the circulatory system.

In various embodiments of the methods, the compositions described herein can be administered via any suitable route of administration known in the art. Such routes of administration can include, for example, intravenous, intramuscular, intraperitoneal, subcutaneous, intrahepatic, transmucosal, transdermal, intraarterial, and sublingual. In some embodiments, the compositions described herein are supplied to target cells (e.g., liver cells or liver precursor cells) via injection directly to the target tissue (e.g., liver tissue). Other suitable routes of administration can be readily determined by the treating physician as necessary.

In some embodiments, a therapeutically effective amount of an engineered nuclease described herein, or a polynucleotide encoding the same, is administered in combination with a donor polynucleotide comprising a template nucleic acid described herein to a subject in need thereof for the treatment of a disease, such as AAT deficiency. As appropriate, the dosage or dosing frequency of the engineered nuclease, the polynucleotide encoding the same, and/or the donor polynucleotide may be adjusted over the course of the treatment, based on the judgment of the administering physician. Appropriate doses will depend, among other factors, on the specifics of any AAV chosen (e.g., serotype), any lipid nanoparticle chosen, on the route of administration, on the subject being treated (i.e., age, weight, sex, and general condition of the subject), and the mode of administration. Thus, the appropriate dosage may vary from patient to patient. An appropriate effective amount can be readily determined by one of skill in the art or treating physician. Dosage treatment may be a single dose schedule or, if multiple doses are required, a multiple dose schedule. Moreover, the subject may be administered as many doses as appropriate. One of skill in the art can readily determine an appropriate number of doses. The dosage may need to be adjusted to take into consideration an alternative route of administration or balance the therapeutic benefit against any side effects.

In some embodiments, the methods further include administration of a polynucleotide comprising a nucleic acid sequence encoding a secretion-impaired hepatotoxin, or encoding tPA, which stimulates hepatocyte regeneration without acting as a hepatotoxin.

In some embodiments, a subject is administered a pharmaceutical composition comprising a polynucleotide comprising a nucleic acid sequence encoding an engineered nuclease described herein, wherein the encoding nucleic acid sequence is administered at a dose of about 1×10¹⁰ gc/kg to about 1×10¹⁴ gc/kg (e.g., about 1×10¹⁰ gc/kg, about 1×10¹¹ gc/kg, about 1×10¹² gc/kg, about 1×10¹³ gc/kg, or about 1×10¹⁴ gc/kg). In some embodiments, a subject is administered a pharmaceutical composition comprising a polynucleotide comprising a nucleic acid sequence encoding an engineered nuclease described herein, wherein the encoding nucleic acid sequence is administered at a dose of about 1×10¹⁰ gc/kg, about 1×10¹¹ gc/kg, about 1×10¹² gc/kg, about 1×10¹³ gc/kg, or about 1×10¹⁴ gc/kg. In some embodiments, a subject is administered a pharmaceutical composition comprising a polynucleotide comprising a nucleic acid sequence encoding an engineered nuclease described herein, wherein the encoding nucleic acid sequence is administered at a dose of about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg.

In some embodiments, a subject is administered a pharmaceutical composition comprising a donor polynucleotide comprising a template nucleic acid described herein, wherein the template nucleic acid is administered at a dose of about 1×10¹⁰ gc/kg to about 1×10¹⁴ gc/kg (e.g., about 1×10¹⁰ gc/kg, about 1×10¹¹ gc/kg, about 1×10¹² gc/kg, about 1×10¹³ gc/kg, or about 1×10¹⁴ gc/kg). In some embodiments, a subject is administered a pharmaceutical composition comprising a donor polynucleotide comprising a template nucleic acid described herein, wherein the template nucleic acid is administered at a dose of about 1×10¹⁰ gc/kg, about 1×10¹¹ gc/kg, about 1×10¹² gc/kg, about 1×10¹³ gc/kg, or about 1×10¹⁴ gc/kg. In some embodiments, a subject is administered a pharmaceutical composition comprising a donor polynucleotide comprising a template nucleic acid described herein, wherein the template nucleic acid is administered at a dose of about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg.

In some embodiments, a subject is administered a lipid nanoparticle formulation comprising a polynucleotide (e.g., mRNA) comprising a nucleic acid sequence encoding an engineered nuclease described herein, wherein the dose of the polynucleotide (e.g., mRNA) is about 0.1 mg/kg to about 3 mg/kg. In some embodiments, a subject is administered a lipid nanoparticle formulation comprising a polynucleotide (e.g., mRNA) comprising a nucleic acid sequence encoding an engineered nuclease described herein, wherein the dose of the polynucleotide (e.g., mRNA) is about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg. In some embodiments, a subject is administered a lipid nanoparticle formulation comprising a polynucleotide (e.g., mRNA) comprising a nucleic acid sequence encoding an engineered nuclease described herein, wherein the dose of the polynucleotide (e.g., mRNA) is about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg.

In some embodiments, a subject is administered a lipid nanoparticle formulation comprising a donor polynucleotide comprising a template nucleic acid described herein, wherein the dose of the donor polynucleotide is about 0.1 mg/kg to about 3 mg/kg. In some embodiments, a subject is administered a lipid nanoparticle formulation comprising a donor polynucleotide comprising a template nucleic acid described herein, wherein the dose of the donor polynucleotide is about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg. In some embodiments, a subject is administered a lipid nanoparticle formulation comprising a donor polynucleotide comprising a template nucleic acid described herein, wherein the dose of the donor polynucleotide is about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg.

### 2.6 Pharmaceutical Compositions

In some embodiments, the present disclosure provides a pharmaceutical composition comprising a pharmaceutically acceptable carrier and an engineered nuclease described herein, or a pharmaceutically acceptable carrier and a polynucleotide that comprises a nucleic acid sequence encoding an engineered nuclease described herein. Such polynucleotides can be, for example, mRNA or DNA as described herein.

In some embodiments, the disclosure provides a pharmaceutical composition comprising a pharmaceutically acceptable carrier and a donor polynucleotide described herein that comprises a template nucleic acid comprising (a) a splicing sequence comprising a splice acceptor sequence capable of pairing with an endogenous splice donor sequence in a SERPINA1 gene; (b) a donor nucleic acid sequence encoding an AAT protein, or a portion thereof; and (c) a termination sequence. In some such examples, the polynucleotide in the pharmaceutical composition can be comprised by a lipid nanoparticle or can be comprised by a recombinant virus (e.g., a recombinant AAV).

Such pharmaceutical compositions are formulated, for example, for systemic administration, or administration to target tissues.

Pharmaceutical compositions of the present disclosure can be useful for treating a subject having AAT deficiency. In some instances, the subject undergoing treatment in accordance with the methods and compositions described herein can be characterized by having a mutation in a SERPINA1 gene, such as a Pi*Z mutation or a Pi*S mutation. A subject having AAT deficiency, or a subject who may be particularly receptive to treatment with the engineered meganucleases and donor polynucleotides described herein, may be identified by ascertaining the presence or absence of one or more such risk factors, diagnostic, or prognostic indicators. The determination may be based on clinical and sonographic findings, including enzymology analyses and/or DNA analyses known in the art.

For example, the subject undergoing treatment can be characterized by bloodstream levels of mutant AAT, e.g., mutant AAT levels of at least 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg of mutant AAT per mL of blood, or more. In certain embodiments, the mutant AAT level is associated with one or more symptoms or pathologies, such as pulmonary edema. Mutant AAT levels may be measured in a biological sample, such as a body fluid including blood, serum, plasma, or urine. In some embodiments, the claimed methods include administration of an engineered nuclease (or nucleic acid encoding the same) and a donor polynucleotide described herein to reduce serum mutant AAT levels in a subject to undetectable levels, or to less than 1% 2%, 5%, 10%, 20%, 30%, 40%, 50%, 60%, 70%, or 80% of the subject's mutant AAT levels prior to treatment, within 1 day, 3 days, 5 days, 7 days, 9 days, 12 days, or 15 days.

Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the present disclosure, engineered meganucleases described herein, polynucleotides encoding the same, or cells expressing the same, are typically admixed with a pharmaceutically acceptable carrier and the resulting composition is administered to a subject. The carrier must be acceptable in the sense of being compatible with any other ingredients in the formulation and must not be deleterious to the subject. The carrier can be a solid or a liquid, or both, and can be formulated with the compound as a unit-dose formulation.

In some embodiments, pharmaceutical compositions of the present disclosure can further comprise one or more additional agents or biological molecules useful in the treatment of a disease in the subject. Likewise, the additional agent(s) and/or biological molecule(s) can be co-administered as a separate composition.

The pharmaceutical compositions described herein can include a therapeutically effective amount of any engineered nuclease disclosed herein, a therapeutically effective amount of a polynucleotide described herein encoding an engineered nuclease described herein, and/or a therapeutically effective amount of a donor polynucleotide described herein. For example, in some embodiments, the pharmaceutical composition can include polynucleotides described herein at any of the doses (e.g., gc/kg of an encoding nucleic acid sequence or mg/kg of mRNA) described herein.

In particular embodiments of the present disclosure, the pharmaceutical composition can comprise one or more recombinant viruses (e.g., recombinant AAVs) described herein that comprise one or more polynucleotides described herein (i.e., packaged within the viral genome). In some embodiments, the pharmaceutical composition comprises two or more recombinant viruses described herein (e.g., recombinant AAVs), each comprising a polynucleotide described herein, wherein a first recombinant virus comprises a polynucleotide comprising a nucleic acid sequence encoding an engineered nuclease described herein having specificity for a recognition sequence within the SERPINA1 gene, and a second recombinant virus that comprises a donor polynucleotide.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the polypeptide, nucleic acid, or vector to elicit a desired response in the individual. As used herein a therapeutically effective result can refer to a reduction mutant AAT concentration in serum, an increase in functional (e.g., wild-type) AAT concentration in serum, an increase in functional (e.g., wild-type) AAT concentration in serum, a decrease in protease activity in the lungs and/or liver, and/or a decrease in the risk of an AATD-related disease, such as lung disease or liver disease.

The pharmaceutical compositions described herein can include an effective amount of an engineered nuclease described herein, or a polynucleotide comprising a nucleic acid sequence encoding an engineered nuclease described herein. In some embodiments, the pharmaceutical composition comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of the encoding nucleic acid sequence. In some embodiments, the pharmaceutical composition comprises at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of the encoding nucleic acid sequence. In some embodiments, the pharmaceutical composition comprises about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of the encoding nucleic acid sequence. In certain embodiments, the pharmaceutical composition comprises about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of the encoding nucleic acid sequence.

The pharmaceutical compositions described herein can include an effective amount of a donor polynucleotide described herein, which comprises a template nucleic acid described herein. In some embodiments, the pharmaceutical composition comprises about 1×10¹⁰ gc/kg to about 1×10¹⁴ gc/kg (e.g., 1×10¹⁰ gc/kg, 1×10¹¹ gc/kg, 1×10¹² gc/kg, 1×10¹³ gc/kg, or 1×10¹⁴ gc/kg) of a template nucleic acid described herein. In some embodiments, the pharmaceutical composition comprises at least about 1×10¹⁰ gc/kg, at least about 1×10¹¹ gc/kg, at least about 1×10¹² gc/kg, at least about 1×10¹³ gc/kg, or at least about 1×10¹⁴ gc/kg of a template nucleic acid described herein. In some embodiments, the pharmaceutical composition comprises about 1×10¹⁰ gc/kg to about 1×10¹¹ gc/kg, about 1×10¹¹ gc/kg to about 1×10¹² gc/kg, about 1×10¹² gc/kg to about 1×10¹³ gc/kg, or about 1×10¹³ gc/kg to about 1×10¹⁴ gc/kg of a template nucleic acid described herein. In certain embodiments, the pharmaceutical composition comprises about 1×10¹² gc/kg to about 9×10¹³ gc/kg (e.g., about 1×10¹² gc/kg, about 2×10¹² gc/kg, about 3×10¹² gc/kg, about 4×10¹² gc/kg, about 5×10¹² gc/kg, about 6×10¹² gc/kg, about 7×10¹² gc/kg, about 8×10¹² gc/kg, about 9×10¹² gc/kg, about 1×10¹³ gc/kg, about 2×10¹³ gc/kg, about 3×10¹³ gc/kg, about 4×10¹³ gc/kg, about 5×10¹³ gc/kg, about 6×10¹³ gc/kg, about 7×10¹³ gc/kg, about 8×10¹³ gc/kg, or about 9×10¹³ gc/kg) of a template nucleic acid described herein.

In particular embodiments of the present disclosure, the pharmaceutical composition can comprise one or more mRNAs described herein encapsulated within lipid nanoparticles.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino) butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethy-1-(cis,cis-9′,1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid may comprise from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid may comprise from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof.

In some embodiments, the non-cationic lipid (e.g., one or more phospholipids and/or cholesterol) may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition may comprise amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Chol 3-β-[N—(N′,N′-dimethylmethane)carbamoyl] cholesterol, TC-Chol 3-β-[N—(N′,N′,N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18) 2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes may contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Particular examples are PEG-modified diacylglycerols and dialkylglycerols.

In some embodiments, the neutral lipids may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition that specifically enhances delivery and uptake in the liver, and specifically within hepatocytes.

In some embodiments, the pharmaceutical composition comprises an effective amount of a lipid nanoparticle formulation, wherein the lipid nanoparticle formulation comprises a polynucleotide (e.g., mRNA) comprising a nucleic acid sequence encoding an engineered nuclease described herein. In some examples, the lipid nanoparticle formulation comprises about 0.1 mg/kg to about 3 mg/kg of the polynucleotide (e.g., mRNA). In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the polynucleotide (e.g., mRNA). In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the polynucleotide (e.g., mRNA).

In some embodiments, the pharmaceutical composition comprises an effective amount of a lipid nanoparticle formulation comprising a donor polynucleotide comprising a template nucleic acid described herein, wherein lipid nanoparticle formulation comprises about 0.1 mg/kg to about 3 mg/kg of the donor polynucleotide. In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the donor polynucleotide. In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the donor polynucleotide.

In some embodiments, pharmaceutical compositions of the present disclosure can further comprise one or more additional agents useful in the treatment of AAT deficiency in the subject.

The present disclosure also provides engineered nucleases described herein, polynucleotides encoding engineered nucleases described herein, or polynucleotides comprising template nucleic acids described herein, for use as a medicament. The present disclosure further provides the use of engineered nucleases described herein, polynucleotide encoding engineered nucleases described herein, or polynucleotides comprising template nucleic acids described herein, in the manufacture of a medicament for treating AAT deficiency, for increasing levels of a functional (e.g., wild-type) AAT protein, or reducing one or more symptoms associated with AAT deficiency.

### 2.7 Methods for Producing Recombinant Viruses

In some embodiments, the present disclosure provides recombinant viruses, such as recombinant AAVs, for use in the methods of the present disclosure. Recombinant AAVs are typically produced in mammalian cell lines such as HEK-293. Because the viral cap and rep genes are removed from the recombinant virus to prevent its self-replication to make room for the therapeutic gene(s) to be delivered, it is necessary to provide these in trans in the packaging cell line. In addition, it is necessary to provide the “helper” (e.g. adenoviral) components necessary to support replication (Cots D et al., (2013) Curr. Gene Ther. 13 (5): 370-81). Frequently, recombinant AAVs are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAV particles are typically produced (manufactured) in cells, precautions must be taken in practicing the current invention to ensure that an engineered nuclease is not expressed in the packaging cells. Because the recombinant viral genomes of the present disclosure may comprise a recognition sequence for the nuclease, any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The nuclease can be placed under the control of a tissue-specific promoter that is not active in the packaging cells. Any tissue specific promoter described herein for expression of the engineered nuclease or for a nucleic acid sequence of interest can be used. For example, if a recombinant virus is developed for delivery of genes encoding an engineered nuclease to liver tissue, a liver-specific promoter can be used. Examples of liver-specific promoters include, without limitation, those liver-specific promoters described elsewhere herein.

Alternatively, the recombinant virus can be packaged in cells from a different species in which the nuclease is not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007), J. Biotechnol. 131 (2): 138-43). A nuclease under the control of a mammalian promoter is unlikely to be expressed in these cells (Airenne et al. (2013), Mol. Ther. 21 (4): 739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a nuclease. Because these introns are not spliced efficiently from pre-mRNA transcripts in insect cells, insect cells will not express a functional nuclease and will package the full-length genome. In contrast, mammalian cells to which the resulting recombinant AAV particles are delivered will properly splice the pre-mRNA and will express functional nuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1 (11): e57).

The engineered nuclease gene can be operably linked to an inducible promoter such that a small-molecule inducer is required for nuclease expression. Examples of inducible promoters include the Tet-On system (Clontech; Chen et al. (2015), BMC Biotechnol. 15(1): 4)) and the RheoSwitch system (Intrexon; Sowa et al. (2011), Spine, 36 (10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the embodiments of the present disclosure using such ligand-inducible transcription activators includes: 1) placing the engineered nuclease gene under the control of a promoter that responds to the corresponding transcription factor, the nuclease gene having (a) binding site(s) for the transcription factor; and 2) including the gene encoding the transcription factor in the packaged viral genome The latter step is necessary because the engineered nuclease will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAV particles are produced in a mammalian cell line that expresses a transcription repressor that prevents expression of the nuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the embodiments described herein, packaging cells are transfected/transduced with a vector encoding a transcription repressor and the nuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector; or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene will be repressed only in the packaging cells expressing the repressor and not in target cells or tissues transduced with the resulting recombinant AAV.

### 2.8 Variants

Embodiments of the present disclosure encompass engineered nucleases described herein, polynucleotides comprising a nucleic acid sequence encoding engineered nucleases described herein, donor polynucleotides comprising template nucleic acids described herein, and variants thereof.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; i.e., for a variant of an engineered nuclease described herein, the ability to bind and cleave a SERPINA1 gene recognition sequence described herein, and for a variant of a donor polynucleotide and template nucleic acid, the ability to insert the template nucleic acid into the engineered meganuclease cleavage site to generate a modified SERPINA1 gene that encodes a full-length and functional (e.g., wild-type) AAT protein. Such variants may result, for example, from human manipulation. Biologically active variants of a native polypeptide of the embodiments described herein, or a portion, domain, or subunit of a polypeptide of the embodiments, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide, as determined by sequence alignment programs and parameters described elsewhere herein. A biologically active variant of a polypeptide of the embodiments may differ from the native sequence by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In the context of variant polypeptides, “corresponding to” means that an amino acid residue in the variant polypeptide (e.g., a variant protein, engineered nuclease, or subunit) is the same amino acid residue (i.e., a separate identical residue) present in the parental polypeptide sequence (e.g., the parental protein, engineered nuclease, or subunit) in the same relative position (i.e., in relation to the remaining amino acids in the parent sequence). By way of example, if a parental engineered meganuclease sequence comprises a serine residue at position 26, a variant engineered meganuclease sequence that “comprises a residue corresponding to” residue 26 will also comprise a serine at a position that is relative (i.e., corresponding) to parental position 26.

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode various polypeptides of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its intended activity. For example, variants of a donor polynucleotide or template nucleic acid described herein would be screened for their ability to be inserted into a cleavage site within the SERPINA1 gene, inhibit expression of the endogenous mutant protein, and allow for expression of a functional (e.g., wild-type) AAT protein.

In some embodiments, the present disclosure provides variants of the donor polynucleotides, template nucleic acids, and components thereof, described herein. In certain embodiments, the donor polynucleotides, template nucleic acids, or components thereof, comprise a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to their native sequence.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Editing of AAT Recognition Sequences in a Reporter Cell Line

### 1. Methods and Materials

The purpose of this experiment was to determine whether AAT meganucleases could bind and cleave their respective human recognition sequences in mammalian cells. Each engineered meganuclease was evaluated using the CHO cell reporter assay previously described (see, WO/2012/167192). To perform the assays, CHO cell reporter lines were produced, which carried a non-functional Green Fluorescent Protein (GFP) gene expression cassette integrated into the genome of the cells. The GFP gene in each cell line contains a direct sequence duplication separated by a pair of recognition sequences such that intracellular cleavage of either recognition sequence by a meganuclease would stimulate a homologous recombination event resulting in a functional GFP gene.

In CHO reporter cell lines developed for this study, several recognition sequences were inserted into the GFP gene including the human AAT 33-34 (SEQ ID NO: 24), AAT 35-36 (SEQ ID NO: 26), AAT 37-38 (SEQ ID NO: 28), AAT 41-42 (SEQ ID NO: 30), and AAT 43-44 (SEQ ID NO: 32) recognition sequences. The second recognition sequence inserted into the GFP gene for each reporter cell line was a CHO-23/24 recognition sequence, which is recognized and cleaved by a control meganuclease called “CHO-23/24.” The CHO-23/24 recognition sequence is used as a positive control and standard measure of activity.

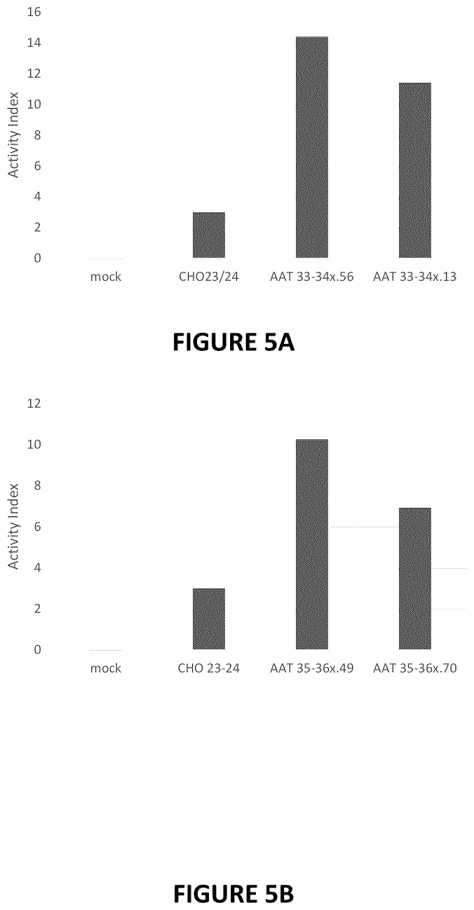

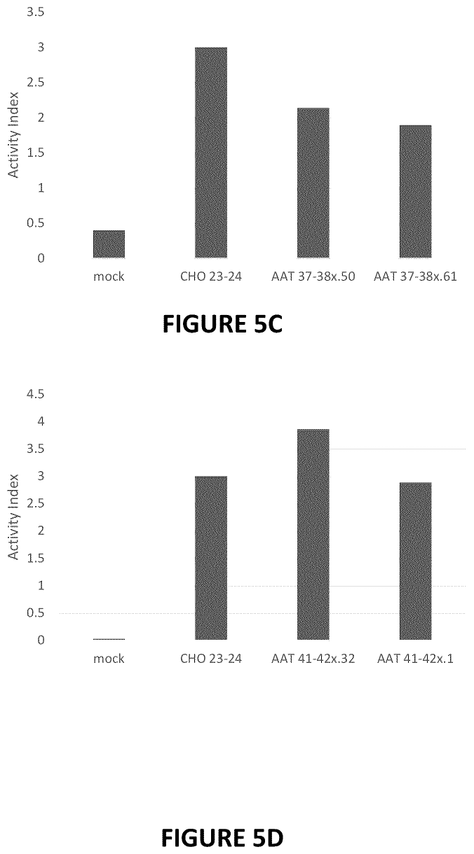

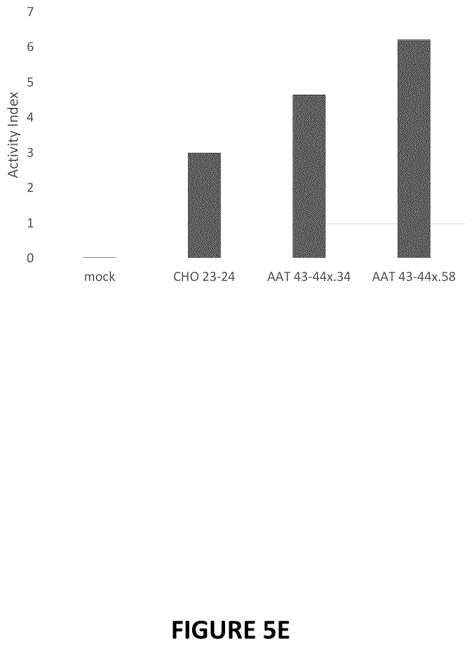

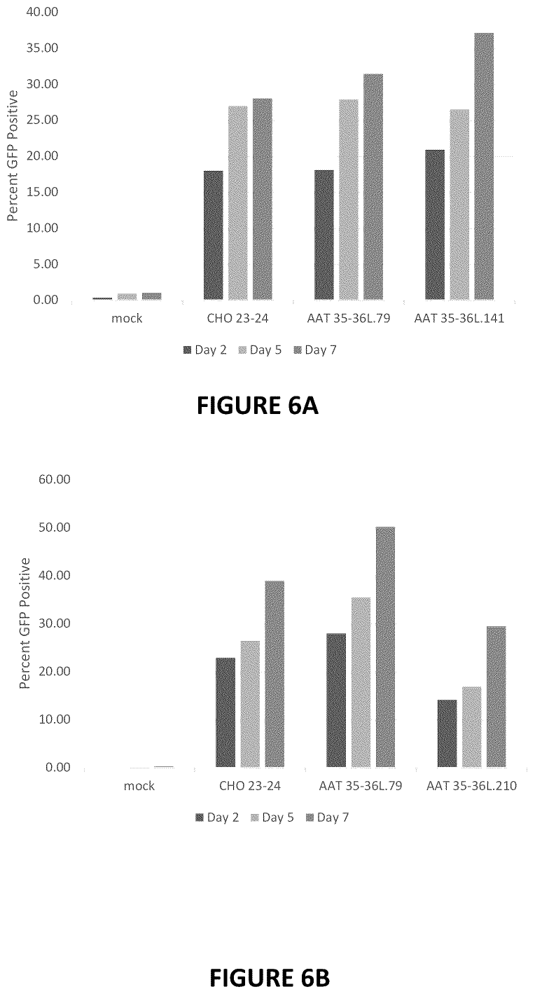

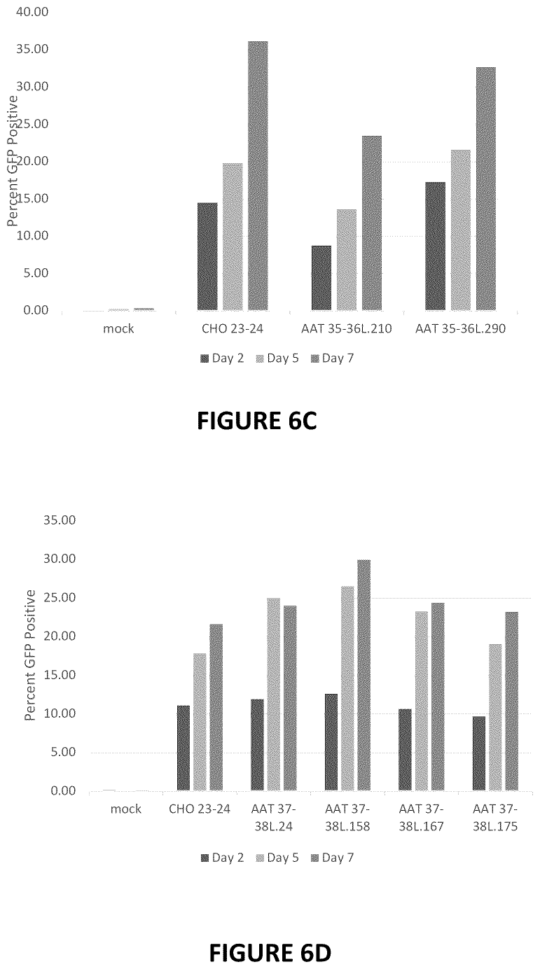

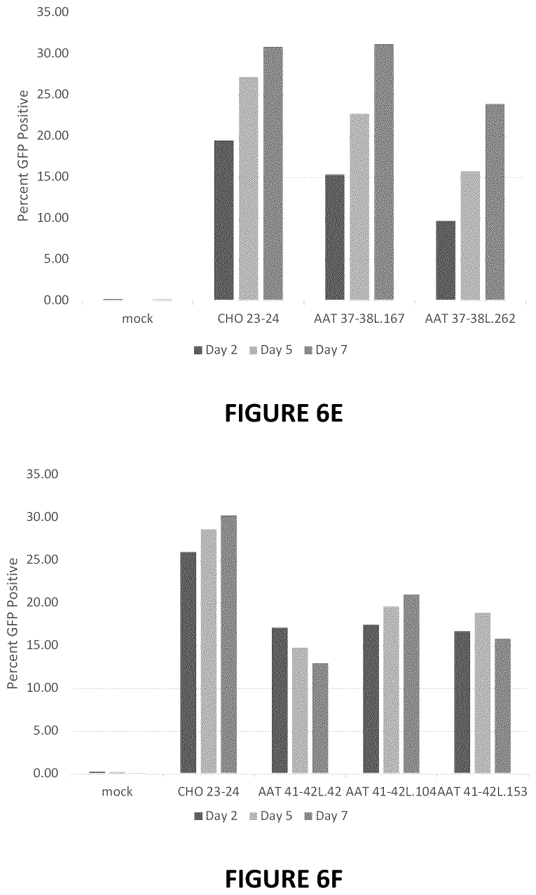

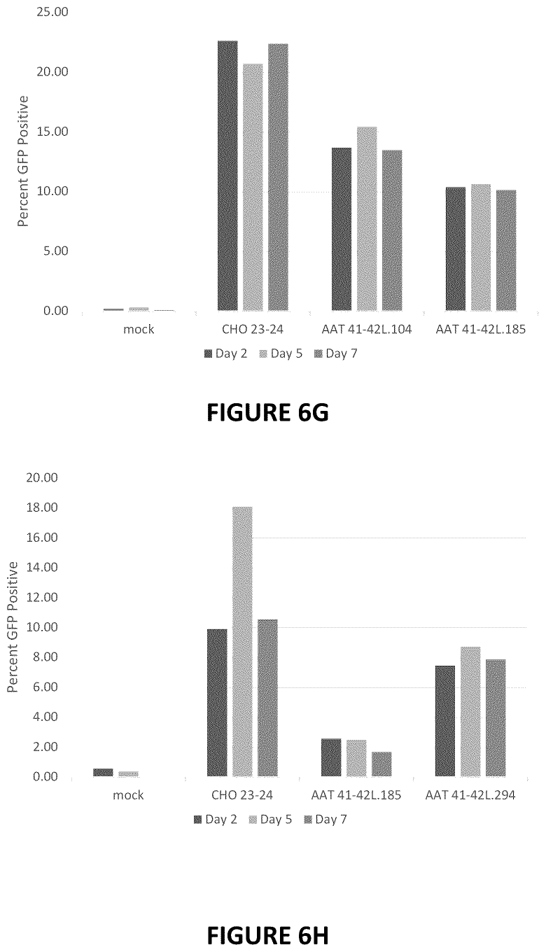

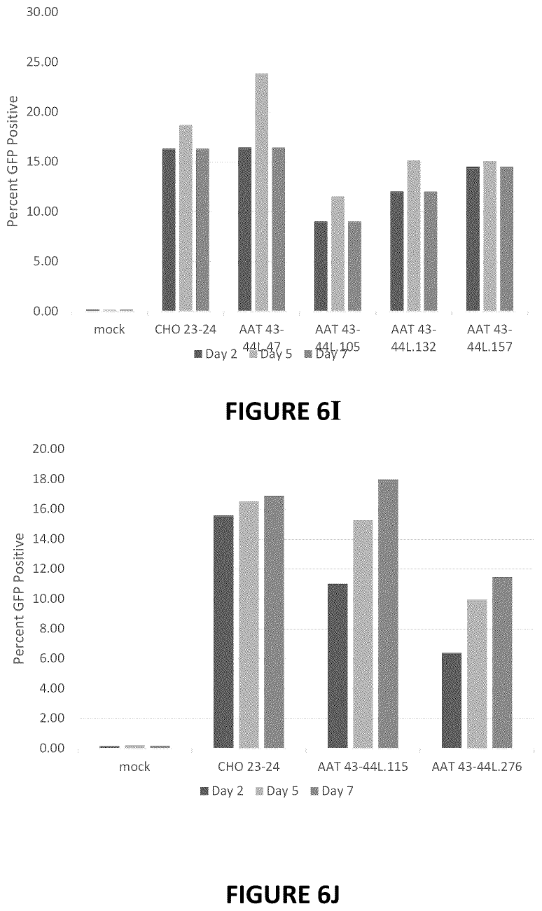

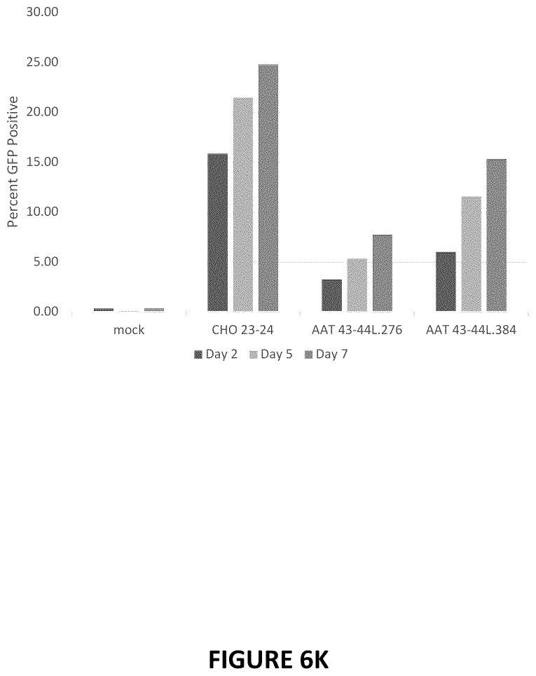

CHO reporter cells were transfected with mRNA encoding the indicated meganucleases shown in FIG. 5 and FIG. 6, which included an N-terminal SV40 nuclear localization sequence (SEQ ID NO: 17), which is included at the N-terminus of all the meganucleases described in the examples (unless otherwise noted). A control sample of CHO reporter cells were transfected with mRNA encoding the CHO-23/24 meganuclease. In each assay, 5e4 CHO reporter cells were transfected with 90 ng of mRNA in a 96-well plate using Lipofectamine® MessengerMax (ThermoFisher) according to the manufacturer's instructions. The transfected CHO cells were evaluated by flow cytometry at 2 days, 5 days, and 7 days post transfection to determine the percentage of GFP-positive cells compared to an untransfected negative control. Data obtained at each time point was normalized to the % GFP positive cells observed using the CHO-23/24 meganuclease to determine an “activity score,” and the normalized data from the earliest time point was subtracted from that of the latest time point to determine a “toxicity score.” The activity and toxicity scores were then added together to determine an “activity index.” The activity index for the indicated meganucleases is provided in FIG. 5 and the % GFP positive cells is provided in FIG. 6.

### 2. Results

As shown, the positive control CHO-23/24 exhibited an activity index of 3. Each of the indicated AAT meganucleases that bound and cleaved the AAT 33-34, AAT 35-36, AAT 41-42, and AAT 43-44 recognition sequences showed as high, or higher, activity in this assay compared to the CHO-23/24 positive control (FIGS. 5A, 5B, 5D, and 5E). The AAT 37-38 meganucleases exhibited a slightly lower activity index relative (approximately 2) to the positive control (FIG. 5C).

Additionally developed generations of meganucleases targeting the AAT 33-34, AAT 35-36, AAT 41-42, and AAT 43-44 recognition sequences were tested in this assay and the results are shown in FIG. 6A-FIG. 6K. The results demonstrated that these further optimized meganucleases developed against these sites were all able to bind and cleave their recognition sequence.

### 3. Conclusions

This assay demonstrated that successive generations of engineered AAT meganucleases could bind and cleave five different recognition sequences present in the human SERPINA1 gene.

### Example 2

### Editing of AAT Recognition Sequences in Human Cell Lines

### 1 Methods and Materials

These studies were conducted using in vitro cell-based systems to evaluate editing efficiencies of different AAT meganucleases targeting the AAT 33-34, AAT 35-36, AAT 37-38, AAT 41-42, and AAT 43-44 recognition sequences by digital PCR using an indel detection assay.

In these experiments, mRNA encoding the AAT 33-34x.13, AAT 33-34x.56, AAT 35-36x.49, AAT 35-36x.70, AAT 37-38x.50, AAT 37-38x.61, AAT 41-42x.1, AAT 41-42x.32, AAT 43-44x.34, and AAT 43-44x.58 meganucleases were electroporated into cells (Hep3B 50 ng or 5 ng) using the Lonza Amaxa 4D system. All meganucleases included an N-terminal SV40 NLS as described in Example 1.

Cells were collected at two days and six days post electroporation for gDNA preparation and evaluated for transfection efficiency using a Beckman Coulter CytoFlex S cytometer. Transfection efficiency exceeded 90%. Two additional time points were collected at between 4 and 9-days post electroporation for gDNA extractions. gDNA was prepared using the Macherey Nagel NucleoSpin Blood QuickPure kit.

Next generation sequencing was used to determine the frequency of insertions and deletions (indel). Genomic DNA was first obtained from Hep3B cells electroporated with or without meganuclease mRNA, and the isolated genomic DNA was used in a polymerase chain reaction (PCR) using primers spanning between ˜220-250 bp with the binding site located in the middle of the amplicon. The PCR 50 ul reaction consisted of repliQa Hifi Toughmix master mix (2×) (Hifi DNA polymerase, dNTPs, MgCl2) (QuantaBio), forward and reverse primers (10 uM) (see Table 1 below for barcoded primers) and genomic DNA (˜200 ng). PCR cycling conditions were as follows: 1 cycle of 98° C. for 2 minutes, 45 cycles of 98° C. for 10 seconds, 60° C. for 1 second, 1 cycle of 72° C. for 30 seconds and a 4° C. hold. Some of the PCR product (5 ul) was run on a 1% agarose gel containing ethidium bromide for DNA visualization and analyzed via a UVP Gel Studio (Analytik jena). Some of the PCR product (1 ul) was used for DNA concentration determination using a Quant-iT PicoGreen dsDNA Assay Kit (ThermoFischer) according to the manufacturer's instructions. Each sample (200 ng) was pooled into one sample for NGS analysis.

TABLE 1
Barcoded primers used for NGS sequencing analysis

| Primer | Barcoded sequence* | SEQUENCE ID |
|---|---|---|
| AAT 31-32 Fwd | TATCCAGTccaagctgtatacggatcacactg | SEQ ID NO: 36 |
| AAT 31-32 Fwd | ATGCACCTccaagctgtatacggatcacactg | SEQ ID NO: 37 |
| AAT 31-32 Fwd | CAGTTCCAccaagctgtatacggatcacactg | SEQ ID NO: 38 |
| AAT 31-32 Fwd | TCTCGCCTccaagctgtatacggatcacactg | SEQ ID NO: 39 |
| AAT 31-32 Rvs | GTGGTTCCtgaaccaggaactgtttcacctgcatc | SEQ ID NO: 40 |
| AAT 31-32 Rvs | CGAACTTCtgaaccaggaactgtttcacctgcatc | SEQ ID NO: 41 |
| AAT 31-32 Rvs | AGGAATGGtgaaccaggaactgtttcacctgcatc | SEQ ID NO: 42 |
| AAT 31-32 Rvs | GTTAGGAAtgaaccaggaactgtttcacctgcatc | SEQ ID NO: 43 |
| AAT 31-32 Rvs | CGTTGAGGtgaaccaggaactgtttcacctgcatc | SEQ ID NO: 44 |
| AAT 31-32 Rvs | CTCCCAAAtgaaccaggaactgtttcacctgcatc | SEQ ID NO: 45 |
| AAT 31-32 Rvs | GACATTTCtgaaccaggaactgtttcacctgcatc | SEQ ID NO: 46 |
| AAT 31-32 Rvs | TGAGAGATtgaaccaggaactgtttcacctgcatc | SEQ ID NO: 47 |
| AAT 33-34 Fwd | TATCCAGTctcttgaacctggccctagtgtc | SEQ ID NO: 48 |
| AAT 33-34 Fwd | ATGCACCTctcttgaacctggccctagtgtc | SEQ ID NO: 49 |
| AAT 33-34 Fwd | CAGTTCCActcttgaacctggccctagtgtc | SEQ ID NO: 50 |
| AAT 33-34 Fwd | TCTCGCCTctcttgaacctggccctagtgtc | SEQ ID NO: 51 |
| AAT 33-34 Rvs | GTGGTTCCaaattaaatccttccaacacttcagagatcaaggtc | SEQ ID NO: 52 |
| AAT 33-34 Rvs | CGAACTTCaaattaaatccttccaacacttcagagatcaaggtc | SEQ ID NO: 53 |
| AAT 33-34 Rvs | AGGAATGGaaattaaatccttccaacacttcagagatcaaggtc | SEQ ID NO: 54 |
| AAT 33-34 Rvs | GTTAGGAAaaattaaatccttccaacacttcagagatcaaggtc | SEQ ID NO: 55 |
| AAT 33-34 Rvs | CGTTGAGGaaattaaatccttccaacacttcagagatcaaggtc | SEQ ID NO: 56 |
| AAT 33-34 Rvs | CTCCCAAAaaattaaatccttccaacacttcagagatcaaggtc | SEQ ID NO: 57 |
| AAT 33-34 Rvs | GACATTTCaaattaaatccttccaacacttcagagatcaaggtc | SEQ ID NO: 58 |
| AAT 33-34 Rvs | TGAGAGATaaattaaatccttccaacacttcagagatcaaggtc | SEQ ID NO: 59 |
| AAT 35-36 Fwd | TATCCAGTctccattgtacagctatgaagctagtg | SEQ ID NO: 60 |
| AAT 35-36 Fwd | ATGCACCTctccattgtacagctatgaagctagtg | SEQ ID NO: 61 |
| AAT 35-36 Fwd | CAGTTCCActccattgtacagctatgaagctagtg | SEQ ID NO: 62 |
| AAT 35-36 Fwd | TCTCGCCTctccattgtacagctatgaagctagtg | SEQ ID NO: 63 |
| AAT 35-36 Rvs | GTGGTTCCgggagcataaaataagggaccaagagc | SEQ ID NO: 64 |
| AAT 35-36 Rvs | CGAACTTCgggagcataaaataagggaccaagagc | SEQ ID NO: 65 |
| AAT 35-36 Rvs | AGGAATGGgggagcataaaataagggaccaagagc | SEQ ID NO: 66 |
| AAT 35-36 Rvs | GTTAGGAAgggagcataaaataagggaccaagagc | SEQ ID NO: 67 |
| AAT 35-36 Rvs | CGTTGAGGgggagcataaaataagggaccaagagc | SEQ ID NO: 68 |
| AAT 35-36 Rvs | CTCCCAAAgggagcataaaataagggaccaagagc | SEQ ID NO: 69 |
| AAT 35-36 Rvs | GACATTTCgggagcataaaataagggaccaagagc | SEQ ID NO: 70 |
| AAT 35-36 Rvs | TGAGAGATgggagcataaaataagggaccaagagc | SEQ ID NO: 71 |
| AAT 37-38 Fwd | TATCCAGTtggcagataaagcgagactctgtc | SEQ ID NO: 72 |
| AAT 37-38 Fwd | ATGCACCTtggcagataaagcgagactctgtc | SEQ ID NO: 73 |
| AAT 37-38 Fwd | CAGTTCCAtggcagataaagcgagactctgtc | SEQ ID NO: 74 |
| AAT 37-38 Fwd | TCTCGCCTtggcagataaagcgagactctgtc | SEQ ID NO: 75 |
| AAT 37-38 Fwd | GCCGAATGtggcagataaagcgagactctgtc | SEQ ID NO: 76 |
| AAT 37-38 Fwd | TACTGCAGtggcagataaagcgagactctgtc | SEQ ID NO: 77 |
| AAT 37-38 Fwd | CATGTTGAtggcagataaagcgagactctgtc | SEQ ID NO: 78 |
| AAT 37-38 Fwd | ATAGAGTCtggcagataaagcgagactctgtc | SEQ ID NO: 79 |
| AAT 37-38 Fwd | TCACGCTCtggcagataaagcgagactctgtc | SEQ ID NO: 80 |
| AAT 37-38 Rvs | GTGGTTCCgggccagaggttgtggcttctag | SEQ ID NO: 81 |
| AAT 37-38 Rvs | CGAACTTCgggccagaggttgtggcttctag | SEQ ID NO: 82 |
| AAT 37-38 Rvs | AGGAATGGgggccagaggttgtggcttctag | SEQ ID NO: 83 |
| AAT 37-38 Rvs | GTTAGGAAgggccagaggttgtggcttctag | SEQ ID NO: 84 |
| AAT 37-38 Rvs | CGTTGAGGgggccagaggttgtggcttctag | SEQ ID NO: 85 |
| AAT 37-38 Rvs | CTCCCAAAgggccagaggttgtggcttctag | SEQ ID NO: 86 |
| AAT 37-38 Rvs | GACATTTCgggccagaggttgtggcttctag | SEQ ID NO: 87 |
| AAT 37-38 Rvs | TGAGAGATgggccagaggttgtggcttctag | SEQ ID NO: 88 |
| AAT 41-42 Fwd | TATCCAGTgctgactgcaggagcatcagc | SEQ ID NO: 89 |
| AAT 41-42 Rvs | GTGGTTCCacagaagggaaatgcatcttgcac | SEQ ID NO: 90 |
| AAT 41-42 Rvs | CGAACTTacagaagggaaatgcatcttgcac | SEQ ID NO: 91 |
| AAT 41-42 Rvs | AGGAATGGacagaagggaaatgcatcttgcac | SEQ ID NO: 92 |
| AAT 41-42 Rvs | GTTAGGAAacagaagggaaatgcatcttgcac | SEQ ID NO: 93 |
| AAT 41-42 Rvs | CGTTGAGGacagaagggaaatgcatcttgcac | SEQ ID NO: 94 |
| AAT 41-42 Rvs | CTCCCAAAacagaagggaaatgcatcttgcac | SEQ ID NO: 95 |
| AAT 41-42 Rvs | GACATTTCacagaagggaaatgcatcttgcac | SEQ ID NO: 96 |
| AAT 41-42 Rvs | TGAGAGATacagaagggaaatgcatcttgcac | SEQ ID NO: 97 |
| AAT 43-44 Fwd | TATCCAGTaggcccattcctcttcttgtgc | SEQ ID NO: 98 |
| AAT 43-44 Fwd | ATGCACCTaggcccattcctcttcttgtgc | SEQ ID NO: 99 |
| AAT 43-44 Fwd | CAGTTCCAaggcccattcctcttcttgtgc | SEQ ID NO: 100 |
| AAT 43-44 Fwd | TCTCGCCTaggcccattcctcttcttgtgc | SEQ ID NO: 101 |
| AAT 43-44 Rvs | GTGGTTCCcaatcccgtgaggtgctaatgc | SEQ ID NO: 102 |
| AAT 43-44 Rvs | CGAACTTcaatcccgtgaggtgctaatgc | SEQ ID NO: 103 |
| AAT 43-44 Rvs | AGGAATGGcaatcccgtgaggtgctaatgc | SEQ ID NO: 104 |
| AAT 43-44 Rvs | GTTAGGAAcaatcccgtgaggtgctaatgc | SEQ ID NO: 105 |
| AAT 43-44 Rvs | CGTTGAGGcaatcccgtgaggtgctaatgc | SEQ ID NO: 106 |
| AAT 43-44 Rvs | CTCCCAAAcaatcccgtgaggtgctaatgc | SEQ ID NO: 107 |
| AAT 43-44 Rvs | GACATTTCcaatcccgtgaggtgctaatgc | SEQ ID NO: 108 |
| AAT 43-44 Rvs | TGAGAGATcaatcccgtgaggtgctaatgc | SEQ ID NO: 109 |

*the uppercase portion of primer indicates the 5′ barcode sequence for each primer.

### 2. Results

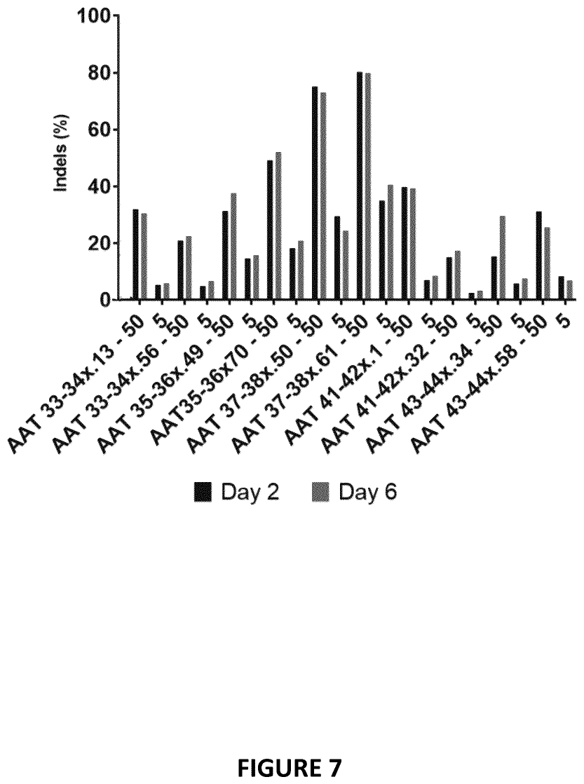

Indel frequencies varied between the AAT meganucleases tested in this study. The AAT 35-36 and AAT 37-38 meganucleases demonstrated the highest indel potency at days 2 and 6 at both the 5 ng and 50 ng doses. AAT 37-38x.61 exhibited the highest indel frequency with 80% indels by day 6 at the 50 ng dose. The AAT 41-42x.32 meganuclease gave the lowest indel frequency at 17% indels by day 6 at the 50 ng dose (FIG. 7).

### 3. Conclusions

The AAT meganucleases evaluated in this study demonstrated indel frequencies between 3% and 40% at the 5 ng dose, and between 15% and 80% at the 50 ng dose. The meganuclease with the highest indel percentage for each binding site was chosen to be further engineered for increased activity and target specificity, while maintaining or improving indel potency. The following meganucleases were chosen for further development: AAT 33-34x.13 and AAT 33-34x.56, AAT 35-36x.70, AAT 37-38x.50, AAT 41-42x.1 and AAT 43-44x.58.

### Example 3

### 1. Methods and Materials

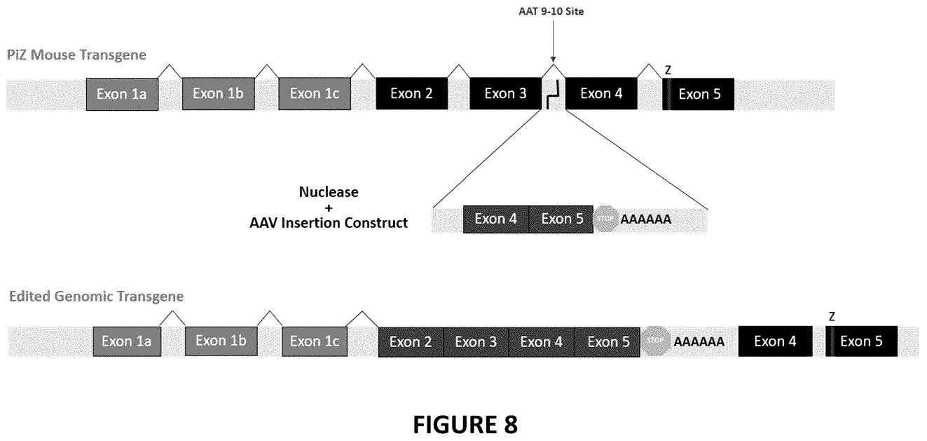

The NSG-Tg (SERPINA*E342K)Z11.03Slcw transgenic mice, abbreviated PiZ, were obtained from The Jackson Laboratory. This transgenic model contains the Z mutant allele of the human alpha1-antitrypsin (AAT) transgene, SERPINA1, which allows for human Z-AAT protein expression. PiZ mice contain numerous Z-AAT transgenes (>10), and this number is variable with age. This mouse model was utilized to determine if it was possible to cut within intron 3 of the endogenous mutant Z-AAT allele and insert exons 4 and 5 of the WT SERPINA1 CDS off of a promoterless AAV8 vector, thereby expressing a WT AAT protein in place of the mutant Z-AAT protein. A schematic of the gene editing approach is provided in FIG. 8. The repair WT AAT sequence contains both a stop codon and poly A signal at the N-terminus, preventing the endogenous Z-AAT allele from being expressed. In addition, a flag tag was added for histological detection of WT AAT expressed by the inserted donor polynucleotide.

A proof-of-concept meganuclease was designed to bind and cleave a recognition sequence, referred to as AAT 9-10 (SEQ ID NO: 18), located within intron 3 of the SERPINA1 gene. This engineered meganuclease is referred to as AAT 9-10x.311. The coding sequence of the AAT 9-10x.311 meganuclease was packaged as a single stranded transgene in an AAV8 capsid and was operably linked to the liver-specific promoter human thyroxine-binding globulin (TBG). The AAV8 serotype was chosen due to extensive data showing liver tropism in monkeys and humans, along with many current liver-directed gene therapy clinical trials using AAV8 showing its safety and tolerability.

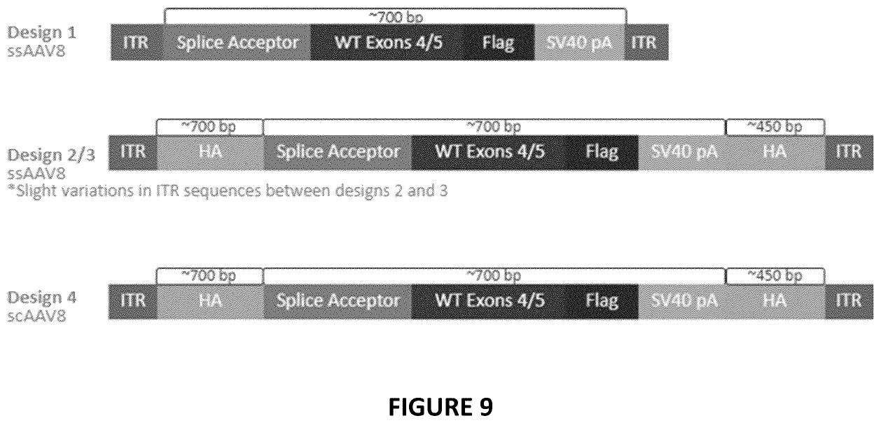

A second AAV8 vector utilized in this study comprised one of four wild-type AAT (WT AAT) donor polynucleotides in its viral genome. As shown in FIG. 9, each WT AAV donor polynucleotide comprised a template nucleic acid that included, from 5′ to 3′, (i) a transferrin intron sequence (i.e., a splicing sequence), which comprises a splice acceptor sequence that can pair with the endogenous splice donor sequence that is downstream and adjacent to exon 3 of the SERPINA1 gene, (iii) exons 4 and 5 of the wild-type human SERPINA1 gene, (iii) a flag tag, and (iv) a termination sequence that included a stop codon and an SV40 polyA sequence. Three of the tested constructs included homology arms flanking the template nucleic acid (˜700 bp 5′ homology arm and ˜450 bp 3′ homology arm; designs 2-4), while and one construct was designed without homology arms to be used as a control for NHEJ-driven insertion frequencies (design 1). The included homology arms had homology to sequences upstream and downstream of the expected cleavage site in the AAT 9-10 recognition sequence. Two of the constructs that included homology arms were packaged as single-stranded genomes (rather than self-complimentary) and varied in the 5′ and 3′ ITR sequences flanking the homology arms (designs 2 and 3). One of the constructs that included homology arms was packaged as a self-complimentary genome (design 4).

PiZ mice were either administered PBS and one of the WT AAT repair AAV8 vectors, or they were co-administered two AAV8 vectors, one encoding the engineered AAT 9-10 meganuclease and one comprising one of the four designed WT AAT repair sequences, via retro-orbital (RO) injections at doses 5e12 VG/kg and 2.5e13 VG/kg, respectively (Table 2). Blood was collected in EDTA tubes from mice at weekly intervals starting at study day-14 and continuing through necropsy at study day 42. The blood was centrifuged at 15,000×g for 5 minutes to isolate plasma, which was placed at −80° C. for mass spectrometry analysis. Livers were harvested at necropsy for genomic analysis. Briefly, animals were perfused with PBS, and a piece of the left median lobe of the liver was placed in RNAlater. A piece was also flash frozen and placed in a freezer set to maintain between −70° C. and −80° C. Liver tissue placed into RNAlater were held overnight at 4° C. and then transferred to a freezer set to maintain a temperature between −70° C. and −80° C. Genomic DNA was extracted from liver samples using the NucleoSpin Tissue kit (Macherey-Nagel) and used for AAT copy number, indel and insertion frequency analysis (ddPCR).

For analysis of human Z-AAT transgene copy number in PiZ mice livers at study day 40, primers P1, F1, and R1 were used to generate a reference amplicon in the human Z-AAT transgene at a recognition site referred to as AAT 13-14 (SEQ ID NO: 20), as well as P2, F2, and R2 to generate a reference amplicon at a meganuclease recognition sequence in the mouse transthyretin gene referred to as TTR 5-6 (SEQ ID NO: 34). Amplifications were multiplexed in a 20 uL reaction containing 1×ddPCR Supermix for Probes (no dUTP, BioRad), 250 nM of each probe, 900 nM of each primer, 5U of HindIII-HF, and about 10 ng cellular gDNA. Droplets were generated using a QX100 droplet generator (BioRad). Cycling conditions for this assay were as follows: 1 cycle of 95° C. (2° C./s ramp) for 10 minutes, 44 cycles of 94° C. (2° C./s ramp) for 10 seconds, 56° C. (2° C./s ramp) for 30 seconds, 72° C. (2° C./s ramp) for 1 minute, 1 cycle of 98° C. for 10 minutes, 4° C. hold.

For ddPCR analysis of functional human WT AAT insertions at the AAT 9-10 site, per copy of human Z-AAT allele, the primers, P3, F3, and R3 at the junction of AAT 9-10 insertion and chromosomal PiZ mouse DNA and primers P1, F1, and R1 were used at the AAT 13-14 site. Amplifications were multiplexed in a 20 uL reaction containing 1×ddPCR Supermix for Probes (no dUTP, BioRad), 250 nM of each probe, 900 nM of each primer, 5U of HindIII-HF, and about 20 ng cellular gDNA. Droplets were generated using a QX100 droplet generator (BioRad). Cycling conditions for this assay were as follows: 1 cycle of 95° C. (2° C./s ramp) for 10 minutes, 44 cycles of 94° C. (2° C./s ramp) for 10 seconds, 59.2° C. (2° C./s ramp) for 30 seconds, 72 C (0.22° C./s ramp) for 1 minute, 1 cycle of 98° C. for 10 minutes, 4° C. hold.

For ddPCR analysis of human WT AAT insertions per diploid cell, primers, P3, F3, and R3 were used at the AAT9-10 site and primers P2, F2, and R2 at the mouse 5-6 TTR site (Table 3). To determine the total insertion (functional and non-functional) and deletion frequency at the AAT 9-10 site, primers P4, F4, and R4 at the AAT 9-10 recognition site. Amplifications were multiplexed in a 20 uL reaction containing 1×ddPCR Supermix for Probes (no dUTP, BioRad), 250 nM of each probe, 900 nM of each primer, 5U of HindIII-HF, and about 10 ng cellular gDNA. Droplets were generated using a QX100 droplet generator (BioRad). Cycling conditions for this assay were as follows: 1 cycle of 95° C. (2° C./s ramp) for 10 minutes, 44 cycles of 94° C. (2° C./s ramp) for 10 seconds, 56° C. (2° C./s ramp) for 30 seconds, 72 C (2° C./s ramp) for 1 minute, 1 cycle of 98° C. for 10 minutes, 4° C. hold.

Primer sequences referred to above are provide in Table 3 below. Droplets were analyzed using a QX200 droplet reader (BioRad) and QuantaSoft analysis software (BioRad) was used to acquire and analyze data. Isolated plasma was analyzed by mass spectrometry analysis for mutant and total AAT protein expression.

TABLE 2
PiZ Mouse Study #1 Design

| | | AAV8 Gene | |
|---|---|---|---|
| | | Insertion | |
| Group # | N | Construct | PBS/AAV8 Meganuclease |
| 1 | 2* | Design 1 | PBS |
| | 3 | | SSAAV8 AAT9-10x.311 |
| 2 | 2 | Design 2 | PBS |
| | 3 | | SSAAV8 AAT9-10x.311 |
| 3 | 2 | Design 3 | PBS |
| | 3 | | ssAAV8 AAT9-10x.311 |
| 4 | 2 | Design 4 | PBS |
| | 3** | | scAAV8 AAT9-10x.311 |

*One mouse in the PBS treated Group 1 was euthanized 3 weeks post-AAV treatment (not test-article related)
*One mouse in the meganuclease treated Group 4 was euthanized 1 week post-AAV treatment (not test article related).

TABLE 3
Primers used for ddPCR analysis

| Primer | | |
|---|---|---|
| Name | Primer Sequence | SEQ ID NO: |
| P1 | CTGCTCAGGCATCTGCATTTCCC | SEQ ID NO: 110 |
| F1 | ATGCCTCGATGGTGAAG | SEQ ID NO: 111 |
| R1 | GGCAAACATTCTGCTTACTATT | SEQ ID NO: 112 |
| P2 | CCTCGCTGGACTGGTATTTGTGTCT | SEQ ID NO: 113 |
| F2 | GACAGGATGGCTTCCCTTC | SEQ ID NO: 114 |
| R2 | GTCTAACTGCCATGTCTGGAT | SEQ ID NO: 115 |
| P3 | ACCTTAGTGATGCCCAGTTGACCC | SEQ ID NO: 116 |
| F3 | CCACAACTAGAATGCAGTGAA | SEQ ID NO: 117 |
| R3 | GAATCACGGGCATCTTCAG | SEQ ID NO: 118 |
| P4 | CTGCCCGCTCACACCAGAC | SEQ ID NO: 119 |
| F4 | AGCACCTCCTGAGGTC | SEQ ID NO: 120 |
| R4 | CACACCAGGCTGAGTG | SEQ ID NO: 121 |

### 2. Results

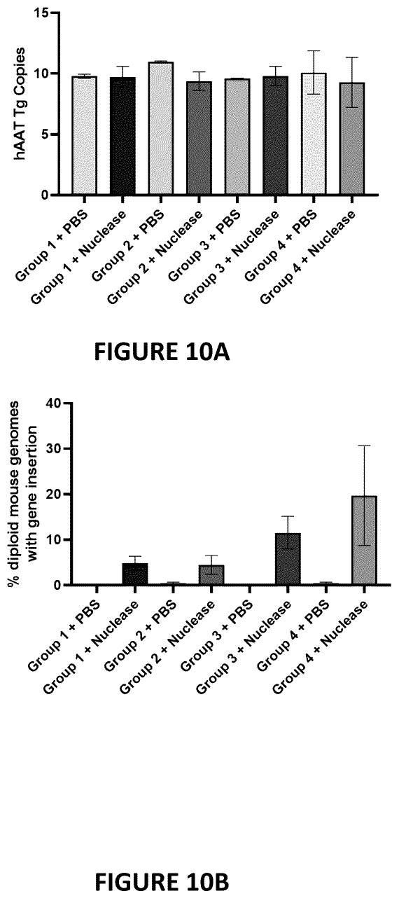

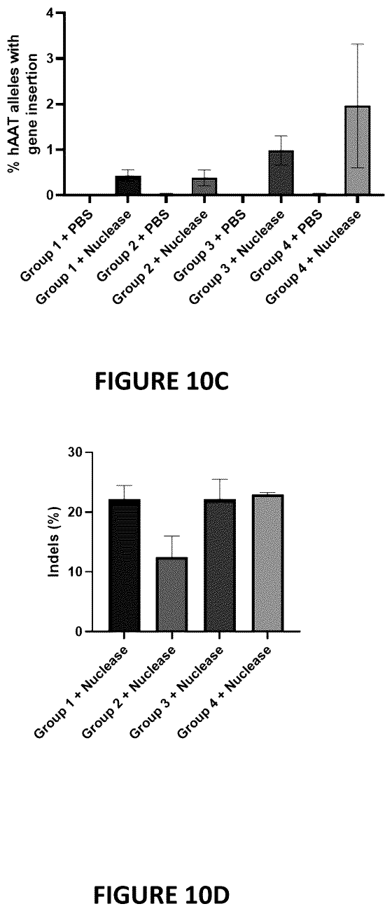

The ddPCR analysis of genomic DNA from mouse liver determined that the hAAT copy number was the same regardless of treatment group at approximately 10 transgene copies per cell at 6 weeks post AAV injections (FIG. 10A). Insertion frequencies of the WT AAT repair sequence were highest in group 4 treated with the scAAV vector with an average insertion rate of 2% of all human AAT transgenes and ˜20% per diploid cell (FIG. 10B and FIG. 10C). The single stranded AAV with ITR sequence B (ITRB) (design 3) had the second highest insertion frequency at ˜1% and ˜10% average insertion rate per transgene copy and diploid cell, respectively. The single stranded AAV construct without homology arms and ITR sequence A (ITRA) (design 1) and with homology arms and ITRA (design 2) had the lowest insertion frequencies at ˜0.5% and 5% per transgene copy and diploid cell, respectively (FIG. 10B and FIG. 10C). Indel analysis showed that groups 1, 3, and 4 all had similar indel levels at 6 weeks post administration of the AAT 9-10 meganuclease with ˜20% indels, while group 2 demonstrated only ˜10% indels (FIG. 10D). Both insertions of the WT AAT genomic sequence and indels would have been picked up by this assay.

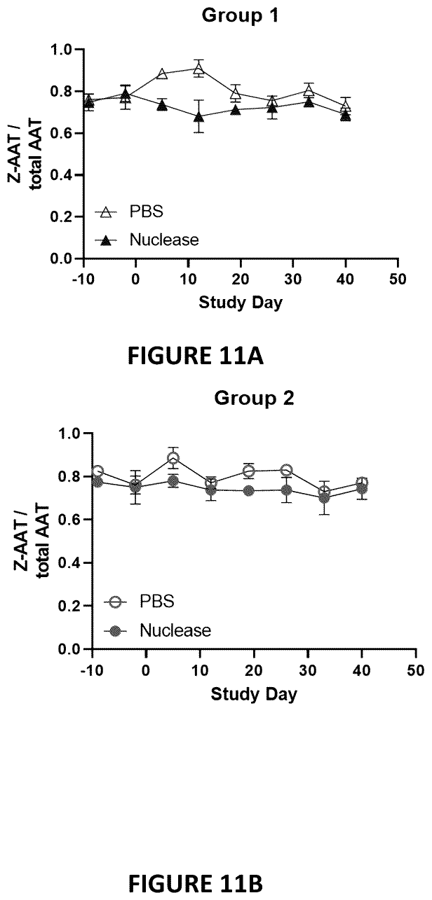

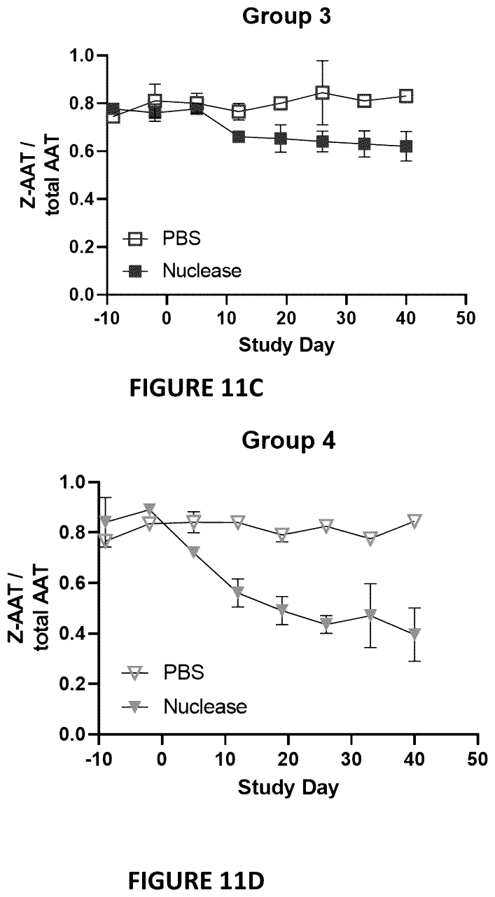

Mass spectrometry was used to determine the level of WT AAT protein circulating in the blood at weekly intervals post AAV injection. WT AAT protein levels were calculated by measuring the loss of the Z allele. Because the meganuclease cuts in an intron, interruption of the Z protein should only occur if a sequence is inserted into the endogenous AAT Z locus and the total protein being made should stay the same. The Z-AAT protein divided by the AAT total protein was calculated for each mouse sample and expressed as a ratio to normalize for the natural decrease of Z-AAT overtime in this model. Therefore, a decrease in this ratio likely indicates an increase in WT protein in the blood. Groups 1 and 2 did not show a significant decrease in Z-AAT through each time point in the study (FIG. 11A and FIG. 11B). Group 4 demonstrated the largest decrease in this ratio at ˜50% by study day 40 of the PBS and pre-dose groups. Group 3 had the second largest decrease in the mutant over total ratio with an average of 20% decrease in Z-AAT protein at study day 40 (FIG. 11C and FIG. 11D). These data are consistent with the insertion data with group 4 having the best insertion of the WT AAT sequence, followed by group 3, and groups 1 and 2 showed similar trends with minimal insertion of the WT AAT sequence.

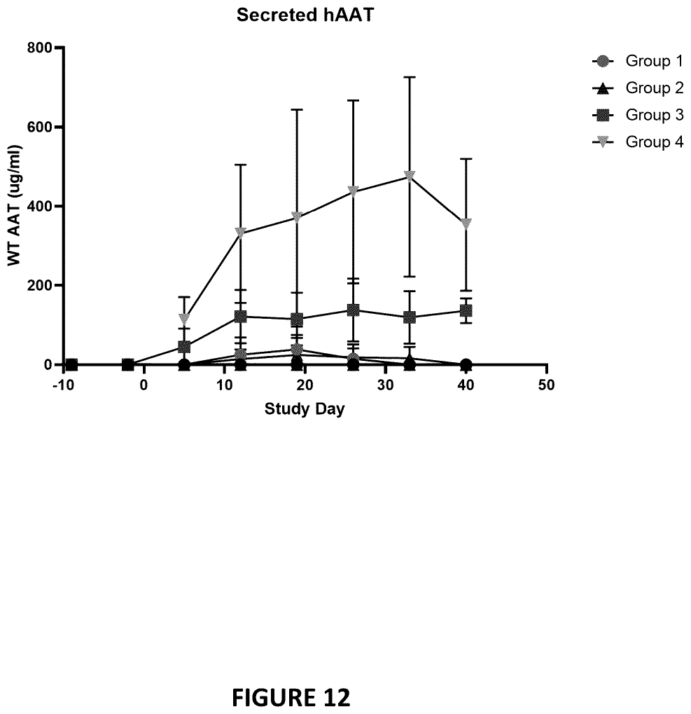

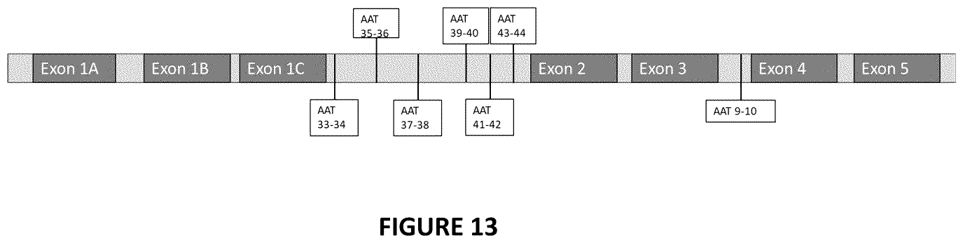

Next, the WT AAT protein was measured directly to determine if the WT AAT protein was indeed being expressed in the blood. The mice in group 4 showed the highest circulating WT AAT protein from week 1 through week 6 with a maximum average of 474 μg/ml of WT AAT protein at study day 33 (FIG. 12). WT AAT was also circulating in group 3 mice throughout the study, but to a lesser extent than group 4, with a maximum average of 129 μg/ml of WT AAT protein at study day 26 (FIG. 12). Groups 1 and 2 had only one animal show circulating WT AAT protein with a maximum average expression of 58 μg/ml on study days 12 and 19, respectively. This data confirmed that WT AAT protein is circulating in PiZ mice after treatment with an AAT gene targeting meganuclease and WT AAT repair template.

### 3. Conclusions

The results from this study demonstrated the ability to insert a portion of the WT AAT coding sequence into an endogenous mutant Z-AAT locus by co-administering two AAVs separately expressing an engineered meganuclease and a WT AAT repair template, such that the WT AAT repair template is inserted into the endogenous SERPINA1 gene and is operably linked to the endogenous SERPINA1 promoter, and that a full-length WT AAT protein is expressed in the PiZ mice.

### Example 4

### Additional In Vivo AAT Gene Editing in a PiZ AAT Mouse Model

### 1. Methods and Materials

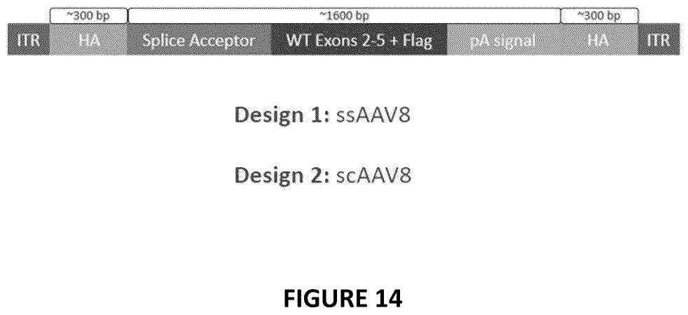

The engineered meganuclease AAT binding site of Example 3 (AAT 9-10) was found to be highly methylated, and therefore, potentially creating epigenetic interference with meganuclease binding and cutting. Thus, additional engineered meganucleases were designed to target new sites within the SERPNA1 gene. All of the new engineered meganucleases target intron 3, which is adjacent to and 3′ downstream of exon 1c, which contains the hepatocyte specific promoter. Furthermore, AAT donor polynucleotides were developed that comprise AAT coding exons 2-5, along with a flag tag, stop codon, and poly-A tail. In this study, PiZ mice were administered PBS or an AAV8 vector encoding the following engineered meganucleases, which were previously described in Example 1: AAT 33-34x.13, AAT 35-36x.70, AAT 37-38x.50, or AAT 43-44x.58. Each meganuclease AAV was administered with PBS or with a second AAV8 vector comprising an AAT donor polynucleotide. PiZ mice were either administered PBS and one of the WT AAT repair AAV8 vectors, or they were co-administered two AAV8 vectors, one encoding the engineered AAT meganuclease and one comprising one of the four designed WT AAT repair sequences, via retro-orbital (RO) injections at doses 2.5e12 VG/kg and 1.25e13 VG/kg, respectively. In one arm of the study, the AAT donor polynucleotide was packaged as either an scAAV8 or a ssAAV8, and will include ˜300 bp homology arms on either end of the transgene (FIG. 14, Table 4). The scAAV8 and ssAAV8 repair constructs were chosen due to their superior insertion frequency and production of WT AAT protein in PiZ mice as described in Example 3. The repair constructs were designed with homology arms for their corresponding binding sites (i.e., AAT 33-34, AAT 35-36, AAT 37-38, or AAT 43-44) with a size of ˜300 bp due to the packaging limitations of scAAV (˜2.4 kb), which allowed for the WT AAT coding repair construct to be within these limitations. Blood was taken from mice at weekly intervals from day-14 through day 42 and plasma was isolated and analyzed via mass spectrometry as previously described in Example 3. Liver tissue was harvested from the mice at day 42 and DNA was isolated from tissue as described in Example 3. Insertion analysis was performed by ddPCR with the following ddPCR parameters: 1 cycle of 95° C. for 10 minutes, 44 cycles of 94° C. for 10 seconds, 57° C. for 30 seconds and 72° C. for 4 minutes, 1 cycle of 98° C. for 10 minutes and a 4° C. hold using the primers and probes in Table 5, assays 1, 2, 3 and 5. AAT transgene copy number analysis was performed as described in Example 3. In addition, immunohistochemistry was performed on liver tissue. Briefly, liver tissue was placed in 10% neutral buffer formalin (NBF) for at least 24 hours and subsequently transferred into 70% ethanol for at least 24 hours and then processed to paraffin blocks for analysis. The following histological analyses was performed: PAS-D staining for Z-globules and flag staining for detection of WT AAT expression from the inserted donor polynucleotide.

TABLE 4
PiZ Mouse Study #2 Design

| | | | PBS/AAV8 |
|---|---|---|---|
| Group# | N | AAV8 Gene Insert Construct Design | meganuclease |
| 1 | 2 | Design 1 (single stranded), BS 33-34 | PBS |
| 2 | 3 | | AAT 33-34x.13 |
| 3 | 3 | BS 35-36 | AAT 35-36x.70 |
| 4 | 3 | BS 37-38 | AAT 37-38x.50 |
| 5 | 3 | BS 43-44 | AAT 43-44x.58 |
| 6 | 2 | Design 2 (self-complementary), BS | PBS |
| 7 | 3 | 33-34 | AAT 33-34x.13 |
| 8 | 3 | BS 35-36 | AAT 35-36x.70 |
| 9 | 3 | BS 37-38 | AAT 37-38x.50 |
| 10 | 3 | BS 43-44 | AAT 43-44x.58 |
| 11 | 2 | PBS | PBS |

The AAV repair template with homology arms used in this study can be inserted into a cut site via homologous directed repair (HDR) or non-homologous end joining (NHEJ). HDR results in correct gene orientation insertion that produces protein or a productive insertion. NHEJ can result in the repair construct being inserted in the forward or reverse orientation and only the forward orientation will result in a productive insertion. The ddPCR assay we used to detect HDR and forward NHEJ measures total productive insertions (Table 5, Assays 1-5). In order to determine the repair mechanism, HDR or NHEJ, of the productive insertions we used the NHEJ reverse (non-productive insertion) assay (Table 5, Assays 6-10). Based on the hypothesis that NHEJ insertion results in 50% forward and 50% reverse gene orientation, we used the NHEJ reverse assay to estimate the ratio of NHEJ forward and HDR insertions from the productive insertions assay.

TABLE 5
Primer and Probe Sequences for Productive and non-Productive Insertion
Assays

| | | | |
|---|---|---|---|
| Assay | | | |
| No. | Primer/Probe | Sequence | SEQ ID NO: |

ddPCR HDR/NHEF Forward (Productive) Insertions

| | | | |
|---|---|---|---|
| 1 | AAT33-34 Fwd | TGGACAAACCACAACTAGAA | 122 |
| 1 | AAT33-34 Rvs | AGCCTGGCTAAATGAAGAG | 123 |
| 1 | AAT33-34 Probe | AGCAAGTTGACGTGGAGCAATCTG | 124 |
| 2 | AAT35-36 Fwd | TGGACAAACCACAACTAGAA | 125 |
| 2 | AAT35-36 Rvs | CTCCCAAGCTGTTCCTTAT | 126 |
| 2 | AAT35-36 Probe | AGGGTGCTGCCTCTGATAGAAGG | 127 |
| 3 | AAT37-38 Fwd | TGGACAAACCACAACTAGAA | 128 |
| 3 | AAT37-38 Rvs | CCACAGTGATCCTTCTACTC | 129 |
| 3 | AAT37-38 Probe | TCTGTGAAGACGGCAGGTTCTACC | 130 |
| 4 | AAT41-42 Fwd | TGGACAAACCACAACTAGAA | 131 |
| 4 | AAT41-42 Rvs | CGTGAGGTGCTAATGCTAATA | 132 |
| 4 | AAT41-42 Probe | AGCTGACCTAATCTCTCTGGCTTTGG | 133 |
| 5 | AAT43-44 Fwd | TGGACAAACCACAACTAGAA | 134 |
| 5 | AAT43-44 Rvs | CTGCAAGACAGAGATGGG | 135 |
| 5 | AAT43-44 Probe | AGTCATCATGTGCCTTGACTCGG | 136 |

ddPCR NHEJ Reverse(non-Productive) Insertions

| | | | |
|---|---|---|---|
| 6 | AAT33-34 Fwd | GAGAGAACGCACCACTTTAC | 137 |
| 6 | AAT33-34 Rvs | AGCCTGGCTAAATGAAGAG | 138 |
| 6 | AAT33-34 Probe | AGCAAGTTGACGTGGAGCAATCTG | 139 |
| 7 | AAT35-36 Fwd | GAGAGAACGCACCACTTTAC | 140 |
| 7 | AAT35-36 Rvs | CTCCCAAGCTGTTCCTTAT | 141 |
| 7 | AAT35-36 Probe | AGGGTGCTGCCTCTGATAGAAGG | 142 |
| 8 | AAT37-38 Fwd | GAGAGAACGCACCACTTTAC | 143 |
| 8 | AAT37-38 Rvs | CCACAGTGATCCTTCTACTC | 144 |
| 8 | AAT37-38 Probe | TCTGTGAAGACGGCAGGTTCTACC | 145 |
| 9 | AAT41-42 Fwd | GAGAGAACGCACCACTTTAC | 146 |
| 9 | AAT41-42 Rvs | CGTGAGGTGCTAATGCTAATA | 147 |
| 9 | AAT41-42 Probe | AGCTGACCTAATCTCTCTGGCTTTGG | 148 |
| 10 | AAT43-44 Fwd | GAGAGAACGCACCACTTTAC | 149 |
| 10 | AAT43-44 Rvs | CTGCAAGACAGAGATGGG | 150 |
| 10 | AAT43-44 Probe | AGTCATCATGTGCCTTGACTCGG | 151 |

### 2. Results

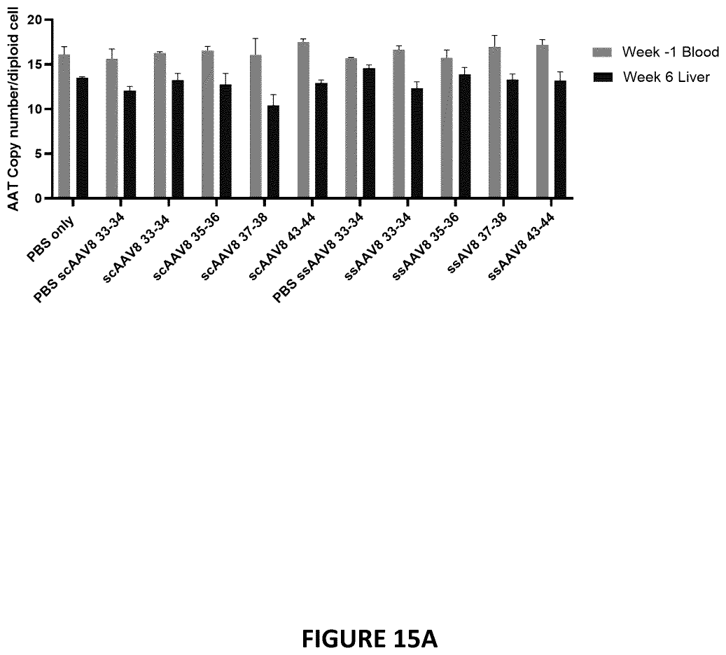

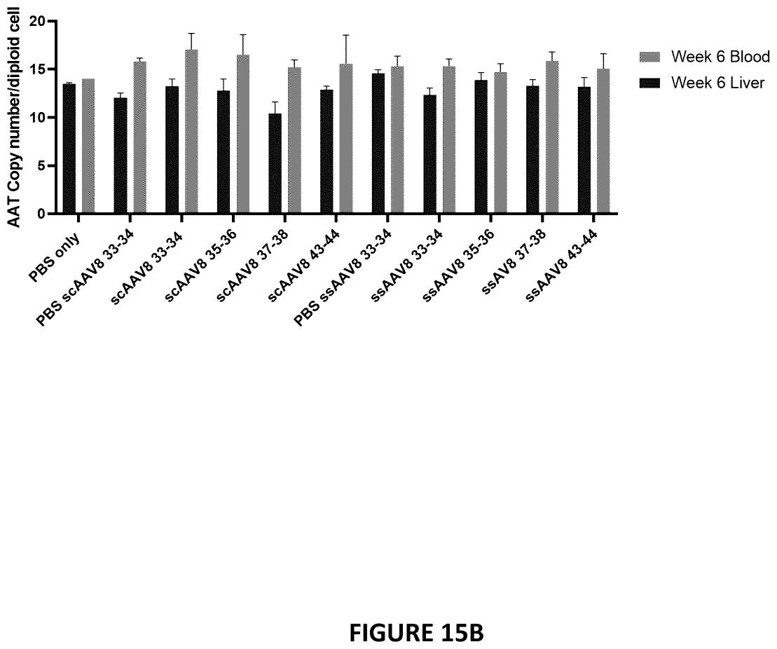

The SERPINA1 transgene copy number was analyzed in PiZ mouse blood from pre-treatment and terminal blood samples and terminal PiZ liver samples (week 6) via ddPCR to see if there was variation in copy number between groups and over time. There were no significant changes in SERPINA1 transgene copy number between groups of PiZ mice between pre and post test article administration in PiZ blood (FIG. 15A). When comparing SERPINA1 transgene copy number between terminal blood and terminal liver samples there is no significant difference in SERPINA1 transgene copy number except for in the group treated with meganuclease AAT 37-38x.50 and the scAAV repair construct (FIG. 15B).

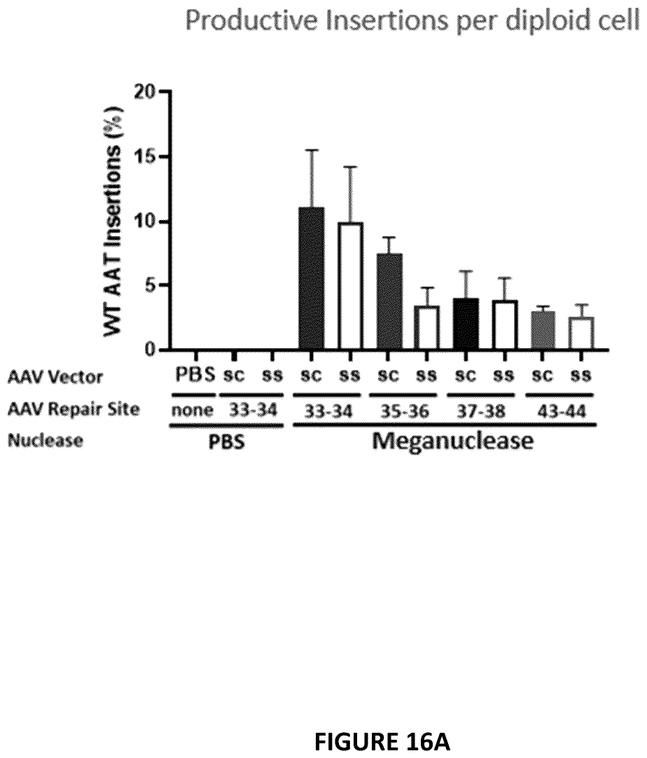

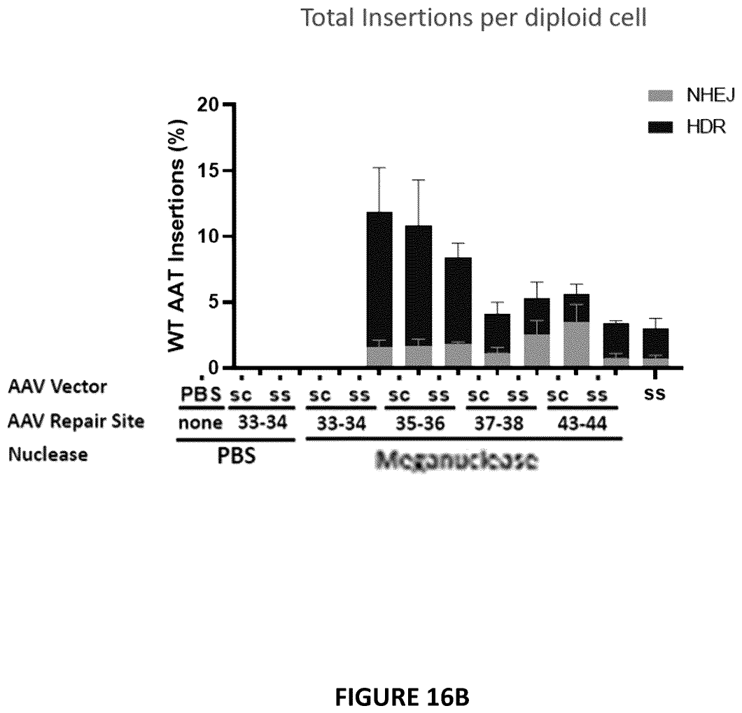

For total productive insertion analysis, we used assays 1, 2, 3 and 5 from Table 5. Insertion analysis determined that productive insertions were highest at the 33-34 site with an average of 10% and 11% insertions for ssAAV and scAAV, respectively. Only the 35-36 site showed a significant difference of insertions between ssAAV and scAAV repair at 3.5% and 7.5% insertions, respectively. An average of 4% insertions was seen for both ssAAV and scAAV for the AAT37-38 site. An average of 3% insertions was observed for both ssAAV and scAAV for the AAT43-44 site (FIG. 16A). We further analyzed if insertions were repaired via NHEJ or HDR using assays 1, 2, 3 and 5 from Table 5 and assays 6, 7, 8 and 10 from Table 5. The data shows that for sites 33-34, 35-36 and 43-44, the majority of repair is orchestrated by HDR mechanisms. Somewhat surprisingly, the 37-38 site shows repair is split between HDR and NHEJ or NHEJ is slightly favored (FIG. 16B).

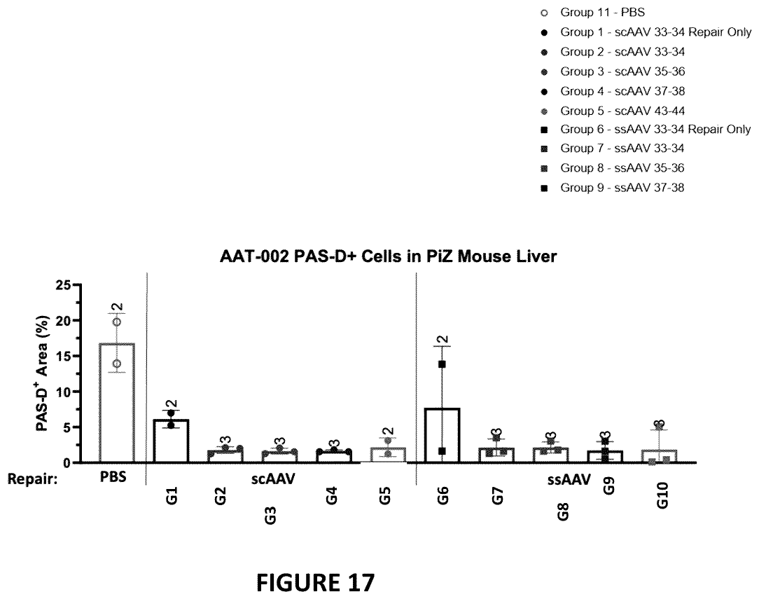

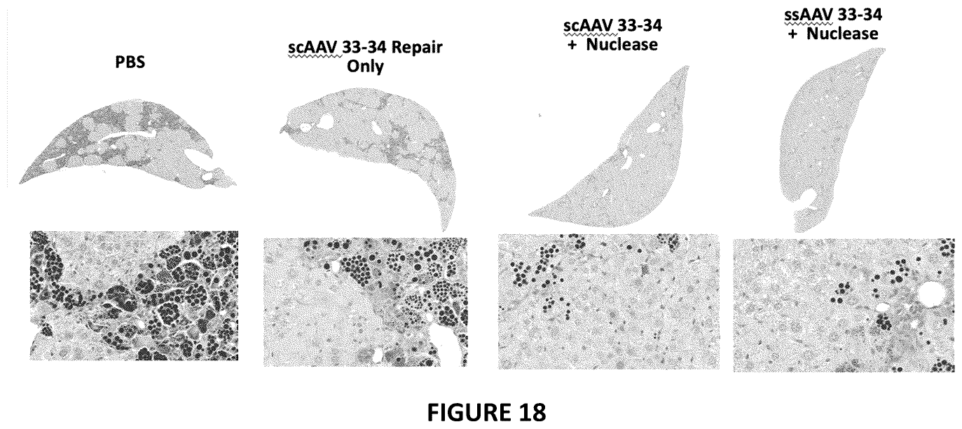

PAS-D staining was used to analyze Z globules within the liver and there is significant reduction of PAS-D positive area in meganuclease treated animals compared to PBS treated animals (FIGS. 17 and 18). There is a slight decrease of PAS-D positive cells in the repair only groups and this may just be variability seen in PiZ mice.

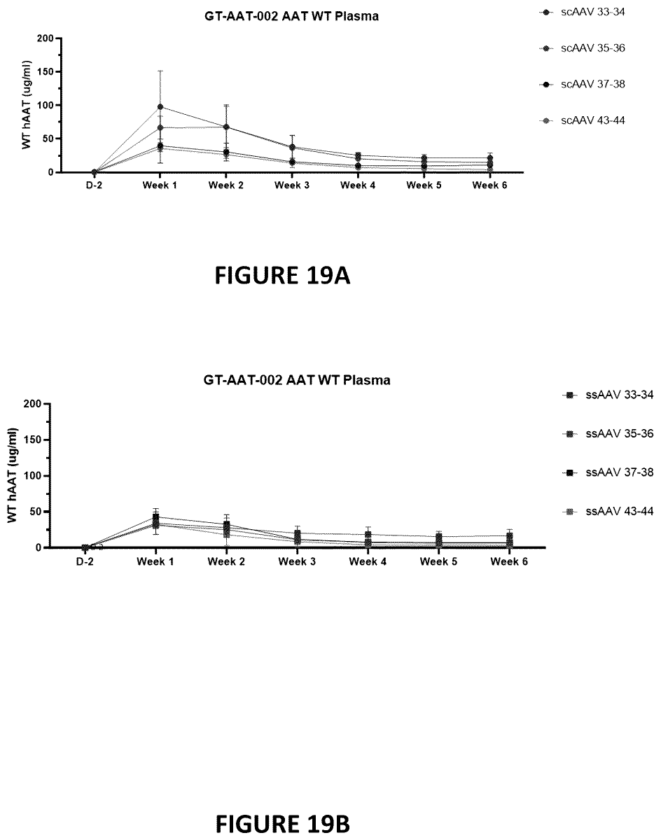

Both WT and Z plasma protein was measured via mass spectrometry. Groups treated with scAAV repair show higher plasma WT AAT protein for sites 33-34 and 35-36 at weeks 1-3 and less so for sites 37-38 and 43-44 (FIG. 19A, 19B). Plasma WT AAT protein peaks at week 1 with the maximum average protein expression of 100 μg/ml in the mice administered the scAAV 33-34 repair template (FIG. 19A).

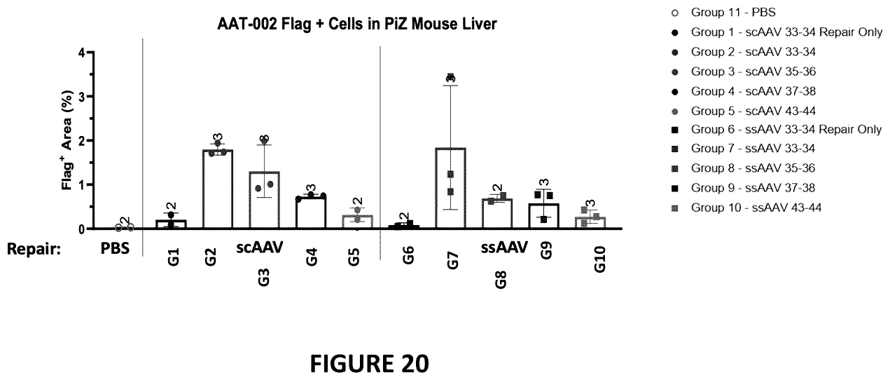

The repair template contains a flag tag so we performed flag immunohistochemistry (IHC) on terminal PiZ mouse liver samples to observe distribution and quantification of our inserted WT AAT repair construct. Flag staining was observed heterogeneously throughout the liver (FIG. 20).

### 3. Conclusions

The AAT-002 study found that the highest level of insertions was around 11% at the 33-34 site with the scAAV repair construct. This group also had the highest plasma WT-AAT protein expression at week 1 post AAV administration at 100 μg/ml. However, AAT-WT plasma protein levels for all groups decreased after week 1 and plateaued below 30 μg/ml after week 4. The high variability of PAS-D staining in the PiZ mice receiving PBS only or repair AAV only made it difficult to conclude that there was a meganuclease-mediated effect on z globules in the liver.

### Example 5

In Vivo AAT LNP delivery of Engineered Meganucleases in a PiZ AAT Mouse Model

### 1. Methods and Materials

PiZ mice were dosed with 0.5-0.9 mg/kg LNP-M formulated with a first-generation 37-38x.50 meganuclease in order to analyze whether an AAV delivered AAT template could be inserted using an LNP to deliver the meganuclease. The study outline is provided in Table 6. The quantifications of human AAT transgene copy number and AAT protein was conducted as described in Example 3. The quantifications of insertions was conducted as described in Example 4 using primers and probes from Table 5, assay 3.

TABLE 6
Example 5 Study Design

| Group | Dose #1 | | Dose #2 |
|---|---|---|---|
| 1 | PBS | PBS | PBS |
| 2 | AAV Repair | LNP-Meganuclease | PBS |
| 3 | | PBS | LNP-Meganuclease |
| 4 | | LNP-Meganuclease | LNP-Meganuclease |
| 5 | | LNP-Meganuclease | PBS |
| 6 | | LNP-Meganuclease | LNP-Meganuclease |
| 7 | PBS | LNP-Meganuclease | PBS |

### 2. Results

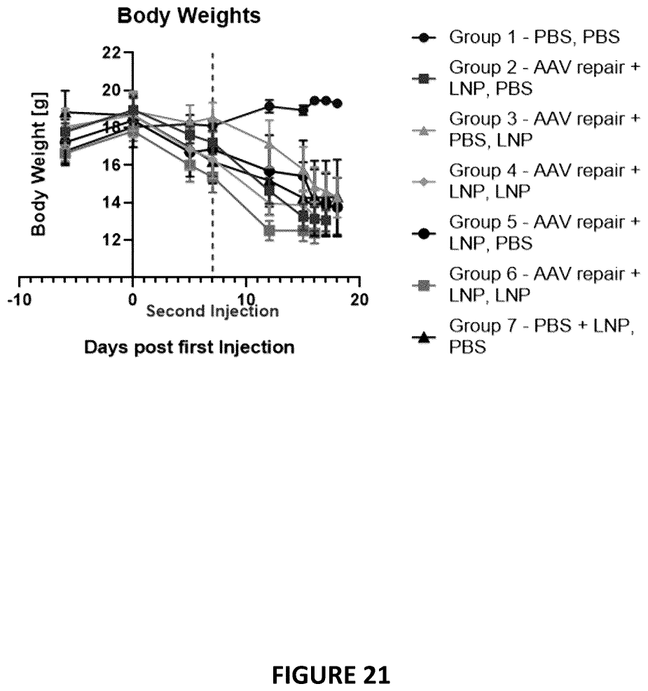

The PiZ mouse study was intended to have three LNP-M doses, at day 0, day 7 and day 14. However, at day 15, the mice in all LNP/meganuclease treated groups showed clinical signs of distress and weight loss. Due to the tolerability issues, all remaining mice were euthanized at day 19. Body weight analysis shows that mice treated with the LNP/meganuclease had a steady decline in body weight post dose in contrast to the PBS and AAV repair alone groups (FIG. 21).

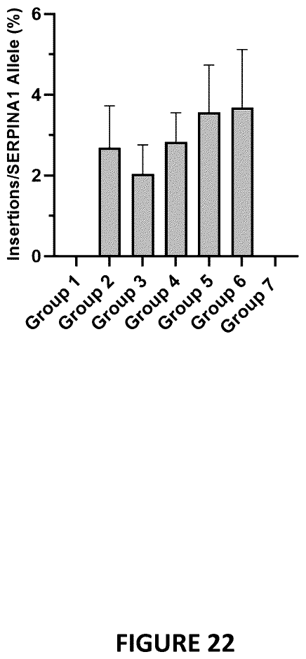

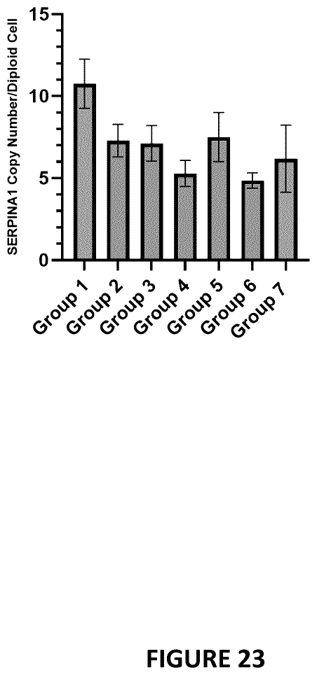

Insertion analysis shows the highest average insertions were around 4% insertions per SERPINA1 allele (FIG. 22). An additional LNP dose did not increase insertion frequency in this study. SERPINA1 copy number demonstrated a significant loss in copy number compared to PBS and LNP only control groups (FIG. 23). An additional LNP dose resulted in an additional loss in SERPINA1 copies.

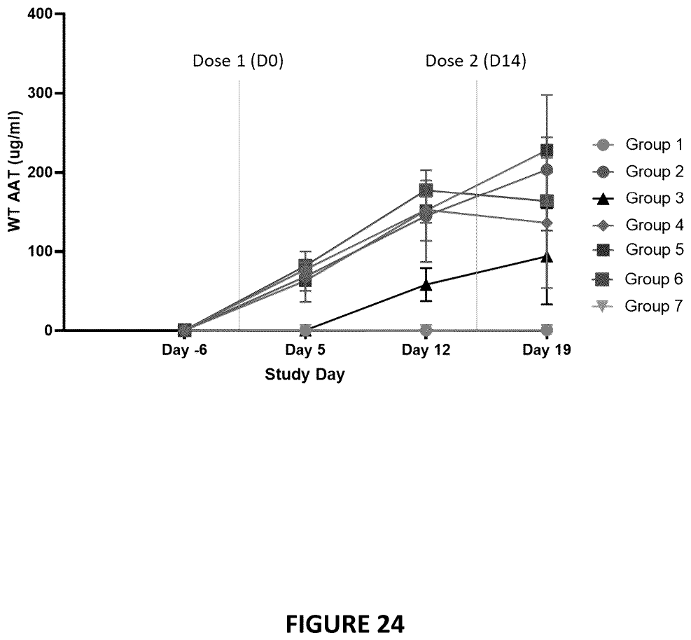

WT protein analysis demonstrated a gradual increase in secreted AAT-WT protein through day 19 for all animals treated with a single dose of LNP-A/meganuclease and reached an average peak protein level of 228 μg/ml (FIG. 24). Animals dosed with two LNP-M/meganuclease doses saw a slight decline in AAT-WT protein the week following the second LNP dose.

### 3. Conclusions

In conclusion, using LNP-M to deliver the generation 1 AAT37-38x.50 meganuclease we demonstrated up to 4% insertions which resulted in a peak secreted WT-AAT protein expression of 228 μg/ml. Tolerability issues were observed in all mice treated with the LNP-M/meganuclease. Due to the timing of clinical observations in these mice, we can infer that the toxicity was likely due to a first-generation meganuclease with likely off target effects.

### Example 6

Editing of AAT Recognition Sequences in Human Cell Lines with Additional Engineered Meganucleases with Off target analysis

### 1. Methods and Materials

These studies were conducted to determine the ability of successive iterations of optimized engineered meganucleases to generate indels at the genomic target site, insertions of a target plasmid into the genomic target site, and the off targeting profile of each meganuclease. The meganucleases in this study were re-engineered from each of the indicated first generation meganucleases. In this study, the engineered meganucleases provided in Table 7 were tested.

TABLE 7
Generations of Engineered Meganucleases

| Generation | Meganuclease |
|---|---|
| 1 | AAT 35-36x.70 |
| 2 | AAT 35-36L.79 |
| 3 | AAT 35-36L.141 |
| 4 | AAT 35-36L.210 |
| 5 | AAT 35-36L.290 |
| 1 | AAT 37-38x.50 |
| 2 | AAT 37-38L.24 |
| 3 | AAT 37-38L.158 |
| 3 | AAT 37-38L.167 |
| 3 | AAT 37-38L.175 |
| 4 | AAT 37-38L.262 |
| 1 | AAT 41-42x.1 |
| 2 | AAT 41-42L.42 |
| 3 | AAT 41-42L.104 |
| 3 | AAT 41-42L.153 |
| 4 | AAT 41-42L.185 |
| 5 | AAT 41-42L.294 |
| 1 | AAT 43-44x.58 |
| 2 | AAT 43-44L.47 |
| 3 | AAT 43-44L.105 |
| 3 | AAT 43-44L.132 |
| 3 | AAT 43-44L.157 |
| 4 | AAT 43-44L.276 |
| 5 | AAT 43-44L.384 |

### Indel and Insertion Analysis

Quantification of indels and AAT template insertion was carried out by electroporating Hek293 cells with 25 ng of meganuclease RNA and 167 ng of DNA repair template and gDNA was collected at times indicated in the figures. DNA was extracted from cells as described in Example 2. The primer and probes used for insertion analysis are provided in Example 4. The primers and probes used for indel analysis are provided in Table 8. The insertion and indel ddPCR cycling conditions were carried out as in Example 4.

TABLE 8
Primers and Probes used in Indel Assay of Example 6
ddPCR AAT Indel Assays

| Assay | | | |
|---|---|---|---|
| # | Primer/Probe | Sequence | SEQ ID |
| 11 | AAT33-34 Fwd | CTGTTTCTCTTGGGTCT | 152 |
| | | CAG | |
| 11 | AAT33-34 Rev | GGGACAGAAGTCAAAG | 153 |
| | | GTTAT | |
| 11 | AAT33-34 Probe | AACAGAGAGGTTGAGC | 154 |
| | | AACTGT | |
| 12 | AAT35-36 Fwd | TGAAGTCATTTACCCCA | 155 |
| | | GGC | |
| 12 | AAT35-36 Rev | CTCACACCATTTCTACC | 156 |
| | | CGG | |
| 12 | AAT35-36 Probe | AATAAACCCAAGTGTG | 157 |
| | | ACCAGGCC | |
| 13 | AAT37-38 Fwd | GCGAGACTCTGTCTCA | 158 |
| | | AA | |
| 13 | AAT37-38 Rev | GGCCATCACTATCCACT | 159 |
| | | T | |
| 13 | AAT37-38 Probe | TCCCCAGTTGTGCAAA | 160 |
| | | G | |
| 14 | AAT41-42 Fwd | CAGGAGCATCAGCCTA | 161 |
| | | T | |
| 14 | AAT41-42 Rev | ATGGCAACAGCTAGAG | 162 |
| | | AG | |
| 14 | AAT41-42 Probe | ATCCTTGTGAGTGTTGG | 163 |
| | | GTGGGAA | |
| 15 | AAT43-44 Fwd | GGCCCGATTCCTGGAT | 164 |
| | | AATC | |
| 15 | AAT43-44 Rev | GCCAGAGAGATTAGGT | 165 |
| | | CAGC | |
| 15 | AAT43-44 Probe | CCAGAATCCTACATCTA | 166 |
| | | GGTCCTGCAC | |

### Off Target Analysis

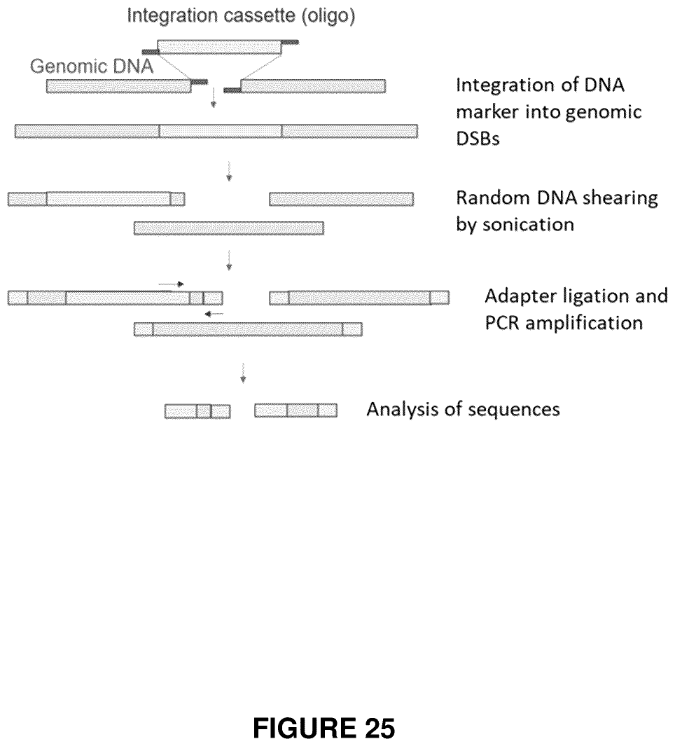

Meganuclease specificity was measured using the oligocapture assay. This is a cell-based, in vitro assay that relies on the integration of a synthetic oligonucleotide (oligo) cassette at DSBs within the genome. Using the oligo as an anchor, genomic DNA to either side of the integration site can be amplified, sequenced, and mapped (FIG. 25). This allows for a minimally biased assessment of potential off-target editing sites of the meganuclease. This technique was adapted from GuideSeq (Tsai et al. (2015) Nat. Biotech. 33:187-97) with specific modification to increase sensitivity and accommodate the 3′ complementary overhangs induced by the meganucleases. The OligoCapture analysis software is sequence agnostic. That is, no a priori assumptions are made regarding which DNA sequences the meganuclease is capable of cutting. In the OligoCapture assay, cells are transfected with meganuclease mRNA and double-stranded DNA oligonucleotides. After 2 days, the cellular genomic DNA was isolated and sheared into smaller sizes. An oligonucleotide adapter was ligated to the sheared DNA and polymerase chain reaction was used to amplify any DNA pieces that contain an adapter at one end and the captured oligonucleotide at the other end. The amplified DNA was purified, and sequencing libraries were prepared and sequenced. The data were filtered and analyzed for valid sites that captured an oligonucleotide to identify potential off-target sites. The sequence reads were aligned to a reference genome, and grouped sequences within thousand-base pair windows scanned for a potential meganuclease cleavage site. HEK293 cells were transfected with mRNA for multiple of the AAT meganucleases at each round of optimization (rounds 1˜4 or 5) gDNA was isolated and processed as written above in the assay description.

### 2. Results

Indels at the 33-34, 35-36, 37-38, 41-42 and 43-43 recognition site for the first generation meganucleases are shown in FIG. 7 as described in Example 2.

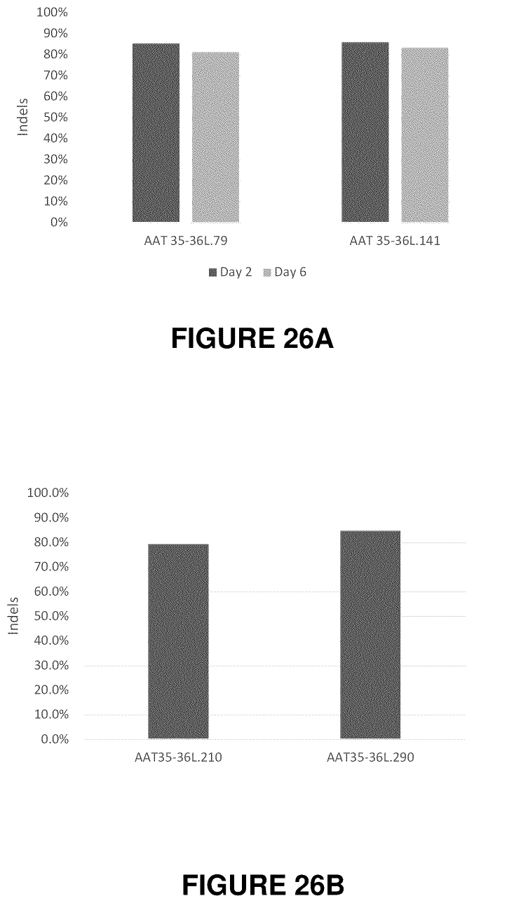

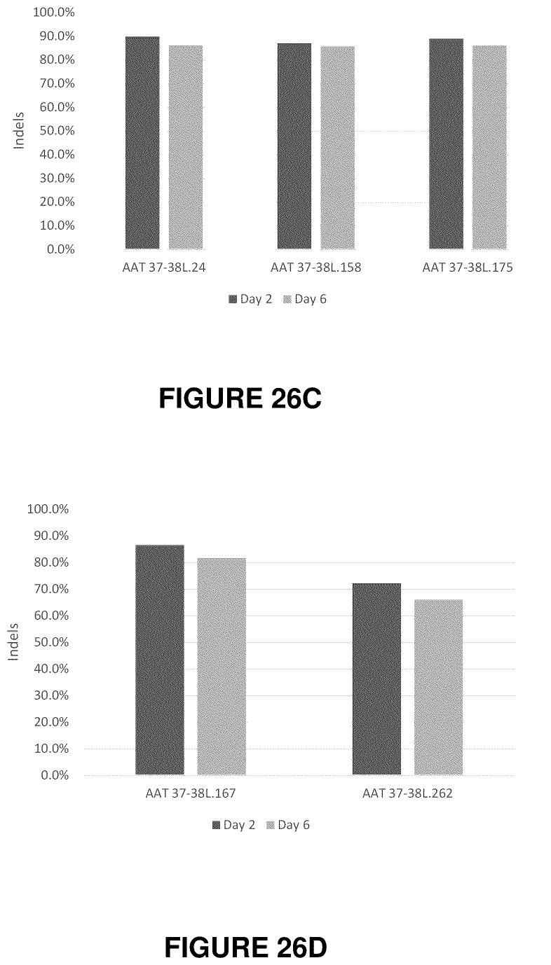

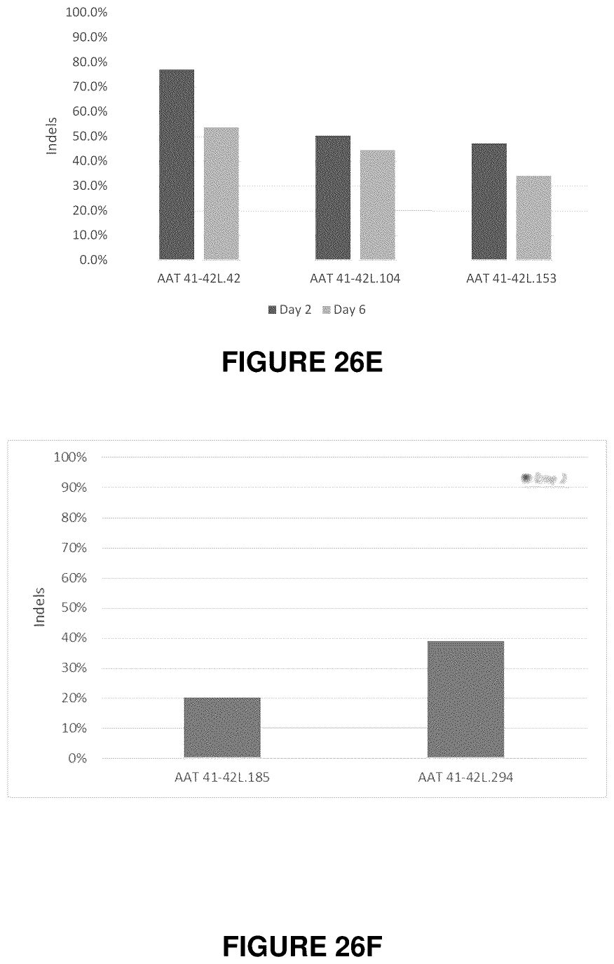

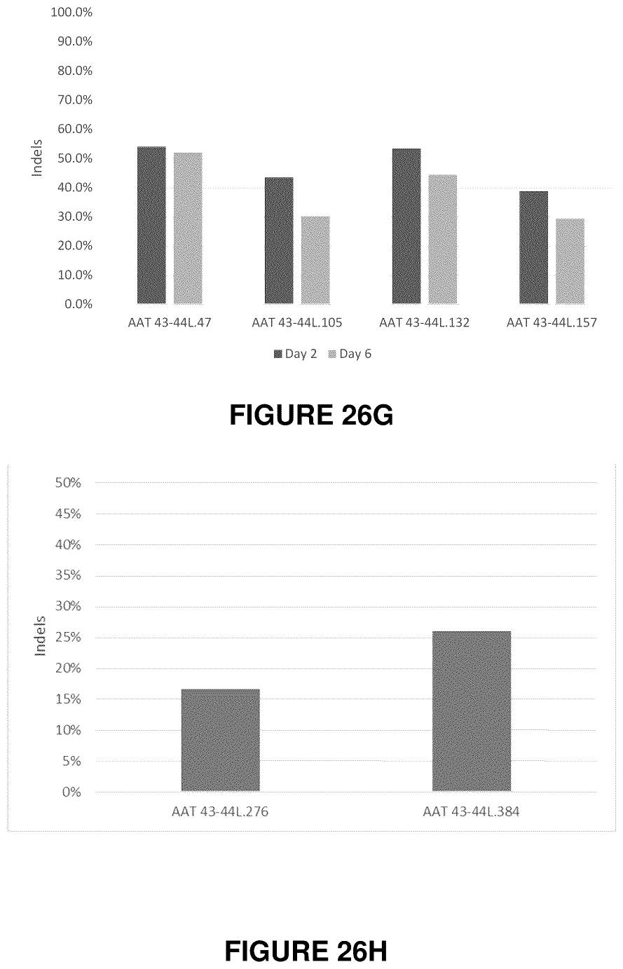

Indels at the 35-36 site for meganuclease generations 2-5 are shown in FIG. 26A and FIG. 26B. These four generations demonstrate indels around 80-85%. The indel activity does not change significantly from generation 2-5.

Indels at the 37-38 recognition site for generations 2-5 are shown in FIG. 26C and FIG. 26D. The AAT37-38 gen 2 meganuclease, AAT37-38L.24 and generation 3 nucleases, AAT37-38L.158 and AAT37-38L.175 all demonstrated 90% indels, Gen 4 meganuclease AAT37-38L.167 demonstrated 86% indels and the generation 5 nucleases show a slight decrease indels at ˜70% indels.

Indels at the AAT41-42 recognition site for generations 2-5 are shown in FIG. 26E and FIG. 26F. The generation 2 AAT41-42L.42 meganuclease demonstrated around 77% indels. Generation 3 nucleases AAT41-42L.104 and AAT41-42L.153 show a decrease in indels from generation 2 at ˜50% indels. The generation 4 AAT41-42L.185 meganuclease demonstrated 20% indels and the generation 5 AAT41-42L.294 meganuclease demonstrated an increase at 40% indels.

Indels at the AAT43-44 recognition site for generations 2-5 are shown in FIG. 26G and FIG. 26H. The generation 2 AAT43-44L.47 meganuclease demonstrated 52% indels. The generation 3 AAT43-44L. 132 also showed 52% indels. The generation 4 meganuclease AAT43-44L. 276 demonstrated 16% indels and the generation 5 AAT43-44L.384 meganuclease showed an increase in indels at 26%.

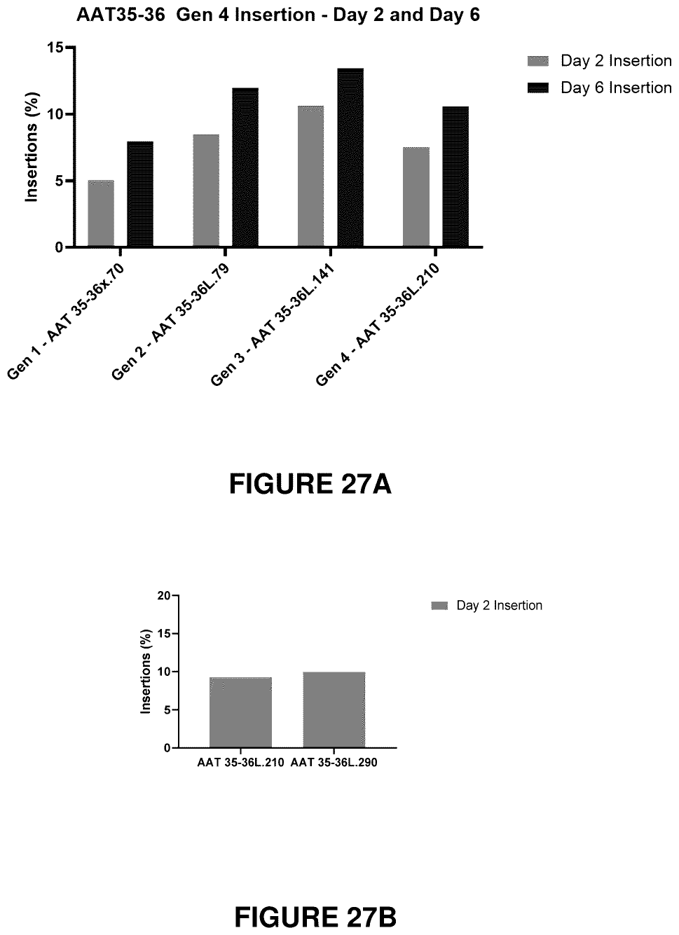

Insertions at the AAT 35-36 recognition site for the first four generations of tested meganucleases are shown in FIG. 27A. FIG. 27B provides the insertion data for a fourth generation meganuclease (AAT 35-36L.210) compared to a fifth generation meganuclease (AAT 35-36L.290). Overall, the insertions for the first-generation AAT 35-36x.70 meganuclease were lower than the later developed second generation to fourth generation meganucleases indicating an improvement in insertion capability in the later generation meganucleases (FIG. 27A). The fifth generation AAT35-36L.290 meganuclease demonstrated similar or higher insertional activity compared to the AAT 35-36L.210 meganuclease (FIG. 27B). While the exact percentage of insertions cannot be compared between independent experiments (e.g., the results of FIG. 27A cannot be directly compared to that of FIG. 27B), it can be inferred that the fifth generation AAT 35-36L.290 meganuclease would have higher insertional activity than the first-generation AAT 35-36x.70 meganuclease because the AAT 35-36L.290 meganuclease had higher activity than the AAT 35-36L.210 meganuclease when run in the same experiment.

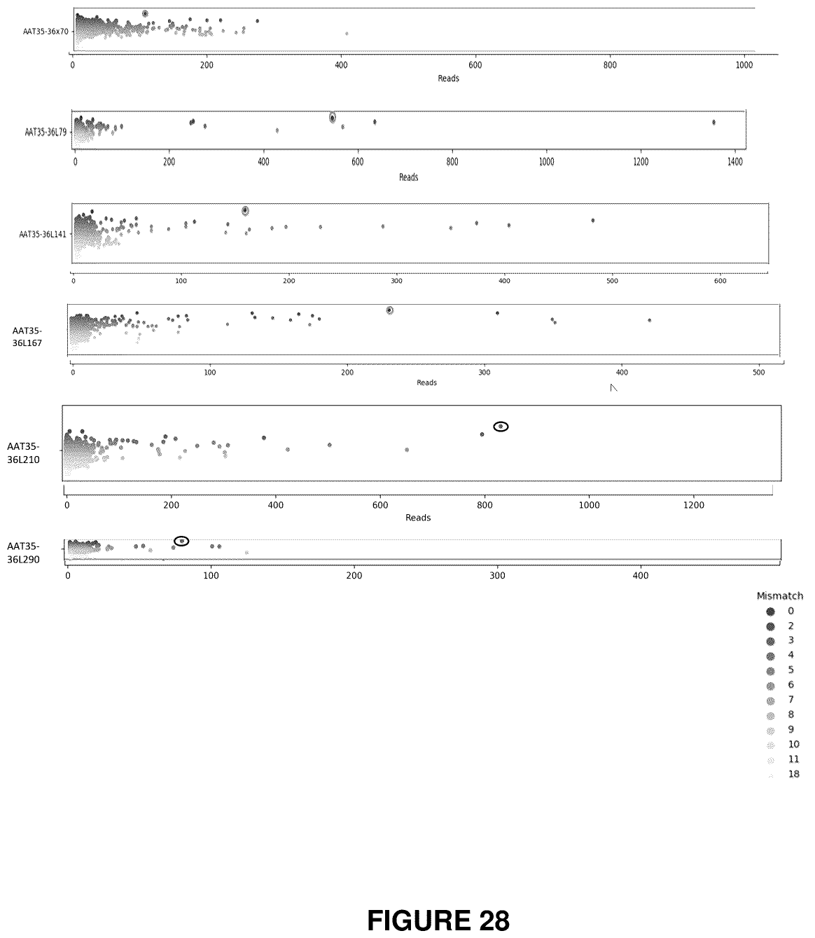

The oligocapture off targeting assay data for the tested AAT 35-36 meganucleases is provided in FIG. 28. The circled dot in each of these plots indicates the on-target site, which had progressively higher read count in the later developed meganucleases compared to the first-generation AAT 35-36x.70 meganucleases with less overall detected off target sites for all the later developed meganucleases.

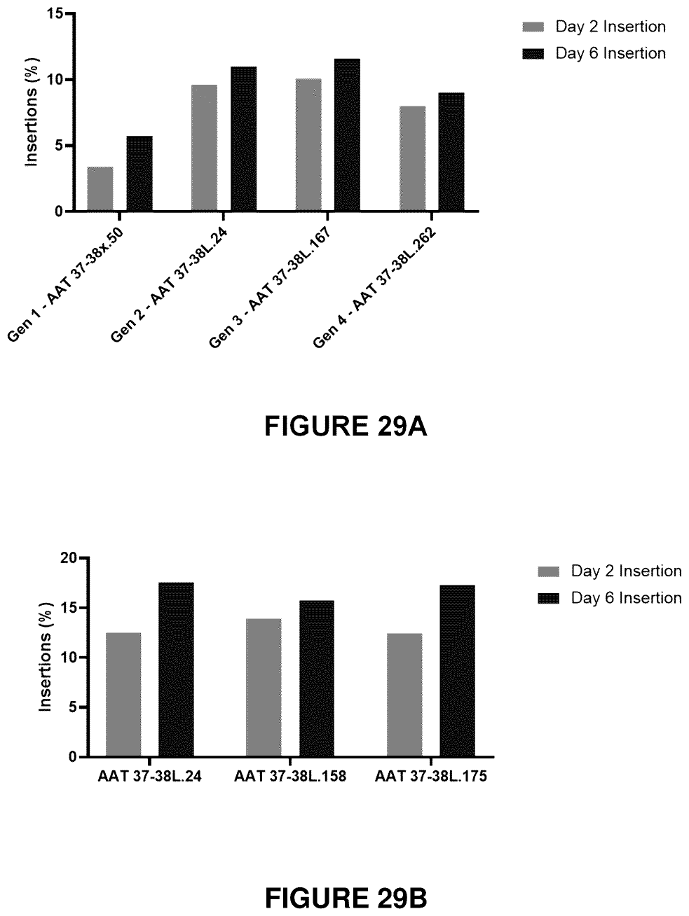

Insertions at the AAT 37-38 recognition site for the first four generations of tested meganucleases are shown in FIG. 29A and one generation 2 and two generation 3 tested meganucleases are shown in FIG. 29B. Consistent with the results observed for the meganucleases developed for the AAT 35-36 site, insertions for the first-generation AAT 37-38x.50 meganuclease was lower than the later developed second generation to fourth generation AAT 37-38 meganucleases again indicating an improvement in insertion capability in the later generation meganucleases (FIG. 29A).

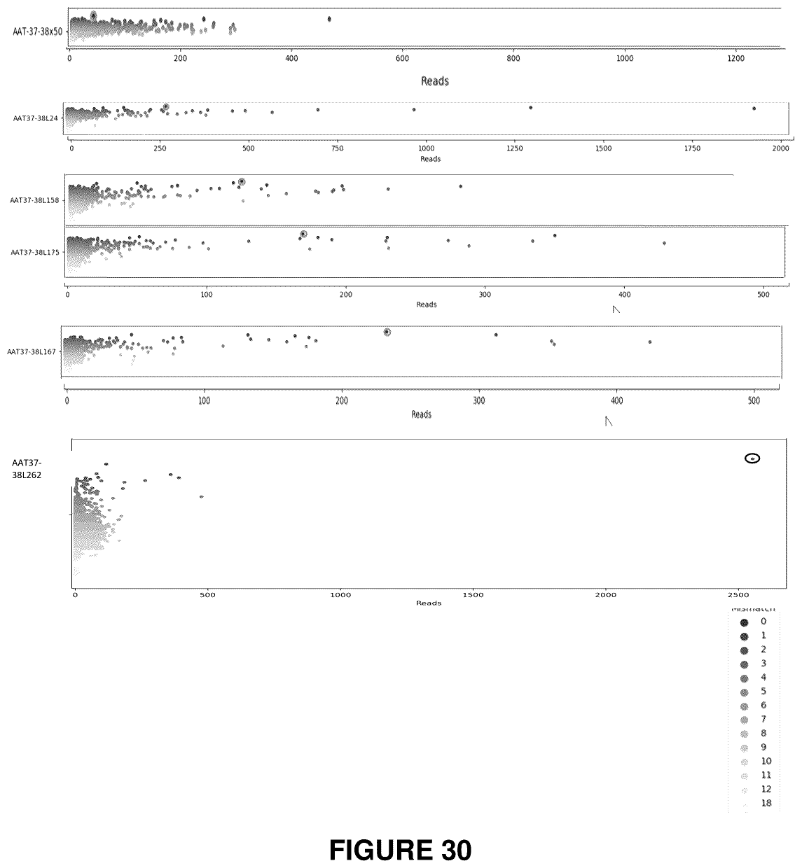

The oligocapture off targeting assay data for the tested AAT 37-38 meganucleases is provided in FIG. 30. The on-target site had a progressively higher read count in the later developed meganucleases compared to the first-generation AAT 37-38x.50 meganuclease with less overall detected off target sites for all of the later developed meganucleases. The AAT 37-38 L.262 meganuclease had the lowest number of off target reads and a high on target read count indicating that this meganuclease was the most specific of the tested meganucleases, which also had better optimized insertional activity compared to the first generation meganuclease.

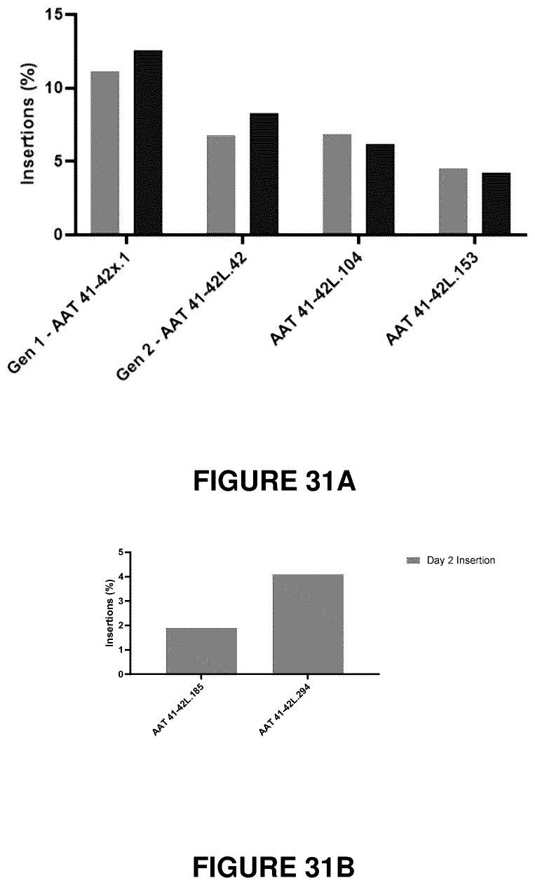

Insertions at the AAT 41-42 recognition site for the first three generations of tested meganucleases are shown in FIG. 31A. These results differ somewhat from the prior findings in that the first-generation AAT 41-42x.1 meganuclease demonstrated higher insertion activity compared to the later developed second and third generation meganucleases. FIG. 31B provides the insertion data of the fifth generation AAT 41-42L.294 meganuclease compared to the fourth generation AAT 41-42L.185 meganuclease, which shows that the fifth generation meganuclease has improved insertion capability compared to the fourth generation meganuclease.

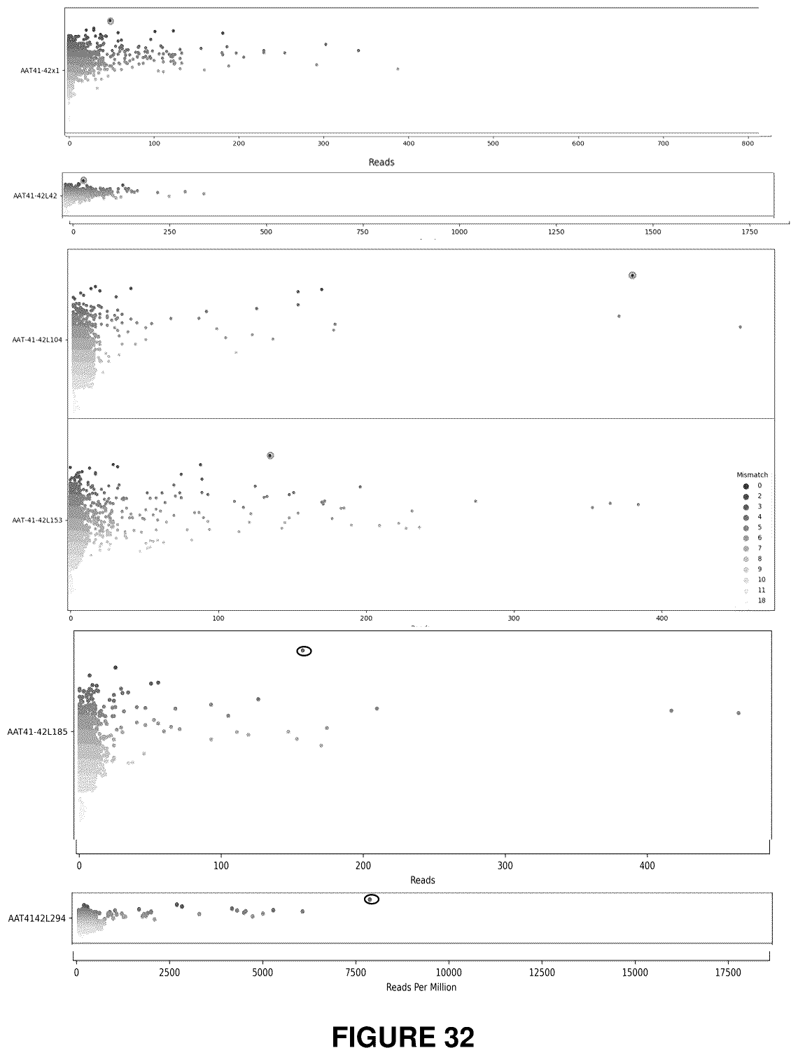

The oligocapture off targeting assay data for the tested AAT 41-42 meganucleases is provided in FIG. 32. The on-target site had a progressively higher read count in the later developed third to fifth generation meganucleases compared to the first-generation AAT 41-42x.1 meganuclease with less overall detected off target sites for all of the later developed meganucleases. The AAT 41-42 L.294 meganuclease had the lowest number of off target reads and a very high on target read count indicating that this meganuclease was the most specific of the tested meganucleases. Thus, although the insertion percentage was higher in the first generation AAT 41-42x.1 meganuclease (FIG. 31A), the later developed third to fifth generation meganucleases had significantly lower off target sites, and therefore, would be expected to have a better safety profile in vivo.

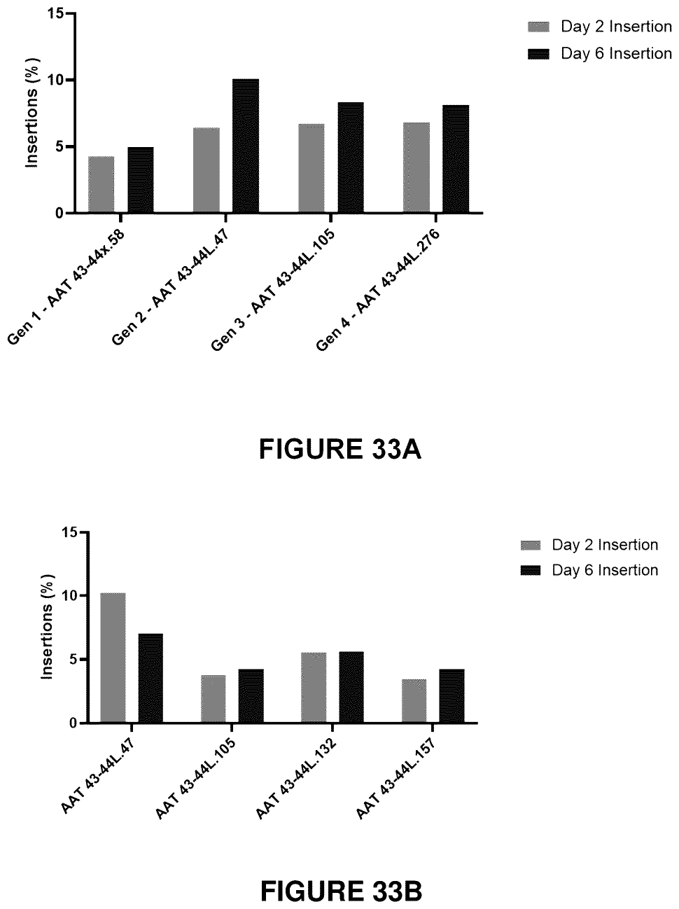

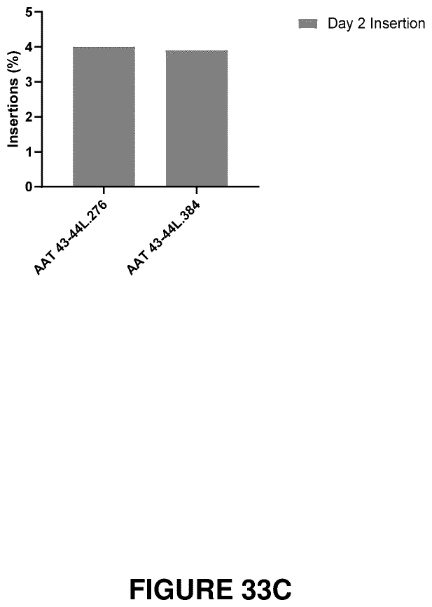

Insertions at the AAT 43-44 recognition site for the first four generations of tested meganucleases are shown in FIG. 33A and the second and third generation of tested meganucleases is shown in FIG. 33B. A comparison of insertional activity between the fourth generation AAT 43-44L.376 and fifth generation AAT 43-44L.384 meganuclease is provided in FIG. 33C. Consistent with the results observed for the meganucleases developed for the AAT 35-36 and AAT 37-38 sites, insertions for the first-generation AAT 43-44x.58 meganuclease was lower than the later developed second generation to fifth generation AAT 37-38 meganucleases again indicating an improvement in insertion capability in the later generation meganucleases (FIG. 33A).

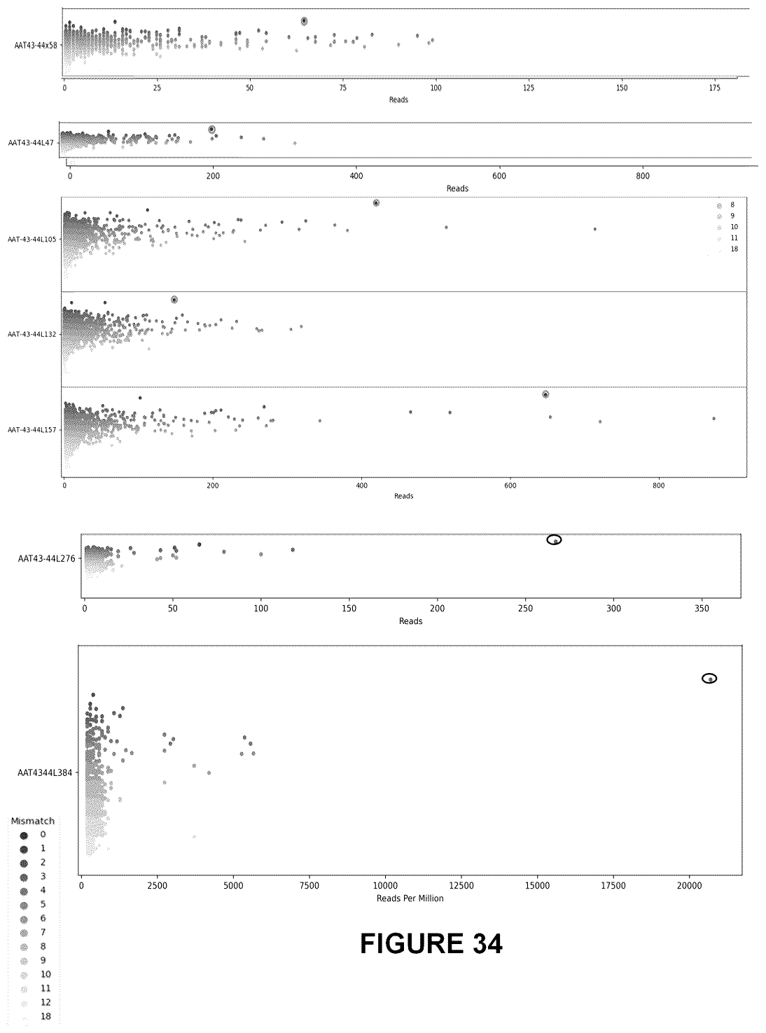

The oligocapture off targeting assay data for the tested AAT 43-44 meganucleases is provided in FIG. 34. Consistent with the oligocapture data for the AAT 35-36, AAT 37-38, and AAT 41-42 sites, the detection of the on-target site had a progressively higher read count in the later developed meganucleases compared to the first-generation AAT 43-44x.58 meganuclease with less overall detected off target sites for the later developed meganucleases. The AAT 43-44L.384 meganuclease had a low number of off target reads and a very high on target read count. In addition, this fifth generation meganuclease retained the same amount of insertional activity as the fourth generation AAT 43-44L.276 meganuclease indicating that it is a significantly more active and specific meganuclease compared to the first generation AAT 43-44x.58 meganuclease.

### 3. Conclusions

The frequency of indels for optimized meganucleases at the 35-36 and 37-38 sites are consistently high and do not vary significantly across the successive generations. In contrast, for both the 41-42 and 43-44 sites, the frequency of indels varies more and tends to decrease from generation 2-4. However, the latest fifth generation meganuclease for both of these sites demonstrated improved indel activity indicating that these meganucleases were more active.

The insertion frequency of a template nucleic acid was improved at the AAT 35-36, AAT 37-38, and AAT 43-44 target sites using optimized meganucleases. In addition, for all sites, the later developed meganucleases were more specific. There was one caveat to this trend with the insertional activity at the AAT 41-42 site. With this site, the earlier generation of meganuclease demonstrated the highest insertional activity. However, that increased activity came at a significant cost in terms of specificity as shown in the oligocapture results. Accordingly, all later generation 3-4 or 3-5 meganucleases for all of the tested recognition sites demonstrated significantly reduced off-target profile as demonstrated in the oligocapture data. These meganucleases were able to balance adequate cleavage and insertional activity as assessed by indel and template insertion frequency with much lower off target cutting indicating the protein engineering to optimize these meganucleases resulted in significant clinically relevant improvements.

### Example 7

### In Vivo AAT Gene Editing in a PiZ AAT Mouse Model

### 1. Methods and Materials

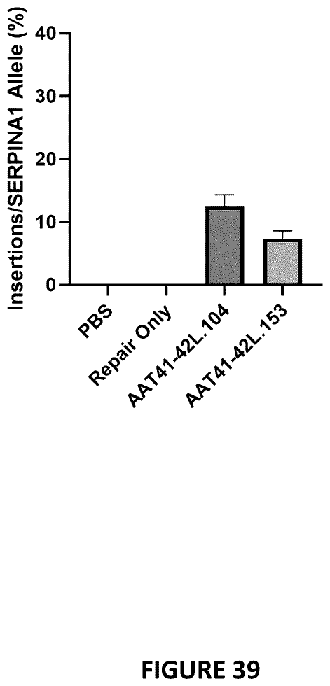

To understand any differences between meganuclease binding sites in the PiZ model, we delivered third generation nucleases at sites 37-38, 41-42 and 43-44 via a single stranded AAV and concurrently delivered the repair template as a self-complimentary AAV (FIG. 39). PiZ mice were either administered PBS and one of the WT AAT repair AAV8 vectors, or they were co-administered two AAV8 vectors, one encoding the engineered AAT meganuclease and one comprising one of the four designed WT AAT repair sequences, via retro-orbital (RO) injections at doses 5e12 VG/kg and 2.5e13 VG/kg, respectively. The study design is provided in Table 9 below.

TABLE 9
Study Design for Example 7.

| | | PBS/AAV8 Generation 3 | |
|---|---|---|---|
| Group # | PBS/AAV8 Repair | Meganuclease | N= |
| 1A | PBS | PBS | 5 |
| 2 | scAAV 37-38 | PBS | 3 |
| 3 | | AAT 37-38L.167 | 5 |
| 4 | | AAT 37-38L.158 | 5 |
| 5 | | AAT 37-38L.175 | 5 |
| 1B | | PBS | 5 |
| 6 | scAAV 41-42 | PBS | 3 |
| | | AAT 41-42L.104 | 5 |
| | | AAT 41-42L.153 | 5 |
| 1C | | PBS | 5 |
| 9 | scAAV 43-44 | PBS | 3 |
| 10 | | AAT 43-44L.105 | 5 |
| 11 | | AAT 43-44L.132 | 5 |
| 12 | | AAT 43-44L.157 | 5 |
| Total | | | 64 |

The repair constructs were designed with homology arms for their corresponding binding sites (i.e. AAT 37-38, AAT41-42 or AAT 43-44) with a size of ˜300 bp due to the packaging limitations of scAAV (˜2.4 kb), which allowed for the WT AAT coding repair construct to be within these limitations. The third generation nucleases used in this study were chosen based on their in vitro insertion and specificity data (FIGS. 27-34).

Blood was collected from mice at weekly intervals from day-7 through day 28 and plasma was isolated and analyzed as previously described in Example 3. Liver tissue was harvested from the mice at day 28. DNA was isolated from tissues as described in Example 3. Insertion analysis was performed as described in Example 4 using the dPCR primers and probes provided in Table 5 utilizing assays 3, 4 and 5. Indel analysis was performed as described in Example 6, Table 8, utilizing assays 13, 14 and 15. The insertion and indel ddPCR cycling conditions were carried out as in Example 4. AAT WT and Z transcript quantification was performed using the ddPCR primers and probes from Table 10 using the following cycling conditions: 1 cycle of 95° C. for 10 minutes, 44 cycles of 94° C. for 10 seconds, 63° C. for 30 seconds and 72° C. for 1 minute, 1 cycle of 98° C. for 10 minutes and a 4° C. hold. The insertion and indel ddPCR cycling conditions were carried out as in Example 4. Liver immunohistochemistry was performed as described in Example 4.

TABLE 10
Primers and Probes used in AAT Transcript ddPCR Assays of Example 7
AAT Transcript ddPCR Assays

| Assay # | Primer/Probe | Sequence | SEQ ID |
|---|---|---|---|
| 18 | AAT Transcript fwd | CTCCAAGGCCGTGCAT | 167 |
| | | AA | |
| 18 | AAT Transcript rev | AGACATGGGTATGGCC | 168 |
| | | TCTA | |
| 18 | WT AAT Transcript Probe | CTGACCATCGACGAGA | 169 |
| | | AAGG | |
| 19 | AAT Transcript fwd | CTCCAAGGCCGTGCAT | 167 |
| | | AA | |
| 19 | AAT Transcript rev | AGACATGGGTATGGCC | 168 |
| | | TCTA | |
| 19 | Z AAT Transcript Probe | CTGACCATCGACAAGA | 170 |
| | | AAGG | |
| 20 | Mouse GAPDH Thermo Fischer Assay-VIC mm99999915_g1 | | |

### 2. Results

All groups tolerated the meganuclease treatment well except for two groups that were treated with the AAT 41-42L.153 and AAT 43-44L.132 meganucleases, which saw tolerability issues and were euthanized 1 and 2 weeks early, respectively.

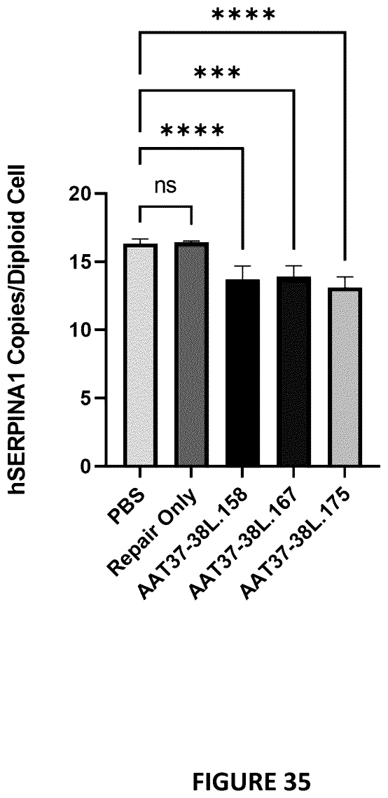

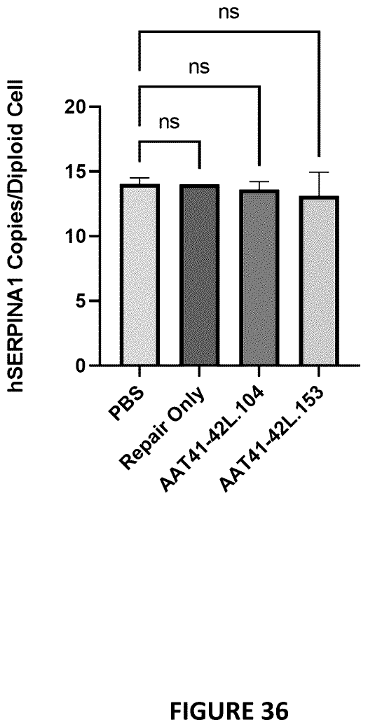

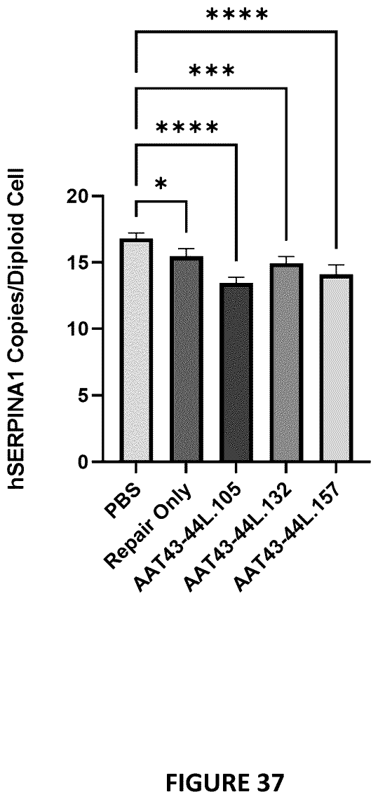

The SERPINA1 transgene copy number was analyzed in PiZ liver samples 4 weeks post AAV administration via ddPCR to see if there was variation in copy number between PiZ mouse cohorts A, B and C. There was a significant difference in SERPINA1 transgene copy number between meganuclease treated groups and the PBS or repair only groups for each 37-38 meganuclease and also for each 43-44 meganuclease with a drop in ˜1-4 transgene copies (FIGS. 35 and 37). There was not a significant difference in SERPINA1 transgene copy number in the 41-42 meganuclease treated PiZ mice compared to PBS or repair only groups (FIG. 36).

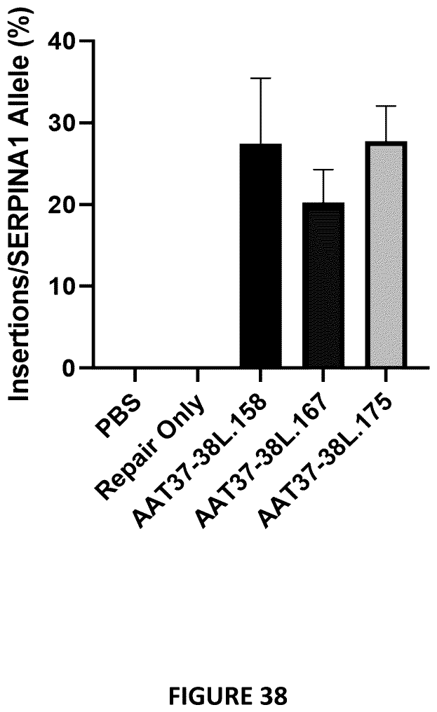

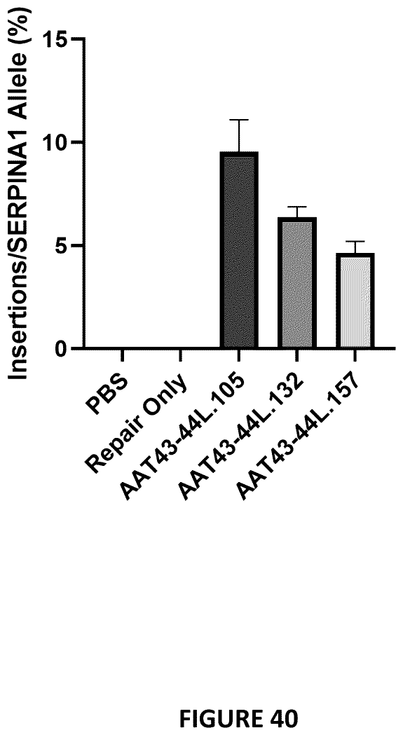

Insertion analysis determined that the AAT 37-38 site showed the highest levels of insertions with an average of up to 28% insertions per SERPINA1 allele for PiZ mice treated with the third generation AAT37-38L.158 meganuclease (FIG. 38). The AAT 41-42L. 104 meganuclease treated PiZ mice saw an average of 13% insertions (FIG. 39) and the AAT43-44L.105 meganuclease demonstrated an average of 14% insertions (FIG. 40).

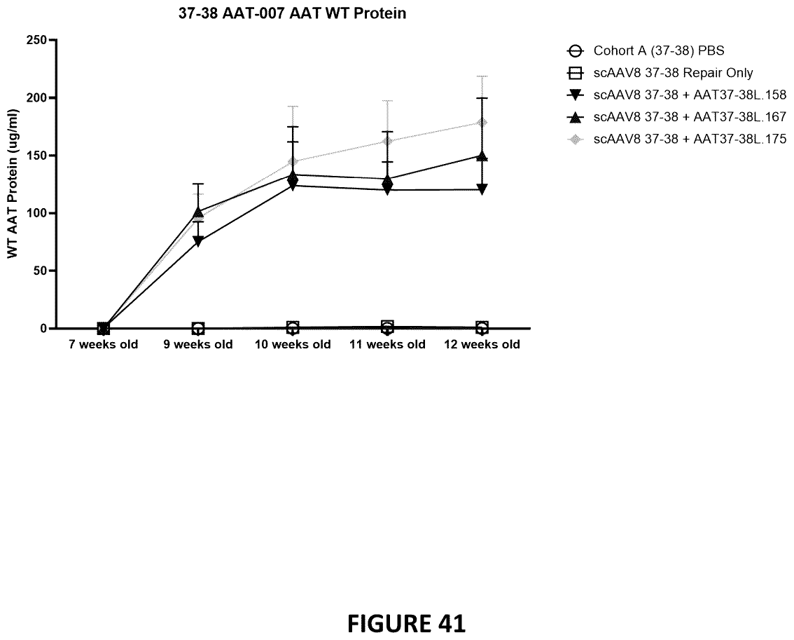

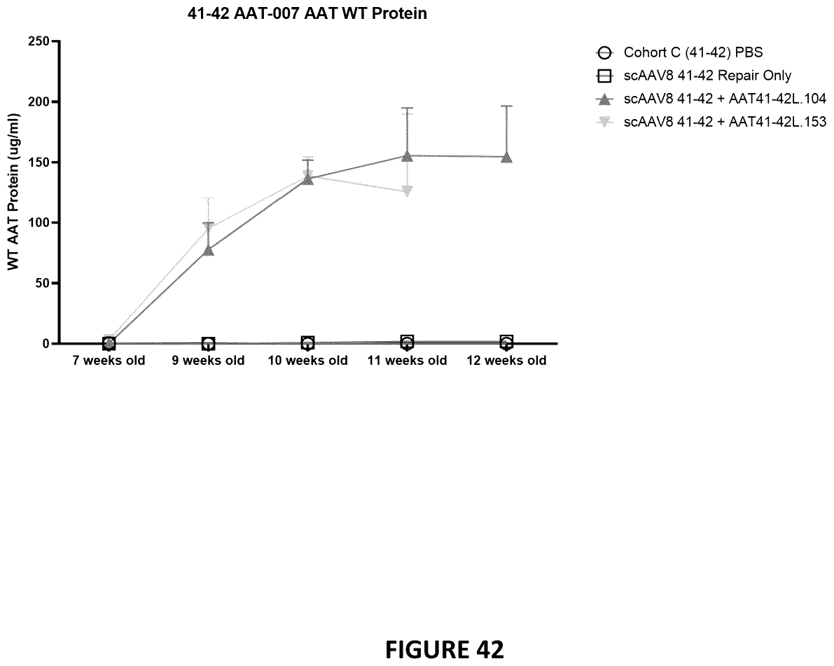

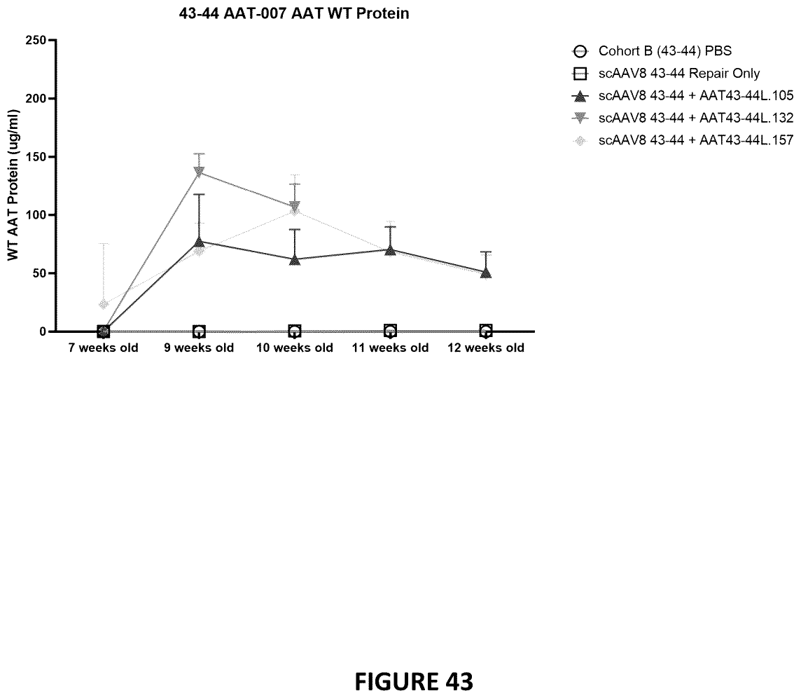

Both WT and Z plasma protein was measured via mass spectrometry. All sites showed an increase in WT AAT protein 1 week after AAV administration. For the AAT37-38 and AAT41-42 nucleases the WT AAT protein peaked at around 3 or 4 weeks post administration with the highest average levels being reaching around 179 μg/ml of protein in the AAT37-38L.175 treated animals (FIG. 41). The AAT41-42L.104 treated animals saw a plateau of secreted WT AAT protein expression at around 150 μg/ml (FIG. 42). The 43-44 meganuclease treated animals saw a gradual decline in secreted WT AAT protein after an initial peak at 1 week with the highest level of WT-AAT expression reaching 137 μg/ml (FIG. 43).

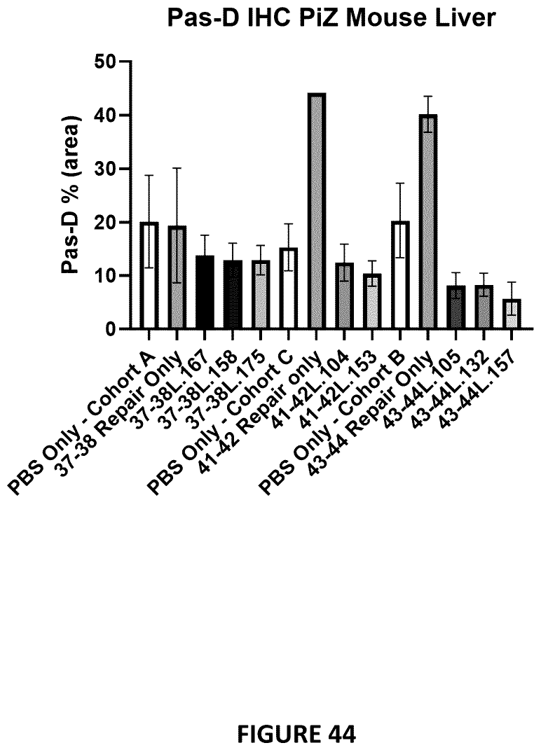

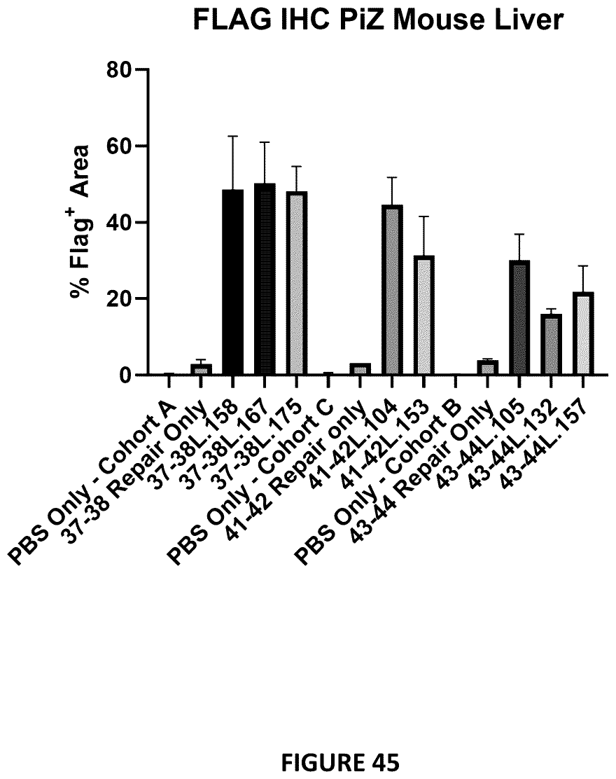

PAS-D and flag immunohistochemistry was performed to identify z-globules and WT-AAT protein in terminal PiZ mouse liver samples. PAS-D staining shows there is a slight decrease in PAS-D staining in all meganuclease treated animals compare to PBS and repair only groups (FIG. 44). The WT-AAT insert contains a flag tag so we can identify the WT AAT protein in the liver without the concern of human and mouse AAT-antibody cross reaction. The flag immunohistochemistry shows an average of approximately 50%, 30-50% and 20-30% of liver cells are positive for flag for sites AAT37-38, AAT41-42 and AAT43-44, respectively (FIG. 45).

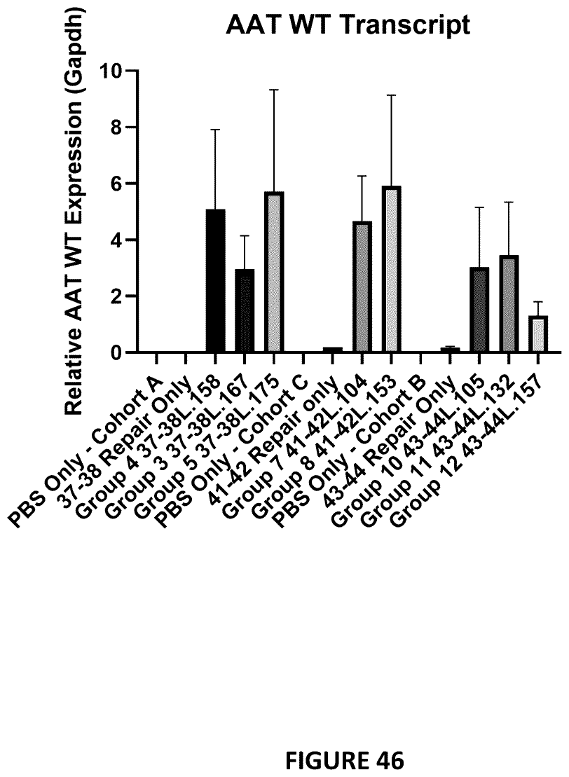

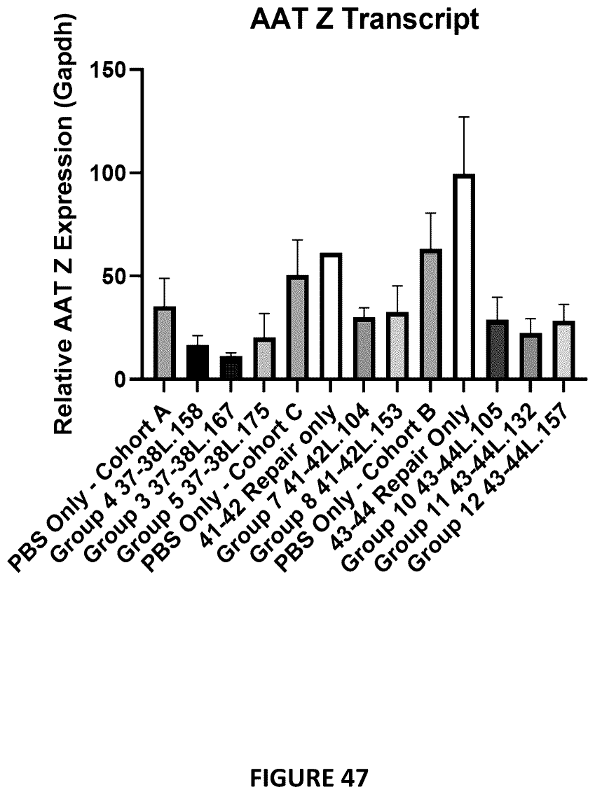

RNA transcript analysis was performed for both AAT-WT and AAT-Z transcript in terminal PiZ mouse livers. WT-AAT transcript shows there is an increase in WT transcript for all meganuclease sites and this trends closely with the insertion and AAT-WT protein data (FIG. 46). The AAT-Z transcript data suggest there may be a meganuclease-mediated decrease in Z-AAT protein, however, similarly to the PAS-D staining, the loss of SERPINA1 Z transgenes cannot be ruled out as a reason for this (FIG. 47). Interestingly, the AAT41-42 site does not see a loss of SERPINA1 Z transgenes yet there is a decrease in AAT-Z transcript, suggesting the loss is meganuclease mediated.

### 3. Conclusions

Overall, AAT-007 demonstrated up to an average of 28% insertions can be achieved by delivering an meganuclease via AAV8 in conjunction with an AAV8 repair template in PiZ mice. The insertions led to up to 179 μg/ml of circulating WT-AAT protein and correlated well with the WT transcript, decrease in PAS-D and decrease in Z transcript.

### Example 8

### 1. Methods and Materials

The PiZ mouse model, as described in Example 3, was used for this study to determine the frequency of insertions after LNP delivery of ARCUS over multiple doses with the AAT WT ssAAV8 repair template. The generation 4 AAT37-38L.262 meganuclease was delivered as mRNA and formulated into an MC3 based LNP formulation (LNP-A). LNP-A was dosed at 0.5, 0.3, 0.1 or 0.05 mg/kg with the AAV repair template dosed at 3e13 vg/kg for all four groups. PBS and repair template only control groups were also included in this study. The study design is provided in Table 11 below. LNP and AAV were delivered together via retro-orbital (RO) injections in PiZ mice.

TABLE 11
Study Design for Example 8

| | | AAV Repair Dose | LNP Dose | |
|---|---|---|---|---|
| | Treatment | (vg/kg) | (mg/kg) | N= |
| 1 | PBS | X | X | 5 |
| 2 | ssAAV8 37-38 Alone | 3e13 | X | 5 |
| 3 | LNP-A AAT37-38L.262/ | 3e13 | 0.5 | 5 |
| 4 | SSAAV8 37-38 | 3e13 | 0.3 | 5 |
| 5 | | 3e13 | 0.1 | 5 |
| 6 | | 3e13 | 0.05 | 5 |
| Total | | | | 30 |

Blood was collected from mice at weekly intervals from day-7 through day 28. Serum was collected at days-7, 2, 9 and 28 for serum chemistry (ALT, AST, ALP, Tbil) analysis. Plasma was isolated on study days 16, 23 and 28 and analyzed as previously described in Example 3. Liver tissue was harvested from the mice at day 28. DNA was isolated from liver tissue as described in Example 3. AAT Transgene copy number quantification was performed as described in Example 3. Insertion quantification was performed as described in Example 4 using primers and probes provided in Table 5, assay 3. Indel quantification was performed as described in Example 6, using primers and probes from Table 8, assay 13. Liver immunohistochemistry was performed as described in Example 4.

### 2. Results

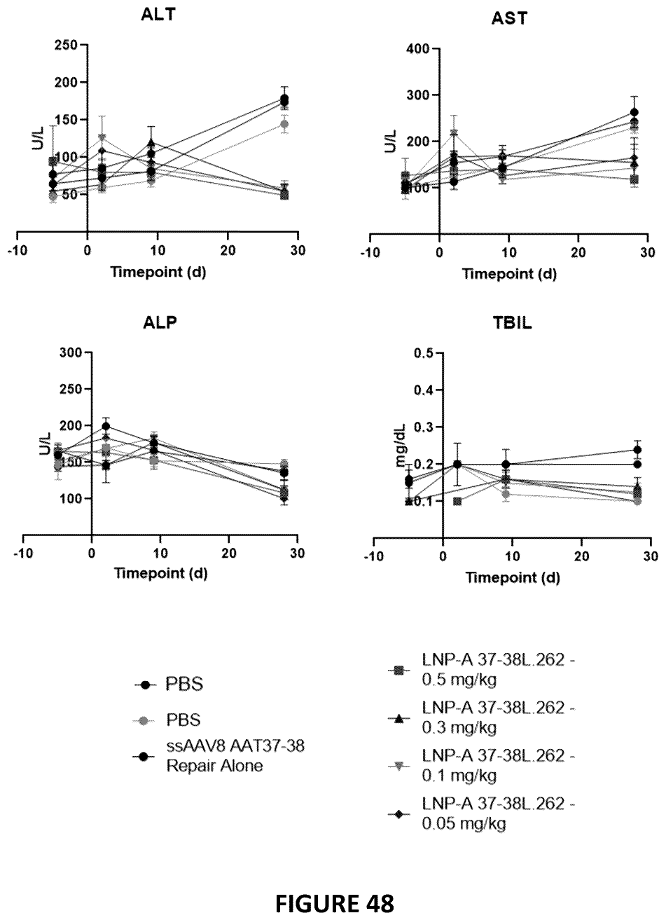

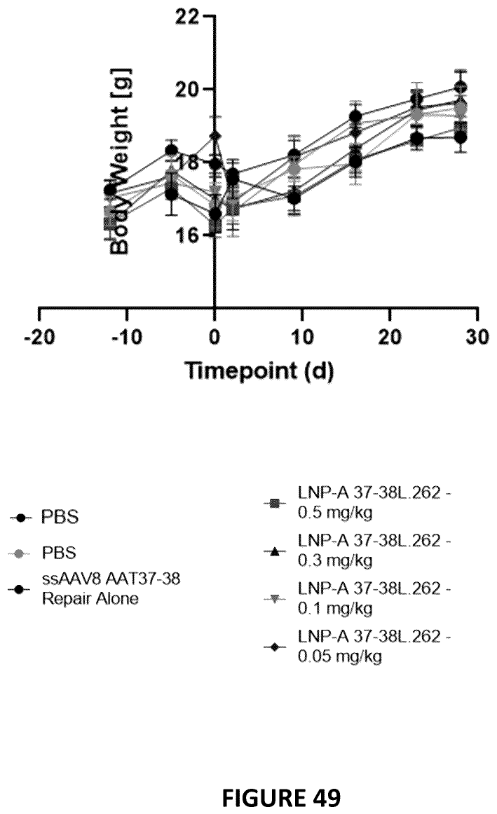

Serum chemistry analysis shows a decrease in ALT, AST, ALP and Tbil in meganuclease treated mice at all LNP doses at day 28 compared to PBS and repair template alone treated mice (FIG. 48). A slight increase in ALT, AST and ALP was observed at 2 days or 1 week post LNP and AAV administration but all meganuclease treated groups serum chemistry levels were back to baseline by day 28. LNP-A formulated with the generation 4 AAT 37-38L.262 meganuclease was well tolerated across all doses and there was no test article related toxicities observed in these mice, which were observed in Example 6 utilizing the earlier developed first generation 37-38 meganuclease. PiZ mouse body weights continued to rise after test article administration indicating the animals were healthy throughout the study (FIG. 49). This indicates that the later developed AAT 37-38 L.262 engineered meganuclease was better tolerated likely due to the greatly reduced off targeting profile established using the oligocapture assay (FIG. 34) in comparison to the first generation AAT 37-38x.50 meganuclease (FIG. 21).

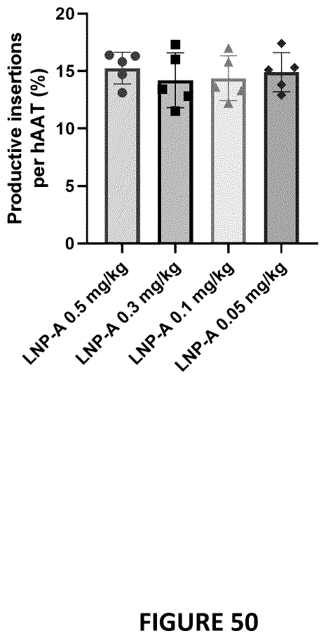

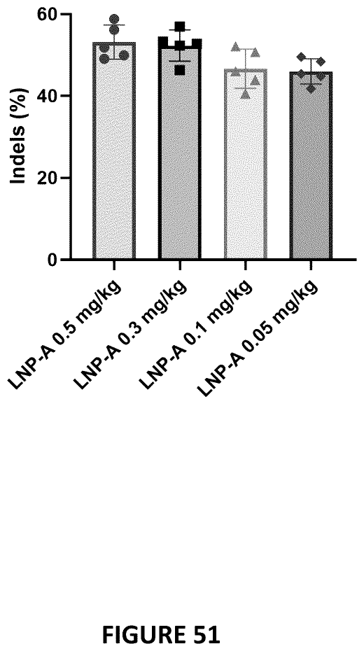

Insertion analysis demonstrated that there is no significant difference between any of the LNP-A doses for insertion of the WT AAT repair with an average insertion frequency of ˜14-15% (FIG. 50). However, indel analysis shows a slight dose mediated response across groups. The high dose (0.5 mg/kg) group shows an average of 53% indels and the low dose group shows an average of 45% indels (FIG. 51).

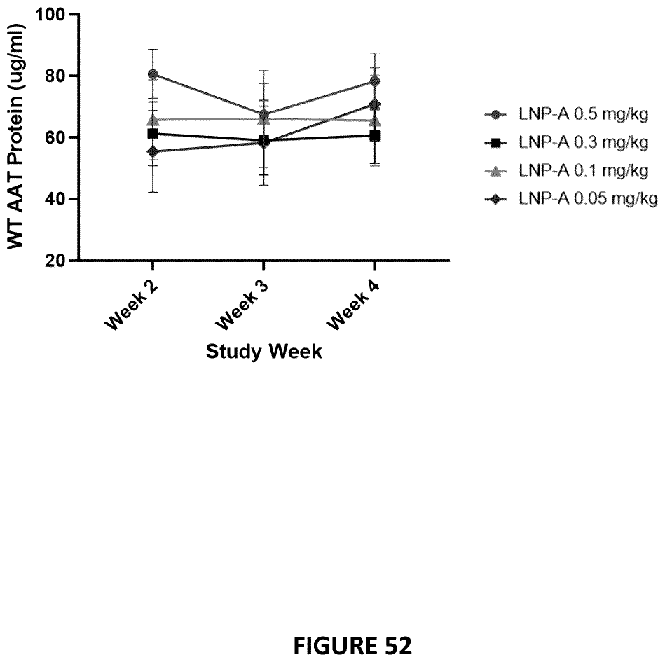

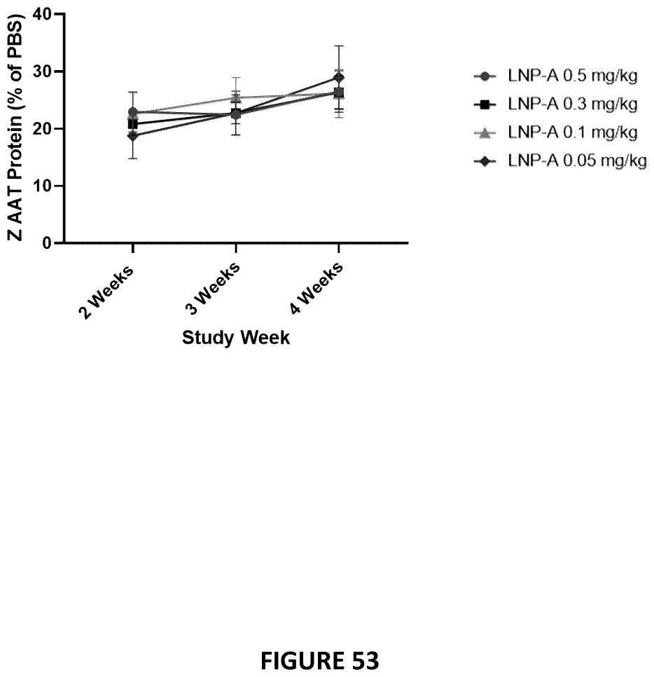

WT AAT protein analysis demonstrated an average of 50-80 μg/ml WT protein for all dose groups (FIG. 52). There was no dose response in WT protein-similar to the insertion data. The 0.5 mg/kg LNP-A treated groups had slightly higher AAT-WT protein levels than the other doses at weeks 2 and 4 (FIG. 52). The decrease in AAT-Z protein also showed no dose response between groups and there was a 20-30% decrease in AAT-Z protein for all groups (FIG. 53).

### 3. Conclusions

In conclusion, we demonstrated up to 15% insertions with LNP-A formulated with the generation 4 AAT37-38L.262 meganuclease and ssAAV8 repair template which resulted in up to 80 μg/ml of AAT-WT secreted protein and a 30% decrease in AAT-Z secreted protein in the PiZ mouse model. In addition, the later developed fourth generation AAT 37-38L.262 was well tolerated with no toxicity issues, which were observed in LNP delivery of the first generation AAT 37-38x.50 meganuclease of Example 4.

### Example 9

### 1. Methods and Materials

The PiZ mouse model, as described in Example 3, was used for this study to determine how the repair backbone and dose affect insertions when the meganuclease delivery method is via either ssAAV or LNP. The generation 4 AAT37-38L.262 meganuclease will be used in combination with the 37-38 repair template, as describe in example 3, as with ssAAV or scAAV. The study design is provided in Table 12 below.

TABLE 12
Study Design for Example 9

| Group | Treatment | LNP Dose (mg/kg) | N= |
|---|---|---|---|
| 1 | PBS | X | 4 |
| 2 | LNP-A AAT | 3.5 | 4 |
| 3 | 37-38L.262 | 3 | 4 |
| 4 | | 1 | 4 |

Blood will be collected from mice at weekly intervals from day-7 through day 56. Serum will be collected at days −7, 2, 9, 29 and day 56 for serum chemistry (ALT, AST, ALP, Tbil) analysis. Plasma will be isolated on study days 16, 23, 36, 43, 51 and 56 and analyzed as previously described in Example 3. Liver tissue will be harvested from the mice at day 56. Liver tissue will be harvested at day 56. DNA will be isolated from liver tissue as described in Example 3. AAT Transgene copy number quantification will be performed as described in Example 3. Insertion quantification will be performed as described in Example 4 using primers and probes provided in Table 5, assay 3. Indel quantification will be performed as described in Example 6, using primers and probes from Table 8, assay 13. Liver immunohistochemistry will be performed as described in Example 4

### Example 10

### In Vivo Tolerability Study of an LNP Containing an Engineered Meganuclease

### 1. Methods and Materials

Sprague Dawley rats were used to assess tolerability of LNP-A formulated with the AAT 37-38L.262 meganuclease at various doses to choose a dose for the NHP study described in Example 11. Blood was collected from rats prior to dosing and post dose at 4 hr, 48 hr and day 7 and analyzed for serum chemistry: ALT, AST, ALP and Tbil and cytokines: MCP-1, IFN-γ, IL-1B, TNF-α, IL-10 and KC/GRO. Livers were harvested at day 7, placed in 10% neutral buffer formalin (NBF) for at least 24 hours and subsequently transferred into 70% ethanol for at least 24 hours and then processed to paraffin blocks. Livers were stained with hematoxylin and eosin for histopathology analysis.

### 2. Results

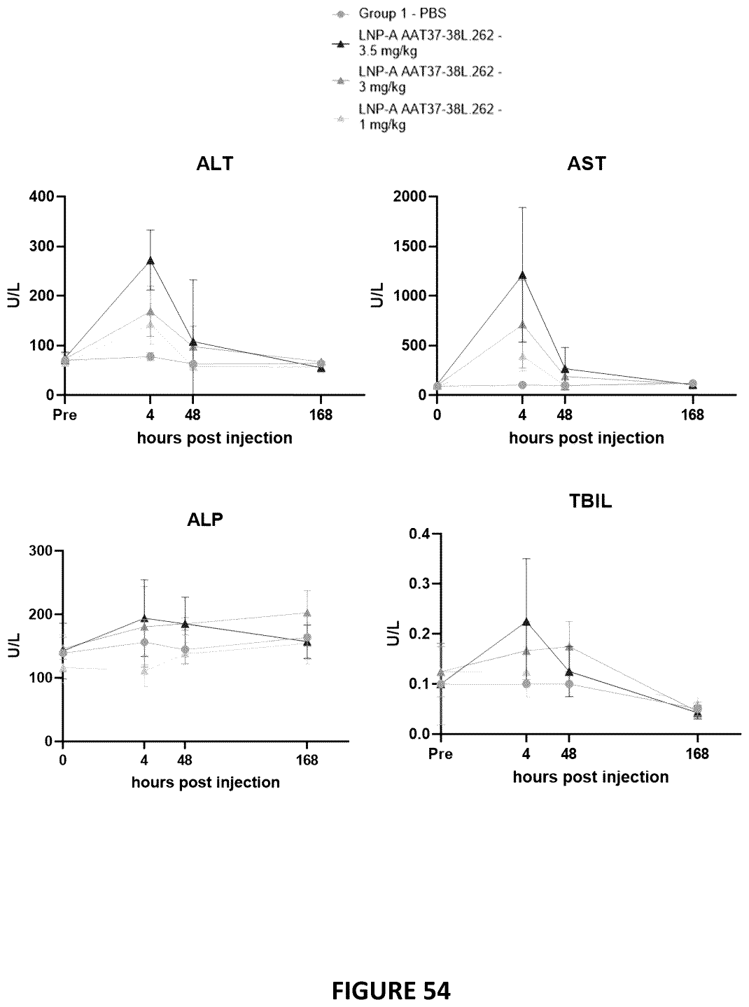

All LNP-A-AAT37-38L.262 doses were well tolerated and the rats survived through the 7-day study. Serum chemistry results show there was a dose response for ALT and AST levels, with the 3.5 mg/kg LNP-A dose reaching an average maximum level of 272 U/L and 1212U/L of ALT and AST at 4 hours post injection, which returns to baseline by day 7 FIG. 54. The ALP and Tbil levels are not significantly different compared to PBS treated animals FIG. 54.

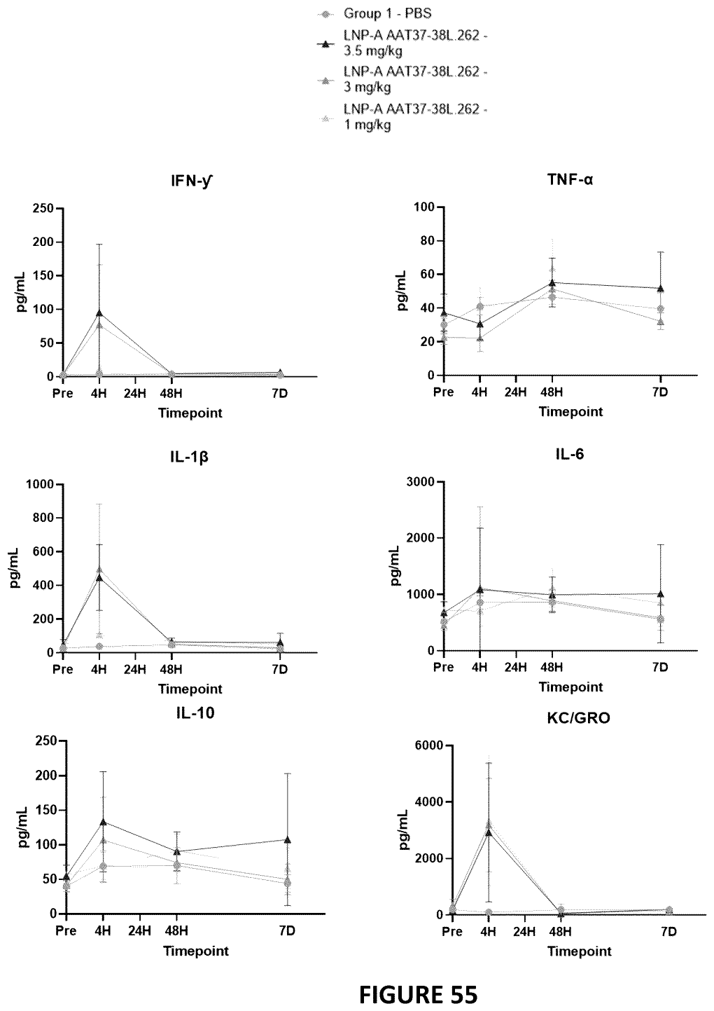

Cytokine analysis showed dose mediated increases for IFN-γ, IL-1B, IL-10 and KC/GRO at the 4-hour timepoint, which returned to baseline by 48 hours (FIG. 55). Interestingly, IL-10 at the 3.5 mg/kg LNP-A dose elevated slightly from 48 hours to day 7. There was no significant difference in TNF-α and IL-6 for all groups with the exception of TNF-α being slightly elevated in the 3 mg/kg group at 48 hours, which returns to baseline by day 7 (FIG. 55). Histopathology analysis found minimal to mild oval cell proliferation in the 3.5 mg/kg and 3 mg/kg LNP-A dosed rats and no lesions were observed in any rats from the 1 mg/kg dose group.

### 3. Conclusions

Overall, the LNP-A AAT37-38L.262 was well-tolerated at doses 3.5 mg/kg, 3 mg/kg and 1 mg/kg. Due to the transient elevation of ALT, AST, IFN-γ, IL-1B, IL-10 and KC/GRO, at 3.5 mg/kg and 3 mg/kg we decided to dose the NHPs at a lower dose of 1.75 mg/kg.

### Example 11

In vivo Gene Editing of Non-Human Primates with Engineered AAT Meganucleases

### 1. Methods and Materials

To analyze the insertion approach described herein in a large animal model, we will dose cynomolgus macaques with PBS, ssAAV8 repair alone (3e13 vg/kg) and the AAT37-38L.262 meganuclease delivered via ssAAV8 (5e12 vg/kg) or LNP-A (1.75 mg/kg) with the ssAAV8 repair (2.5e13 vg/kg and 3e13 vg/kg for AAV and LNP, respectively). The LNP dose was chosen based on the data of Example 9 and the AAV doses were chosen based on previous NHP studies showing a total of 3e13 vg/kg was well tolerated. Animals will receive prednisolone throughout the study in order to prevent the immune system from clearing secreted human W-AAT protein from the inserted repair. There will be a day 30 biopsy to understand how the insertion approach described herein is working at an earlier timepoint in a large animal model. All animals will be humanely euthanized at 3 months post dose. Blood will be collected throughout the study for serum chemistry and human WT-AAT protein analysis. Livers will be analyzed for insertions and indels via ddPCR similarly to the assays described in Example 4 and Example 6, respectively.

In addition, immunohistochemistry will be performed on liver tissue. Briefly, liver tissue will be placed in 10% neutral buffer formalin (NBF) for at least 24 hours and subsequently transferred into 70% ethanol for at least 24 hours and then processed to paraffin blocks for analysis. Hematoxylin and Eosin (H&E) and flag immunohistochemistry will be performed on the liver section for histopathology analysis and WT AAT expression from the inserted donor polynucleotide, respectively.

The study design for Example 11 is provided in Table 13 below.

TABLE 13
Study Design of Example 11.

| | | Repair | | LNP | |
|---|---|---|---|---|---|
| | | Dose | Meganuclease | Meganuclease | |
| Group | Treatment | (vg/kg) | Dose (vg/kg) | Dose (mg/kg) | N= |
| 1 | PBS | X | X | X | 1 |
| 2 | SSAAV8 | 3e13 | X | X | 2 |
| | AAT37-38 | | | | |
| | Repair Only | | | | |
| 3 | SSAAV8AAT37- | 2.5e13 | 5e12 | X | 3 |
| | 38L.262/ssAAV8 | | | | |
| | 37-38 | | | | |
| 4 | LNP-A | 3e13 | X | 1.75 | 3 |
| | 37-38L.262/ | | | | |
| | ssAAV8 | | | | |
| | 37-38 | | | | |

Sequence Listing

SEQ ID NO: 1
MNTKYNKEFLLYLAGFVDGDGSIIAQIKPNQSYKFKHQLSLAFQVTQKTQRRWFLD
KLVDEIGVGYVRDRGSVSDYILSEIKPLHNFLTQLQPFLKLKQKQANLVLKIIWRLPS
AKESPDKFLEVCTWVDQIAALNDSKTRKTTSETVRAVLDSLSEKKKSSP
SEQ ID NO: 2
LAGLIDADG
SEQ ID NO: 3
GGCGGCAGTAAGTCTTCAGCATCAGGCATTTTGGGGTGACTCAGTAAATGGTAG
ATCTTGCTACCAGTGGAACAGCCACTAAGGATTCTGCAGTGAGAGCAGAGGGCC
AGCTAAGTGGTACTCTCCCAGAGACTGTCTGACTCACGCCACCCCCTCCACCTTG
GACACAGGACGCTGTGGTTTCTGAGCCAGGTACAATGACTCCTTTCGGTAAGTGC
AGTGGAAGCTGTACACTGCCCAGGCAAAGCGTCCGGGCAGCGTAGGCGGGCGAC
TCAGATCCCAGCCAGTGGACTTAGCCCCTGTTTGCTCCTCCGATAACTGGGGTGA
CCTTGGTTAATATTCACCAGCAGCCTCCCCCGTTGCCCCTCTGGATCCACTGCTTA
AATACGGACGAGGACAGGGCCCTGTCTCCTCAGCTTCAGGCACCACCACTGACC
TGGGACAGTGAATCGTAAGTATGCCTTTCACTGCGAGAGGTTCTGGAGAGGCTTC
TGAGCTCCCCATGGCCCAGGCAGGCAGCAGGTCTGGGGCAGGAGGGGGGTTGTG
GAGTGGGTATCCGCCTGCTGAGGTGCAGGGCAGATGGAGAGGCTGCAGCTGAGC
TCCTATTTTCATAATAACAGCAGCCATGAGGGTTGTGTCCTGTTTCCCAGTCCTGC
CCGGTCCCCCCTCGGTACCTCCTGGTGGATACACTGGTTCCTGTAAGCAGAAGTG
GATGAGGGTGTCTAGGTCTGCAGTCCTGGCACCCCAGGATGGGGGACACCAGCC
AAGATACAGCAACAGCAACAAAGCGCAGCCATTTCTTTCTGTTTGCACAGCTCCT
CTGTCTGTCGGGGGCTCCTGTCTGTTGTCTCCTATAAGCCTCACCACCTCTCCTAC
TGCTTGGGCATGCATCTTTCTCCCCTTCTATAGATGAGGAGGTTAAGGTCCAGAG
AGGGGTGGGGAGGAACGCCGGCTCACATTCTCCATCCCCTCCAGATATGACCAG
GAACAGACCTGTGCCAGGCCTCAGCCTTACATCAAAATGGGCCTCCCCATGCACC
GTGGACCTCTGGGCCCTCCTGTCCCAGTGGAGGACAGGAAGCTGTGAGGGGCAC
TGTCACCCAGGGCTCAAGCTGGCATTCCTGAATAATCGCTCTGCACCAGGCCACG
GCTAAGCTCAGTGCGTGATTAAGCCTCATAACCCTCCAAGGCAGTTACTAGTGTG
ATTCCCATTTTACAGATGAGGAAGATGGGGACAGAGAGGTGAATAACTGGCCCC
AAATCACACACCATCCATAATTCGGGCTCAGGCACCTGGCTCCAGTCCCCAAACT
CTTGAACCTGGCCCTAGTGTCACTGTTTCTCTTGGGTCTCAGGCGCTGGATGGGG
AACAGGAAACCTGGGCTGGACTTGAGGCCTCTCTGATGCTCGGTGACTTCAGAC
AGTTGCTCAACCTCTCTGTTCTCTTGGGCAAAACATGATAACCTTTGACTTCTGTC
CCCTCCCCTCACCCCACCCGACCTTGATCTCTGAAGTGTTGGAAGGATTTAATTTT
TCCTGCACTGAGTTTTGGAGACAGGTCAAAAAGATGACCAAGGCCAAGGTGGCC
AGTTTCCTATAGAACGCCTCTAAAAGACCTGCAGCAATAGCAGCAAGAACTGGT
ATTCTCGAGAACTTGCTGCGCAGCAGGCACTTCTTGGCATTTTATGTGTATTTAAT
TTCACAATAGCTCTATGACAAAGTCCACCTTTCTCATCTCCAGGAAACTGAGGTT
CAGAGAGGTTAAGTAACTTGTCCAAGGTCACACAGCTAATAGCAAGTTGACGTG
GAGCAATCTGGCCTCAGAGCCTTTAATTTTAGCCACAGACTGATGCTCCCCTCTT
CATTTAGCCAGGCTGCCTCTGAAGTTTTCTGATTCAAGACTTCTGGCTTCAGCTTT
GTACACAGAGATGATTCAATGTCAGGTTTTGGAGTGAAATCTGTTTAATCCCAGA
CAAAACATTTAGGATTACATCTCAGTTTTGTAAGCAAGTAGCTCTGTGATTTTTA
GTGAGTTATTTAATGCTCTTTGGGGCTCAATTTTTCTATCTATAAAATAGGGCTAA
TAATTTGCACCTTATAGGGTAAGCTTTGAGGACAGATTAGATGATACGGTGCCTG
TAAAACACCAGGTGTTAGTAAGTGTGGCAATGATGGTGACGCTGAGGCTGATGT
TTGCTTAGCATAGGGTTAGGCAGCTGGCAGGCAGTAAACAGTTGGATAATTTAAT
GGAAAATTTGCCAAACTCAGATGCTGTTCACTGCTGAGCAGGAGCCCCTTCCTGC
TGAAATGGTCCTGGGGAGTGCAGCAGGCTCTCCGGGAAGAAATCTACCATCTCT
CGGGCAGGAGCTCAACCTGTGTGCAGGTACAGGGAGGGCTTCCTCACCTGGTGC
CCACTCATGCATTACGTCAGTTATTCCTCATCCCTGTCCAAAGGATTCTTTTCTCC
ATTGTACAGCTATGAAGCTAGTGCTCAAAGAAGTGAAGTCATTTACCCCAGGCCC
CCTGCCAGTAAGTGACAGGGCCTGGTCACACTTGGGTTTATTTATTGCCCAGTTC
AACAGGTTGTTTGACCATAGGCGAGATTCTCTTCCCTGCACCCTGCCGGGTTGCT
CTTGGTCCCTTATTTTATGCTCCCGGGTAGAAATGGTGTGAGATTAGGCAGGGAG
TGGCTCGCTTCCCTGTCCCTGGCCCCGCAAAGAGTGCTCCCACCTGCCCCGATCC
CAGAAATGTCACCATGAAGCCTTCATTCTTTTGGTTTAAAGCTTGGCCTCAGTGT
CCGTACACCATGGGGTACTTGGCCAGATGGCGACTTTCTCCTCTCCAGTCGCCCT
CCCAGGCACTAGCTTTTAGGAGTGCAGGGTGCTGCCTCTGATAGAAGGGCCAGG
AGAGAGCAGGTTTTGGAGTCCTGATGTTATAAGGAACAGCTTGGGAGGCATAAT
GAACCCAACATGATGCTTGAGACCAATGTCACAGCCCAATTCTGACATTCATCAT
CTGAGATCTGAGGACACAGCTGTCTCAGTTCATGATCTGAGTGCTGGGAAAGCC
AAGACTTGTTCCAGCTTTGTCACTGACTTGCTGTATAGCCTCAACAAGGCCCTGA
CCCTCTCTGGGCTTCAAACTCTTCACTGTGAAAGGAGGAAACCAGAGTAGGTGAT
GTGACACCAGGAAAGATGGATGGGTGTGGGGGAATGTGCTCCTCCCAGCTGTCA
CCCCCTCGCCACCCTCCCTGCACCAGCCTCTCCACCTCCTTTGAGCCCAGAATTCC
CCTGTCTAGGAGGGCACCTGTCTCATGCCTAGCCATGGGAATTCTCCATCTGTTTT
GCTACATTGAACCCAGATGCCATTCTAACCAAGAATCCTGGCTGGGTGCAGGGG
CTCTCGCCTGTAACCCCAGCACTTTGGGAGGCCAAGGCAGGCGGATCAAGAGGT
CAGGAGTTCAAGACCTGCCTGGCCAACACGGTGAAACCTCAGCTCTACTAAAAA
TACAAAAATTAGCCAGGCGTGGTGGCACACGCCTGTAATCCCAGCTATTTGGGA
AGCTGAGACAGAAGAATTTCTTGAACCCGGGAGGTGGAGGTTTCAGTGAGCCGA
GATCACGCCACTGCACTCCACCCTGGCAGATAAAGCGAGACTCTGTCTCAAAAA
AAACCCAAAAACCTATGTTAGTGTACAGAGGGCCCCAGTGAAGTCTTCTCCCAG
CCCCACTTTGCACAACTGGGGAGAGTGAGGCCCCAGGACCAGAGGATTCTTGCT
AAAGGCCAAGTGGATAGTGATGGCCCTGCCAGGGCTAGAAGCCACAACCTCTGG
CCCTGAGGCCACTCAGCATATTTAGTGTCCCCACCCTGCAGAGGCCCAACTCCCT
CCTGACCACTGAGCCCTGTAATGATGGGGGAATTTCCATAAGCCATGAAGGACT
GCACAAAGTTCAGTTGGGAAGTGAAAGAGAAATTAAAGGGAGATGGAAATATA
CAGCACTAATTTTAGCACCGTCTTTAGTTCTAACAACACTAGCTAGCTGAAGAAA
AATACAAACATGTATTATGTAATGTGTGGTCTGTTCCATTTGGATTACTTAGAGG
CACGAGGGCCAGGAGAAAGGTGGTGGAGAGAAACCAGCTTTGCACTTCATTTGT
TGCTTTATTGGAAGGAAACTTTTAAAAGTCCAAGGGGGTTGAAGAATCTCAATAT
TTGTTATTTCCAGCTTTTTTTCTCCAGTTTTTCATTTCCCAAATTCAAGGACACCTT
TTTCTTTGTATTTTGTTAAGATGATGGTTTTGGTTTTGTGACTAGTAGTTAACAAT
GTGGCTGCCGGGCATATTCTCCTCAGCTAGGACCTCAGTTTTCCCATCTGTGAAG
ACGGCAGGTTCTACCTAGGGGGCTGCAGGCTGGTGGTCCGAAGCCTGGGCATAT
CTGGAGTAGAAGGATCACTGTGGGGCAGGGCAGGTTCTGTGTTGCTGTGGATGA
CGTTGACTTTGACCATTGCTCGGCAGAGCCTGCTCTCGCTGGTTCAGCCACAGGC
CCCACCACTCCCTATTGTCTCAGCCCCGGGTATGAAACATGTATTCCTCACTGGC
CTATCACCTGAAGCCTTTGAATTTGCAACACCTGCCAACCCCTCCCTCAAAAGAG
TTGCCCTCTCAGATCCTTTTGATGTAAGGTTTGGTGTTGAGACTTATTTCACTAAA
TTCTCATACATAAACATCACTTTATGTATGAGGCAAAATGAGGACCAGGGAGAT
GAATGACTTGTCCTGGCTCATACACCTGGAAAGTGACAGAGTCAGATTAGATCCC
AGGTCTATCTGAAGTTAAAAGAGGTGTCTTTTCACTTCCCACCTCCTCCATCTACT
TTAAAGCAGCACAAACCCCTGCTTTCAAGGAGAGATGAGCGTCTCTAAAGCCCC
TGACAGCAAGAGCCCAGAACTGGGACACCATTAGTGACCCAGACGGCAGGTAAG
CTGACTGCAGGAGCATCAGCCTATTCTTGTGTCTGGGACCACAGAGCATTGTGGG
GACAGCCCCGTCTCTTGGGAAAAAAACCCTAAGGGCTGAGGATCCTTGTGAGTG
TTGGGTGGGAACAGCTCCCAGGAGGTTTAATCACAGCCCCTCCATGCTCTCTAGC
TGTTGCCATTGTGCAAGATGCATTTCCCTTCTGTGCAGCAGTTTCCCTGGCCACTA
AATAGTGGGATTAGATAGAAGCCCTCCAAGGGCTTCCAGCTTGACATGATTCTTG
ATTCTGATCTGGCCCGATTCCTGGATAATCGTGGGCAGGCCCATTCCTCTTCTTGT
GCCTCATTTTCTTCTTTTGTAAAACAATGGCTGTACCATTTGCATCTTAGGGTCAT
TGCAGATGTAAGTGTTGCTGTCCAGAGCCTGGGTGCAGGACCTAGATGTAGGATT
CTGGTTCTGCTACTTCCTCAGTGACATTGAATAGCTGACCTAATCTCTCTGGCTTT
GGTTTCTTCATCTGTAAAAGAAGGATATTAGCATTAGCACCTCACGGGATTGTTA
CAAGAAAGCAATGAATTAACACATGTGAGCACGGAGAACAGTGCTTGGCATATG
GTAAGCACTACGTACATTTTGCTATTCTTCTGATTCTTTCAGTGTTACTGATGTCG
GCAAGTACTTGGCACAGGCTGGTTTAATAATCCCTAGGCACTTCCACGTGGTGTC
AATCCCTGATCACTGGGAGTCATCATGTGCCTTGACTCGGGGCCTGGCCCCCCCA
TCTCTGTCTTGCAGGACAATGCCGTCTTCTGTCTCGTGGGGCATCCTCCTGCTGGC
AGGCCTGTGCTGCCTGGTCCCTGTCTCCCTGGCTGAGGATCCCCAGGGAGATGCT
GCCCAGAAGACAGATACATCCCACCATGATCAGGATCACCCAACCTTCAACAAG
ATCACCCCCAACCTGGCTGAGTTCGCCTTCAGCCTATACCGCCAGCTGGCACACC
AGTCCAACAGCACCAATATCTTCTTCTCCCCAGTGAGCATCGCTACAGCCTTTGC
AATGCTCTCCCTGGGGACCAAGGCTGACACTCACGATGAAATCCTGGAGGGCCT
GAATTTCAACCTCACGGAGATTCCGGAGGCTCAGATCCATGAAGGCTTCCAGGA
ACTCCTCCGTACCCTCAACCAGCCAGACAGCCAGCTCCAGCTGACCACCGGCAAT
GGCCTGTTCCTCAGCGAGGGCCTGAAGCTAGTGGATAAGTTTTTGGAGGATGTTA
AAAAGTTGTACCACTCAGAAGCCTTCACTGTCAACTTCGGGGACACCGAAGAGG
CCAAGAAACAGATCAACGATTACGTGGAGAAGGGTACTCAAGGGAAAATTGTGG
ATTTGGTCAAGGAGCTTGACAGAGACACAGTTTTTGCTCTGGTGAATTACATCTT
CTTTAAAGGCAAATGGGAGAGACCCTTTGAAGTCAAGGACACCGAGGAAGAGG
ACTTCCACGTGGACCAGGTGACCACCGTGAAGGTGCCTATGATGAAGCGTTTAG
GCATGTTTAACATCCAGCACTGTAAGAAGCTGTCCAGCTGGGTGCTGCTGATGAA
ATACCTGGGCAATGCCACCGCCATCTTCTTCCTGCCTGATGAGGGGAAACTACAG
CACCTGGAAAATGAACTCACCCACGATATCATCACCAAGTTCCTGGAAAATGAA
GACAGAAGGTCTGCCAGCTTACATTTACCCAAACTGTCCATTACTGGAACCTATG
ATCTGAAGAGCGTCCTGGGTCAACTGGGCATCACTAAGGTCTTCAGCAATGGGG
CTGACCTCTCCGGGGTCACAGAGGAGGCACCCCTGAAGCTCTCCAAGGCCGTGC
ATAAGGCTGTGCTGACCATCGACGAGAAAGGGACTGAAGCTGCTGGGGCCATGT
TTTTAGAGGCCATACCCATGTCTATCCCCCCCGAGGTCAAGTTCAACAAACCCTT
TGTCTTCTTAATGATTGAACAAAATACCAAGTCTCCCCTCTTCATGGGAAAAGTG
GTGAATCCCACCCAAAAATAA
SEQ ID NO: 4
GGCGGCAGTAAGTCTTCAGCATCAGGCATTTTGGGGTGACTCAGTAAATGGTAG
ATCTTGCTACCAGTGGAACAGCCACTAAGGATTCTGCAGTGAGAGCAGAGGGCC
AGCTAAGTGGTACTCTCCCAGAGACTGTCTGACTCACGCCACCCCCTCCACCTTG
GACACAGGACGCTGTGGTTTCTGAGCCAGGTACAATGACTCCTTTCGGTAAGTGC
AGTGGAAGCTGTACACTGCCCAGGCAAAGCGTCCGGGCAGCGTAGGCGGGCGAC
TCAGATCCCAGCCAGTGGACTTAGCCCCTGTTTGCTCCTCCGATAACTGGGGTGA
CCTTGGTTAATATTCACCAGCAGCCTCCCCCGTTGCCCCTCTGGATCCACTGCTTA
AATACGGACGAGGACAGGGCCCTGTCTCCTCAGCTTCAGGCACCACCACTGACC
TGGGACAGTGAATCGTAAGTATGCCTTTCACTGCGAGAGGTTCTGGAGAGGCTTC
TGAGCTCCCCATGGCCCAGGCAGGCAGCAGGTCTGGGGCAGGAGGGGGGTTGTG
GAGTGGGTATCCGCCTGCTGAGGTGCAGGGCAGATGGAGAGGCTGCAGCTGAGC
TCCTATTTTCATAATAACAGCAGCCATGAGGGTTGTGTCCTGTTTCCCAGTCCTGC
CCGGTCCCCCCTCGGTACCTCCTGGTGGATACACTGGTTCCTGTAAGCAGAAGTG
GATGAGGGTGTCTAGGTCTGCAGTCCTGGCACCCCAGGATGGGGGACACCAGCC
AAGATACAGCAACAGCAACAAAGCGCAGCCATTTCTTTCTGTTTGCACAGCTCCT
CTGTCTGTCGGGGGCTCCTGTCTGTTGTCTCCTATAAGCCTCACCACCTCTCCTAC
TGCTTGGGCATGCATCTTTCTCCCCTTCTATAGATGAGGAGGTTAAGGTCCAGAG
AGGGGTGGGGAGGAACGCCGGCTCACATTCTCCATCCCCTCCAGATATGACCAG
GAACAGACCTGTGCCAGGCCTCAGCCTTACATCAAAATGGGCCTCCCCATGCACC
GTGGACCTCTGGGCCCTCCTGTCCCAGTGGAGGACAGGAAGCTGTGAGGGGCAC
TGTCACCCAGGGCTCAAGCTGGCATTCCTGAATAATCGCTCTGCACCAGGCCACG
GCTAAGCTCAGTGCGTGATTAAGCCTCATAACCCTCCAAGGCAGTTACTAGTGTG
ATTCCCATTTTACAGATGAGGAAGATGGGGACAGAGAGGTGAATAACTGGCCCC
AAATCACACACCATCCATAATTCGGGCTCAGGCACCTGGCTCCAGTCCCCAAACT
CTTGAACCTGGCCCTAGTGTCACTGTTTCTCTTGGGTCTCAGGCGCTGGATGGGG
AACAGGAAACCTGGGCTGGACTTGAGGCCTCTCTGATGCTCGGTGACTTCAGAC
AGTTGCTCAACCTCTCTGTTCTCTTGGGCAAAACATGATAACCTTTGACTTCTGTC
CCCTCCCCTCACCCCACCCGACCTTGATCTCTGAAGTGTTGGAAGGATTTAATTTT
TCCTGCACTGAGTTTTGGAGACAGGTCAAAAAGATGACCAAGGCCAAGGTGGCC
AGTTTCCTATAGAACGCCTCTAAAAGACCTGCAGCAATAGCAGCAAGAACTGGT
ATTCTCGAGAACTTGCTGCGCAGCAGGCACTTCTTGGCATTTTATGTGTATTTAAT
TTCACAATAGCTCTATGACAAAGTCCACCTTTCTCATCTCCAGGAAACTGAGGTT
CAGAGAGGTTAAGTAACTTGTCCAAGGTCACACAGCTAATAGCAAGTTGACGTG
GAGCAATCTGGCCTCAGAGCCTTTAATTTTAGCCACAGACTGATGCTCCCCTCTT
CATTTAGCCAGGCTGCCTCTGAAGTTTTCTGATTCAAGACTTCTGGCTTCAGCTTT
GTACACAGAGATGATTCAATGTCAGGTTTTGGAGTGAAATCTGTTTAATCCCAGA
CAAAACATTTAGGATTACATCTCAGTTTTGTAAGCAAGTAGCTCTGTGATTTTTA
GTGAGTTATTTAATGCTCTTTGGGGCTCAATTTTTCTATCTATAAAATAGGGCTAA
TAATTTGCACCTTATAGGGTAAGCTTTGAGGACAGATTAGATGATACGGTGCCTG
TAAAACACCAGGTGTTAGTAAGTGTGGCAATGATGGTGACGCTGAGGCTGATGT
TTGCTTAGCATAGGGTTAGGCAGCTGGCAGGCAGTAAACAGTTGGATAATTTAAT
GGAAAATTTGCCAAACTCAGATGCTGTTCACTGCTGAGCAGGAGCCCCTTCCTGC
TGAAATGGTCCTGGGGAGTGCAGCAGGCTCTCCGGGAAGAAATCTACCATCTCT
CGGGCAGGAGCTCAACCTGTGTGCAGGTACAGGGAGGGCTTCCTCACCTGGTGC
CCACTCATGCATTACGTCAGTTATTCCTCATCCCTGTCCAAAGGATTCTTTTCTCC
ATTGTACAGCTATGAAGCTAGTGCTCAAAGAAGTGAAGTCATTTACCCCAGGCCC
CCTGCCAGTAAGTGACAGGGCCTGGTCACACTTGGGTTTATTTATTGCCCAGTTC
AACAGGTTGTTTGACCATAGGCGAGATTCTCTTCCCTGCACCCTGCCGGGTTGCT
CTTGGTCCCTTATTTTATGCTCCCGGGTAGAAATGGTGTGAGATTAGGCAGGGAG
TGGCTCGCTTCCCTGTCCCTGGCCCCGCAAAGAGTGCTCCCACCTGCCCCGATCC
CAGAAATGTCACCATGAAGCCTTCATTCTTTTGGTTTAAAGCTTGGCCTCAGTGT
CCGTACACCATGGGGTACTTGGCCAGATGGCGACTTTCTCCTCTCCAGTCGCCCT
CCCAGGCACTAGCTTTTAGGAGTGCAGGGTGCTGCCTCTGATAGAAGGGCCAGG
AGAGAGCAGGTTTTGGAGTCCTGATGTTATAAGGAACAGCTTGGGAGGCATAAT
GAACCCAACATGATGCTTGAGACCAATGTCACAGCCCAATTCTGACATTCATCAT
CTGAGATCTGAGGACACAGCTGTCTCAGTTCATGATCTGAGTGCTGGGAAAGCC
AAGACTTGTTCCAGCTTTGTCACTGACTTGCTGTATAGCCTCAACAAGGCCCTGA
CCCTCTCTGGGCTTCAAACTCTTCACTGTGAAAGGAGGAAACCAGAGTAGGTGAT
GTGACACCAGGAAAGATGGATGGGTGTGGGGGAATGTGCTCCTCCCAGCTGTCA
CCCCCTCGCCACCCTCCCTGCACCAGCCTCTCCACCTCCTTTGAGCCCAGAATTCC
CCTGTCTAGGAGGGCACCTGTCTCATGCCTAGCCATGGGAATTCTCCATCTGTTTT
GCTACATTGAACCCAGATGCCATTCTAACCAAGAATCCTGGCTGGGTGCAGGGG
CTCTCGCCTGTAACCCCAGCACTTTGGGAGGCCAAGGCAGGCGGATCAAGAGGT
CAGGAGTTCAAGACCTGCCTGGCCAACACGGTGAAACCTCAGCTCTACTAAAAA
TACAAAAATTAGCCAGGCGTGGTGGCACACGCCTGTAATCCCAGCTATTTGGGA
AGCTGAGACAGAAGAATTTCTTGAACCCGGGAGGTGGAGGTTTCAGTGAGCCGA
GATCACGCCACTGCACTCCACCCTGGCAGATAAAGCGAGACTCTGTCTCAAAAA
AAACCCAAAAACCTATGTTAGTGTACAGAGGGCCCCAGTGAAGTCTTCTCCCAG
CCCCACTTTGCACAACTGGGGAGAGTGAGGCCCCAGGACCAGAGGATTCTTGCT
AAAGGCCAAGTGGATAGTGATGGCCCTGCCAGGGCTAGAAGCCACAACCTCTGG
CCCTGAGGCCACTCAGCATATTTAGTGTCCCCACCCTGCAGAGGCCCAACTCCCT
CCTGACCACTGAGCCCTGTAATGATGGGGGAATTTCCATAAGCCATGAAGGACT
GCACAAAGTTCAGTTGGGAAGTGAAAGAGAAATTAAAGGGAGATGGAAATATA
CAGCACTAATTTTAGCACCGTCTTTAGTTCTAACAACACTAGCTAGCTGAAGAAA
AATACAAACATGTATTATGTAATGTGTGGTCTGTTCCATTTGGATTACTTAGAGG
CACGAGGGCCAGGAGAAAGGTGGTGGAGAGAAACCAGCTTTGCACTTCATTTGT
TGCTTTATTGGAAGGAAACTTTTAAAAGTCCAAGGGGGTTGAAGAATCTCAATAT
TTGTTATTTCCAGCTTTTTTTCTCCAGTTTTTCATTTCCCAAATTCAAGGACACCTT
TTTCTTTGTATTTTGTTAAGATGATGGTTTTGGTTTTGTGACTAGTAGTTAACAAT
GTGGCTGCCGGGCATATTCTCCTCAGCTAGGACCTCAGTTTTCCCATCTGTGAAG
ACGGCAGGTTCTACCTAGGGGGCTGCAGGCTGGTGGTCCGAAGCCTGGGCATAT
CTGGAGTAGAAGGATCACTGTGGGGCAGGGCAGGTTCTGTGTTGCTGTGGATGA
CGTTGACTTTGACCATTGCTCGGCAGAGCCTGCTCTCGCTGGTTCAGCCACAGGC
CCCACCACTCCCTATTGTCTCAGCCCCGGGTATGAAACATGTATTCCTCACTGGC
CTATCACCTGAAGCCTTTGAATTTGCAACACCTGCCAACCCCTCCCTCAAAAGAG
TTGCCCTCTCAGATCCTTTTGATGTAAGGTTTGGTGTTGAGACTTATTTCACTAAA
TTCTCATACATAAACATCACTTTATGTATGAGGCAAAATGAGGACCAGGGAGAT
GAATGACTTGTCCTGGCTCATACACCTGGAAAGTGACAGAGTCAGATTAGATCCC
AGGTCTATCTGAAGTTAAAAGAGGTGTCTTTTCACTTCCCACCTCCTCCATCTACT
TTAAAGCAGCACAAACCCCTGCTTTCAAGGAGAGATGAGCGTCTCTAAAGCCCC
TGACAGCAAGAGCCCAGAACTGGGACACCATTAGTGACCCAGACGGCAGGTAAG
CTGACTGCAGGAGCATCAGCCTATTCTTGTGTCTGGGACCACAGAGCATTGTGGG
GACAGCCCCGTCTCTTGGGAAAAAAACCCTAAGGGCTGAGGATCCTTGTGAGTG
TTGGGTGGGAACAGCTCCCAGGAGGTTTAATCACAGCCCCTCCATGCTCTCTAGC
TGTTGCCATTGTGCAAGATGCATTTCCCTTCTGTGCAGCAGTTTCCCTGGCCACTA
AATAGTGGGATTAGATAGAAGCCCTCCAAGGGCTTCCAGCTTGACATGATTCTTG
ATTCTGATCTGGCCCGATTCCTGGATAATCGTGGGCAGGCCCATTCCTCTTCTTGT
GCCTCATTTTCTTCTTTTGTAAAACAATGGCTGTACCATTTGCATCTTAGGGTCAT
TGCAGATGTAAGTGTTGCTGTCCAGAGCCTGGGTGCAGGACCTAGATGTAGGATT
CTGGTTCTGCTACTTCCTCAGTGACATTGAATAGCTGACCTAATCTCTCTGGCTTT
GGTTTCTTCATCTGTAAAAGAAGGATATTAGCATTAGCACCTCACGGGATTGTTA
CAAGAAAGCAATGAATTAACACATGTGAGCACGGAGAACAGTGCTTGGCATATG
GTAAGCACTACGTACATTTTGCTATTCTTCTGATTCTTTCAGTGTTACTGATGTCG
GCAAGTACTTGGCACAGGCTGGTTTAATAATCCCTAGGCACTTCCACGTGGTGTC
AATCCCTGATCACTGGGAGTCATCATGTGCCTTGACTCGGGGCCTGGCCCCCCCA
TCTCTGTCTTGCAGGACAATGCCGTCTTCTGTCTCGTGGGGCATCCTCCTGCTGGC
AGGCCTGTGCTGCCTGGTCCCTGTCTCCCTGGCTGAGGATCCCCAGGGAGATGCT
GCCCAGAAGACAGATACATCCCACCATGATCAGGATCACCCAACCTTCAACAAG
ATCACCCCCAACCTGGCTGAGTTCGCCTTCAGCCTATACCGCCAGCTGGCACACC
AGTCCAACAGCACCAATATCTTCTTCTCCCCAGTGAGCATCGCTACAGCCTTTGC
AATGCTCTCCCTGGGGACCAAGGCTGACACTCACGATGAAATCCTGGAGGGCCT
GAATTTCAACCTCACGGAGATTCCGGAGGCTCAGATCCATGAAGGCTTCCAGGA
ACTCCTCCGTACCCTCAACCAGCCAGACAGCCAGCTCCAGCTGACCACCGGCAAT
GGCCTGTTCCTCAGCGAGGGCCTGAAGCTAGTGGATAAGTTTTTGGAGGATGTTA
AAAAGTTGTACCACTCAGAAGCCTTCACTGTCAACTTCGGGGACACCGAAGAGG
CCAAGAAACAGATCAACGATTACGTGGAGAAGGGTACTCAAGGGAAAATTGTGG
ATTTGGTCAAGGAGCTTGACAGAGACACAGTTTTTGCTCTGGTGAATTACATCTT
CTTTAAAGGTAAGGTTGCTCAACCAGCCTGAGCTGTTCCCATAGAAACAAGCAA
AAATATTCTCAAACCATCAGTTCTTGAACTCTCCTTGGCAATGCATTATGGGCCA
TAGCAATGCTTTTCAGCGTGGATTCTTCAGTTTTCTACACACAAACACTAAAATG
TTTTCCATCATTGAGTAATTTGAGGAAATAATAGATTAAACTGTCAAAACTACTG
ACAGCTCTGCAGAACTTTTCAGAGCCTTTAATGTCCTTGTGTATACTGTATATGTA
GAATATATAATGCTTAGAACTATAGAACAAATTGTAATACACTGCATAAAGGGA
TAGTTTCATGGAACATACTTTACACGACTCTAGTGTCCCAGAATCAGTATCAGTT
TTGCAATCTGAAAGACCTGGGTTCAAATCCTGCCTCTAACACAATTAGCTTTTGA
CAAAAACAATGCATTCTACCTCTTTGAGGTGCTAATTTCTCATCTTAGCATGGAC
AAAATACCATTCTTGCTGTCAGGTTTTTTTAGGATTAAACAAATGACAAAGACTG
TGGGGATGGTGTGTGGCATACAGCAGGTGATGGACTCTTCTGTATCTCAGGCTGC
CTTCCTGCCCCTGAGGGGTTAAAATGCCAGGGTCCTGGGGGCCCCAGGGCATTCT
AAGCCAGCTCCCACTGTCCCAGGAAAACAGCATAGGGGAGGGGAGGTGGGAGG
CAAGGCCAGGGGCTGCTTCCTCCACTCTGAGGCTCCCTTGCTCTTGAGGCAAAGG
AGGGCAGTGGAGAGCAGCCAGGCTGCAGTCAGCACAGCTAAAGTCCTGGCTCTG
CTGTGGCCTTAGTGGGGGCCCAGGTCCCTCTCCAGCCCCAGTCTCCTCCTTCTGTC
CAATGAGAAAGCTGGGATCAGGGGTCCCTGAGGCCCCTGTCCACTCTGCATGCCT
CGATGGTGAAGCTCTGTTGGTATGGCAGAGGGGAGGCTGCTCAGGCATCTGCAT
TTCCCCTGCCAATCTAGAGGATGAGGAAAGCTCTCAGGAATAGTAAGCAGAATG
TTTGCCCTGGATGAATAACTGAGCTGCCAATTAACAAGGGGCAGGGAGCCTTAG
ACAGAAGGTACCAAATATGCCTGATGCTCCAACATTTTATTTGTAATATCCAAGA
CACCCTCAAATAAACATATGATTCCAATAAAAATGCACAGCCACGATGGCATCT
CTTAGCCTGACATCGCCACGATGTAGAAATTCTGCATCTTCCTCTAGTTTTGAATT
ATCCCCACACAATCTTTTTCGGCAGCTTGGATGGTCAGTTTCAGCACCTTTTACAG
ATGATGAAGCTGAGCCTCGAGGGATGTGTGTCGTCAAGGGGGCTCAGGGCTTCT
CAGGGAGGGGACTCATGGTTTCTTTATTCTGCTACACTCTTCCAAACCTTCACTCA
CCCCTGGTGATGCCCACCTTCCCCTCTCTCCAGGCAAATGGGAGAGACCCTTTGA
AGTCAAGGACACCGAGGAAGAGGACTTCCACGTGGACCAGGTGACCACCGTGAA
GGTGCCTATGATGAAGCGTTTAGGCATGTTTAACATCCAGCACTGTAAGAAGCTG
TCCAGCTGGGTGCTGCTGATGAAATACCTGGGCAATGCCACCGCCATCTTCTTCC
TGCCTGATGAGGGGAAACTACAGCACCTGGAAAATGAACTCACCCACGATATCA
TCACCAAGTTCCTGGAAAATGAAGACAGAAGGTGATTCCCCAACCTGAGGGTGA
CCAAGAAGCTGCCCACACCTCTTAGCCATGTTGGGACTGAGGCCCATCAGGACT
GGCCAGAGGGCTGAGGAGGGTGAACCCCACATCCCTGGGTCACTGCTACTCTGT
ATAAACTTGGCTTCCAGAATGAGGCCACCACTGAGTTCAGGCAGCGCCATCCAT
GCTCCATGAGGAGGACAGTACCCAGGGGTGAGGAGGTAAAGGTCTCGTCCCTGG
GGACTTCCCACTCCAGTGTGGACACTGTCCCTTCCCAATATCCAGTGCCCAGGGC
AGGGACAGCAGCACCACCACACGTTCTGGCAGAACCAAAAAGGAACAGATGGG
CTTCCTGGCAAAGGCAGCAGTGGAGTGTGGAGTTCAAGGGTAGAATGTCCCTGG
GGGGACGGGGGAAGAGCCTGTGTGGCAAGGCCCAGAAAAGCAAGGTTCGGAAT
TGGAACAGCCAGGCCATGTTCGCAGAAGGCTTGCGTTTCTCTGTCACTTTATCGG
TGCTGTTAGATTGGGTGTCCTGTAGTAAGTGATACTTAAACATGAGCCACACATT
AGTGTATGTGTGTGCATTCGTGATTATGCCCATGCCCTGCTGATCTAGTTCGTTTT
GTACACTGTAAAACCAAGATGAAAATACAAAAGGTGTCGGGTTCATAATAGGAA
TCGAGGCTGGAATTTCTCTGTTCCATGCCAGCACCTCCTGAGGTCTCTGCTCCAG
GGGTTGAGAAAGAACAAAGAGGCTGAGAGGGTAACGGATCAGAGAGCCCAGAG
CCAAGCTGCCCGCTCACACCAGACCCTGCTCAGGGTGGCATTGTCTCCCCATGGA
AAACCAGAGAGGAGCACTCAGCCTGGTGTGGTCACTCTTCTCTTATCCACTAAAC
GGTTGTCACTGGGCACTGCCACCAGCCCCGTGTTTCTCTGGGTGTAGGGCCCTGG
GGATGTTACAGGCTGGGGGCCAGGTGACCCAACACTACAGGGCAAGATGAGACA
GGCTTCCAGGACACCTAGAATATCAGAGGAGGTGGCATTTCAAGCTTTTGTGATT
CATTCGATGTTAACATTCTTTGACTCAATGTAGAAGAGCTAAAAGTAGAACAAAC
CAAAGCCGAGTTCCCATCTTAGTGTGGGTGGAGGACACAGGAGTAAGTGGCAGA
AATAATCAGAAAAGAAAACACTTGCACTGTGGTGGGTCCCAGAAGAACAAGAG
GAATGCTGTGCCATGCCTTGAATTTCTTTTCTGCACGACAGGTCTGCCAGCTTAC
ATTTACCCAAACTGTCCATTACTGGAACCTATGATCTGAAGAGCGTCCTGGGTCA
ACTGGGCATCACTAAGGTCTTCAGCAATGGGGCTGACCTCTCCGGGGTCACAGA
GGAGGCACCCCTGAAGCTCTCCAAGGTGAGATCACCCTGACGACCTTGTTGCACC
CTGGTATCTGTAGGGAAGAATGTGTGGGGGCTGCAGCTCTGTCCTGAGGCTGAG
GAAGGGGCCGAGGGAAACAAATGAAGACCCAGGCTGAGCTCCTGAAGATGCCC
GTGATTCACTGACACGGGACGTGGTCAAACAGCAAAGCCAGGCAGGGGACTGCT
GTGCAGCTGGCACTTTCGGGGCCTCCCTTGAGGTTGTGTCACTGACCCTGAATTT
CAACTTTGCCCAAGACCTTCTAGACATTGGGCCTTGATTTATCCATACTGACACA
GAAAGGTTTGGGCTAAGTTGTTTCAAAGGAATTTCTGACTCCTTCGATCTGTGAG
ATTTGGTGTCTGAATTAATGAATGATTTCAGCTAAAGATGACACTTATTTTGGAA
AACTAAAGGCGACCAATGAACAACTGCAGTTCCATGAATGGCTGCATTATCTTG
GGGTCTGGGCACTGTGAAGGTCACTGCCAGGGTCCGTGTCCTCAAGGAGCTTCA
AGCCGTGTACTAGAAAGGAGAGAGCCCTGGAGGCAGACGTGGAGTGACGATGCT
CTTCCCTGTTCTGAGTTGTGGGTGCACCTGAGCAGGGGGAGAGGCGCTTGTCAGG
AAGATGGACAGAGGGGAGCCAGCCCCATCAGCCAAAGCCTTGAGGAGGAGCAA
GGCCTATGTGACAGGGAGGGAGAGGATGTGCAGGGCCAGGGCCGTCCAGGGGG
AGTGAGCGCTTCCTGGGAGGTGTCCACGTGAGCCTTGCTCGAGGCCTGGGATCA
GCCTTACAACGTGTCTCTGCTTCTCTCCCCTCCAGGCCGTGCATAAGGCTGTGCTG
ACCATCGACGAGAAAGGGACTGAAGCTGCTGGGGCCATGTTTTTAGAGGCCATA
CCCATGTCTATCCCCCCCGAGGTCAAGTTCAACAAACCCTTTGTCTTCTTAATGAT
TGAACAAAATACCAAGTCTCCCCTCTTCATGGGAAAAGTGGTGAATCCCACCCA
AAAATAA
SEQ ID NO: 5
CAGCCTCCCCCGTTGCCCCTCTGGATCCACTGCTTAAATACGGACGAGGACAGGG
CCCTGTCTCCTCAGCTTCAGGCACCACCACTGACCTGGGACAGTGAATCGTAAGT
ATGCCTTTCACTGCGAGAGGTTCTGGAGAGGCTTCTGAGCTCCCCATGGCCCAGG
CAGGCAGCAGGTCTGGGGCAGGAGGGGGGTTGTGGAGTGGGTATCCGCCTGCTG
AGGTGCAGGGCAGATGGAGAGGCTGCAGCTGAGCTCCTATTTTCATAATAACAG
CAGCCATGAGGGTTGTGTCCTGTTTCCCAGTCCTGCCCGGTCCCCCCTCGGTACCT
CCTGGTGGATACACTGGTTCCTGTAAGCAGAAGTGGATGAGGGTGTCTAGGTCTG
CAGTCCTGGCACCCCAGGATGGGGGACACCAGCCAAGATACAGCAACAGCAACA
AAGCGCAGCCATTTCTTTCTGTTTGCACAGCTCCTCTGTCTGTCGGGGGCTCCTGT
CTGTTGTCTCCTATAAGCCTCACCACCTCTCCTACTGCTTGGGCATGCATCTTTCT
CCCCTTCTATAGATGAGGAGGTTAAGGTCCAGAGAGGGGTGGGGAGGAACGCCG
GCTCACATTCTCCATCCCCTCCAGATATGACCAGGAACAGACCTGTGCCAGGCCT
CAGCCTTACATCAAAATGGGCCTCCCCATGCACCGTGGACCTCTGGGCCCTCCTG
TCCCAGTGGAGGACAGGAAGCTGTGAGGGGCACTGTCACCCAGGGCTCAAGCTG
GCATTCCTGAATAATCGCTCTGCACCAGGCCACGGCTAAGCTCAGTGCGTGATTA
AGCCTCATAACCCTCCAAGGCAGTTACTAGTGTGATTCCCATTTTACAGATGAGG
AAGATGGGGACAGAGAGGTGAATAACTGGCCCCAAATCACACACCATCCATAAT
TCGGGCTCAGGCACCTGGCTCCAGTCCCCAAACTCTTGAACCTGGCCCTAGTGTC
ACTGTTTCTCTTGGGTCTCAGGCGCTGGATGGGGAACAGGAAACCTGGGCTGGA
CTTGAGGCCTCTCTGATGCTCGGTGACTTCAGACAGTTGCTCAACCTCTCTGTTCT
CTTGGGCAAAACATGATAACCTTTGACTTCTGTCCCCTCCCCTCACCCCACCCGA
CCTTGATCTCTGAAGTGTTGGAAGGATTTAATTTTTCCTGCACTGAGTTTTGGAGA
CAGGTCAAAAAGATGACCAAGGCCAAGGTGGCCAGTTTCCTATAGAACGCCTCT
AAAAGACCTGCAGCAATAGCAGCAAGAACTGGTATTCTCGAGAACTTGCTGCGC
AGCAGGCACTTCTTGGCATTTTATGTGTATTTAATTTCACAATAGCTCTATGACAA
AGTCCACCTTTCTCATCTCCAGGAAACTGAGGTTCAGAGAGGTTAAGTAACTTGT
CCAAGGTCACACAGCTAATAGCAAGTTGACGTGGAGCAATCTGGCCTCAGAGCC
TTTAATTTTAGCCACAGACTGATGCTCCCCTCTTCATTTAGCCAGGCTGCCTCTGA
AGTTTTCTGATTCAAGACTTCTGGCTTCAGCTTTGTACACAGAGATGATTCAATGT
CAGGTTTTGGAGTGAAATCTGTTTAATCCCAGACAAAACATTTAGGATTACATCT
CAGTTTTGTAAGCAAGTAGCTCTGTGATTTTTAGTGAGTTATTTAATGCTCTTTGG
GGCTCAATTTTTCTATCTATAAAATAGGGCTAATAATTTGCACCTTATAGGGTAA
GCTTTGAGGACAGATTAGATGATACGGTGCCTGTAAAACACCAGGTGTTAGTAA
GTGTGGCAATGATGGTGACGCTGAGGCTGATGTTTGCTTAGCATAGGGTTAGGCA
GCTGGCAGGCAGTAAACAGTTGGATAATTTAATGGAAAATTTGCCAAACTCAGA
TGCTGTTCACTGCTGAGCAGGAGCCCCTTCCTGCTGAAATGGTCCTGGGGAGTGC
AGCAGGCTCTCCGGGAAGAAATCTACCATCTCTCGGGCAGGAGCTCAACCTGTG
TGCAGGTACAGGGAGGGCTTCCTCACCTGGTGCCCACTCATGCATTACGTCAGTT
ATTCCTCATCCCTGTCCAAAGGATTCTTTTCTCCATTGTACAGCTATGAAGCTAGT
GCTCAAAGAAGTGAAGTCATTTACCCCAGGCCCCCTGCCAGTAAGTGACAGGGC
CTGGTCACACTTGGGTTTATTTATTGCCCAGTTCAACAGGTTGTTTGACCATAGGC
GAGATTCTCTTCCCTGCACCCTGCCGGGTTGCTCTTGGTCCCTTATTTTATGCTCC
CGGGTAGAAATGGTGTGAGATTAGGCAGGGAGTGGCTCGCTTCCCTGTCCCTGG
CCCCGCAAAGAGTGCTCCCACCTGCCCCGATCCCAGAAATGTCACCATGAAGCCT
TCATTCTTTTGGTTTAAAGCTTGGCCTCAGTGTCCGTACACCATGGGGTACTTGGC
CAGATGGCGACTTTCTCCTCTCCAGTCGCCCTCCCAGGCACTAGCTTTTAGGAGT
GCAGGGTGCTGCCTCTGATAGAAGGGCCAGGAGAGAGCAGGTTTTGGAGTCCTG
ATGTTATAAGGAACAGCTTGGGAGGCATAATGAACCCAACATGATGCTTGAGAC
CAATGTCACAGCCCAATTCTGACATTCATCATCTGAGATCTGAGGACACAGCTGT
CTCAGTTCATGATCTGAGTGCTGGGAAAGCCAAGACTTGTTCCAGCTTTGTCACT
GACTTGCTGTATAGCCTCAACAAGGCCCTGACCCTCTCTGGGCTTCAAACTCTTC
ACTGTGAAAGGAGGAAACCAGAGTAGGTGATGTGACACCAGGAAAGATGGATG
GGTGTGGGGGAATGTGCTCCTCCCAGCTGTCACCCCCTCGCCACCCTCCCTGCAC
CAGCCTCTCCACCTCCTTTGAGCCCAGAATTCCCCTGTCTAGGAGGGCACCTGTC
TCATGCCTAGCCATGGGAATTCTCCATCTGTTTTGCTACATTGAACCCAGATGCC
ATTCTAACCAAGAATCCTGGCTGGGTGCAGGGGCTCTCGCCTGTAACCCCAGCAC
TTTGGGAGGCCAAGGCAGGCGGATCAAGAGGTCAGGAGTTCAAGACCTGCCTGG
CCAACACGGTGAAACCTCAGCTCTACTAAAAATACAAAAATTAGCCAGGCGTGG
TGGCACACGCCTGTAATCCCAGCTATTTGGGAAGCTGAGACAGAAGAATTTCTTG
AACCCGGGAGGTGGAGGTTTCAGTGAGCCGAGATCACGCCACTGCACTCCACCC
TGGCAGATAAAGCGAGACTCTGTCTCAAAAAAAACCCAAAAACCTATGTTAGTG
TACAGAGGGCCCCAGTGAAGTCTTCTCCCAGCCCCACTTTGCACAACTGGGGAG
AGTGAGGCCCCAGGACCAGAGGATTCTTGCTAAAGGCCAAGTGGATAGTGATGG
CCCTGCCAGGGCTAGAAGCCACAACCTCTGGCCCTGAGGCCACTCAGCATATTTA
GTGTCCCCACCCTGCAGAGGCCCAACTCCCTCCTGACCACTGAGCCCTGTAATGA
TGGGGGAATTTCCATAAGCCATGAAGGACTGCACAAAGTTCAGTTGGGAAGTGA
AAGAGAAATTAAAGGGAGATGGAAATATACAGCACTAATTTTAGCACCGTCTTT
AGTTCTAACAACACTAGCTAGCTGAAGAAAAATACAAACATGTATTATGTAATGT
GTGGTCTGTTCCATTTGGATTACTTAGAGGCACGAGGGCCAGGAGAAAGGTGGT
GGAGAGAAACCAGCTTTGCACTTCATTTGTTGCTTTATTGGAAGGAAACTTTTAA
AAGTCCAAGGGGGTTGAAGAATCTCAATATTTGTTATTTCCAGCTTTTTTTCTCCA
GTTTTTCATTTCCCAAATTCAAGGACACCTTTTTCTTTGTATTTTGTTAAGATGAT
GGTTTTGGTTTTGTGACTAGTAGTTAACAATGTGGCTGCCGGGCATATTCTCCTCA
GCTAGGACCTCAGTTTTCCCATCTGTGAAGACGGCAGGTTCTACCTAGGGGGCTG
CAGGCTGGTGGTCCGAAGCCTGGGCATATCTGGAGTAGAAGGATCACTGTGGGG
CAGGGCAGGTTCTGTGTTGCTGTGGATGACGTTGACTTTGACCATTGCTCGGCAG
AGCCTGCTCTCGCTGGTTCAGCCACAGGCCCCACCACTCCCTATTGTCTCAGCCC
CGGGTATGAAACATGTATTCCTCACTGGCCTATCACCTGAAGCCTTTGAATTTGC
AACACCTGCCAACCCCTCCCTCAAAAGAGTTGCCCTCTCAGATCCTTTTGATGTA
AGGTTTGGTGTTGAGACTTATTTCACTAAATTCTCATACATAAACATCACTTTATG
TATGAGGCAAAATGAGGACCAGGGAGATGAATGACTTGTCCTGGCTCATACACC
TGGAAAGTGACAGAGTCAGATTAGATCCCAGGTCTATCTGAAGTTAAAAGAGGT
GTCTTTTCACTTCCCACCTCCTCCATCTACTTTAAAGCAGCACAAACCCCTGCTTT
CAAGGAGAGATGAGCGTCTCTAAAGCCCCTGACAGCAAGAGCCCAGAACTGGGA
CACCATTAGTGACCCAGACGGCAGGTAAGCTGACTGCAGGAGCATCAGCCTATT
CTTGTGTCTGGGACCACAGAGCATTGTGGGGACAGCCCCGTCTCTTGGGAAAAA
AACCCTAAGGGCTGAGGATCCTTGTGAGTGTTGGGTGGGAACAGCTCCCAGGAG
GTTTAATCACAGCCCCTCCATGCTCTCTAGCTGTTGCCATTGTGCAAGATGCATTT
CCCTTCTGTGCAGCAGTTTCCCTGGCCACTAAATAGTGGGATTAGATAGAAGCCC
TCCAAGGGCTTCCAGCTTGACATGATTCTTGATTCTGATCTGGCCCGATTCCTGG
ATAATCGTGGGCAGGCCCATTCCTCTTCTTGTGCCTCATTTTCTTCTTTTGTAAAA
CAATGGCTGTACCATTTGCATCTTAGGGTCATTGCAGATGTAAGTGTTGCTGTCC
AGAGCCTGGGTGCAGGACCTAGATGTAGGATTCTGGTTCTGCTACTTCCTCAGTG
ACATTGAATAGCTGACCTAATCTCTCTGGCTTTGGTTTCTTCATCTGTAAAAGAAG
GATATTAGCATTAGCACCTCACGGGATTGTTACAAGAAAGCAATGAATTAACAC
ATGTGAGCACGGAGAACAGTGCTTGGCATATGGTAAGCACTACGTACATTTTGCT
ATTCTTCTGATTCTTTCAGTGTTACTGATGTCGGCAAGTACTTGGCACAGGCTGGT
TTAATAATCCCTAGGCACTTCCACGTGGTGTCAATCCCTGATCACTGGGAGTCAT
CATGTGCCTTGACTCGGGGCCTGGCCCCCCCATCTCTGTCTTGCAGGACAATGCC
GTCTTCTGTCTCGTGGGGCATCCTCCTGCTGGCAGGCCTGTGCTGCCTGGTCCCTG
TCTCCCTGGCTGAGGATCCCCAGGGAGATGCTGCCCAGAAGACAGATACATCCC
ACCATGATCAGGATCACCCAACCTTCAACAAGATCACCCCCAACCTGGCTGAGTT
CGCCTTCAGCCTATACCGCCAGCTGGCACACCAGTCCAACAGCACCAATATCTTC
TTCTCCCCAGTGAGCATCGCTACAGCCTTTGCAATGCTCTCCCTGGGGACCAAGG
CTGACACTCACGATGAAATCCTGGAGGGCCTGAATTTCAACCTCACGGAGATTCC
GGAGGCTCAGATCCATGAAGGCTTCCAGGAACTCCTCCGTACCCTCAACCAGCC
AGACAGCCAGCTCCAGCTGACCACCGGCAATGGCCTGTTCCTCAGCGAGGGCCT
GAAGCTAGTGGATAAGTTTTTGGAGGATGTTAAAAAGTTGTACCACTCAGAAGC
CTTCACTGTCAACTTCGGGGACACCGAAGAGGCCAAGAAACAGATCAACGATTA
CGTGGAGAAGGGTACTCAAGGGAAAATTGTGGATTTGGTCAAGGAGCTTGACAG
AGACACAGTTTTTGCTCTGGTGAATTACATCTTCTTTAAAGGCAAATGGGAGAGA
CCCTTTGAAGTCAAGGACACCGAGGAAGAGGACTTCCACGTGGACCAGGTGACC
ACCGTGAAGGTGCCTATGATGAAGCGTTTAGGCATGTTTAACATCCAGCACTGTA
AGAAGCTGTCCAGCTGGGTGCTGCTGATGAAATACCTGGGCAATGCCACCGCCA
TCTTCTTCCTGCCTGATGAGGGGAAACTACAGCACCTGGAAAATGAACTCACCCA
CGATATCATCACCAAGTTCCTGGAAAATGAAGACAGAAGGTCTGCCAGCTTACA
TTTACCCAAACTGTCCATTACTGGAACCTATGATCTGAAGAGCGTCCTGGGTCAA
CTGGGCATCACTAAGGTCTTCAGCAATGGGGCTGACCTCTCCGGGGTCACAGAG
GAGGCACCCCTGAAGCTCTCCAAGGCCGTGCATAAGGCTGTGCTGACCATCGAC
GAGAAAGGGACTGAAGCTGCTGGGGCCATGTTTTTAGAGGCCATACCCATGTCT
ATCCCCCCCGAGGTCAAGTTCAACAAACCCTTTGTCTTCTTAATGATTGAACAAA
ATACCAAGTCTCCCCTCTTCATGGGAAAAGTGGTGAATCCCACCCAAAAATAA
SEQ ID NO: 6
CAGCCTCCCCCGTTGCCCCTCTGGATCCACTGCTTAAATACGGACGAGGACAGGG
CCCTGTCTCCTCAGCTTCAGGCACCACCACTGACCTGGGACAGTGAATCGTAAGT
ATGCCTTTCACTGCGAGAGGTTCTGGAGAGGCTTCTGAGCTCCCCATGGCCCAGG
CAGGCAGCAGGTCTGGGGCAGGAGGGGGGTTGTGGAGTGGGTATCCGCCTGCTG
AGGTGCAGGGCAGATGGAGAGGCTGCAGCTGAGCTCCTATTTTCATAATAACAG
CAGCCATGAGGGTTGTGTCCTGTTTCCCAGTCCTGCCCGGTCCCCCCTCGGTACCT
CCTGGTGGATACACTGGTTCCTGTAAGCAGAAGTGGATGAGGGTGTCTAGGTCTG
CAGTCCTGGCACCCCAGGATGGGGGACACCAGCCAAGATACAGCAACAGCAACA
AAGCGCAGCCATTTCTTTCTGTTTGCACAGCTCCTCTGTCTGTCGGGGGCTCCTGT
CTGTTGTCTCCTATAAGCCTCACCACCTCTCCTACTGCTTGGGCATGCATCTTTCT
CCCCTTCTATAGATGAGGAGGTTAAGGTCCAGAGAGGGGGGGGAGGAACGCCG
GCTCACATTCTCCATCCCCTCCAGATATGACCAGGAACAGACCTGTGCCAGGCCT
CAGCCTTACATCAAAATGGGCCTCCCCATGCACCGTGGACCTCTGGGCCCTCCTG
TCCCAGTGGAGGACAGGAAGCTGTGAGGGGCACTGTCACCCAGGGCTCAAGCTG
GCATTCCTGAATAATCGCTCTGCACCAGGCCACGGCTAAGCTCAGTGCGTGATTA
AGCCTCATAACCCTCCAAGGCAGTTACTAGTGTGATTCCCATTTTACAGATGAGG
AAGATGGGGACAGAGAGGTGAATAACTGGCCCCAAATCACACACCATCCATAAT
TCGGGCTCAGGCACCTGGCTCCAGTCCCCAAACTCTTGAACCTGGCCCTAGTGTC
ACTGTTTCTCTTGGGTCTCAGGCGCTGGATGGGGAACAGGAAACCTGGGCTGGA
CTTGAGGCCTCTCTGATGCTCGGTGACTTCAGACAGTTGCTCAACCTCTCTGTTCT
CTTGGGCAAAACATGATAACCTTTGACTTCTGTCCCCTCCCCTCACCCCACCCGA
CCTTGATCTCTGAAGTGTTGGAAGGATTTAATTTTTCCTGCACTGAGTTTTGGAGA
CAGGTCAAAAAGATGACCAAGGCCAAGGTGGCCAGTTTCCTATAGAACGCCTCT
AAAAGACCTGCAGCAATAGCAGCAAGAACTGGTATTCTCGAGAACTTGCTGCGC
AGCAGGCACTTCTTGGCATTTTATGTGTATTTAATTTCACAATAGCTCTATGACAA
AGTCCACCTTTCTCATCTCCAGGAAACTGAGGTTCAGAGAGGTTAAGTAACTTGT
CCAAGGTCACACAGCTAATAGCAAGTTGACGTGGAGCAATCTGGCCTCAGAGCC
TTTAATTTTAGCCACAGACTGATGCTCCCCTCTTCATTTAGCCAGGCTGCCTCTGA
AGTTTTCTGATTCAAGACTTCTGGCTTCAGCTTTGTACACAGAGATGATTCAATGT
CAGGTTTTGGAGTGAAATCTGTTTAATCCCAGACAAAACATTTAGGATTACATCT
CAGTTTTGTAAGCAAGTAGCTCTGTGATTTTTAGTGAGTTATTTAATGCTCTTTGG
GGCTCAATTTTTCTATCTATAAAATAGGGCTAATAATTTGCACCTTATAGGGTAA
GCTTTGAGGACAGATTAGATGATACGGTGCCTGTAAAACACCAGGTGTTAGTAA
GTGTGGCAATGATGGTGACGCTGAGGCTGATGTTTGCTTAGCATAGGGTTAGGCA
GCTGGCAGGCAGTAAACAGTTGGATAATTTAATGGAAAATTTGCCAAACTCAGA
TGCTGTTCACTGCTGAGCAGGAGCCCCTTCCTGCTGAAATGGTCCTGGGGAGTGC
AGCAGGCTCTCCGGGAAGAAATCTACCATCTCTCGGGCAGGAGCTCAACCTGTG
TGCAGGTACAGGGAGGGCTTCCTCACCTGGTGCCCACTCATGCATTACGTCAGTT
ATTCCTCATCCCTGTCCAAAGGATTCTTTTCTCCATTGTACAGCTATGAAGCTAGT
GCTCAAAGAAGTGAAGTCATTTACCCCAGGCCCCCTGCCAGTAAGTGACAGGGC
CTGGTCACACTTGGGTTTATTTATTGCCCAGTTCAACAGGTTGTTTGACCATAGGC
GAGATTCTCTTCCCTGCACCCTGCCGGGTTGCTCTTGGTCCCTTATTTTATGCTCC
CGGGTAGAAATGGTGTGAGATTAGGCAGGGAGTGGCTCGCTTCCCTGTCCCTGG
CCCCGCAAAGAGTGCTCCCACCTGCCCCGATCCCAGAAATGTCACCATGAAGCCT
TCATTCTTTTGGTTTAAAGCTTGGCCTCAGTGTCCGTACACCATGGGGTACTTGGC
CAGATGGCGACTTTCTCCTCTCCAGTCGCCCTCCCAGGCACTAGCTTTTAGGAGT
GCAGGGTGCTGCCTCTGATAGAAGGGCCAGGAGAGAGCAGGTTTTGGAGTCCTG
ATGTTATAAGGAACAGCTTGGGAGGCATAATGAACCCAACATGATGCTTGAGAC
CAATGTCACAGCCCAATTCTGACATTCATCATCTGAGATCTGAGGACACAGCTGT
CTCAGTTCATGATCTGAGTGCTGGGAAAGCCAAGACTTGTTCCAGCTTTGTCACT
GACTTGCTGTATAGCCTCAACAAGGCCCTGACCCTCTCTGGGCTTCAAACTCTTC
ACTGTGAAAGGAGGAAACCAGAGTAGGTGATGTGACACCAGGAAAGATGGATG
GGTGTGGGGGAATGTGCTCCTCCCAGCTGTCACCCCCTCGCCACCCTCCCTGCAC
CAGCCTCTCCACCTCCTTTGAGCCCAGAATTCCCCTGTCTAGGAGGGCACCTGTC
TCATGCCTAGCCATGGGAATTCTCCATCTGTTTTGCTACATTGAACCCAGATGCC
ATTCTAACCAAGAATCCTGGCTGGGTGCAGGGGCTCTCGCCTGTAACCCCAGCAC
TTTGGGAGGCCAAGGCAGGCGGATCAAGAGGTCAGGAGTTCAAGACCTGCCTGG
CCAACACGGTGAAACCTCAGCTCTACTAAAAATACAAAAATTAGCCAGGCGTGG
TGGCACACGCCTGTAATCCCAGCTATTTGGGAAGCTGAGACAGAAGAATTTCTTG
AACCCGGGAGGTGGAGGTTTCAGTGAGCCGAGATCACGCCACTGCACTCCACCC
TGGCAGATAAAGCGAGACTCTGTCTCAAAAAAAACCCAAAAACCTATGTTAGTG
TACAGAGGGCCCCAGTGAAGTCTTCTCCCAGCCCCACTTTGCACAACTGGGGAG
AGTGAGGCCCCAGGACCAGAGGATTCTTGCTAAAGGCCAAGTGGATAGTGATGG
CCCTGCCAGGGCTAGAAGCCACAACCTCTGGCCCTGAGGCCACTCAGCATATTTA
GTGTCCCCACCCTGCAGAGGCCCAACTCCCTCCTGACCACTGAGCCCTGTAATGA
TGGGGGAATTTCCATAAGCCATGAAGGACTGCACAAAGTTCAGTTGGGAAGTGA
AAGAGAAATTAAAGGGAGATGGAAATATACAGCACTAATTTTAGCACCGTCTTT
AGTTCTAACAACACTAGCTAGCTGAAGAAAAATACAAACATGTATTATGTAATGT
GTGGTCTGTTCCATTTGGATTACTTAGAGGCACGAGGGCCAGGAGAAAGGTGGT
GGAGAGAAACCAGCTTTGCACTTCATTTGTTGCTTTATTGGAAGGAAACTTTTAA
AAGTCCAAGGGGGTTGAAGAATCTCAATATTTGTTATTTCCAGCTTTTTTTCTCCA
GTTTTTCATTTCCCAAATTCAAGGACACCTTTTTCTTTGTATTTTGTTAAGATGAT
GGTTTTGGTTTTGTGACTAGTAGTTAACAATGTGGCTGCCGGGCATATTCTCCTCA
GCTAGGACCTCAGTTTTCCCATCTGTGAAGACGGCAGGTTCTACCTAGGGGGCTG
CAGGCTGGTGGTCCGAAGCCTGGGCATATCTGGAGTAGAAGGATCACTGTGGGG
CAGGGCAGGTTCTGTGTTGCTGTGGATGACGTTGACTTTGACCATTGCTCGGCAG
AGCCTGCTCTCGCTGGTTCAGCCACAGGCCCCACCACTCCCTATTGTCTCAGCCC
CGGGTATGAAACATGTATTCCTCACTGGCCTATCACCTGAAGCCTTTGAATTTGC
AACACCTGCCAACCCCTCCCTCAAAAGAGTTGCCCTCTCAGATCCTTTTGATGTA
AGGTTTGGTGTTGAGACTTATTTCACTAAATTCTCATACATAAACATCACTTTATG
TATGAGGCAAAATGAGGACCAGGGAGATGAATGACTTGTCCTGGCTCATACACC
TGGAAAGTGACAGAGTCAGATTAGATCCCAGGTCTATCTGAAGTTAAAAGAGGT
GTCTTTTCACTTCCCACCTCCTCCATCTACTTTAAAGCAGCACAAACCCCTGCTTT
CAAGGAGAGATGAGCGTCTCTAAAGCCCCTGACAGCAAGAGCCCAGAACTGGGA
CACCATTAGTGACCCAGACGGCAGGTAAGCTGACTGCAGGAGCATCAGCCTATT
CTTGTGTCTGGGACCACAGAGCATTGTGGGGACAGCCCCGTCTCTTGGGAAAAA
AACCCTAAGGGCTGAGGATCCTTGTGAGTGTTGGGTGGGAACAGCTCCCAGGAG
GTTTAATCACAGCCCCTCCATGCTCTCTAGCTGTTGCCATTGTGCAAGATGCATTT
CCCTTCTGTGCAGCAGTTTCCCTGGCCACTAAATAGTGGGATTAGATAGAAGCCC
TCCAAGGGCTTCCAGCTTGACATGATTCTTGATTCTGATCTGGCCCGATTCCTGG
ATAATCGTGGGCAGGCCCATTCCTCTTCTTGTGCCTCATTTTCTTCTTTTGTAAAA
CAATGGCTGTACCATTTGCATCTTAGGGTCATTGCAGATGTAAGTGTTGCTGTCC
AGAGCCTGGGTGCAGGACCTAGATGTAGGATTCTGGTTCTGCTACTTCCTCAGTG
ACATTGAATAGCTGACCTAATCTCTCTGGCTTTGGTTTCTTCATCTGTAAAAGAAG
GATATTAGCATTAGCACCTCACGGGATTGTTACAAGAAAGCAATGAATTAACAC
ATGTGAGCACGGAGAACAGTGCTTGGCATATGGTAAGCACTACGTACATTTTGCT
ATTCTTCTGATTCTTTCAGTGTTACTGATGTCGGCAAGTACTTGGCACAGGCTGGT
TTAATAATCCCTAGGCACTTCCACGTGGTGTCAATCCCTGATCACTGGGAGTCAT
CATGTGCCTTGACTCGGGGCCTGGCCCCCCCATCTCTGTCTTGCAGGACAATGCC
GTCTTCTGTCTCGTGGGGCATCCTCCTGCTGGCAGGCCTGTGCTGCCTGGTCCCTG
TCTCCCTGGCTGAGGATCCCCAGGGAGATGCTGCCCAGAAGACAGATACATCCC
ACCATGATCAGGATCACCCAACCTTCAACAAGATCACCCCCAACCTGGCTGAGTT
CGCCTTCAGCCTATACCGCCAGCTGGCACACCAGTCCAACAGCACCAATATCTTC
TTCTCCCCAGTGAGCATCGCTACAGCCTTTGCAATGCTCTCCCTGGGGACCAAGG
CTGACACTCACGATGAAATCCTGGAGGGCCTGAATTTCAACCTCACGGAGATTCC
GGAGGCTCAGATCCATGAAGGCTTCCAGGAACTCCTCCGTACCCTCAACCAGCC
AGACAGCCAGCTCCAGCTGACCACCGGCAATGGCCTGTTCCTCAGCGAGGGCCT
GAAGCTAGTGGATAAGTTTTTGGAGGATGTTAAAAAGTTGTACCACTCAGAAGC
CTTCACTGTCAACTTCGGGGACACCGAAGAGGCCAAGAAACAGATCAACGATTA
CGTGGAGAAGGGTACTCAAGGGAAAATTGTGGATTTGGTCAAGGAGCTTGACAG
AGACACAGTTTTTGCTCTGGTGAATTACATCTTCTTTAAAGGCAAATGGGAGAGA
CCCTTTGAAGTCAAGGACACCGAGGAAGAGGACTTCCACGTGGACCAGGTGACC
ACCGTGAAGGTGCCTATGATGAAGCGTTTAGGCATGTTTAACATCCAGCACTGTA
AGAAGCTGTCCAGCTGGGTGCTGCTGATGAAATACCTGGGCAATGCCACCGCCA
TCTTCTTCCTGCCTGATGAGGGGAAACTACAGCACCTGGAAAATGAACTCACCCA
CGATATCATCACCAAGTTCCTGGAAAATGAAGACAGAAGGTCTGCCAGCTTACA
TTTACCCAAACTGTCCATTACTGGAACCTATGATCTGAAGAGCGTCCTGGGTCAA
CTGGGCATCACTAAGGTCTTCAGCAATGGGGCTGACCTCTCCGGGGTCACAGAG
GAGGCACCCCTGAAGCTCTCCAAGGCCGTGCATAAGGCTGTGCTGACCATCGAC
GAGAAAGGGACTGAAGCTGCTGGGGCCATGTTTTTAGAGGCCATACCCATGTCT
ATCCCCCCCGAGGTCAAGTTCAACAAACCCTTTGTCTTCTTAATGATTGAACAAA
ATACCAAGTCTCCCCTCTTCATGGGAAAAGTGGTGAATCCCACCCAAAAATAA
SEQ ID NO: 7
ATGCCGTCTTCTGTCTCGTGGGGCATCCTCCTGCTGGCAGGCCTGTGCTGCCTGGT
CCCTGTCTCCCTGGCTGAGGATCCCCAGGGAGATGCTGCCCAGAAGACAGATAC
ATCCCACCATGATCAGGATCACCCAACCTTCAACAAGATCACCCCCAACCTGGCT
GAGTTCGCCTTCAGCCTATACCGCCAGCTGGCACACCAGTCCAACAGCACCAATA
TCTTCTTCTCCCCAGTGAGCATCGCTACAGCCTTTGCAATGCTCTCCCTGGGGACC
AAGGCTGACACTCACGATGAAATCCTGGAGGGCCTGAATTTCAACCTCACGGAG
ATTCCGGAGGCTCAGATCCATGAAGGCTTCCAGGAACTCCTCCGTACCCTCAACC
AGCCAGACAGCCAGCTCCAGCTGACCACCGGCAATGGCCTGTTCCTCAGCGAGG
GCCTGAAGCTAGTGGATAAGTTTTTGGAGGATGTTAAAAAGTTGTACCACTCAGA
AGCCTTCACTGTCAACTTCGGGGACACCGAAGAGGCCAAGAAACAGATCAACGA
TTACGTGGAGAAGGGTACTCAAGGGAAAATTGTGGATTTGGTCAAGGAGCTTGA
CAGAGACACAGTTTTTGCTCTGGTGAATTACATCTTCTTTAAAGGCAAATGGGAG
AGACCCTTTGAAGTCAAGGACACCGAGGAAGAGGACTTCCACGTGGACCAGGTG
ACCACCGTGAAGGTGCCTATGATGAAGCGTTTAGGCATGTTTAACATCCAGCACT
GTAAGAAGCTGTCCAGCTGGGTGCTGCTGATGAAATACCTGGGCAATGCCACCG
CCATCTTCTTCCTGCCTGATGAGGGGAAACTACAGCACCTGGAAAATGAACTCAC
CCACGATATCATCACCAAGTTCCTGGAAAATGAAGACAGAAGGTCTGCCAGCTT
ACATTTACCCAAACTGTCCATTACTGGAACCTATGATCTGAAGAGCGTCCTGGGT
CAACTGGGCATCACTAAGGTCTTCAGCAATGGGGCTGACCTCTCCGGGGTCACA
GAGGAGGCACCCCTGAAGCTCTCCAAGGCCGTGCATAAGGCTGTGCTGACCATC
GACGAGAAAGGGACTGAAGCTGCTGGGGCCATGTTTTTAGAGGCCATACCCATG
TCTATCCCCCCCGAGGTCAAGTTCAACAAACCCTTTGTCTTCTTAATGATTGAAC
AAAATACCAAGTCTCCCCTCTTCATGGGAAAAGTGGTGAATCCCACCCAAAAAT
AA
SEQ ID NO: 8
ATGCCGTCTTCTGTCTCGTGGGGCATCCTCCTGCTGGCAGGCCTGTGCTGCCTGGT
CCCTGTCTCCCTGGCTGAGGATCCCCAGGGAGATGCTGCCCAGAAGACAGATAC
ATCCCACCATGATCAGGATCACCCAACCTTCAACAAGATCACCCCCAACCTGGCT
GAGTTCGCCTTCAGCCTATACCGCCAGCTGGCACACCAGTCCAACAGCACCAATA
TCTTCTTCTCCCCAGTGAGCATCGCTACAGCCTTTGCAATGCTCTCCCTGGGGACC
AAGGCTGACACTCACGATGAAATCCTGGAGGGCCTGAATTTCAACCTCACGGAG
ATTCCGGAGGCTCAGATCCATGAAGGCTTCCAGGAACTCCTCCGTACCCTCAACC
AGCCAGACAGCCAGCTCCAGCTGACCACCGGCAATGGCCTGTTCCTCAGCGAGG
GCCTGAAGCTAGTGGATAAGTTTTTGGAGGATGTTAAAAAGTTGTACCACTCAGA
AGCCTTCACTGTCAACTTCGGGGACACCGAAGAGGCCAAGAAACAGATCAACGA
TTACGTGGAGAAGGGTACTCAAGGGAAAATTGTGGATTTGGTCAAGGAGCTTGA
CAGAGACACAGTTTTTGCTCTGGTGAATTACATCTTCTTTAAAGGTAAGGTTGCT
CAACCAGCCTGAGCTGTTCCCATAGAAACAAGCAAAAATATTCTCAAACCATCA
GTTCTTGAACTCTCCTTGGCAATGCATTATGGGCCATAGCAATGCTTTTCAGCGT
GGATTCTTCAGTTTTCTACACACAAACACTAAAATGTTTTCCATCATTGAGTAATT
TGAGGAAATAATAGATTAAACTGTCAAAACTACTGACAGCTCTGCAGAACTTTTC
AGAGCCTTTAATGTCCTTGTGTATACTGTATATGTAGAATATATAATGCTTAGAA
CTATAGAACAAATTGTAATACACTGCATAAAGGGATAGTTTCATGGAACATACTT
TACACGACTCTAGTGTCCCAGAATCAGTATCAGTTTTGCAATCTGAAAGACCTGG
GTTCAAATCCTGCCTCTAACACAATTAGCTTTTGACAAAAACAATGCATTCTACC
TCTTTGAGGTGCTAATTTCTCATCTTAGCATGGACAAAATACCATTCTTGCTGTCA
GGTTTTTTTAGGATTAAACAAATGACAAAGACTGTGGGGATGGTGTGTGGCATAC
AGCAGGTGATGGACTCTTCTGTATCTCAGGCTGCCTTCCTGCCCCTGAGGGGTTA
AAATGCCAGGGTCCTGGGGGCCCCAGGGCATTCTAAGCCAGCTCCCACTGTCCC
AGGAAAACAGCATAGGGGAGGGGAGGTGGGAGGCAAGGCCAGGGGCTGCTTCC
TCCACTCTGAGGCTCCCTTGCTCTTGAGGCAAAGGAGGGCAGTGGAGAGCAGCC
AGGCTGCAGTCAGCACAGCTAAAGTCCTGGCTCTGCTGTGGCCTTAGTGGGGGCC
CAGGTCCCTCTCCAGCCCCAGTCTCCTCCTTCTGTCCAATGAGAAAGCTGGGATC
AGGGGTCCCTGAGGCCCCTGTCCACTCTGCATGCCTCGATGGTGAAGCTCTGTTG
GTATGGCAGAGGGGAGGCTGCTCAGGCATCTGCATTTCCCCTGCCAATCTAGAG
GATGAGGAAAGCTCTCAGGAATAGTAAGCAGAATGTTTGCCCTGGATGAATAAC
TGAGCTGCCAATTAACAAGGGGCAGGGAGCCTTAGACAGAAGGTACCAAATATG
CCTGATGCTCCAACATTTTATTTGTAATATCCAAGACACCCTCAAATAAACATAT
GATTCCAATAAAAATGCACAGCCACGATGGCATCTCTTAGCCTGACATCGCCACG
ATGTAGAAATTCTGCATCTTCCTCTAGTTTTGAATTATCCCCACACAATCTTTTTC
GGCAGCTTGGATGGTCAGTTTCAGCACCTTTTACAGATGATGAAGCTGAGCCTCG
AGGGATGTGTGTCGTCAAGGGGGCTCAGGGCTTCTCAGGGAGGGGACTCATGGT
TTCTTTATTCTGCTACACTCTTCCAAACCTTCACTCACCCCTGGTGATGCCCACCT
TCCCCTCTCTCCAGGCAAATGGGAGAGACCCTTTGAAGTCAAGGACACCGAGGA
AGAGGACTTCCACGTGGACCAGGTGACCACCGTGAAGGTGCCTATGATGAAGCG
TTTAGGCATGTTTAACATCCAGCACTGTAAGAAGCTGTCCAGCTGGGTGCTGCTG
ATGAAATACCTGGGCAATGCCACCGCCATCTTCTTCCTGCCTGATGAGGGGAAAC
TACAGCACCTGGAAAATGAACTCACCCACGATATCATCACCAAGTTCCTGGAAA
ATGAAGACAGAAGGTGATTCCCCAACCTGAGGGTGACCAAGAAGCTGCCCACAC
CTCTTAGCCATGTTGGGACTGAGGCCCATCAGGACTGGCCAGAGGGCTGAGGAG
GGTGAACCCCACATCCCTGGGTCACTGCTACTCTGTATAAACTTGGCTTCCAGAA
TGAGGCCACCACTGAGTTCAGGCAGCGCCATCCATGCTCCATGAGGAGGACAGT
ACCCAGGGGTGAGGAGGTAAAGGTCTCGTCCCTGGGGACTTCCCACTCCAGTGT
GGACACTGTCCCTTCCCAATATCCAGTGCCCAGGGCAGGGACAGCAGCACCACC
ACACGTTCTGGCAGAACCAAAAAGGAACAGATGGGCTTCCTGGCAAAGGCAGCA
GTGGAGTGTGGAGTTCAAGGGTAGAATGTCCCTGGGGGGACGGGGGAAGAGCCT
GTGTGGCAAGGCCCAGAAAAGCAAGGTTCGGAATTGGAACAGCCAGGCCATGTT
CGCAGAAGGCTTGCGTTTCTCTGTCACTTTATCGGTGCTGTTAGATTGGGTGTCCT
GTAGTAAGTGATACTTAAACATGAGCCACACATTAGTGTATGTGTGTGCATTCGT
GATTATGCCCATGCCCTGCTGATCTAGTTCGTTTTGTACACTGTAAAACCAAGAT
GAAAATACAAAAGGTGTCGGGTTCATAATAGGAATCGAGGCTGGAATTTCTCTG
TTCCATGCCAGCACCTCCTGAGGTCTCTGCTCCAGGGGTTGAGAAAGAACAAAG
AGGCTGAGAGGGTAACGGATCAGAGAGCCCAGAGCCAAGCTGCCCGCTCACACC
AGACCCTGCTCAGGGTGGCATTGTCTCCCCATGGAAAACCAGAGAGGAGCACTC
AGCCTGGTGTGGTCACTCTTCTCTTATCCACTAAACGGTTGTCACTGGGCACTGC
CACCAGCCCCGTGTTTCTCTGGGTGTAGGGCCCTGGGGATGTTACAGGCTGGGGG
CCAGGTGACCCAACACTACAGGGCAAGATGAGACAGGCTTCCAGGACACCTAGA
ATATCAGAGGAGGTGGCATTTCAAGCTTTTGTGATTCATTCGATGTTAACATTCTT
TGACTCAATGTAGAAGAGCTAAAAGTAGAACAAACCAAAGCCGAGTTCCCATCT
TAGTGTGGGTGGAGGACACAGGAGTAAGTGGCAGAAATAATCAGAAAAGAAAA
CACTTGCACTGTGGTGGGTCCCAGAAGAACAAGAGGAATGCTGTGCCATGCCTT
GAATTTCTTTTCTGCACGACAGGTCTGCCAGCTTACATTTACCCAAACTGTCCATT
ACTGGAACCTATGATCTGAAGAGCGTCCTGGGTCAACTGGGCATCACTAAGGTCT
TCAGCAATGGGGCTGACCTCTCCGGGGTCACAGAGGAGGCACCCCTGAAGCTCT
CCAAGGTGAGATCACCCTGACGACCTTGTTGCACCCTGGTATCTGTAGGGAAGA
ATGTGTGGGGGCTGCAGCTCTGTCCTGAGGCTGAGGAAGGGGCCGAGGGAAACA
AATGAAGACCCAGGCTGAGCTCCTGAAGATGCCCGTGATTCACTGACACGGGAC
GTGGTCAAACAGCAAAGCCAGGCAGGGGACTGCTGTGCAGCTGGCACTTTCGGG
GCCTCCCTTGAGGTTGTGTCACTGACCCTGAATTTCAACTTTGCCCAAGACCTTCT
AGACATTGGGCCTTGATTTATCCATACTGACACAGAAAGGTTTGGGCTAAGTTGT
TTCAAAGGAATTTCTGACTCCTTCGATCTGTGAGATTTGGTGTCTGAATTAATGA
ATGATTTCAGCTAAAGATGACACTTATTTTGGAAAACTAAAGGCGACCAATGAA
CAACTGCAGTTCCATGAATGGCTGCATTATCTTGGGGTCTGGGCACTGTGAAGGT
CACTGCCAGGGTCCGTGTCCTCAAGGAGCTTCAAGCCGTGTACTAGAAAGGAGA
GAGCCCTGGAGGCAGACGTGGAGTGACGATGCTCTTCCCTGTTCTGAGTTGTGGG
TGCACCTGAGCAGGGGGAGAGGCGCTTGTCAGGAAGATGGACAGAGGGGAGCC
AGCCCCATCAGCCAAAGCCTTGAGGAGGAGCAAGGCCTATGTGACAGGGAGGG
AGAGGATGTGCAGGGCCAGGGCCGTCCAGGGGGAGTGAGCGCTTCCTGGGAGGT
GTCCACGTGAGCCTTGCTCGAGGCCTGGGATCAGCCTTACAACGTGTCTCTGCTT
CTCTCCCCTCCAGGCCGTGCATAAGGCTGTGCTGACCATCGACGAGAAAGGGAC
TGAAGCTGCTGGGGCCATGTTTTTAGAGGCCATACCCATGTCTATCCCCCCCGAG
GTCAAGTTCAACAAACCCTTTGTCTTCTTAATGATTGAACAAAATACCAAGTCTC
CCCTCTTCATGGGAAAAGTGGTGAATCCCACCCAAAAATAA
SEQ ID NO: 9
GCAAATGGGAGAGACCCTTTGAAGTCAAGGACACCGAGGAAGAGGACTTCCACG
TGGACCAGGTGACCACCGTGAAGGTGCCTATGATGAAGCGTTTAGGCATGTTTA
ACATCCAGCACTGTAAGAAGCTGTCCAGCTGGGTGCTGCTGATGAAATACCTGG
GCAATGCCACCGCCATCTTCTTCCTGCCTGATGAGGGGAAACTACAGCACCTGGA
AAATGAACTCACCCACGATATCATCACCAAGTTCCTGGAAAATGAAGACAGAAG
GTCTGCCAGCTTACATTTACCCAAACTGTCCATTACTGGAACCTATGATCTGAAG
AGCGTCCTGGGTCAACTGGGCATCACTAAGGTCTTCAGCAATGGGGCTGACCTCT
CCGGGGTCACAGAGGAGGCACCCCTGAAGCTCTCCAAGGCCGTGCATAAGGCTG
TGCTGACCATCGACGAGAAAGGGACTGAAGCTGCTGGGGCCATGTTTTTAGAGG
CCATACCCATGTCTATCCCCCCCGAGGTCAAGTTCAACAAACCCTTTGTCTTCTTA
ATGATTGAACAAAATACCAAGTCTCCCCTCTTCATGGGAAAAGTGGTGAATCCCA
CCCAAAAATAA
SEQ ID NO: 10
GCAAATGGGAGAGACCCTTTGAAGTCAAGGACACCGAGGAAGAGGACTTCCACG
TGGACCAGGTGACCACCGTGAAGGTGCCTATGATGAAGCGTTTAGGCATGTTTA
ACATCCAGCACTGTAAGAAGCTGTCCAGCTGGGTGCTGCTGATGAAATACCTGG
GCAATGCCACCGCCATCTTCTTCCTGCCTGATGAGGGGAAACTACAGCACCTGGA
AAATGAACTCACCCACGATATCATCACCAAGTTCCTGGAAAATGAAGACAGAAG
GTGATTCCCCAACCTGAGGGTGACCAAGAAGCTGCCCACACCTCTTAGCCATGTT
GGGACTGAGGCCCATCAGGACTGGCCAGAGGGCTGAGGAGGGTGAACCCCACAT
CCCTGGGTCACTGCTACTCTGTATAAACTTGGCTTCCAGAATGAGGCCACCACTG
AGTTCAGGCAGCGCCATCCATGCTCCATGAGGAGGACAGTACCCAGGGGTGAGG
AGGTAAAGGTCTCGTCCCTGGGGACTTCCCACTCCAGTGTGGACACTGTCCCTTC
CCAATATCCAGTGCCCAGGGCAGGGACAGCAGCACCACCACACGTTCTGGCAGA
ACCAAAAAGGAACAGATGGGCTTCCTGGCAAAGGCAGCAGTGGAGTGTGGAGTT
CAAGGGTAGAATGTCCCTGGGGGGACGGGGGAAGAGCCTGTGTGGCAAGGCCC
AGAAAAGCAAGGTTCGGAATTGGAACAGCCAGGCCATGTTCGCAGAAGGCTTGC
GTTTCTCTGTCACTTTATCGGTGCTGTTAGATTGGGTGTCCTGTAGTAAGTGATAC
TTAAACATGAGCCACACATTAGTGTATGTGTGTGCATTCGTGATTATGCCCATGC
CCTGCTGATCTAGTTCGTTTTGTACACTGTAAAACCAAGATGAAAATACAAAAGG
TGTCGGGTTCATAATAGGAATCGAGGCTGGAATTTCTCTGTTCCATGCCAGCACC
TCCTGAGGTCTCTGCTCCAGGGGTTGAGAAAGAACAAAGAGGCTGAGAGGGTAA
CGGATCAGAGAGCCCAGAGCCAAGCTGCCCGCTCACACCAGACCCTGCTCAGGG
TGGCATTGTCTCCCCATGGAAAACCAGAGAGGAGCACTCAGCCTGGTGTGGTCA
CTCTTCTCTTATCCACTAAACGGTTGTCACTGGGCACTGCCACCAGCCCCGTGTTT
CTCTGGGTGTAGGGCCCTGGGGATGTTACAGGCTGGGGGCCAGGTGACCCAACA
CTACAGGGCAAGATGAGACAGGCTTCCAGGACACCTAGAATATCAGAGGAGGTG
GCATTTCAAGCTTTTGTGATTCATTCGATGTTAACATTCTTTGACTCAATGTAGAA
GAGCTAAAAGTAGAACAAACCAAAGCCGAGTTCCCATCTTAGTGTGGGTGGAGG
ACACAGGAGTAAGTGGCAGAAATAATCAGAAAAGAAAACACTTGCACTGTGGTG
GGTCCCAGAAGAACAAGAGGAATGCTGTGCCATGCCTTGAATTTCTTTTCTGCAC
GACAGGTCTGCCAGCTTACATTTACCCAAACTGTCCATTACTGGAACCTATGATC
TGAAGAGCGTCCTGGGTCAACTGGGCATCACTAAGGTCTTCAGCAATGGGGCTG
ACCTCTCCGGGGTCACAGAGGAGGCACCCCTGAAGCTCTCCAAGGTGAGATCAC
CCTGACGACCTTGTTGCACCCTGGTATCTGTAGGGAAGAATGTGTGGGGGCTGCA
GCTCTGTCCTGAGGCTGAGGAAGGGGCCGAGGGAAACAAATGAAGACCCAGGCT
GAGCTCCTGAAGATGCCCGTGATTCACTGACACGGGACGTGGTCAAACAGCAAA
GCCAGGCAGGGGACTGCTGTGCAGCTGGCACTTTCGGGGCCTCCCTTGAGGTTGT
GTCACTGACCCTGAATTTCAACTTTGCCCAAGACCTTCTAGACATTGGGCCTTGA
TTTATCCATACTGACACAGAAAGGTTTGGGCTAAGTTGTTTCAAAGGAATTTCTG
ACTCCTTCGATCTGTGAGATTTGGTGTCTGAATTAATGAATGATTTCAGCTAAAG
ATGACACTTATTTTGGAAAACTAAAGGCGACCAATGAACAACTGCAGTTCCATG
AATGGCTGCATTATCTTGGGGTCTGGGCACTGTGAAGGTCACTGCCAGGGTCCGT
GTCCTCAAGGAGCTTCAAGCCGTGTACTAGAAAGGAGAGAGCCCTGGAGGCAGA
CGTGGAGTGACGATGCTCTTCCCTGTTCTGAGTTGTGGGTGCACCTGAGCAGGGG
GAGAGGCGCTTGTCAGGAAGATGGACAGAGGGGAGCCAGCCCCATCAGCCAAA
GCCTTGAGGAGGAGCAAGGCCTATGTGACAGGGAGGGAGAGGATGTGCAGGGC
CAGGGCCGTCCAGGGGGAGTGAGCGCTTCCTGGGAGGTGTCCACGTGAGCCTTG
CTCGAGGCCTGGGATCAGCCTTACAACGTGTCTCTGCTTCTCTCCCCTCCAGGCC
GTGCATAAGGCTGTGCTGACCATCGACGAGAAAGGGACTGAAGCTGCTGGGGCC
ATGTTTTTAGAGGCCATACCCATGTCTATCCCCCCCGAGGTCAAGTTCAACAAAC
CCTTTGTCTTCTTAATGATTGAACAAAATACCAAGTCTCCCCTCTTCATGGGAAA
AGTGGTGAATCCCACCCAAAAATAA
SEQ ID NO: 11
GTCTGCCAGCTTACATTTACCCAAACTGTCCATTACTGGAACCTATGATCTGAAG
AGCGTCCTGGGTCAACTGGGCATCACTAAGGTCTTCAGCAATGGGGCTGACCTCT
CCGGGGTCACAGAGGAGGCACCCCTGAAGCTCTCCAAGGCCGTGCATAAGGCTG
TGCTGACCATCGACGAGAAAGGGACTGAAGCTGCTGGGGCCATGTTTTTAGAGG
CCATACCCATGTCTATCCCCCCCGAGGTCAAGTTCAACAAACCCTTTGTCTTCTTA
ATGATTGAACAAAATACCAAGTCTCCCCTCTTCATGGGAAAAGTGGTGAATCCCA
CCCAAAAATAA
SEQ ID NO: 12
GTCTGCCAGCTTACATTTACCCAAACTGTCCATTACTGGAACCTATGATCTGAAG
AGCGTCCTGGGTCAACTGGGCATCACTAAGGTCTTCAGCAATGGGGCTGACCTCT
CCGGGGTCACAGAGGAGGCACCCCTGAAGCTCTCCAAGGTGAGATCACCCTGAC
GACCTTGTTGCACCCTGGTATCTGTAGGGAAGAATGTGTGGGGGCTGCAGCTCTG
TCCTGAGGCTGAGGAAGGGGCCGAGGGAAACAAATGAAGACCCAGGCTGAGCT
CCTGAAGATGCCCGTGATTCACTGACACGGGACGTGGTCAAACAGCAAAGCCAG
GCAGGGGACTGCTGTGCAGCTGGCACTTTCGGGGCCTCCCTTGAGGTTGTGTCAC
TGACCCTGAATTTCAACTTTGCCCAAGACCTTCTAGACATTGGGCCTTGATTTATC
CATACTGACACAGAAAGGTTTGGGCTAAGTTGTTTCAAAGGAATTTCTGACTCCT
TCGATCTGTGAGATTTGGTGTCTGAATTAATGAATGATTTCAGCTAAAGATGACA
CTTATTTTGGAAAACTAAAGGCGACCAATGAACAACTGCAGTTCCATGAATGGCT
GCATTATCTTGGGGTCTGGGCACTGTGAAGGTCACTGCCAGGGTCCGTGTCCTCA
AGGAGCTTCAAGCCGTGTACTAGAAAGGAGAGAGCCCTGGAGGCAGACGTGGA
GTGACGATGCTCTTCCCTGTTCTGAGTTGTGGGTGCACCTGAGCAGGGGGAGAGG
CGCTTGTCAGGAAGATGGACAGAGGGGAGCCAGCCCCATCAGCCAAAGCCTTGA
GGAGGAGCAAGGCCTATGTGACAGGGAGGGAGAGGATGTGCAGGGCCAGGGCC
GTCCAGGGGGAGTGAGCGCTTCCTGGGAGGTGTCCACGTGAGCCTTGCTCGAGG
CCTGGGATCAGCCTTACAACGTGTCTCTGCTTCTCTCCCCTCCAGGCCGTGCATA
AGGCTGTGCTGACCATCGACGAGAAAGGGACTGAAGCTGCTGGGGCCATGTTTT
TAGAGGCCATACCCATGTCTATCCCCCCCGAGGTCAAGTTCAACAAACCCTTTGT
CTTCTTAATGATTGAACAAAATACCAAGTCTCCCCTCTTCATGGGAAAAGTGGTG
AATCCCACCCAAAAATAA
SEQ ID NO: 13
GCCGTGCATAAGGCTGTGCTGACCATCGACGAGAAAGGGACTGAAGCTGCTGGG
GCCATGTTTTTAGAGGCCATACCCATGTCTATCCCCCCCGAGGTCAAGTTCAACA
AACCCTTTGTCTTCTTAATGATTGAACAAAATACCAAGTCTCCCCTCTTCATGGG
AAAAGTGGTGAATCCCACCCAAAAATAA
SEQ ID NO: 14
MPSSVSWGILLLAGLCCLVPVSLAEDPQGDAAQKTDTSHHDQDHPTFNKITPNLAEF
AFSLYRQLAHQSNSTNIFFSPVSIATAFAMLSLGTKADTHDEILEGLNFNLTEIPEAQIH
EGFQELLRTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEVKDTEEED
FHVDQVTTVKVPMMKRLGMFNIQHCKKLSSWVLLMKYLGNATAIFFLPDEGKLQH
LENELTHDIITKFLENEDRRSASLHLPKLSITGTYDLKSVLGQLGITKVFSNGADLSGV
TEEAPLKLSKAVHKAVLTIDEKGTEAAGAMFLEAIPMSIPPEVKFNKPFVFLMIEQNT
KSPLFMGKVVNPTQK
SEQ ID NO: 15
TGGGCAGGAACTGGGCACTGTGCCCAGGGCATGCACTGCCTCCACGCAGCAACC
CTCAGAGTCCTGAGCTGAACCAAGAAGGAGGAGGGGGTCGGGCCTCCGAGGAA
GGCCTAGCCGCTGCTGCTGCCAGGAATTCCAGGTTGGAGGGGCGGCAACCTCCT
GCCAGCCTTCAGGCCACTCTCCTGTGCCTGCCAGAAGAGACAGAGCTTGAGGAG
AGCTTGAGGAGAGCAGGAAAGGTGGGACATTGCTGCTGCTGCTCACTCAGTTCC
ACAGGTGGGAGGGACAGCAGGGCTTAGAGTGGGGGTCATTGTGCAGATGGGAA
AACAAAGGCCCAGAGAGGGGAAGAAATGCCCAGGAGCTACCGAGGGCAGGCGA
CCTCAACCACAGCCCAGTGCTGGAGCTGTGAGTGGATGTAGAGCAGCGGAATAT
CCATTCAGCCAGCTCAGGGGAAGGACAGGGGCCCTGAAGCCAGGGGATGGAGCT
GCAGGGAAGGGAGCTCAGAGAGAAGGGGAGGGGAGTCTGAGCTCAGTTTCCCG
CTGCCTGAAAGGAGGGTGGTACCTACTCCCTTCACAGGGTAACTGAATGAGAGA
CTGCCTGGAGGAAAGCTCTTCAAGTGTGGCCCACCCCACCCCAGTGACACCAGC
CCCTGACACGGGGGAGGGAGGGCAGCATCAGGAGGGGCTTTCTGGGCACACCCA
GTACCCGTCTCTGAGCTTTCCTTGAACTGTTGCATTTTAATCCTCACAGCAGCTCA
ACAAGGTACATACCGTCACCATCCCCATTTTACAGATAGGGAAATTGAGGCTCG
GAGCGGTTAAACAACTCACCTGAGGCCTCACAGCCAGTAAGTGGGTTCCCTGGT
CTGAATGTGTGTGCTGGAGGATCCTGTGGGTCACTCGCCTGGTAGAGCCCCAAGG
TGGAGGCATAAATGGGACTGGTGAATGACAGAAGGGGCAAAAATGCACTCATCC
ATTCACTCTGCAAGTATCTACGGCACGTACGCCAGCTCCCAAGCAGGTTTGCGGG
TTGCACAGCGGGCGATGCAATCTGATTTAGGCTTTTAAAGGGATTGCAATCAAGT
GGGGCCCCACTAGCCTCAACCCTGTACCTCCCCTCCCCTCCACCCCCAGCAGTCT
CCAAAGGCCTCCAACAACCCCAGAGTGGGGGCCATGTATCCAAAGAAACTCCAA
GCTGTATACGGATCACACTGGTTTTCCAGGAGCAAAAACAGAAACAGGCCTGAG
GCTGGTCAAAATTGAACCTCCTCCTGCTCTGAGCAGCCTGGGGGGCAGACTAAG
CAGAGGGCTGTGCAGACCCACATAAAGAGCCTACTGTGTGCCAGGCACTTCACC
CGAGGCACTTCACAAGCATGCTTGGGAATGAAACTTCCAACTCTTTGGGATGCAG
GTGAAACAGTTCCTGGTTCAGAGAGGTGAAGCGGCCTGCCTGAGGCAGCACAGC
TCTTCTTTACAGATGTGCTTCCCCACCTCTACCCTGTCTCACGGCCCCCCATGCCA
GCCTGACGGTTGTGTCTGCCTCAGTCATGCTCCATTTTTCCATCGGGACCATCAA
GAGGGTGTTTGTGTCTAAGGCTGACTGGGTAACTTTGGATGAGCGGTCTCTCCGC
TCTGAGCCTGTTTCCTCATCTGTCAAATGGGCTCTAACCCACTCTGATCTCCCAGG
GCGGCAGTAAGTCTTCAGCATCAGGCATTTTGGGGTGACTCAGTAAATGGTAGAT
CTTGCTACCAGTGGAACAGCCACTAAGGATTCTGCAGTGAGAGCAGAGGGCCAG
CTAAGTGGTACTCTCCCAGAGACTGTCTGACTCACGCCACCCCCTCCACCTTGGA
CACAGGACGCTGTGGTTTCTGAGCCAGGTACAATGACTCCTTTCGGTAAGTGCAG
TGGAAGCTGTACACTGCCCAGGCAAAGCGTCCGGGCAGCGTAGGCGGGCGACTC
AGATCCCAGCCAGTGGACTTAGCCCCTGTTTGCTCCTCCGATAACTGGGGTGACC
TTGGTTAATATTCACCAGCAGCCTCCCCCGTTGCCCCTCTGGATCCACTGCTTAAA
TACGGACGAGGACAGGGCCCTGTCTCCTCAGCTTCAGGCACCACCACTGACCTG
GGACAGTGAATCGTAAGTATGCCTTTCACTGCGAGAGGTTCTGGAGAGGCTTCTG
AGCTCCCCATGGCCCAGGCAGGCAGCAGGTCTGGGGCAGGAGGGGGGTTGTGGA
GTGGGTATCCGCCTGCTGAGGTGCAGGGCAGATGGAGAGGCTGCAGCTGAGCTC
CTATTTTCATAATAACAGCAGCCATGAGGGTTGTGTCCTGTTTCCCAGTCCTGCCC
GGTCCCCCCTCGGTACCTCCTGGTGGATACACTGGTTCCTGTAAGCAGAAGTGGA
TGAGGGTGTCTAGGTCTGCAGTCCTGGCACCCCAGGATGGGGGACACCAGCCAA
GATACAGCAACAGCAACAAAGCGCAGCCATTTCTTTCTGTTTGCACAGCTCCTCT
GTCTGTCGGGGGCTCCTGTCTGTTGTCTCCTATAAGCCTCACCACCTCTCCTACTG
CTTGGGCATGCATCTTTCTCCCCTTCTATAGATGAGGAGGTTAAGGTCCAGAGAG
GGGTGGGGAGGAACGCCGGCTCACATTCTCCATCCCCTCCAGATATGACCAGGA
ACAGACCTGTGCCAGGCCTCAGCCTTACATCAAAATGGGCCTCCCCATGCACCGT
GGACCTCTGGGCCCTCCTGTCCCAGTGGAGGACAGGAAGCTGTGAGGGGCACTG
TCACCCAGGGCTCAAGCTGGCATTCCTGAATAATCGCTCTGCACCAGGCCACGGC
TAAGCTCAGTGCGTGATTAAGCCTCATAACCCTCCAAGGCAGTTACTAGTGTGAT
TCCCATTTTACAGATGAGGAAGATGGGGACAGAGAGGTGAATAACTGGCCCCAA
ATCACACACCATCCATAATTCGGGCTCAGGCACCTGGCTCCAGTCCCCAAACTCT
TGAACCTGGCCCTAGTGTCACTGTTTCTCTTGGGTCTCAGGCGCTGGATGGGGAA
CAGGAAACCTGGGCTGGACTTGAGGCCTCTCTGATGCTCGGTGACTTCAGACAGT
TGCTCAACCTCTCTGTTCTCTTGGGCAAAACATGATAACCTTTGACTTCTGTCCCC
TCCCCTCACCCCACCCGACCTTGATCTCTGAAGTGTTGGAAGGATTTAATTTTTCC
TGCACTGAGTTTTGGAGACAGGTCAAAAAGATGACCAAGGCCAAGGTGGCCAGT
TTCCTATAGAACGCCTCTAAAAGACCTGCAGCAATAGCAGCAAGAACTGGTATT
CTCGAGAACTTGCTGCGCAGCAGGCACTTCTTGGCATTTTATGTGTATTTAATTTC
ACAATAGCTCTATGACAAAGTCCACCTTTCTCATCTCCAGGAAACTGAGGTTCAG
AGAGGTTAAGTAACTTGTCCAAGGTCACACAGCTAATAGCAAGTTGACGTGGAG
CAATCTGGCCTCAGAGCCTTTAATTTTAGCCACAGACTGATGCTCCCCTCTTCATT
TAGCCAGGCTGCCTCTGAAGTTTTCTGATTCAAGACTTCTGGCTTCAGCTTTGTAC
ACAGAGATGATTCAATGTCAGGTTTTGGAGTGAAATCTGTTTAATCCCAGACAAA
ACATTTAGGATTACATCTCAGTTTTGTAAGCAAGTAGCTCTGTGATTTTTAGTGAG
TTATTTAATGCTCTTTGGGGCTCAATTTTTCTATCTATAAAATAGGGCTAATAATT
TGCACCTTATAGGGTAAGCTTTGAGGACAGATTAGATGATACGGTGCCTGTAAAA
CACCAGGTGTTAGTAAGTGTGGCAATGATGGTGACGCTGAGGCTGATGTTTGCTT
AGCATAGGGTTAGGCAGCTGGCAGGCAGTAAACAGTTGGATAATTTAATGGAAA
ATTTGCCAAACTCAGATGCTGTTCACTGCTGAGCAGGAGCCCCTTCCTGCTGAAA
TGGTCCTGGGGAGTGCAGCAGGCTCTCCGGGAAGAAATCTACCATCTCTCGGGC
AGGAGCTCAACCTGTGTGCAGGTACAGGGAGGGCTTCCTCACCTGGTGCCCACTC
ATGCATTACGTCAGTTATTCCTCATCCCTGTCCAAAGGATTCTTTTCTCCATTGTA
CAGCTATGAAGCTAGTGCTCAAAGAAGTGAAGTCATTTACCCCAGGCCCCCTGCC
AGTAAGTGACAGGGCCTGGTCACACTTGGGTTTATTTATTGCCCAGTTCAACAGG
TTGTTTGACCATAGGCGAGATTCTCTTCCCTGCACCCTGCCGGGTTGCTCTTGGTC
CCTTATTTTATGCTCCCGGGTAGAAATGGTGTGAGATTAGGCAGGGAGTGGCTCG
CTTCCCTGTCCCTGGCCCCGCAAAGAGTGCTCCCACCTGCCCCGATCCCAGAAAT
GTCACCATGAAGCCTTCATTCTTTTGGTTTAAAGCTTGGCCTCAGTGTCCGTACAC
CATGGGGTACTTGGCCAGATGGCGACTTTCTCCTCTCCAGTCGCCCTCCCAGGCA
CTAGCTTTTAGGAGTGCAGGGTGCTGCCTCTGATAGAAGGGCCAGGAGAGAGCA
GGTTTTGGAGTCCTGATGTTATAAGGAACAGCTTGGGAGGCATAATGAACCCAA
CATGATGCTTGAGACCAATGTCACAGCCCAATTCTGACATTCATCATCTGAGATC
TGAGGACACAGCTGTCTCAGTTCATGATCTGAGTGCTGGGAAAGCCAAGACTTGT
TCCAGCTTTGTCACTGACTTGCTGTATAGCCTCAACAAGGCCCTGACCCTCTCTG
GGCTTCAAACTCTTCACTGTGAAAGGAGGAAACCAGAGTAGGTGATGTGACACC
AGGAAAGATGGATGGGTGTGGGGGAATGTGCTCCTCCCAGCTGTCACCCCCTCG
CCACCCTCCCTGCACCAGCCTCTCCACCTCCTTTGAGCCCAGAATTCCCCTGTCTA
GGAGGGCACCTGTCTCATGCCTAGCCATGGGAATTCTCCATCTGTTTTGCTACAT
TGAACCCAGATGCCATTCTAACCAAGAATCCTGGCTGGGTGCAGGGGCTCTCGCC
TGTAACCCCAGCACTTTGGGAGGCCAAGGCAGGCGGATCAAGAGGTCAGGAGTT
CAAGACCTGCCTGGCCAACACGGTGAAACCTCAGCTCTACTAAAAATACAAAAA
TTAGCCAGGCGTGGTGGCACACGCCTGTAATCCCAGCTATTTGGGAAGCTGAGA
CAGAAGAATTTCTTGAACCCGGGAGGTGGAGGTTTCAGTGAGCCGAGATCACGC
CACTGCACTCCACCCTGGCAGATAAAGCGAGACTCTGTCTCAAAAAAAACCCAA
AAACCTATGTTAGTGTACAGAGGGCCCCAGTGAAGTCTTCTCCCAGCCCCACTTT
GCACAACTGGGGAGAGTGAGGCCCCAGGACCAGAGGATTCTTGCTAAAGGCCAA
GTGGATAGTGATGGCCCTGCCAGGGCTAGAAGCCACAACCTCTGGCCCTGAGGC
CACTCAGCATATTTAGTGTCCCCACCCTGCAGAGGCCCAACTCCCTCCTGACCAC
TGAGCCCTGTAATGATGGGGGAATTTCCATAAGCCATGAAGGACTGCACAAAGT
TCAGTTGGGAAGTGAAAGAGAAATTAAAGGGAGATGGAAATATACAGCACTAAT
TTTAGCACCGTCTTTAGTTCTAACAACACTAGCTAGCTGAAGAAAAATACAAACA
TGTATTATGTAATGTGTGGTCTGTTCCATTTGGATTACTTAGAGGCACGAGGGCC
AGGAGAAAGGTGGTGGAGAGAAACCAGCTTTGCACTTCATTTGTTGCTTTATTGG
AAGGAAACTTTTAAAAGTCCAAGGGGGTTGAAGAATCTCAATATTTGTTATTTCC
AGCTTTTTTTCTCCAGTTTTTCATTTCCCAAATTCAAGGACACCTTTTTCTTTGTAT
TTTGTTAAGATGATGGTTTTGGTTTTGTGACTAGTAGTTAACAATGTGGCTGCCGG
GCATATTCTCCTCAGCTAGGACCTCAGTTTTCCCATCTGTGAAGACGGCAGGTTC
TACCTAGGGGGCTGCAGGCTGGTGGTCCGAAGCCTGGGCATATCTGGAGTAGAA
GGATCACTGTGGGGCAGGGCAGGTTCTGTGTTGCTGTGGATGACGTTGACTTTGA
CCATTGCTCGGCAGAGCCTGCTCTCGCTGGTTCAGCCACAGGCCCCACCACTCCC
TATTGTCTCAGCCCCGGGTATGAAACATGTATTCCTCACTGGCCTATCACCTGAA
GCCTTTGAATTTGCAACACCTGCCAACCCCTCCCTCAAAAGAGTTGCCCTCTCAG
ATCCTTTTGATGTAAGGTTTGGTGTTGAGACTTATTTCACTAAATTCTCATACATA
AACATCACTTTATGTATGAGGCAAAATGAGGACCAGGGAGATGAATGACTTGTC
CTGGCTCATACACCTGGAAAGTGACAGAGTCAGATTAGATCCCAGGTCTATCTGA
AGTTAAAAGAGGTGTCTTTTCACTTCCCACCTCCTCCATCTACTTTAAAGCAGCA
CAAACCCCTGCTTTCAAGGAGAGATGAGCGTCTCTAAAGCCCCTGACAGCAAGA
GCCCAGAACTGGGACACCATTAGTGACCCAGACGGCAGGTAAGCTGACTGCAGG
AGCATCAGCCTATTCTTGTGTCTGGGACCACAGAGCATTGTGGGGACAGCCCCGT
CTCTTGGGAAAAAAACCCTAAGGGCTGAGGATCCTTGTGAGTGTTGGGTGGGAA
CAGCTCCCAGGAGGTTTAATCACAGCCCCTCCATGCTCTCTAGCTGTTGCCATTG
TGCAAGATGCATTTCCCTTCTGTGCAGCAGTTTCCCTGGCCACTAAATAGTGGGA
TTAGATAGAAGCCCTCCAAGGGCTTCCAGCTTGACATGATTCTTGATTCTGATCT
GGCCCGATTCCTGGATAATCGTGGGCAGGCCCATTCCTCTTCTTGTGCCTCATTTT
CTTCTTTTGTAAAACAATGGCTGTACCATTTGCATCTTAGGGTCATTGCAGATGTA
AGTGTTGCTGTCCAGAGCCTGGGTGCAGGACCTAGATGTAGGATTCTGGTTCTGC
TACTTCCTCAGTGACATTGAATAGCTGACCTAATCTCTCTGGCTTTGGTTTCTTCA
TCTGTAAAAGAAGGATATTAGCATTAGCACCTCACGGGATTGTTACAAGAAAGC
AATGAATTAACACATGTGAGCACGGAGAACAGTGCTTGGCATATGGTAAGCACT
ACGTACATTTTGCTATTCTTCTGATTCTTTCAGTGTTACTGATGTCGGCAAGTACT
TGGCACAGGCTGGTTTAATAATCCCTAGGCACTTCCACGTGGTGTCAATCCCTGA
TCACTGGGAGTCATCATGTGCCTTGACTCGGGGCCTGGCCCCCCCATCTCTGTCTT
GCAGGACAATGCCGTCTTCTGTCTCGTGGGGCATCCTCCTGCTGGCAGGCCTGTG
CTGCCTGGTCCCTGTCTCCCTGGCTGAGGATCCCCAGGGAGATGCTGCCCAGAAG
ACAGATACATCCCACCATGATCAGGATCACCCAACCTTCAACAAGATCACCCCC
AACCTGGCTGAGTTCGCCTTCAGCCTATACCGCCAGCTGGCACACCAGTCCAACA
GCACCAATATCTTCTTCTCCCCAGTGAGCATCGCTACAGCCTTTGCAATGCTCTCC
CTGGGGACCAAGGCTGACACTCACGATGAAATCCTGGAGGGCCTGAATTTCAAC
CTCACGGAGATTCCGGAGGCTCAGATCCATGAAGGCTTCCAGGAACTCCTCCGTA
CCCTCAACCAGCCAGACAGCCAGCTCCAGCTGACCACCGGCAATGGCCTGTTCCT
CAGCGAGGGCCTGAAGCTAGTGGATAAGTTTTTGGAGGATGTTAAAAAGTTGTA
CCACTCAGAAGCCTTCACTGTCAACTTCGGGGACACCGAAGAGGCCAAGAAACA
GATCAACGATTACGTGGAGAAGGGTACTCAAGGGAAAATTGTGGATTTGGTCAA
GGAGCTTGACAGAGACACAGTTTTTGCTCTGGTGAATTACATCTTCTTTAAAGGT
AAGGTTGCTCAACCAGCCTGAGCTGTTCCCATAGAAACAAGCAAAAATATTCTC
AAACCATCAGTTCTTGAACTCTCCTTGGCAATGCATTATGGGCCATAGCAATGCT
TTTCAGCGTGGATTCTTCAGTTTTCTACACACAAACACTAAAATGTTTTCCATCAT
TGAGTAATTTGAGGAAATAATAGATTAAACTGTCAAAACTACTGACAGCTCTGCA
GAACTTTTCAGAGCCTTTAATGTCCTTGTGTATACTGTATATGTAGAATATATAAT
GCTTAGAACTATAGAACAAATTGTAATACACTGCATAAAGGGATAGTTTCATGG
AACATACTTTACACGACTCTAGTGTCCCAGAATCAGTATCAGTTTTGCAATCTGA
AAGACCTGGGTTCAAATCCTGCCTCTAACACAATTAGCTTTTGACAAAAACAATG
CATTCTACCTCTTTGAGGTGCTAATTTCTCATCTTAGCATGGACAAAATACCATTC
TTGCTGTCAGGTTTTTTTAGGATTAAACAAATGACAAAGACTGTGGGGATGGTGT
GTGGCATACAGCAGGTGATGGACTCTTCTGTATCTCAGGCTGCCTTCCTGCCCCT
GAGGGGTTAAAATGCCAGGGTCCTGGGGGCCCCAGGGCATTCTAAGCCAGCTCC
CACTGTCCCAGGAAAACAGCATAGGGGAGGGGAGGTGGGAGGCAAGGCCAGGG
GCTGCTTCCTCCACTCTGAGGCTCCCTTGCTCTTGAGGCAAAGGAGGGCAGTGGA
GAGCAGCCAGGCTGCAGTCAGCACAGCTAAAGTCCTGGCTCTGCTGTGGCCTTA
GTGGGGGCCCAGGTCCCTCTCCAGCCCCAGTCTCCTCCTTCTGTCCAATGAGAAA
GCTGGGATCAGGGGTCCCTGAGGCCCCTGTCCACTCTGCATGCCTCGATGGTGAA
GCTCTGTTGGTATGGCAGAGGGGAGGCTGCTCAGGCATCTGCATTTCCCCTGCCA
ATCTAGAGGATGAGGAAAGCTCTCAGGAATAGTAAGCAGAATGTTTGCCCTGGA
TGAATAACTGAGCTGCCAATTAACAAGGGGCAGGGAGCCTTAGACAGAAGGTAC
CAAATATGCCTGATGCTCCAACATTTTATTTGTAATATCCAAGACACCCTCAAAT
AAACATATGATTCCAATAAAAATGCACAGCCACGATGGCATCTCTTAGCCTGAC
ATCGCCACGATGTAGAAATTCTGCATCTTCCTCTAGTTTTGAATTATCCCCACACA
ATCTTTTTCGGCAGCTTGGATGGTCAGTTTCAGCACCTTTTACAGATGATGAAGCT
GAGCCTCGAGGGATGTGTGTCGTCAAGGGGGCTCAGGGCTTCTCAGGGAGGGGA
CTCATGGTTTCTTTATTCTGCTACACTCTTCCAAACCTTCACTCACCCCTGGTGAT
GCCCACCTTCCCCTCTCTCCAGGCAAATGGGAGAGACCCTTTGAAGTCAAGGACA
CCGAGGAAGAGGACTTCCACGTGGACCAGGTGACCACCGTGAAGGTGCCTATGA
TGAAGCGTTTAGGCATGTTTAACATCCAGCACTGTAAGAAGCTGTCCAGCTGGGT
GCTGCTGATGAAATACCTGGGCAATGCCACCGCCATCTTCTTCCTGCCTGATGAG
GGGAAACTACAGCACCTGGAAAATGAACTCACCCACGATATCATCACCAAGTTC
CTGGAAAATGAAGACAGAAGGTGATTCCCCAACCTGAGGGTGACCAAGAAGCTG
CCCACACCTCTTAGCCATGTTGGGACTGAGGCCCATCAGGACTGGCCAGAGGGC
TGAGGAGGGTGAACCCCACATCCCTGGGTCACTGCTACTCTGTATAAACTTGGCT
TCCAGAATGAGGCCACCACTGAGTTCAGGCAGCGCCATCCATGCTCCATGAGGA
GGACAGTACCCAGGGGTGAGGAGGTAAAGGTCTCGTCCCTGGGGACTTCCCACT
CCAGTGTGGACACTGTCCCTTCCCAATATCCAGTGCCCAGGGCAGGGACAGCAG
CACCACCACACGTTCTGGCAGAACCAAAAAGGAACAGATGGGCTTCCTGGCAAA
GGCAGCAGTGGAGTGTGGAGTTCAAGGGTAGAATGTCCCTGGGGGGACGGGGG
AAGAGCCTGTGTGGCAAGGCCCAGAAAAGCAAGGTTCGGAATTGGAACAGCCA
GGCCATGTTCGCAGAAGGCTTGCGTTTCTCTGTCACTTTATCGGTGCTGTTAGATT
GGGTGTCCTGTAGTAAGTGATACTTAAACATGAGCCACACATTAGTGTATGTGTG
TGCATTCGTGATTATGCCCATGCCCTGCTGATCTAGTTCGTTTTGTACACTGTAAA
ACCAAGATGAAAATACAAAAGGTGTCGGGTTCATAATAGGAATCGAGGCTGGAA
TTTCTCTGTTCCATGCCAGCACCTCCTGAGGTCTCTGCTCCAGGGGTTGAGAAAG
AACAAAGAGGCTGAGAGGGTAACGGATCAGAGAGCCCAGAGCCAAGCTGCCCG
CTCACACCAGACCCTGCTCAGGGTGGCATTGTCTCCCCATGGAAAACCAGAGAG
GAGCACTCAGCCTGGTGTGGTCACTCTTCTCTTATCCACTAAACGGTTGTCACTG
GGCACTGCCACCAGCCCCGTGTTTCTCTGGGTGTAGGGCCCTGGGGATGTTACAG
GCTGGGGGCCAGGTGACCCAACACTACAGGGCAAGATGAGACAGGCTTCCAGGA
CACCTAGAATATCAGAGGAGGTGGCATTTCAAGCTTTTGTGATTCATTCGATGTT
AACATTCTTTGACTCAATGTAGAAGAGCTAAAAGTAGAACAAACCAAAGCCGAG
TTCCCATCTTAGTGTGGGTGGAGGACACAGGAGTAAGTGGCAGAAATAATCAGA
AAAGAAAACACTTGCACTGTGGTGGGTCCCAGAAGAACAAGAGGAATGCTGTGC
CATGCCTTGAATTTCTTTTCTGCACGACAGGTCTGCCAGCTTACATTTACCCAAAC
TGTCCATTACTGGAACCTATGATCTGAAGAGCGTCCTGGGTCAACTGGGCATCAC
TAAGGTCTTCAGCAATGGGGCTGACCTCTCCGGGGTCACAGAGGAGGCACCCCT
GAAGCTCTCCAAGGTGAGATCACCCTGACGACCTTGTTGCACCCTGGTATCTGTA
GGGAAGAATGTGTGGGGGCTGCAGCTCTGTCCTGAGGCTGAGGAAGGGGCCGAG
GGAAACAAATGAAGACCCAGGCTGAGCTCCTGAAGATGCCCGTGATTCACTGAC
ACGGGACGTGGTCAAACAGCAAAGCCAGGCAGGGGACTGCTGTGCAGCTGGCAC
TTTCGGGGCCTCCCTTGAGGTTGTGTCACTGACCCTGAATTTCAACTTTGCCCAAG
ACCTTCTAGACATTGGGCCTTGATTTATCCATACTGACACAGAAAGGTTTGGGCT
AAGTTGTTTCAAAGGAATTTCTGACTCCTTCGATCTGTGAGATTTGGTGTCTGAAT
TAATGAATGATTTCAGCTAAAGATGACACTTATTTTGGAAAACTAAAGGCGACCA
ATGAACAACTGCAGTTCCATGAATGGCTGCATTATCTTGGGGTCTGGGCACTGTG
AAGGTCACTGCCAGGGTCCGTGTCCTCAAGGAGCTTCAAGCCGTGTACTAGAAA
GGAGAGAGCCCTGGAGGCAGACGTGGAGTGACGATGCTCTTCCCTGTTCTGAGT
TGTGGGTGCACCTGAGCAGGGGGAGAGGCGCTTGTCAGGAAGATGGACAGAGG
GGAGCCAGCCCCATCAGCCAAAGCCTTGAGGAGGAGCAAGGCCTATGTGACAGG
GAGGGAGAGGATGTGCAGGGCCAGGGCCGTCCAGGGGGAGTGAGCGCTTCCTG
GGAGGTGTCCACGTGAGCCTTGCTCGAGGCCTGGGATCAGCCTTACAACGTGTCT
CTGCTTCTCTCCCCTCCAGGCCGTGCATAAGGCTGTGCTGACCATCGACGAGAAA
GGGACTGAAGCTGCTGGGGCCATGTTTTTAGAGGCCATACCCATGTCTATCCCCC
CCGAGGTCAAGTTCAACAAACCCTTTGTCTTCTTAATGATTGAACAAAATACCAA
GTCTCCCCTCTTCATGGGAAAAGTGGTGAATCCCACCCAAAAATAA
SEQ ID NO: 16
TGGGCAGGAACTGGGCACTGTGCCCAGGGCATGCACTGCCTCCACGCAGCAACC
CTCAGAGTCCTGAGCTGAACCAAGAAGGAGGAGGGGGTCGGGCCTCCGAGGAA
GGCCTAGCCGCTGCTGCTGCCAGGAATTCCAGGTTGGAGGGGCGGCAACCTCCT
GCCAGCCTTCAGGCCACTCTCCTGTGCCTGCCAGAAGAGACAGAGCTTGAGGAG
AGCTTGAGGAGAGCAGGAAAGGTGGGACATTGCTGCTGCTGCTCACTCAGTTCC
ACAGGTGGGAGGGACAGCAGGGCTTAGAGTGGGGGTCATTGTGCAGATGGGAA
AACAAAGGCCCAGAGAGGGGAAGAAATGCCCAGGAGCTACCGAGGGCAGGCGA
CCTCAACCACAGCCCAGTGCTGGAGCTGTGAGTGGATGTAGAGCAGCGGAATAT
CCATTCAGCCAGCTCAGGGGAAGGACAGGGGCCCTGAAGCCAGGGGATGGAGCT
GCAGGGAAGGGAGCTCAGAGAGAAGGGGAGGGGAGTCTGAGCTCAGTTTCCCG
CTGCCTGAAAGGAGGGTGGTACCTACTCCCTTCACAGGGTAACTGAATGAGAGA
CTGCCTGGAGGAAAGCTCTTCAAGTGTGGCCCACCCCACCCCAGTGACACCAGC
CCCTGACACGGGGGAGGGAGGGCAGCATCAGGAGGGGCTTTCTGGGCACACCCA
GTACCCGTCTCTGAGCTTTCCTTGAACTGTTGCATTTTAATCCTCACAGCAGCTCA
ACAAGGTACATACCGTCACCATCCCCATTTTACAGATAGGGAAATTGAGGCTCG
GAGCGGTTAAACAACTCACCTGAGGCCTCACAGCCAGTAAGTGGGTTCCCTGGT
CTGAATGTGTGTGCTGGAGGATCCTGTGGGTCACTCGCCTGGTAGAGCCCCAAGG
TGGAGGCATAAATGGGACTGGTGAATGACAGAAGGGGCAAAAATGCACTCATCC
ATTCACTCTGCAAGTATCTACGGCACGTACGCCAGCTCCCAAGCAGGTTTGCGGG
TTGCACAGCGGGCGATGCAATCTGATTTAGGCTTTTAAAGGGATTGCAATCAAGT
GGGGCCCCACTAGCCTCAACCCTGTACCTCCCCTCCCCTCCACCCCCAGCAGTCT
CCAAAGGCCTCCAACAACCCCAGAGTGGGGGCCATGTATCCAAAGAAACTCCAA
GCTGTATACGGATCACACTGGTTTTCCAGGAGCAAAAACAGAAACAGGCCTGAG
GCTGGTCAAAATTGAACCTCCTCCTGCTCTGAGCAGCCTGGGGGGCAGACTAAG
CAGAGGGCTGTGCAGACCCACATAAAGAGCCTACTGTGTGCCAGGCACTTCACC
CGAGGCACTTCACAAGCATGCTTGGGAATGAAACTTCCAACTCTTTGGGATGCAG
GTGAAACAGTTCCTGGTTCAGAGAGGTGAAGCGGCCTGCCTGAGGCAGCACAGC
TCTTCTTTACAGATGTGCTTCCCCACCTCTACCCTGTCTCACGGCCCCCCATGCCA
GCCTGACGGTTGTGTCTGCCTCAGTCATGCTCCATTTTTCCATCGGGACCATCAA
GAGGGTGTTTGTGTCTAAGGCTGACTGGGTAACTTTGGATGAGCGGTCTCTCCGC
TCTGAGCCTGTTTCCTCATCTGTCAAATGGGCTCTAACCCACTCTGATCTCCCAGG
GCGGCAGTAAGTCTTCAGCATCAGGCATTTTGGGGTGACTCAGTAAATGGTAGAT
CTTGCTACCAGTGGAACAGCCACTAAGGATTCTGCAGTGAGAGCAGAGGGCCAG
CTAAGTGGTACTCTCCCAGAGACTGTCTGACTCACGCCACCCCCTCCACCTTGGA
CACAGGACGCTGTGGTTTCTGAGCCAGGTACAATGACTCCTTTCGGTAAGTGCAG
TGGAAGCTGTACACTGCCCAGGCAAAGCGTCCGGGCAGCGTAGGCGGGCGACTC
AGATCCCAGCCAGTGGACTTAGCCCCTGTTTGCTCCTCCGATAACTGGGGTGACC
TTGGTTAATATTCACCAGCAGCCTCCCCCGTTGCCCCTCTGGATCCACTGCTTAAA
TACGGACGAGGACAGGGCCCTGTCTCCTCAGCTTCAGGCACCACCACTGACCTG
GGACAGTGAATCGTAAGTATGCCTTTCACTGCGAGAGGTTCTGGAGAGGCTTCTG
AGCTCCCCATGGCCCAGGCAGGCAGCAGGTCTGGGGCAGGAGGGGGGTTGTGGA
GTGGGTATCCGCCTGCTGAGGTGCAGGGCAGATGGAGAGGCTGCAGCTGAGCTC
CTATTTTCATAATAACAGCAGCCATGAGGGTTGTGTCCTGTTTCCCAGTCCTGCCC
GGTCCCCCCTCGGTACCTCCTGGTGGATACACTGGTTCCTGTAAGCAGAAGTGGA
TGAGGGTGTCTAGGTCTGCAGTCCTGGCACCCCAGGATGGGGGACACCAGCCAA
GATACAGCAACAGCAACAAAGCGCAGCCATTTCTTTCTGTTTGCACAGCTCCTCT
GTCTGTCGGGGGCTCCTGTCTGTTGTCTCCTATAAGCCTCACCACCTCTCCTACTG
CTTGGGCATGCATCTTTCTCCCCTTCTATAGATGAGGAGGTTAAGGTCCAGAGAG
GGGTGGGGAGGAACGCCGGCTCACATTCTCCATCCCCTCCAGATATGACCAGGA
ACAGACCTGTGCCAGGCCTCAGCCTTACATCAAAATGGGCCTCCCCATGCACCGT
GGACCTCTGGGCCCTCCTGTCCCAGTGGAGGACAGGAAGCTGTGAGGGGCACTG
TCACCCAGGGCTCAAGCTGGCATTCCTGAATAATCGCTCTGCACCAGGCCACGGC
TAAGCTCAGTGCGTGATTAAGCCTCATAACCCTCCAAGGCAGTTACTAGTGTGAT
TCCCATTTTACAGATGAGGAAGATGGGGACAGAGAGGTGAATAACTGGCCCCAA
ATCACACACCATCCATAATTCGGGCTCAGGCACCTGGCTCCAGTCCCCAAACTCT
TGAACCTGGCCCTAGTGTCACTGTTTCTCTTGGGTCTCAGGCGCTGGATGGGGAA
CAGGAAACCTGGGCTGGACTTGAGGCCTCTCTGATGCTCGGTGACTTCAGACAGT
TGCTCAACCTCTCTGTTCTCTTGGGCAAAACATGATAACCTTTGACTTCTGTCCCC
TCCCCTCACCCCACCCGACCTTGATCTCTGAAGTGTTGGAAGGATTTAATTTTTCC
TGCACTGAGTTTTGGAGACAGGTCAAAAAGATGACCAAGGCCAAGGTGGCCAGT
TTCCTATAGAACGCCTCTAAAAGACCTGCAGCAATAGCAGCAAGAACTGGTATT
CTCGAGAACTTGCTGCGCAGCAGGCACTTCTTGGCATTTTATGTGTATTTAATTTC
ACAATAGCTCTATGACAAAGTCCACCTTTCTCATCTCCAGGAAACTGAGGTTCAG
AGAGGTTAAGTAACTTGTCCAAGGTCACACAGCTAATAGCAAGTTGACGTGGAG
CAATCTGGCCTCAGAGCCTTTAATTTTAGCCACAGACTGATGCTCCCCTCTTCATT
TAGCCAGGCTGCCTCTGAAGTTTTCTGATTCAAGACTTCTGGCTTCAGCTTTGTAC
ACAGAGATGATTCAATGTCAGGTTTTGGAGTGAAATCTGTTTAATCCCAGACAAA
ACATTTAGGATTACATCTCAGTTTTGTAAGCAAGTAGCTCTGTGATTTTTAGTGAG
TTATTTAATGCTCTTTGGGGCTCAATTTTTCTATCTATAAAATAGGGCTAATAATT
TGCACCTTATAGGGTAAGCTTTGAGGACAGATTAGATGATACGGTGCCTGTAAAA
CACCAGGTGTTAGTAAGTGTGGCAATGATGGTGACGCTGAGGCTGATGTTTGCTT
AGCATAGGGTTAGGCAGCTGGCAGGCAGTAAACAGTTGGATAATTTAATGGAAA
ATTTGCCAAACTCAGATGCTGTTCACTGCTGAGCAGGAGCCCCTTCCTGCTGAAA
TGGTCCTGGGGAGTGCAGCAGGCTCTCCGGGAAGAAATCTACCATCTCTCGGGC
AGGAGCTCAACCTGTGTGCAGGTACAGGGAGGGCTTCCTCACCTGGTGCCCACTC
ATGCATTACGTCAGTTATTCCTCATCCCTGTCCAAAGGATTCTTTTCTCCATTGTA
CAGCTATGAAGCTAGTGCTCAAAGAAGTGAAGTCATTTACCCCAGGCCCCCTGCC
AGTAAGTGACAGGGCCTGGTCACACTTGGGTTTATTTATTGCCCAGTTCAACAGG
TTGTTTGACCATAGGCGAGATTCTCTTCCCTGCACCCTGCCGGGTTGCTCTTGGTC
CCTTATTTTATGCTCCCGGGTAGAAATGGTGTGAGATTAGGCAGGGAGTGGCTCG
CTTCCCTGTCCCTGGCCCCGCAAAGAGTGCTCCCACCTGCCCCGATCCCAGAAAT
GTCACCATGAAGCCTTCATTCTTTTGGTTTAAAGCTTGGCCTCAGTGTCCGTACAC
CATGGGGTACTTGGCCAGATGGCGACTTTCTCCTCTCCAGTCGCCCTCCCAGGCA
CTAGCTTTTAGGAGTGCAGGGTGCTGCCTCTGATAGAAGGGCCAGGAGAGAGCA
GGTTTTGGAGTCCTGATGTTATAAGGAACAGCTTGGGAGGCATAATGAACCCAA
CATGATGCTTGAGACCAATGTCACAGCCCAATTCTGACATTCATCATCTGAGATC
TGAGGACACAGCTGTCTCAGTTCATGATCTGAGTGCTGGGAAAGCCAAGACTTGT
TCCAGCTTTGTCACTGACTTGCTGTATAGCCTCAACAAGGCCCTGACCCTCTCTG
GGCTTCAAACTCTTCACTGTGAAAGGAGGAAACCAGAGTAGGTGATGTGACACC
AGGAAAGATGGATGGGTGTGGGGGAATGTGCTCCTCCCAGCTGTCACCCCCTCG
CCACCCTCCCTGCACCAGCCTCTCCACCTCCTTTGAGCCCAGAATTCCCCTGTCTA
GGAGGGCACCTGTCTCATGCCTAGCCATGGGAATTCTCCATCTGTTTTGCTACAT
TGAACCCAGATGCCATTCTAACCAAGAATCCTGGCTGGGTGCAGGGGCTCTCGCC
TGTAACCCCAGCACTTTGGGAGGCCAAGGCAGGCGGATCAAGAGGTCAGGAGTT
CAAGACCTGCCTGGCCAACACGGTGAAACCTCAGCTCTACTAAAAATACAAAAA
TTAGCCAGGCGTGGTGGCACACGCCTGTAATCCCAGCTATTTGGGAAGCTGAGA
CAGAAGAATTTCTTGAACCCGGGAGGTGGAGGTTTCAGTGAGCCGAGATCACGC
CACTGCACTCCACCCTGGCAGATAAAGCGAGACTCTGTCTCAAAAAAAACCCAA
AAACCTATGTTAGTGTACAGAGGGCCCCAGTGAAGTCTTCTCCCAGCCCCACTTT
GCACAACTGGGGAGAGTGAGGCCCCAGGACCAGAGGATTCTTGCTAAAGGCCAA
GTGGATAGTGATGGCCCTGCCAGGGCTAGAAGCCACAACCTCTGGCCCTGAGGC
CACTCAGCATATTTAGTGTCCCCACCCTGCAGAGGCCCAACTCCCTCCTGACCAC
TGAGCCCTGTAATGATGGGGGAATTTCCATAAGCCATGAAGGACTGCACAAAGT
TCAGTTGGGAAGTGAAAGAGAAATTAAAGGGAGATGGAAATATACAGCACTAAT
TTTAGCACCGTCTTTAGTTCTAACAACACTAGCTAGCTGAAGAAAAATACAAACA
TGTATTATGTAATGTGTGGTCTGTTCCATTTGGATTACTTAGAGGCACGAGGGCC
AGGAGAAAGGTGGTGGAGAGAAACCAGCTTTGCACTTCATTTGTTGCTTTATTGG
AAGGAAACTTTTAAAAGTCCAAGGGGGTTGAAGAATCTCAATATTTGTTATTTCC
AGCTTTTTTTCTCCAGTTTTTCATTTCCCAAATTCAAGGACACCTTTTTCTTTGTAT
TTTGTTAAGATGATGGTTTTGGTTTTGTGACTAGTAGTTAACAATGTGGCTGCCGG
GCATATTCTCCTCAGCTAGGACCTCAGTTTTCCCATCTGTGAAGACGGCAGGTTC
TACCTAGGGGGCTGCAGGCTGGTGGTCCGAAGCCTGGGCATATCTGGAGTAGAA
GGATCACTGTGGGGCAGGGCAGGTTCTGTGTTGCTGTGGATGACGTTGACTTTGA
CCATTGCTCGGCAGAGCCTGCTCTCGCTGGTTCAGCCACAGGCCCCACCACTCCC
TATTGTCTCAGCCCCGGGTATGAAACATGTATTCCTCACTGGCCTATCACCTGAA
GCCTTTGAATTTGCAACACCTGCCAACCCCTCCCTCAAAAGAGTTGCCCTCTCAG
ATCCTTTTGATGTAAGGTTTGGTGTTGAGACTTATTTCACTAAATTCTCATACATA
AACATCACTTTATGTATGAGGCAAAATGAGGACCAGGGAGATGAATGACTTGTC
CTGGCTCATACACCTGGAAAGTGACAGAGTCAGATTAGATCCCAGGTCTATCTGA
AGTTAAAAGAGGTGTCTTTTCACTTCCCACCTCCTCCATCTACTTTAAAGCAGCA
CAAACCCCTGCTTTCAAGGAGAGATGAGCGTCTCTAAAGCCCCTGACAGCAAGA
GCCCAGAACTGGGACACCATTAGTGACCCAGACGGCAGGTAAGCTGACTGCAGG
AGCATCAGCCTATTCTTGTGTCTGGGACCACAGAGCATTGTGGGGACAGCCCCGT
CTCTTGGGAAAAAAACCCTAAGGGCTGAGGATCCTTGTGAGTGTTGGGTGGGAA
CAGCTCCCAGGAGGTTTAATCACAGCCCCTCCATGCTCTCTAGCTGTTGCCATTG
TGCAAGATGCATTTCCCTTCTGTGCAGCAGTTTCCCTGGCCACTAAATAGTGGGA
TTAGATAGAAGCCCTCCAAGGGCTTCCAGCTTGACATGATTCTTGATTCTGATCT
GGCCCGATTCCTGGATAATCGTGGGCAGGCCCATTCCTCTTCTTGTGCCTCATTTT
CTTCTTTTGTAAAACAATGGCTGTACCATTTGCATCTTAGGGTCATTGCAGATGTA
AGTGTTGCTGTCCAGAGCCTGGGTGCAGGACCTAGATGTAGGATTCTGGTTCTGC
TACTTCCTCAGTGACATTGAATAGCTGACCTAATCTCTCTGGCTTTGGTTTCTTCA
TCTGTAAAAGAAGGATATTAGCATTAGCACCTCACGGGATTGTTACAAGAAAGC
AATGAATTAACACATGTGAGCACGGAGAACAGTGCTTGGCATATGGTAAGCACT
ACGTACATTTTGCTATTCTTCTGATTCTTTCAGTGTTACTGATGTCGGCAAGTACT
TGGCACAGGCTGGTTTAATAATCCCTAGGCACTTCCACGTGGTGTCAATCCCTGA
TCACTGGGAGTCATCATGTGCCTTGACTCGGGGCCTGGCCCCCCCATCTCTGTCTT
GCAGGACAATGCCGTCTTCTGTCTCGTGGGGCATCCTCCTGCTGGCAGGCCTGTG
CTGCCTGGTCCCTGTCTCCCTGGCTGAGGATCCCCAGGGAGATGCTGCCCAGAAG
ACAGATACATCCCACCATGATCAGGATCACCCAACCTTCAACAAGATCACCCCC
AACCTGGCTGAGTTCGCCTTCAGCCTATACCGCCAGCTGGCACACCAGTCCAACA
GCACCAATATCTTCTTCTCCCCAGTGAGCATCGCTACAGCCTTTGCAATGCTCTCC
CTGGGGACCAAGGCTGACACTCACGATGAAATCCTGGAGGGCCTGAATTTCAAC
CTCACGGAGATTCCGGAGGCTCAGATCCATGAAGGCTTCCAGGAACTCCTCCGTA
CCCTCAACCAGCCAGACAGCCAGCTCCAGCTGACCACCGGCAATGGCCTGTTCCT
CAGCGAGGGCCTGAAGCTAGTGGATAAGTTTTTGGAGGATGTTAAAAAGTTGTA
CCACTCAGAAGCCTTCACTGTCAACTTCGGGGACACCGAAGAGGCCAAGAAACA
GATCAACGATTACGTGGAGAAGGGTACTCAAGGGAAAATTGTGGATTTGGTCAA
GGAGCTTGACAGAGACACAGTTTTTGCTCTGGTGAATTACATCTTCTTTAAAGGC
AAATGGGAGAGACCCTTTGAAGTCAAGGACACCGAGGAAGAGGACTTCCACGTG
GACCAGGTGACCACCGTGAAGGTGCCTATGATGAAGCGTTTAGGCATGTTTAAC
ATCCAGCACTGTAAGAAGCTGTCCAGCTGGGTGCTGCTGATGAAATACCTGGGC
AATGCCACCGCCATCTTCTTCCTGCCTGATGAGGGGAAACTACAGCACCTGGAAA
ATGAACTCACCCACGATATCATCACCAAGTTCCTGGAAAATGAAGACAGAAGGT
CTGCCAGCTTACATTTACCCAAACTGTCCATTACTGGAACCTATGATCTGAAGAG
CGTCCTGGGTCAACTGGGCATCACTAAGGTCTTCAGCAATGGGGCTGACCTCTCC
GGGGTCACAGAGGAGGCACCCCTGAAGCTCTCCAAGGCCGTGCATAAGGCTGTG
CTGACCATCGACGAGAAAGGGACTGAAGCTGCTGGGGCCATGTTTTTAGAGGCC
ATACCCATGTCTATCCCCCCCGAGGTCAAGTTCAACAAACCCTTTGTCTTCTTAAT
GATTGAACAAAATACCAAGTCTCCCCTCTTCATGGGAAAAGTGGTGAATCCCACC
CAAAAATAA
SEQ ID NO: 17
MAPKKKRKVH
SEQ ID NO: 18
TGCCCGCTCACACCAGACCCTG
SEQ ID NO: 19
ACGGGCGAGTGTGGTCTGGGAC
SEQ ID NO: 20
GCTGCTCAGGCATCTGCATTTC
SEQ ID NO: 21
CGACGAGTCCGTAGACGTAAAG
SEQ ID NO: 22
CAGAGGGCTGTGCAGACCCACA
SEQ ID NO: 23
GTCTCCCGACACGTCTGGGTGT
SEQ ID NO: 24
TTCAGACAGTTGCTCAACCTCT
SEQ ID NO: 25
AAGTCTGTCAACGAGTTGGAGA
SEQ ID NO: 26
GGCCTGGTCACACTTGGGTTTA
SEQ ID NO: 27
CCGGACCAGTGTGAACCCAAAT
SEQ ID NO: 28
CCCCACTTTGCACAACTGGGGA
SEQ ID NO: 29
GGGGTGAAACGTGTTGACCCCT
SEQ ID NO: 30
TGAGGATCCTTGTGAGTGTTGG
SEQ ID NO: 31
ACTCCTAGGAACACTCACAACC
SEQ ID NO: 32
AGGACCTAGATGTAGGATTCTG
SEQ ID NO: 33
TCCTGGATCTACATCCTAAGAC
SEQ ID NO: 34
GCTGGACTGGTATTTGTGTCTG
SEQ ID NO: 35
CGACCTGACCATAAACACAGAC
SEQ ID NO: 36
TATCCAGTCCAAGCTGTATACGGATCACACTG
SEQ ID NO: 37
ATGCACCTCCAAGCTGTATACGGATCACACTG
SEQ ID NO: 38
CAGTTCCACCAAGCTGTATACGGATCACACTG
SEQ ID NO: 39
TCTCGCCTCCAAGCTGTATACGGATCACACTG
SEQ ID NO: 40
GTGGTTCCTGAACCAGGAACTGTTTCACCTGCATC
SEQ ID NO: 41
CGAACTTCTGAACCAGGAACTGTTTCACCTGCATC
SEQ ID NO: 42
AGGAATGGTGAACCAGGAACTGTTTCACCTGCATC
SEQ ID NO: 43
GTTAGGAATGAACCAGGAACTGTTTCACCTGCATC
SEQ ID NO: 44
CGTTGAGGTGAACCAGGAACTGTTTCACCTGCATC
SEQ ID NO: 45
CTCCCAAATGAACCAGGAACTGTTTCACCTGCATC
SEQ ID NO: 46
GACATTTCTGAACCAGGAACTGTTTCACCTGCATC
SEQ ID NO: 47
TGAGAGATTGAACCAGGAACTGTTTCACCTGCATC
SEQ ID NO: 48
TATCCAGTCTCTTGAACCTGGCCCTAGTGTC
SEQ ID NO: 49
ATGCACCTCTCTTGAACCTGGCCCTAGTGTC
SEQ ID NO: 50
CAGTTCCACTCTTGAACCTGGCCCTAGTGTC
SEQ ID NO: 51
TCTCGCCTCTCTTGAACCTGGCCCTAGTGTC
SEQ ID NO: 52
GTGGTTCCAAATTAAATCCTTCCAACACTTCAGAGATCAAGGTC
SEQ ID NO: 53
CGAACTTCAAATTAAATCCTTCCAACACTTCAGAGATCAAGGTC
SEQ ID NO: 54
AGGAATGGAAATTAAATCCTTCCAACACTTCAGAGATCAAGGTC
SEQ ID NO: 55
GTTAGGAAAAATTAAATCCTTCCAACACTTCAGAGATCAAGGTC
SEQ ID NO: 56
CGTTGAGGAAATTAAATCCTTCCAACACTTCAGAGATCAAGGTC
SEQ ID NO: 57
CTCCCAAAAAATTAAATCCTTCCAACACTTCAGAGATCAAGGTC
SEQ ID NO: 58
GACATTTCAAATTAAATCCTTCCAACACTTCAGAGATCAAGGTC
SEQ ID NO: 59
TGAGAGATAAATTAAATCCTTCCAACACTTCAGAGATCAAGGTC
SEQ ID NO: 60
TATCCAGTCTCCATTGTACAGCTATGAAGCTAGTG
SEQ ID NO: 61
ATGCACCTCTCCATTGTACAGCTATGAAGCTAGTG
SEQ ID NO: 62
CAGTTCCACTCCATTGTACAGCTATGAAGCTAGTG
SEQ ID NO: 63
TCTCGCCTCTCCATTGTACAGCTATGAAGCTAGTG
SEQ ID NO: 64
GTGGTTCCGGGAGCATAAAATAAGGGACCAAGAGC
SEQ ID NO: 65
CGAACTTCGGGAGCATAAAATAAGGGACCAAGAGC
SEQ ID NO: 66
AGGAATGGGGGAGCATAAAATAAGGGACCAAGAGC
SEQ ID NO: 67
GTTAGGAAGGGAGCATAAAATAAGGGACCAAGAGC
SEQ ID NO: 68
CGTTGAGGGGGAGCATAAAATAAGGGACCAAGAGC
SEQ ID NO: 69
CTCCCAAAGGGAGCATAAAATAAGGGACCAAGAGC
SEQ ID NO: 70
GACATTTCGGGAGCATAAAATAAGGGACCAAGAGC
SEQ ID NO: 71
TGAGAGATGGGAGCATAAAATAAGGGACCAAGAGC
SEQ ID NO: 72
TATCCAGTTGGCAGATAAAGCGAGACTCTGTC
SEQ ID NO: 73
ATGCACCTTGGCAGATAAAGCGAGACTCTGTC
SEQ ID NO: 74
CAGTTCCATGGCAGATAAAGCGAGACTCTGTC
SEQ ID NO: 75
TCTCGCCTTGGCAGATAAAGCGAGACTCTGTC
SEQ ID NO: 76
GCCGAATGTGGCAGATAAAGCGAGACTCTGTC
SEQ ID NO: 77
TACTGCAGTGGCAGATAAAGCGAGACTCTGTC
SEQ ID NO: 78
CATGTTGATGGCAGATAAAGCGAGACTCTGTC
SEQ ID NO: 79
ATAGAGTCTGGCAGATAAAGCGAGACTCTGTC
SEQ ID NO: 80
TCACGCTCTGGCAGATAAAGCGAGACTCTGTC
SEQ ID NO: 81
GTGGTTCCGGGCCAGAGGTTGTGGCTTCTAG
SEQ ID NO: 82
CGAACTTCGGGCCAGAGGTTGTGGCTTCTAG
SEQ ID NO: 83
AGGAATGGGGGCCAGAGGTTGTGGCTTCTAG
SEQ ID NO: 84
GTTAGGAAGGGCCAGAGGTTGTGGCTTCTAG
SEQ ID NO: 85
CGTTGAGGGGGCCAGAGGTTGTGGCTTCTAG
SEQ ID NO: 86
CTCCCAAAGGGCCAGAGGTTGTGGCTTCTAG
SEQ ID NO: 87
GACATTTCGGGCCAGAGGTTGTGGCTTCTAG
SEQ ID NO: 88
TGAGAGATGGGCCAGAGGTTGTGGCTTCTAG
SEQ ID NO: 89
TATCCAGTGCTGACTGCAGGAGCATCAGC
SEQ ID NO: 90
GTGGTTCCACAGAAGGGAAATGCATCTTGCAC
SEQ ID NO: 91
CGAACTTACAGAAGGGAAATGCATCTTGCAC
SEQ ID NO: 92
AGGAATGGACAGAAGGGAAATGCATCTTGCAC
SEQ ID NO: 93
GTTAGGAAACAGAAGGGAAATGCATCTTGCAC
SEQ ID NO: 94
CGTTGAGGACAGAAGGGAAATGCATCTTGCAC
SEQ ID NO: 95
CTCCCAAAACAGAAGGGAAATGCATCTTGCAC
SEQ ID NO: 96
GACATTTCACAGAAGGGAAATGCATCTTGCAC
SEQ ID NO: 97
TGAGAGATACAGAAGGGAAATGCATCTTGCAC
SEQ ID NO: 98
TATCCAGTAGGCCCATTCCTCTTCTTGTGC
SEQ ID NO: 99
ATGCACCTAGGCCCATTCCTCTTCTTGTGC
SEQ ID NO: 100
CAGTTCCAAGGCCCATTCCTCTTCTTGTGC
SEQ ID NO: 101
TCTCGCCTAGGCCCATTCCTCTTCTTGTGC
SEQ ID NO: 102
GTGGTTCCCAATCCCGTGAGGTGCTAATGC
SEQ ID NO: 103
CGAACTTCAATCCCGTGAGGTGCTAATGC
SEQ ID NO: 104
AGGAATGGCAATCCCGTGAGGTGCTAATGC
SEQ ID NO: 105
GTTAGGAACAATCCCGTGAGGTGCTAATGC
SEQ ID NO: 106
CGTTGAGGCAATCCCGTGAGGTGCTAATGC
SEQ ID NO: 107
CTCCCAAACAATCCCGTGAGGTGCTAATGC
SEQ ID NO: 108
GACATTTCCAATCCCGTGAGGTGCTAATGC
SEQ ID NO: 109
TGAGAGATCAATCCCGTGAGGTGCTAATGC
SEQ ID NO: 110
CTGCTCAGGCATCTGCATTTCCC
SEQ ID NO: 111
ATGCCTCGATGGTGAAG
SEQ ID NO: 112
GGCAAACATTCTGCTTACTATT
SEQ ID NO: 113
CCTCGCTGGACTGGTATTTGTGTCT
SEQ ID NO: 114
GACAGGATGGCTTCCCTTC
SEQ ID NO: 115
GTCTAACTGCCATGTCTGGAT
SEQ ID NO: 116
ACCTTAGTGATGCCCAGTTGACCC
SEQ ID NO: 117
CCACAACTAGAATGCAGTGAA
SEQ ID NO: 118
GAATCACGGGCATCTTCAG
SEQ ID NO: 119
CTGCCCGCTCACACCAGAC
SEQ ID NO: 120
AGCACCTCCTGAGGTC
SEQ ID NO: 121
CACACCAGGCTGAGTG
SEQ ID NO: 122
TGGACAAACCACAACTAGAA
SEQ ID NO: 123
AGCCTGGCTAAATGAAGAG
SEQ ID NO: 124
AGCAAGTTGACGTGGAGCAATCTG
SEQ ID NO: 125
TGGACAAACCACAACTAGAA
SEQ ID NO: 126
CTCCCAAGCTGTTCCTTAT
SEQ ID NO: 127
AGGGTGCTGCCTCTGATAGAAGG
SEQ ID NO: 128
TGGACAAACCACAACTAGAA
SEQ ID NO: 129
CCACAGTGATCCTTCTACTC
SEQ ID NO: 130
TCTGTGAAGACGGCAGGTTCTACC
SEQ ID NO: 131
TGGACAAACCACAACTAGAA
SEQ ID NO: 132
CGTGAGGTGCTAATGCTAATA
SEQ ID NO: 133
AGCTGACCTAATCTCTCTGGCTTTGG
SEQ ID NO: 134
TGGACAAACCACAACTAGAA
SEQ ID NO: 135
CTGCAAGACAGAGATGGG
SEQ ID NO: 136
AGTCATCATGTGCCTTGACTCGG
SEQ ID NO: 137
GAGAGAACGCACCACTTTAC
SEQ ID NO: 138
AGCCTGGCTAAATGAAGAG
SEQ ID NO: 139
AGCAAGTTGACGTGGAGCAATCTG
SEQ ID NO: 140
GAGAGAACGCACCACTTTAC
SEQ ID NO: 141
CTCCCAAGCTGTTCCTTAT
SEQ ID NO: 142
AGGGTGCTGCCTCTGATAGAAGG
SEQ ID NO: 143
GAGAGAACGCACCACTTTAC
SEQ ID NO: 144
CCACAGTGATCCTTCTACTC
SEQ ID NO: 145
TCTGTGAAGACGGCAGGTTCTACC
SEQ ID NO: 146
GAGAGAACGCACCACTTTAC
SEO ID NO: 147
CGTGAGGTGCTAATGCTAATA
SEQ ID NO: 148
AGCTGACCTAATCTCTCTGGCTTTGG
SEQ ID NO: 149
GAGAGAACGCACCACTTTAC
SEQ ID NO: 150
CTGCAAGACAGAGATGGG
SEQ ID NO: 151
AGTCATCATGTGCCTTGACTCGG
SEQ ID NO: 152
CTGTTTCTCTTGGGTCTCAG
SEQ ID NO: 153
GGGACAGAAGTCAAAGGTTAT
SEQ ID NO: 154
AACAGAGAGGTTGAGCAACTGT
SEQ ID NO: 155
TGAAGTCATTTACCCCAGGC
SEQ ID NO: 156
CTCACACCATTTCTACCCGG
SEQ ID NO: 157
AATAAACCCAAGTGTGACCAGGCC
SEQ ID NO: 158
GCGAGACTCTGTCTCAAA
SEQ ID NO: 159
GGCCATCACTATCCACTT
SEQ ID NO: 160
TCCCCAGTTGTGCAAAG
SEQ ID NO: 161
CAGGAGCATCAGCCTAT
SEQ ID NO: 162
ATGGCAACAGCTAGAGAG
SEQ ID NO: 163
ATCCTTGTGAGTGTTGGGTGGGAA
SEQ ID NO: 164
GGCCCGATTCCTGGATAATC
SEQ ID NO: 165
GCCAGAGAGATTAGGTCAGC
SEQ ID NO: 166
CCAGAATCCTACATCTAGGTCCTGCAC
SEQ ID NO: 167
CTCCAAGGCCGTGCATAA
SEQ ID NO: 168
AGACATGGGTATGGCCTCTA
SEQ ID NO: 169
CTGACCATCGACGAGAAAGG
SEQ ID NO: 170
CTGACCATCGACAAGAAAGG

1. A polynucleotide comprising a template nucleic acid, wherein said template nucleic acid comprises, from 5′ to 3′:
(a) a splicing sequence comprising a splice acceptor sequence capable of pairing with an endogenous splice donor sequence in a SERPINA1 gene;
(b) a donor nucleic acid sequence encoding an alpha-1 antitrypsin (AAT) protein, or a portion thereof; and
(c) a termination sequence.

2. The polynucleotide of claim 1, wherein said polynucleotide comprises a 5′ homology arm and a 3′ homology arm flanking said template nucleic acid.

3. The polynucleotide of claim 1 or claim 2, wherein said polynucleotide does not comprise an exogenous promoter.

4. The polynucleotide of any one of claims 1-3, wherein said splicing sequence comprises a branch point.

5. The polynucleotide of any one of claims 1-4, wherein said splicing sequence is a naturally-occurring splicing sequence.

6. The polynucleotide of any one of claims 1-5, wherein said splicing sequence comprises an SV40 splicing sequence, a CMV splicing sequence, or a transferrin gene splicing sequence.

7. The polynucleotide of any one of claims 1-6, wherein said splicing sequence is a synthetic splicing sequence.

8. The polynucleotide of any one of claims 1-7, wherein said termination sequence comprises a stop codon.

9. The polynucleotide of any one of claims 1-8, wherein said termination sequence comprises a polyA sequence.

10. The polynucleotide of any one of claims 1-9, wherein said termination sequence comprises a stop codon and a polyA sequence.

11. The polynucleotide of any one of claims 1-10, wherein said AAT protein, or portion thereof, encoded by said donor nucleic acid sequence is a wild-type AAT protein, or a portion thereof.

12. The polynucleotide of any one of claims 1-11, wherein said donor nucleic acid sequence comprises one or more exons of a wild-type SERPINA1 gene.

13. The polynucleotide of any one of claims 1-12, wherein said donor nucleic acid sequence comprises one or more exons of a SERPINA1 gene that have been codon-modified but encode a wild-type AAT protein, or a portion thereof.

14. The polynucleotide of any one of claims 1-13, wherein said donor nucleic acid sequence encodes an AAT protein encoded by exons 2, 3, 4, and 5 of a SERPINA1 gene, and wherein said splice acceptor sequence is capable of pairing with an endogenous splice donor sequence that is positioned 3′ downstream and adjacent to exon 1a in a SERPINA1 gene.

15. The polynucleotide of any one of claims 1-14, wherein said donor nucleic acid sequence comprises exons 1b, 1c, 2, 3, 4, and 5 of a SERPINA1 gene, or codon-modified variants of one or more of exons 1b, 1c, 2, 3, 4, and 5 of a SERPINA1 gene.

16. The polynucleotide of any one of claims 1-15, wherein said donor nucleic acid sequence does not comprise one or more of introns 1b, 1c, 2, 3, and 4 of a SERPINA1 gene.

17. The polynucleotide of any one of claims 1-15, wherein said donor nucleic acid sequence comprises one or more of introns 1b, 1c, 2, 3, and 4 of a SERPINA1 gene.

18. The polynucleotide of any one of claims 1-15, wherein said donor nucleic acid sequence comprises introns 1b, 1c, 2, 3, and 4 of a SERPINA1 gene.

19. The polynucleotide of any one of claims 1-15, wherein said donor nucleic acid sequence comprises a nucleic acid sequence having at least 80% sequence identity to a sequence set forth in SEQ ID NO: 3.

20. The polynucleotide of any one of claims 1-15, wherein said donor nucleic acid sequence comprises a nucleic acid sequence set forth in SEQ ID NO: 3.

21. The polynucleotide of any one of claims 1-15, wherein said donor nucleic acid sequence comprises a nucleic acid sequence having at least 80% sequence identity to a sequence set forth in SEQ ID NO: 4.

22. The polynucleotide of any one of claims 1-15, wherein said donor nucleic acid sequence comprises a nucleic acid sequence set forth in SEQ ID NO: 4.

23. The polynucleotide of any one of claims 1-13, wherein said donor nucleic acid sequence encodes an AAT protein encoded by exons 2, 3, 4, and 5 of a SERPINA1 gene, and wherein said splice acceptor sequence is capable of pairing with an endogenous splice donor sequence that is positioned 3′ downstream and adjacent to exon 1b in a SERPINA1 gene.

24. The polynucleotide of claim 23, wherein said donor nucleic acid sequence comprises exons 1c, 2, 3, 4, and 5 of a SERPINA1 gene, or codon-modified variants of one or more of exons 1c, 2, 3, 4, and 5 of a SERPINA1 gene.

25. The polynucleotide of claim 23 or claim 24, wherein said donor nucleic acid sequence does not comprise one or more of introns 1c, 2, 3, and 4 of a SERPINA1 gene.

26. The polynucleotide of claim 23 or claim 24, wherein said donor nucleic acid sequence comprises one or more of introns 1c, 2, 3, and 4 of a SERPINA1 gene.

27. The polynucleotide of claim 23 or claim 24, wherein said donor nucleic acid sequence comprises introns 1c, 2, 3, and 4 of a SERPINA1 gene.

28. The polynucleotide of claim 23 or claim 24, wherein said donor nucleic acid sequence comprises a nucleic acid sequence having at least 80% sequence identity to a sequence set forth in SEQ ID NO: 5.

29. The polynucleotide of claim 23 or claim 24, wherein said donor nucleic acid sequence comprises a nucleic acid sequence set forth in SEQ ID NO: 5.

30. The polynucleotide of claim 23 or claim 24, wherein said donor nucleic acid sequence comprises a nucleic acid sequence having at least 80% sequence identity to a sequence set forth in SEQ ID NO: 6.

31. The polynucleotide of claim 23 or claim 24, wherein said donor nucleic acid sequence comprises a nucleic acid sequence set forth in SEQ ID NO: 6.

32. The polynucleotide of any one of claims 1-13, wherein said donor nucleic acid sequence encodes an AAT protein encoded by exons 2, 3, 4, and 5 of a SERPINA1 gene, and wherein said splice acceptor sequence is capable of pairing with an endogenous splice donor sequence that is positioned 3′ downstream and adjacent to exon 1c in a SERPINA1 gene.

33. The polynucleotide of claim 32, wherein said donor nucleic acid sequence comprises exons 2, 3, 4, and 5 of a SERPINA1 gene, or codon-modified variants of one or more of exons 2, 3, 4, and 5 of a SERPINA1 gene.

34. The polynucleotide of claim 32 or claim 33, wherein said donor nucleic acid sequence does not comprise one or more of introns 2, 3, and 4 of a SERPINA1 gene.

35. The polynucleotide of claim 32 or claim 33, wherein said donor nucleic acid sequence comprises one or more of introns 2, 3, and 4 of a SERPINA1 gene.

36. The polynucleotide of claim 32 or claim 33, wherein said donor nucleic acid sequence comprises introns 2, 3, and 4 of a SERPINA1 gene.

37. The polynucleotide of claim 32 or claim 33, wherein said donor nucleic acid sequence comprises a nucleic acid sequence having at least 80% sequence identity to a sequence set forth in SEQ ID NO: 7.

38. The polynucleotide of claim 32 or claim 33, wherein said donor nucleic acid sequence comprises a nucleic acid sequence set forth in SEQ ID NO: 7.

39. The polynucleotide of claim 32 or claim 33, wherein said donor nucleic acid sequence comprises a nucleic acid sequence having at least 80% sequence identity to a sequence set forth in SEQ ID NO: 8.

40. The polynucleotide of claim 32 or claim 33, wherein said donor nucleic acid sequence comprises a nucleic acid sequence set forth in SEQ ID NO: 8.

41. The polynucleotide of any one of claims 1-13, wherein said donor nucleic acid sequence encodes a portion of an AAT protein encoded by exons 3, 4, and 5 of a SERPINA1 gene, and wherein said splice acceptor sequence is capable of pairing with an endogenous splice donor sequence that is positioned 3′ downstream and adjacent to exon 2 in a SERPINA1 gene.

42. The polynucleotide of claim 41, wherein said donor nucleic acid sequence comprises exons 3, 4, and 5 of a SERPINA1 gene, or codon-modified variants of one or more of exons 3, 4, and 5 of a SERPINA1 gene.

43. The polynucleotide of claim 41 or claim 42, wherein said donor nucleic acid sequence does not comprise one or more of introns 3 and 4 of a SERPINA1 gene.

44. The polynucleotide of claim 41 or claim 42, wherein said donor nucleic acid sequence comprises one or more of introns 3 and 4 of a SERPINA1 gene.

45. The polynucleotide of claim 41 or claim 42, wherein said donor nucleic acid sequence comprises introns 3 and 4 of a SERPINA1 gene.

46. The polynucleotide of claim 41 or claim 42, wherein said donor nucleic acid sequence comprises a nucleic acid sequence having at least 80% sequence identity to a sequence set forth in SEQ ID NO: 9.

47. The polynucleotide of claim 41 or claim 42, wherein said donor nucleic acid sequence comprises a nucleic acid sequence set forth in SEQ ID NO: 9.

48. The polynucleotide of claim 41 or claim 42, wherein said donor nucleic acid sequence comprises a nucleic acid sequence having at least 80% sequence identity to a sequence set forth in SEQ ID NO: 10.

49. The polynucleotide of claim 41 or claim 42, wherein said donor nucleic acid sequence comprises a nucleic acid sequence set forth in SEQ ID NO: 10.

50. The polynucleotide of any one of claims 1-13, wherein said donor nucleic acid sequence encodes a portion of an AAT protein encoded by exons 4 and 5 of a SERPINA1 gene, and wherein said splice acceptor sequence is capable of pairing with an endogenous splice donor sequence that is positioned 3′ downstream and adjacent to exon 3 in a SERPINA1 gene.

51. The polynucleotide of claim 50, wherein said donor nucleic acid sequence comprises exons 4 and 5 of a SERPINA1 gene, or codon-modified variants of one or more of exons 4 and 5 of a SERPINA1 gene.

52. The polynucleotide of claim 50 or claim 51, wherein said donor nucleic acid sequence does not comprise intron 4 of a SERPINA1 gene.

53. The polynucleotide of claim 50 or claim 51, wherein said donor nucleic acid sequence comprises intron 4 of a SERPINA1 gene.

54. The polynucleotide of claim 50 or claim 51, wherein said donor nucleic acid sequence comprises a nucleic acid sequence having at least 80% sequence identity to a sequence set forth in SEQ ID NO: 11.

55. The polynucleotide of claim 50 or claim 51, wherein said donor nucleic acid sequence comprises a nucleic acid sequence set forth in SEQ ID NO: 11.

56. The polynucleotide of claim 50 or claim 51, wherein said donor nucleic acid sequence comprises a nucleic acid sequence having at least 80% sequence identity to a sequence set forth in SEQ ID NO: 12.

57. The polynucleotide of claim 50 or claim 51, wherein said donor nucleic acid sequence comprises a nucleic acid sequence set forth in SEQ ID NO: 12.

58. The polynucleotide of any one of claims 1-13, wherein said donor nucleic acid sequence encodes a portion of an AAT protein encoded by exon 5 of a SERPINA1 gene, and wherein said splice acceptor sequence is capable of pairing with an endogenous splice donor sequence that is positioned 3′ downstream and adjacent to exon 4 in a SERPINA1 gene.

59. The polynucleotide of claim 58, wherein said donor nucleic acid sequence comprises exon 5 of a SERPINA1 gene, or a codon-modified variant of exon 5 of a SERPINA1 gene.

60. The polynucleotide of claim 58 or claim 59, wherein said donor nucleic acid sequence comprises a nucleic acid sequence having at least 80% sequence identity to a sequence set forth in SEQ ID NO: 13.

61. The polynucleotide of claim 58 or claim 59, wherein said donor nucleic acid sequence comprises a nucleic acid sequence set forth in SEQ ID NO: 13.

62. The polynucleotide of any one of claims 1-61, wherein said template nucleic acid is a bidirectional template nucleic acid.

63. The polynucleotide of claim 62, wherein said donor nucleic acid sequence further comprises a reverse segment that is 3′ downstream of said termination sequence, wherein said reverse segment comprises, from 5′ to 3′:
(a) a reverse complement of a second termination sequence;
(b) a reverse complement of a second donor nucleic acid sequence encoding an AAT protein, or a portion thereof; and
(c) a reverse complement of a second splicing sequence comprising a splice acceptor sequence capable of pairing with an endogenous splice donor sequence in a SERPINA1 gene.

64. The polynucleotide of claim 62 or claim 63, wherein said second termination sequence is identical to said termination sequence of any one of claims 1-61.

65. The polynucleotide of any one of claim 62 or claim 63, wherein said second termination sequence differs from said termination sequence of any one of claims 1-61.

66. The polynucleotide of any one of claims 62-65, wherein said second donor nucleic acid sequence is identical to said donor nucleic acid sequence of any one of claims 1-65.

67. The polynucleotide of any one of claims 62-65, wherein said second donor nucleic acid sequence differs from said donor nucleic acid sequence of any one of claims 1-65, but encodes the same AAT protein, or portion thereof.

68. The polynucleotide of any one of claims 62-67, wherein said second splicing sequence is identical to said splicing sequence of any one of claims 1-67.

69. The polynucleotide of any one of claims 62-67, wherein said second splicing sequence differs from said splicing sequence of any one of claims 1-67, but is still capable of pairing with the same endogenous splice donor sequence in a SERPINA1 gene.

70. A recombinant DNA construct comprising said polynucleotide of any one of claims 1-69.

71. The recombinant DNA construct of claim 70, wherein said recombinant DNA construct encodes a recombinant virus comprising said polynucleotide.

72. The recombinant DNA construct of claim 70 or claim 71, wherein said recombinant virus is a recombinant adenovirus, a recombinant lentivirus, a recombinant retrovirus, or a recombinant adeno-associated virus (AAV).

73. The recombinant DNA construct of claim 72, wherein said recombinant virus is a recombinant AAV.

74. The recombinant DNA construct of claim 72 or claim 73, wherein said recombinant AAV has an AAV8 capsid.

75. A recombinant virus comprising said polynucleotide of any one of claims 1-69.

76. The recombinant virus of claim 75, wherein said recombinant virus is a recombinant adenovirus, a recombinant lentivirus, a recombinant retrovirus, or a recombinant AAV.

77. The recombinant virus of claim 75 or claim 76, wherein said recombinant virus is a recombinant AAV.

78. The recombinant virus of claim 77, wherein said recombinant AAV has an AAV8 capsid.

79. The recombinant virus of claim 77 or claim 78, wherein said polynucleotide is flanked by inverted terminal repeat (ITR) sequences.

80. A lipid nanoparticle composition comprising lipid nanoparticles comprising said polynucleotide of any one of claims 1-69.

81. A lipid nanoparticle composition comprising lipid nanoparticles comprising said recombinant DNA construct of any one of claims 70-74.

82. A pharmaceutical composition comprising a pharmaceutically acceptable carrier and said polynucleotide of any one of claims 1-69.

83. A pharmaceutical composition comprising a pharmaceutically acceptable carrier and said recombinant DNA construct of any one of claims 70-74.

84. A pharmaceutical composition comprising a pharmaceutically acceptable carrier and said recombinant virus of any one of claims 75-79.

85. A pharmaceutical composition comprising a pharmaceutically acceptable carrier and said lipid nanoparticle composition of claim 80 or claim 81.

86. A method for producing a genetically-modified eukaryotic cell comprising a modified SERPINA1 gene, said method comprising introducing into a eukaryotic cell:
(a) said polynucleotide comprising a template nucleic acid of any one of claims 1-69; and
(b) an engineered nuclease, or a second polynucleotide comprising a nucleic acid sequence encoding an engineered nuclease that is expressed in said eukaryotic cell;
wherein said engineered nuclease binds and cleaves a recognition sequence within an endogenous SERPINA1 gene to generate a cleavage site,
and wherein said template nucleic acid is inserted into said cleavage site to generate said modified SERPINA1 gene.

87. The method of claim 86, wherein said endogenous SERPINA1 gene comprises at least one mutation relative to a wild-type SERPINA1 gene and encodes a mutant AAT protein.

88. The method of claim 86 or claim 87, wherein said endogenous SERPINA1 gene comprises a Z allele mutation in exon 5.

89. The method of any one of claims 86-88, wherein said endogenous SERPINA1 gene comprises an S allele mutation in exon 3.

90. The method of any one of claims 86-89, wherein insertion of said template nucleic acid into said cleavage site disrupts expression of an endogenous AAT protein encoded by said endogenous SERPINA1 gene.

91. The method of any one of claims 86-90, wherein said template nucleic acid is inserted in-frame in said SERPINA1 gene.

92. The method of any one of claims 86-91, wherein said donor nucleic acid sequence of said template nucleic acid is operably linked to an endogenous SERPINA1 promoter following insertion of said template nucleic acid into said cleavage site.

93. The method of any one of claims 86-92, wherein said template nucleic acid does not comprise an exogenous promoter.

94. The method of any one of claims 86-93, wherein said modified SERPINA1 gene encodes a full-length AAT protein that does not comprise a Z allele mutation or an S allele mutation.

95. The method of any one of claims 86-94, wherein said modified SERPINA1 gene encodes a full-length wild-type AAT protein.

96. The method of any one of claims 86-95, wherein said modified SERPINA1 gene comprises a nucleic acid sequence of a wild-type SERPINA1 gene.

97. The method of any one of claims 86-94, wherein said modified SERPINA1 gene comprises one or more codon-modified exons and/or introns and encodes a wild-type AAT protein.

98. The method of any one of claims 86-94, wherein said modified SERPINA1 gene comprises a nucleic acid sequence having at least 80% sequence identity to a sequence set forth in SEQ ID NO: 16.

99. The method of any one of claims 86-94, wherein said modified SERPINA1 gene comprises a nucleic acid sequence set forth in SEQ ID NO: 16.

100. The method of any one of claims 86-99, wherein said engineered nuclease is an engineered meganuclease, a CRISPR system nuclease, a TALEN, a compact TALEN, a zinc finger nuclease, or a megaTAL.

101. The method of any one of claims 86-100, wherein said engineered nuclease is an engineered meganuclease.

102. The method of any one of claims 86-101, wherein said polynucleotide comprises a 5′ homology arm and a 3′ homology arm flanking said template nucleic acid that are homologous to sequences flanking said cleavage site.

103. The method of any one of claims 86-102, wherein said template nucleic acid is inserted into said cleavage site by homologous recombination.

104. The method of any one of claims 86-103, wherein said recognition sequence is positioned within intron 1a of said SERPINA1 gene.

105. The method of claim 104, wherein said polynucleotide comprising a template nucleic acid is said polynucleotide of any one of claims 14-22.

106. The method of any one of claims 86-103, wherein said recognition sequence is positioned within intron 1b of said SERPINA1 gene.

107. The method of claim 106, wherein said polynucleotide comprising a template nucleic acid is said polynucleotide of any one of claims 23-31.

108. The method of any one of claims 86-103, wherein said recognition sequence is positioned within intron 1c of said SERPINA1 gene.

109. The method of claim 108, wherein said polynucleotide comprising a template nucleic acid is said polynucleotide of any one of claims 32-40.

110. The method of any one of claims 86-103, wherein said recognition sequence is positioned within intron 2 of said SERPINA1 gene.

111. The method of claim 110, wherein said polynucleotide comprising a template nucleic acid is said polynucleotide of any one of claims 41-49.

112. The method of any one of claims 86-103, wherein said recognition sequence is positioned within intron 3 of said SERPINA1 gene.

113. The method of claim 112, wherein said polynucleotide comprising a template nucleic acid is said polynucleotide of any one of claims 50-57.

114. The method of any one of claims 86-103, wherein said recognition sequence is positioned within intron 4 of said SERPINA1 gene.

115. The method of claim 114, wherein said polynucleotide comprising a template nucleic acid is said polynucleotide of any one of claims 58-61.

116. The method of any one of claims 86-115, wherein said polynucleotide comprising a template nucleic acid is introduced into said eukaryotic cell by a first recombinant virus and said second polynucleotide is introduced into said eukaryotic cell by a second recombinant virus.

117. The method of claim 116, wherein said first recombinant virus and/or said second recombinant virus is a recombinant AAV.

118. The method of claim 117, wherein said first recombinant AAV and/or said second recombinant AAV has a capsid of serotype AAV8.

119. The method of claim 117 or claim 118, wherein said polynucleotide comprising a template nucleic acid and said second polynucleotide are flanked by ITR sequences.

120. The method of any one of claims 86-115, wherein said polynucleotide comprising a template nucleic acid is introduced into said eukaryotic cell by a recombinant virus, and wherein said engineered nuclease or said second polynucleotide is introduced into said eukaryotic cell by a lipid nanoparticle.

121. The method of claim 120, wherein said recombinant virus is a recombinant AAV.

122. The method of claim 121, wherein said recombinant AAV has a capsid of serotype AAV8.

123. The method of claim 121 or claim 122, wherein said polynucleotide comprising a template nucleic acid is flanked by ITR sequences.

124. The method of any one of claims 120-123, wherein said second polynucleotide is an mRNA.

125. The method of any one of claims 120-123, wherein said second polynucleotide is a double-stranded DNA encapsulated by said lipid nanoparticle.

126. The method of any one of claims 86-115, wherein said polynucleotide comprising a template nucleic acid is introduced into said eukaryotic cell by a lipid nanoparticle, and wherein said second polynucleotide is introduced into said eukaryotic cell by a recombinant virus.

127. The method of claim 126, wherein said polynucleotide comprising a template nucleic acid is a double-stranded DNA encapsulated by said lipid nanoparticle.

128. The method of claim 126 or claim 127, wherein said recombinant virus is a recombinant AAV.

129. The method of claim 128, wherein said recombinant AAV has a capsid of serotype AAV8.

130. The method of claim 128 or claim 129, wherein said second polynucleotide is flanked by ITR sequences.

131. The method of any one of claims 86-115, wherein said polynucleotide comprising a template nucleic acid is introduced into said eukaryotic cell by a first lipid nanoparticle, and said engineered nuclease or said second polynucleotide is introduced into said eukaryotic cell by a second lipid nanoparticle.

132. The method of claim 131, wherein said polynucleotide comprising a template nucleic acid is a double-stranded DNA encapsulated by said first lipid nanoparticle.

133. The method of claim 131 or claim 132, wherein said second polynucleotide is an mRNA encapsulated by said second lipid nanoparticle.

134. The method of claim 131 or claim 132 wherein said second polynucleotide is a double-stranded DNA encapsulated by said second lipid nanoparticle.

135. The method of any one of claims 86-134, wherein said eukaryotic cell is a mammalian cell.

136. The method of claim 135, wherein said mammalian cell is a human cell.

137. The method of claim 135 or claim 136, wherein said mammalian cell is a liver cell.

138. The method of claim 135 or claim 136, wherein said mammalian cell is a liver progenitor cell or stem cell.

139. A method for modifying a SERPINA1 gene in a target cell in a subject, said method comprising delivering to said target cell:
(a) said polynucleotide comprising a template nucleic acid of any one of claims 1-69; and
(b) an engineered nuclease, or a second polynucleotide comprising a nucleic acid sequence encoding an engineered nuclease that is expressed in said target cell;
wherein said engineered nuclease binds and cleaves a recognition sequence within an endogenous SERPINA1 gene in said target cell to generate a cleavage site,
and wherein said template nucleic acid is inserted into said cleavage site to generate said modified SERPINA1 gene.

140. The method of claim 139, wherein said endogenous SERPINA1 gene comprises at least one mutation relative to a wild-type SERPINA1 gene and encodes a mutant AAT protein.

141. The method of claim 139 or claim 140, wherein said endogenous SERPINA1 gene comprises a Z allele mutation in exon 5.

142. The method of any one of claims 139-141, wherein said endogenous SERPINA1 gene comprises an S allele mutation in exon 3.

143. The method of any one of claims 139-142, wherein insertion of said template nucleic acid into said cleavage site disrupts expression of an endogenous AAT protein encoded by said endogenous SERPINA1 gene.

144. The method of any one of claims 139-143, wherein said template nucleic acid is inserted in-frame in said SERPINA1 gene.

145. The method of any one of claims 139-144, wherein said donor nucleic acid sequence of said template nucleic acid is operably linked to an endogenous SERPINA1 promoter following insertion of said template nucleic acid into said cleavage site.

146. The method of any one of claims 139-145, wherein said template nucleic acid does not comprise an exogenous promoter.

147. The method of any one of claims 139-146, wherein said modified SERPINA1 gene encodes a full-length AAT protein that does not comprise a Z allele mutation or an S allele mutation.

148. The method of any one of claims 139-147, wherein said modified SERPINA1 gene encodes a full-length wild-type AAT protein.

149. The method of any one of claims 139-148, wherein said modified SERPINA1 gene comprises a nucleic acid sequence of a wild-type SERPINA1 gene.

150. The method of any one of claims 139-147, wherein said modified SERPINA1 gene comprises one or more codon-modified exons and/or introns and encodes a wild-type AAT protein.

151. The method of any one of claims 139-147, wherein said modified SERPINA1 gene comprises a nucleic acid sequence having at least 80% sequence identity to a sequence set forth in SEQ ID NO: 16.

152. The method of any one of claims 139-147, wherein said modified SERPINA1 gene comprises a nucleic acid sequence set forth in SEQ ID NO: 16.

153. The method of any one of claims 139-152, wherein said engineered nuclease is an engineered meganuclease, a CRISPR system nuclease, a TALEN, a compact TALEN, a zinc finger nuclease, or a megaTAL.

154. The method of any one of claims 139-153, wherein said engineered nuclease is an engineered meganuclease.

155. The method of any one of claims 139-154, wherein said polynucleotide comprises a 5′ homology arm and a 3′ homology arm flanking said template nucleic acid that are homologous to sequences flanking said cleavage site.

156. The method of any one of claims 139-155, wherein said template nucleic acid is inserted into said cleavage site by homologous recombination.

157. The method of any one of claims 139-156, wherein said recognition sequence is positioned within intron 1a of said SERPINA1 gene.

158. The method of claim 157, wherein said polynucleotide comprising a template nucleic acid is said polynucleotide of any one of claims 14-22.

159. The method of any one of claims 139-156, wherein said recognition sequence is positioned within intron 1b of said SERPINA1 gene.

160. The method of claim 159, wherein said polynucleotide comprising a template nucleic acid is said polynucleotide of any one of claims 23-31.

161. The method of any one of claims 139-156, wherein said recognition sequence is positioned within intron 1c of said SERPINA1 gene.

162. The method of claim 161, wherein said polynucleotide comprising a template nucleic acid is said polynucleotide of any one of claims 32-40.

163. The method of any one of claims 139-156, wherein said recognition sequence is positioned within intron 2 of said SERPINA1 gene.

164. The method of claim 163, wherein said polynucleotide comprising a template nucleic acid is said polynucleotide of any one of claims 41-49.

165. The method of any one of claims 139-156, wherein said recognition sequence is positioned within intron 3 of said SERPINA1 gene.

166. The method of claim 165, wherein said polynucleotide comprising a template nucleic acid is said polynucleotide of any one of claims 50-57.

167. The method of any one of claims 139-156, wherein said recognition sequence is positioned within intron 4 of said SERPINA1 gene.

168. The method of claim 167, wherein said polynucleotide comprising a template nucleic acid is said polynucleotide of any one of claims 58-61.

169. The method of any one of claims 139-168, wherein said polynucleotide comprising a template nucleic acid is delivered to said target cell by a first recombinant virus and said second polynucleotide is delivered to said eukaryotic cell by a second recombinant virus.

170. The method of claim 169, wherein said first recombinant virus and/or said second recombinant virus is a recombinant AAV.

171. The method of claim 170, wherein said first recombinant AAV and/or said second recombinant AAV has a capsid of serotype AAV8.

172. The method of claim 170 or claim 171, wherein said polynucleotide comprising a template nucleic acid and said second polynucleotide are flanked by ITR sequences.

173. The method of any one of claims 139-168, wherein said polynucleotide comprising a template nucleic acid is delivered to said target cell by a recombinant virus, and wherein said engineered nuclease or said second polynucleotide is delivered to said target cell by a lipid nanoparticle.

174. The method of claim 173, wherein said recombinant virus is a recombinant AAV.

175. The method of claim 174, wherein said recombinant AAV has a capsid of serotype AAV8.

176. The method of claim 174 or claim 175, wherein said polynucleotide comprising a template nucleic acid is flanked by ITR sequences.

177. The method of any one of claims 173-176, wherein said second polynucleotide is an mRNA.

178. The method of any one of claims 173-176, wherein said second polynucleotide is a double-stranded DNA encapsulated by said lipid nanoparticle.

179. The method of any one of claims 139-168, wherein said polynucleotide comprising a template nucleic acid is delivered to said target cell by a lipid nanoparticle, and wherein said second polynucleotide is delivered to said target cell by a recombinant virus.

180. The method of claim 179, wherein said polynucleotide comprising a template nucleic acid is a double-stranded DNA encapsulated by said lipid nanoparticle.

181. The method of claim 179 or claim 180, wherein said recombinant virus is a recombinant AAV.

182. The method of claim 181, wherein said recombinant AAV has a capsid of serotype AAV8.

183. The method of claim 181 or claim 182, wherein said second polynucleotide is flanked by ITR sequences.

184. The method of any one of claims 139-168, wherein said polynucleotide comprising a template nucleic acid is delivered to said target cell by a first lipid nanoparticle, and said engineered nuclease or said second polynucleotide is delivered to said target cell by a second lipid nanoparticle.

185. The method of claim 184, wherein said polynucleotide comprising a template nucleic acid is a double-stranded DNA encapsulated by said first lipid nanoparticle.

186. The method of claim 184 or claim 185, wherein said second polynucleotide is an mRNA encapsulated by said second lipid nanoparticle.

187. The method of claim 184 or claim 185 wherein said second polynucleotide is a double-stranded DNA encapsulated by said second lipid nanoparticle.

188. The method of any one of claims 139-187, wherein said target cell is a mammalian cell.

189. The method of claim 188, wherein said mammalian cell is a human cell.

190. The method of claim 188 or claim 189, wherein said mammalian cell is a liver cell.

191. The method of claim 188 or claim 189, wherein said mammalian cell is a liver progenitor cell or stem cell.

192. A method for treating AAT deficiency in a subject in need thereof, said method comprising administering to said subject:
(a) a pharmaceutical composition comprising an effective amount of said polynucleotide comprising a template nucleic acid of any one of claims 1-69; and
(b) a pharmaceutical composition comprising an effective amount of an engineered nuclease or a second polynucleotide comprising a nucleic acid sequence encoding an engineered nuclease;
wherein said polynucleotide comprising a template nucleic acid, and said engineered nuclease or said second polynucleotide, are delivered to a target cell in said subject, and wherein said engineered nuclease is expressed in said target cell if encoded by said second polynucleotide,
wherein said engineered nuclease binds and cleaves a recognition sequence within an endogenous SERPINA1 gene in said target cell to generate a cleavage site,
and wherein said template nucleic acid is inserted into said cleavage site to generate said modified SERPINA1 gene.

193. The method of claim 192, wherein said endogenous SERPINA1 gene comprises at least one mutation relative to a wild-type SERPINA1 gene and encodes a mutant AAT protein.

194. The method of claim 192 or claim 193, wherein said endogenous SERPINA1 gene comprises a Z allele mutation in exon 5.

195. The method of any one of claims 192-194, wherein said endogenous SERPINA1 gene comprises an S allele mutation in exon 3.

196. The method of any one of claims 192-195, wherein insertion of said template nucleic acid into said cleavage site disrupts expression of an endogenous AAT protein encoded by said endogenous SERPINA1 gene.

197. The method of any one of claims 192-196, wherein said template nucleic acid is inserted in-frame in said SERPINA1 gene.

198. The method of any one of claims 192-197, wherein said donor nucleic acid sequence of said template nucleic acid is operably linked to an endogenous SERPINA1 promoter following insertion of said template nucleic acid into said cleavage site.

199. The method of any one of claims 192-198, wherein said template nucleic acid does not comprise an exogenous promoter.

200. The method of any one of claims 192-199, wherein said modified SERPINA1 gene encodes a full-length AAT protein that does not comprise a Z allele mutation or an S allele mutation.

201. The method of any one of claims 192-200, wherein said modified SERPINA1 gene encodes a full-length wild-type AAT protein.

202. The method of any one of claims 192-201, wherein said modified SERPINA1 gene comprises a nucleic acid sequence of a wild-type SERPINA1 gene.

203. The method of any one of claims 192-200, wherein said modified SERPINA1 gene comprises one or more codon-modified exons and/or introns and encodes a wild-type AAT protein.

204. The method of any one of claims 192-200, wherein said modified SERPINA1 gene comprises a nucleic acid sequence having at least 80% sequence identity to a sequence set forth in SEQ ID NO: 16.

205. The method of any one of claims 192-200, wherein said modified SERPINA1 gene comprises a nucleic acid sequence set forth in SEQ ID NO: 16.

206. The method of any one of claims 192-205, wherein said engineered nuclease is an engineered meganuclease, a CRISPR system nuclease, a TALEN, a compact TALEN, a zinc finger nuclease, or a megaTAL.

207. The method of any one of claims 192-206, wherein said engineered nuclease is an engineered meganuclease.

208. The method of any one of claims 192-207, wherein said polynucleotide comprises a 5′ homology arm and a 3′ homology arm flanking said template nucleic acid that are homologous to sequences flanking said cleavage site.

209. The method of any one of claims 192-208, wherein said template nucleic acid is inserted into said cleavage site by homologous recombination.

210. The method of any one of claims 192-209, wherein said recognition sequence is positioned within intron 1a of said SERPINA1 gene.

211. The method of claim 210, wherein said polynucleotide comprising a template nucleic acid is said polynucleotide of any one of claims 14-22.

212. The method of any one of claims 192-209, wherein said recognition sequence is positioned within intron 1b of said SERPINA1 gene.

213. The method of claim 212, wherein said polynucleotide comprising a template nucleic acid is said polynucleotide of any one of claims 23-31.

214. The method of any one of claims 192-209, wherein said recognition sequence is positioned within intron 1c of said SERPINA1 gene.

215. The method of claim 214, wherein said polynucleotide comprising a template nucleic acid is said polynucleotide of any one of claims 32-40.

216. The method of any one of claims 192-209, wherein said recognition sequence is positioned within intron 2 of said SERPINA1 gene.

217. The method of claim 216, wherein said polynucleotide comprising a template nucleic acid is said polynucleotide of any one of claims 41-49.

218. The method of any one of claims 192-209, wherein said recognition sequence is positioned within intron 3 of said SERPINA1 gene.

219. The method of claim 218, wherein said polynucleotide comprising a template nucleic acid is said polynucleotide of any one of claims 50-57.

220. The method of any one of claims 192-209, wherein said recognition sequence is positioned within intron 4 of said SERPINA1 gene.

221. The method of claim 220, wherein said polynucleotide comprising a template nucleic acid is said polynucleotide of any one of claims 58-61.

222. The method of any one of claims 192-221, wherein said polynucleotide comprising a template nucleic acid is administered to said subject in a first recombinant virus and said second polynucleotide is administered to said subject in a second recombinant virus.

223. The method of claim 222, wherein said first recombinant virus and/or said second recombinant virus is a recombinant AAV.

224. The method of claim 223, wherein said first recombinant AAV and/or said second recombinant AAV has a capsid of serotype AAV8.

225. The method of claim 223 or claim 224, wherein said polynucleotide comprising a template nucleic acid and said second polynucleotide are flanked by ITR sequences.

226. The method of any one of claims 192-221, wherein said polynucleotide comprising a template nucleic acid is administered to said subject in a recombinant virus, and wherein said engineered nuclease or said second polynucleotide is administered to said subject in a lipid nanoparticle.

227. The method of claim 226, wherein said recombinant virus is a recombinant AAV.

228. The method of claim 227, wherein said recombinant AAV has a capsid of serotype AAV8.

229. The method of claim 227 or claim 228, wherein said polynucleotide comprising a template nucleic acid is flanked by ITR sequences.

230. The method of any one of claims 226-229, wherein said second polynucleotide is an mRNA.

231. The method of any one of claims 226-229, wherein said second polynucleotide is a double-stranded DNA encapsulated by said lipid nanoparticle.

232. The method of any one of claims 192-221, wherein said polynucleotide comprising a template nucleic acid is administered to said subject using a lipid nanoparticle, and wherein said second polynucleotide is administered to said subject using a recombinant virus.

233. The method of claim 232, wherein said polynucleotide comprising a template nucleic acid is a double-stranded DNA encapsulated by said lipid nanoparticle.

234. The method of claim 232 or claim 233, wherein said recombinant virus is a recombinant AAV.

235. The method of claim 234, wherein said recombinant AAV has a capsid of serotype AAV8.

236. The method of claim 234 or claim 235, wherein said second polynucleotide is flanked by ITR sequences.

237. The method of any one of claims 192-221, wherein said polynucleotide comprising a template nucleic acid is administered to said subject using a first lipid nanoparticle, and said engineered nuclease or said second polynucleotide is administered to said subject using a second lipid nanoparticle.

238. The method of claim 237, wherein said polynucleotide comprising a template nucleic acid is a double-stranded DNA encapsulated by said first lipid nanoparticle.

239. The method of claim 237 or claim 238, wherein said second polynucleotide is an mRNA encapsulated by said second lipid nanoparticle.

240. The method of claim 237 or claim 238, wherein said second polynucleotide is a double-stranded DNA encapsulated by said second lipid nanoparticle.

241. The method of any one of claims 192-240, wherein said subject is a human.

242. The method of any one of claims 192-241, wherein said target cell is a liver cell.

243. The method of any one of claims 192-241, wherein said target cell is a liver progenitor cell or stem cell.


SEQUENCE LISTING (170 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, synthetic construct
LAGLIDADG

SEQ ID NO: 3 - DNA, 7030 bp, synthetic construct
  source 1..7030: mol_type = other DNA
ggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 4 - DNA, 10562 bp, synthetic construct
  source 1..10562: mol_type = other DNA
ggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 5 - DNA, 6683 bp, synthetic construct
  source 1..6683: mol_type = other DNA
cagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 6 - DNA, 6683 bp, synthetic construct; identical to SEQ ID NO: 5
  source 1..6683: mol_type = other DNA

SEQ ID NO: 7 - DNA, 1257 bp, synthetic construct
  source 1..1257: mol_type = other DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 8 - DNA, 4789 bp, synthetic construct
  source 1..4789: mol_type = other DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 9 - DNA, 611 bp, synthetic construct
  source 1..611: mol_type = other DNA
gcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 10 - DNA, 2693 bp, synthetic construct
  source 1..2693: mol_type = other DNA
gcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 11 - DNA, 340 bp, synthetic construct
  source 1..340: mol_type = other DNA
gtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 12 - DNA, 1163 bp, synthetic construct
  source 1..1163: mol_type = other DNA
gtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 13 - DNA, 192 bp, synthetic construct
  source 1..192: mol_type = other DNA
gccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 14 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 15 - DNA, 12244 bp, synthetic construct
  source 1..12244: mol_type = other DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggtgggagggacagcagggcttagagtgggggtcattgtgcagatgggaaaacaaaggcccagagaggggaagaaatgcccaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcgggcgatgcaatctgatttaggcttttaaagggattgcaatcaagtggggccccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 16 - DNA, 8712 bp, synthetic construct
  source 1..8712: mol_type = other DNA
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggtgggagggacagcagggcttagagtgggggtcattgtgcagatgggaaaacaaaggcccagagaggggaagaaatgcccaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcgggcgatgcaatctgatttaggcttttaaagggattgcaatcaagtggggccccactagcctcaaccctgtacctcccctcccctccacccccagcagtctccaaaggcctccaacaaccccagagtgggggccatgtatccaaagaaactccaagctgtatacggatcacactggttttccaggagcaaaaacagaaacaggcctgaggctggtcaaaattgaacctcctcctgctctgagcagcctggggggcagactaagcagagggctgtgcagacccacataaagagcctactgtgtgccaggcacttcacccgaggcacttcacaagcatgcttgggaatgaaacttccaactctttgggatgcaggtgaaacagttcctggttcagagaggtgaagcggcctgcctgaggcagcacagctcttctttacagatgtgcttccccacctctaccctgtctcacggccccccatgccagcctgacggttgtgtctgcctcagtcatgctccatttttccatcgggaccatcaagagggtgtttgtgtctaaggctgactgggtaactttggatgagcggtctctccgctctgagcctgtttcctcatctgtcaaatgggctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgagaggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 17 - protein, 10 aa, synthetic construct
MAPKKKRKVH

SEQ ID NO: 18 - DNA, 22 bp, synthetic construct
  source 1..22: mol_type = other DNA
tgcccgctcacaccagaccctg

SEQ ID NO: 19 - DNA, 22 bp, synthetic construct
  source 1..22: mol_type = other DNA
acgggcgagtgtggtctgggac

SEQ ID NO: 20 - DNA, 22 bp, synthetic construct
  source 1..22: mol_type = other DNA
gctgctcaggcatctgcatttc

SEQ ID NO: 21 - DNA, 22 bp, synthetic construct
  source 1..22: mol_type = other DNA
cgacgagtccgtagacgtaaag

SEQ ID NO: 22 - DNA, 22 bp, synthetic construct
  source 1..22: mol_type = other DNA
cagagggctgtgcagacccaca

SEQ ID NO: 23 - DNA, 22 bp, synthetic construct
  source 1..22: mol_type = other DNA
gtctcccgacacgtctgggtgt

SEQ ID NO: 24 - DNA, 22 bp, synthetic construct
  source 1..22: mol_type = other DNA
ttcagacagttgctcaacctct

SEQ ID NO: 25 - DNA, 22 bp, synthetic construct
  source 1..22: mol_type = other DNA
aagtctgtcaacgagttggaga

SEQ ID NO: 26 - DNA, 22 bp, synthetic construct
  source 1..22: mol_type = other DNA
ggcctggtcacacttgggttta

SEQ ID NO: 27 - DNA, 22 bp, synthetic construct
  source 1..22: mol_type = other DNA
ccggaccagtgtgaacccaaat

SEQ ID NO: 28 - DNA, 22 bp, synthetic construct
  source 1..22: mol_type = other DNA
ccccactttgcacaactgggga

SEQ ID NO: 29 - DNA, 22 bp, synthetic construct
  source 1..22: mol_type = other DNA
ggggtgaaacgtgttgacccct

SEQ ID NO: 30 - DNA, 22 bp, synthetic construct
  source 1..22: mol_type = other DNA
tgaggatccttgtgagtgttgg

SEQ ID NO: 31 - DNA, 22 bp, synthetic construct
  source 1..22: mol_type = other DNA
actcctaggaacactcacaacc

SEQ ID NO: 32 - DNA, 22 bp, synthetic construct
  source 1..22: mol_type = other DNA
aggacctagatgtaggattctg

SEQ ID NO: 33 - DNA, 22 bp, synthetic construct
  source 1..22: mol_type = other DNA
tcctggatctacatcctaagac

SEQ ID NO: 34 - DNA, 22 bp, synthetic construct
  source 1..22: mol_type = other DNA
gctggactggtatttgtgtctg

SEQ ID NO: 35 - DNA, 22 bp, synthetic construct
  source 1..22: mol_type = other DNA
cgacctgaccataaacacagac

SEQ ID NO: 36 - DNA, 32 bp, synthetic construct
  source 1..32: mol_type = other DNA
tatccagtccaagctgtatacggatcacactg

SEQ ID NO: 37 - DNA, 32 bp, synthetic construct
  source 1..32: mol_type = other DNA
atgcacctccaagctgtatacggatcacactg

SEQ ID NO: 38 - DNA, 32 bp, synthetic construct
  source 1..32: mol_type = other DNA
cagttccaccaagctgtatacggatcacactg

SEQ ID NO: 39 - DNA, 32 bp, synthetic construct
  source 1..32: mol_type = other DNA
tctcgcctccaagctgtatacggatcacactg

SEQ ID NO: 40 - DNA, 35 bp, synthetic construct
  source 1..35: mol_type = other DNA
gtggttcctgaaccaggaactgtttcacctgcatc

SEQ ID NO: 41 - DNA, 35 bp, synthetic construct
  source 1..35: mol_type = other DNA
cgaacttctgaaccaggaactgtttcacctgcatc

SEQ ID NO: 42 - DNA, 35 bp, synthetic construct
  source 1..35: mol_type = other DNA
aggaatggtgaaccaggaactgtttcacctgcatc

SEQ ID NO: 43 - DNA, 35 bp, synthetic construct
  source 1..35: mol_type = other DNA
gttaggaatgaaccaggaactgtttcacctgcatc

SEQ ID NO: 44 - DNA, 35 bp, synthetic construct
  source 1..35: mol_type = other DNA
cgttgaggtgaaccaggaactgtttcacctgcatc

SEQ ID NO: 45 - DNA, 35 bp, synthetic construct
  source 1..35: mol_type = other DNA
ctcccaaatgaaccaggaactgtttcacctgcatc

SEQ ID NO: 46 - DNA, 35 bp, synthetic construct
  source 1..35: mol_type = other DNA
gacatttctgaaccaggaactgtttcacctgcatc

SEQ ID NO: 47 - DNA, 35 bp, synthetic construct
  source 1..35: mol_type = other DNA
tgagagattgaaccaggaactgtttcacctgcatc

SEQ ID NO: 48 - DNA, 31 bp, synthetic construct
  source 1..31: mol_type = other DNA
tatccagtctcttgaacctggccctagtgtc

SEQ ID NO: 49 - DNA, 31 bp, synthetic construct
  source 1..31: mol_type = other DNA
atgcacctctcttgaacctggccctagtgtc

SEQ ID NO: 50 - DNA, 31 bp, synthetic construct
  source 1..31: mol_type = other DNA
cagttccactcttgaacctggccctagtgtc

SEQ ID NO: 51 - DNA, 31 bp, synthetic construct
  source 1..31: mol_type = other DNA
tctcgcctctcttgaacctggccctagtgtc

SEQ ID NO: 52 - DNA, 44 bp, synthetic construct
  source 1..44: mol_type = other DNA
gtggttccaaattaaatccttccaacacttcagagatcaaggtc

SEQ ID NO: 53 - DNA, 44 bp, synthetic construct
  source 1..44: mol_type = other DNA
cgaacttcaaattaaatccttccaacacttcagagatcaaggtc

SEQ ID NO: 54 - DNA, 44 bp, synthetic construct
  source 1..44: mol_type = other DNA
aggaatggaaattaaatccttccaacacttcagagatcaaggtc

SEQ ID NO: 55 - DNA, 44 bp, synthetic construct
  source 1..44: mol_type = other DNA
gttaggaaaaattaaatccttccaacacttcagagatcaaggtc

SEQ ID NO: 56 - DNA, 44 bp, synthetic construct
  source 1..44: mol_type = other DNA
cgttgaggaaattaaatccttccaacacttcagagatcaaggtc

SEQ ID NO: 57 - DNA, 44 bp, synthetic construct
  source 1..44: mol_type = other DNA
ctcccaaaaaattaaatccttccaacacttcagagatcaaggtc

SEQ ID NO: 58 - DNA, 44 bp, synthetic construct
  source 1..44: mol_type = other DNA
gacatttcaaattaaatccttccaacacttcagagatcaaggtc

SEQ ID NO: 59 - DNA, 44 bp, synthetic construct
  source 1..44: mol_type = other DNA
tgagagataaattaaatccttccaacacttcagagatcaaggtc

SEQ ID NO: 60 - DNA, 35 bp, synthetic construct
  source 1..35: mol_type = other DNA
tatccagtctccattgtacagctatgaagctagtg

SEQ ID NO: 61 - DNA, 35 bp, synthetic construct
  source 1..35: mol_type = other DNA
atgcacctctccattgtacagctatgaagctagtg

SEQ ID NO: 62 - DNA, 35 bp, synthetic construct
  source 1..35: mol_type = other DNA
cagttccactccattgtacagctatgaagctagtg

SEQ ID NO: 63 - DNA, 35 bp, synthetic construct
  source 1..35: mol_type = other DNA
tctcgcctctccattgtacagctatgaagctagtg

SEQ ID NO: 64 - DNA, 35 bp, synthetic construct
  source 1..35: mol_type = other DNA
gtggttccgggagcataaaataagggaccaagagc

SEQ ID NO: 65 - DNA, 35 bp, synthetic construct
  source 1..35: mol_type = other DNA
cgaacttcgggagcataaaataagggaccaagagc

SEQ ID NO: 66 - DNA, 35 bp, synthetic construct
  source 1..35: mol_type = other DNA
aggaatgggggagcataaaataagggaccaagagc

SEQ ID NO: 67 - DNA, 35 bp, synthetic construct
  source 1..35: mol_type = other DNA
gttaggaagggagcataaaataagggaccaagagc

SEQ ID NO: 68 - DNA, 35 bp, synthetic construct
  source 1..35: mol_type = other DNA
cgttgagggggagcataaaataagggaccaagagc

SEQ ID NO: 69 - DNA, 35 bp, synthetic construct
  source 1..35: mol_type = other DNA
ctcccaaagggagcataaaataagggaccaagagc

SEQ ID NO: 70 - DNA, 35 bp, synthetic construct
  source 1..35: mol_type = other DNA
gacatttcgggagcataaaataagggaccaagagc

SEQ ID NO: 71 - DNA, 35 bp, synthetic construct
  source 1..35: mol_type = other DNA
tgagagatgggagcataaaataagggaccaagagc

SEQ ID NO: 72 - DNA, 32 bp, synthetic construct
  source 1..32: mol_type = other DNA
tatccagttggcagataaagcgagactctgtc

SEQ ID NO: 73 - DNA, 32 bp, synthetic construct
  source 1..32: mol_type = other DNA
atgcaccttggcagataaagcgagactctgtc

SEQ ID NO: 74 - DNA, 32 bp, synthetic construct
  source 1..32: mol_type = other DNA
cagttccatggcagataaagcgagactctgtc

SEQ ID NO: 75 - DNA, 32 bp, synthetic construct
  source 1..32: mol_type = other DNA
tctcgccttggcagataaagcgagactctgtc

SEQ ID NO: 76 - DNA, 32 bp, synthetic construct
  source 1..32: mol_type = other DNA
gccgaatgtggcagataaagcgagactctgtc

SEQ ID NO: 77 - DNA, 32 bp, synthetic construct
  source 1..32: mol_type = other DNA
tactgcagtggcagataaagcgagactctgtc

SEQ ID NO: 78 - DNA, 32 bp, synthetic construct
  source 1..32: mol_type = other DNA
catgttgatggcagataaagcgagactctgtc

SEQ ID NO: 79 - DNA, 32 bp, synthetic construct
  source 1..32: mol_type = other DNA
atagagtctggcagataaagcgagactctgtc

SEQ ID NO: 80 - DNA, 32 bp, synthetic construct
  source 1..32: mol_type = other DNA
tcacgctctggcagataaagcgagactctgtc

SEQ ID NO: 81 - DNA, 31 bp, synthetic construct
  source 1..31: mol_type = other DNA
gtggttccgggccagaggttgtggcttctag

SEQ ID NO: 82 - DNA, 31 bp, synthetic construct
  source 1..31: mol_type = other DNA
cgaacttcgggccagaggttgtggcttctag

SEQ ID NO: 83 - DNA, 31 bp, synthetic construct
  source 1..31: mol_type = other DNA
aggaatgggggccagaggttgtggcttctag

SEQ ID NO: 84 - DNA, 31 bp, synthetic construct
  source 1..31: mol_type = other DNA
gttaggaagggccagaggttgtggcttctag

SEQ ID NO: 85 - DNA, 31 bp, synthetic construct
  source 1..31: mol_type = other DNA
cgttgagggggccagaggttgtggcttctag

SEQ ID NO: 86 - DNA, 31 bp, synthetic construct
  source 1..31: mol_type = other DNA
ctcccaaagggccagaggttgtggcttctag

SEQ ID NO: 87 - DNA, 31 bp, synthetic construct
  source 1..31: mol_type = other DNA
gacatttcgggccagaggttgtggcttctag

SEQ ID NO: 88 - DNA, 31 bp, synthetic construct
  source 1..31: mol_type = other DNA
tgagagatgggccagaggttgtggcttctag

SEQ ID NO: 89 - DNA, 29 bp, synthetic construct
  source 1..29: mol_type = other DNA
tatccagtgctgactgcaggagcatcagc

SEQ ID NO: 90 - DNA, 32 bp, synthetic construct
  source 1..32: mol_type = other DNA
gtggttccacagaagggaaatgcatcttgcac

SEQ ID NO: 91 - DNA, 31 bp, synthetic construct
  source 1..31: mol_type = other DNA
cgaacttacagaagggaaatgcatcttgcac

SEQ ID NO: 92 - DNA, 32 bp, synthetic construct
  source 1..32: mol_type = other DNA
aggaatggacagaagggaaatgcatcttgcac

SEQ ID NO: 93 - DNA, 32 bp, synthetic construct
  source 1..32: mol_type = other DNA
gttaggaaacagaagggaaatgcatcttgcac

SEQ ID NO: 94 - DNA, 32 bp, synthetic construct
  source 1..32: mol_type = other DNA
cgttgaggacagaagggaaatgcatcttgcac

SEQ ID NO: 95 - DNA, 32 bp, synthetic construct
  source 1..32: mol_type = other DNA
ctcccaaaacagaagggaaatgcatcttgcac

SEQ ID NO: 96 - DNA, 32 bp, synthetic construct
  source 1..32: mol_type = other DNA
gacatttcacagaagggaaatgcatcttgcac

SEQ ID NO: 97 - DNA, 32 bp, synthetic construct
  source 1..32: mol_type = other DNA
tgagagatacagaagggaaatgcatcttgcac

SEQ ID NO: 98 - DNA, 30 bp, synthetic construct
  source 1..30: mol_type = other DNA
tatccagtaggcccattcctcttcttgtgc

SEQ ID NO: 99 - DNA, 30 bp, synthetic construct
  source 1..30: mol_type = other DNA
atgcacctaggcccattcctcttcttgtgc

SEQ ID NO: 100 - DNA, 30 bp, synthetic construct
  source 1..30: mol_type = other DNA
cagttccaaggcccattcctcttcttgtgc

SEQ ID NO: 101 - DNA, 30 bp, synthetic construct
  source 1..30: mol_type = other DNA
tctcgcctaggcccattcctcttcttgtgc

SEQ ID NO: 102 - DNA, 30 bp, synthetic construct
  source 1..30: mol_type = other DNA
gtggttcccaatcccgtgaggtgctaatgc

SEQ ID NO: 103 - DNA, 29 bp, synthetic construct
  source 1..29: mol_type = other DNA
cgaacttcaatcccgtgaggtgctaatgc

SEQ ID NO: 104 - DNA, 30 bp, synthetic construct
  source 1..30: mol_type = other DNA
aggaatggcaatcccgtgaggtgctaatgc

SEQ ID NO: 105 - DNA, 30 bp, synthetic construct
  source 1..30: mol_type = other DNA
gttaggaacaatcccgtgaggtgctaatgc

SEQ ID NO: 106 - DNA, 30 bp, synthetic construct
  source 1..30: mol_type = other DNA
cgttgaggcaatcccgtgaggtgctaatgc

SEQ ID NO: 107 - DNA, 30 bp, synthetic construct
  source 1..30: mol_type = other DNA
ctcccaaacaatcccgtgaggtgctaatgc

SEQ ID NO: 108 - DNA, 30 bp, synthetic construct
  source 1..30: mol_type = other DNA
gacatttccaatcccgtgaggtgctaatgc

SEQ ID NO: 109 - DNA, 30 bp, synthetic construct
  source 1..30: mol_type = other DNA
tgagagatcaatcccgtgaggtgctaatgc

SEQ ID NO: 110 - DNA, 23 bp, synthetic construct
  source 1..23: mol_type = other DNA
ctgctcaggcatctgcatttccc

SEQ ID NO: 111 - DNA, 17 bp, synthetic construct
  source 1..17: mol_type = other DNA
atgcctcgatggtgaag

SEQ ID NO: 112 - DNA, 22 bp, synthetic construct
  source 1..22: mol_type = other DNA
ggcaaacattctgcttactatt

SEQ ID NO: 113 - DNA, 25 bp, synthetic construct
  source 1..25: mol_type = other DNA
cctcgctggactggtatttgtgtct

SEQ ID NO: 114 - DNA, 19 bp, synthetic construct
  source 1..19: mol_type = other DNA
gacaggatggcttcccttc

SEQ ID NO: 115 - DNA, 21 bp, synthetic construct
  source 1..21: mol_type = other DNA
gtctaactgccatgtctggat

SEQ ID NO: 116 - DNA, 24 bp, synthetic construct
  source 1..24: mol_type = other DNA
accttagtgatgcccagttgaccc

SEQ ID NO: 117 - DNA, 21 bp, synthetic construct
  source 1..21: mol_type = other DNA
ccacaactagaatgcagtgaa

SEQ ID NO: 118 - DNA, 19 bp, synthetic construct
  source 1..19: mol_type = other DNA
gaatcacgggcatcttcag

SEQ ID NO: 119 - DNA, 19 bp, synthetic construct
  source 1..19: mol_type = other DNA
ctgcccgctcacaccagac

SEQ ID NO: 120 - DNA, 16 bp, synthetic construct
  source 1..16: mol_type = other DNA
agcacctcctgaggtc

SEQ ID NO: 121 - DNA, 16 bp, synthetic construct
  source 1..16: mol_type = other DNA
cacaccaggctgagtg

SEQ ID NO: 122 - DNA, 20 bp, synthetic construct
  source 1..20: mol_type = other DNA
tggacaaaccacaactagaa

SEQ ID NO: 123 - DNA, 19 bp, synthetic construct
  source 1..19: mol_type = other DNA
agcctggctaaatgaagag

SEQ ID NO: 124 - DNA, 24 bp, synthetic construct
  source 1..24: mol_type = other DNA
agcaagttgacgtggagcaatctg

SEQ ID NO: 125 - DNA, 20 bp, synthetic construct
  source 1..20: mol_type = other DNA
tggacaaaccacaactagaa

SEQ ID NO: 126 - DNA, 19 bp, synthetic construct
  source 1..19: mol_type = other DNA
ctcccaagctgttccttat

SEQ ID NO: 127 - DNA, 23 bp, synthetic construct
  source 1..23: mol_type = other DNA
agggtgctgcctctgatagaagg

SEQ ID NO: 128 - DNA, 20 bp, synthetic construct
  source 1..20: mol_type = other DNA
tggacaaaccacaactagaa

SEQ ID NO: 129 - DNA, 20 bp, synthetic construct
  source 1..20: mol_type = other DNA
ccacagtgatccttctactc

SEQ ID NO: 130 - DNA, 24 bp, synthetic construct
  source 1..24: mol_type = other DNA
tctgtgaagacggcaggttctacc

SEQ ID NO: 131 - DNA, 20 bp, synthetic construct
  source 1..20: mol_type = other DNA
tggacaaaccacaactagaa

SEQ ID NO: 132 - DNA, 21 bp, synthetic construct
  source 1..21: mol_type = other DNA
cgtgaggtgctaatgctaata

SEQ ID NO: 133 - DNA, 26 bp, synthetic construct
  source 1..26: mol_type = other DNA
agctgacctaatctctctggctttgg

SEQ ID NO: 134 - DNA, 20 bp, synthetic construct
  source 1..20: mol_type = other DNA
tggacaaaccacaactagaa

SEQ ID NO: 135 - DNA, 18 bp, synthetic construct
  source 1..18: mol_type = other DNA
ctgcaagacagagatggg

SEQ ID NO: 136 - DNA, 23 bp, synthetic construct
  source 1..23: mol_type = other DNA
agtcatcatgtgccttgactcgg

SEQ ID NO: 137 - DNA, 20 bp, synthetic construct
  source 1..20: mol_type = other DNA
gagagaacgcaccactttac

SEQ ID NO: 138 - DNA, 19 bp, synthetic construct
  source 1..19: mol_type = other DNA
agcctggctaaatgaagag

SEQ ID NO: 139 - DNA, 24 bp, synthetic construct
  source 1..24: mol_type = other DNA
agcaagttgacgtggagcaatctg

SEQ ID NO: 140 - DNA, 20 bp, synthetic construct
  source 1..20: mol_type = other DNA
gagagaacgcaccactttac

SEQ ID NO: 141 - DNA, 19 bp, synthetic construct
  source 1..19: mol_type = other DNA
ctcccaagctgttccttat

SEQ ID NO: 142 - DNA, 23 bp, synthetic construct
  source 1..23: mol_type = other DNA
agggtgctgcctctgatagaagg

SEQ ID NO: 143 - DNA, 20 bp, synthetic construct
  source 1..20: mol_type = other DNA
gagagaacgcaccactttac

SEQ ID NO: 144 - DNA, 20 bp, synthetic construct
  source 1..20: mol_type = other DNA
ccacagtgatccttctactc

SEQ ID NO: 145 - DNA, 24 bp, synthetic construct
  source 1..24: mol_type = other DNA
tctgtgaagacggcaggttctacc

SEQ ID NO: 146 - DNA, 20 bp, synthetic construct
  source 1..20: mol_type = other DNA
gagagaacgcaccactttac

SEQ ID NO: 147 - DNA, 21 bp, synthetic construct
  source 1..21: mol_type = other DNA
cgtgaggtgctaatgctaata

SEQ ID NO: 148 - DNA, 26 bp, synthetic construct
  source 1..26: mol_type = other DNA
agctgacctaatctctctggctttgg

SEQ ID NO: 149 - DNA, 20 bp, synthetic construct
  source 1..20: mol_type = other DNA
gagagaacgcaccactttac

SEQ ID NO: 150 - DNA, 18 bp, synthetic construct
  source 1..18: mol_type = other DNA
ctgcaagacagagatggg

SEQ ID NO: 151 - DNA, 23 bp, synthetic construct
  source 1..23: mol_type = other DNA
agtcatcatgtgccttgactcgg

SEQ ID NO: 152 - DNA, 20 bp, synthetic construct
  source 1..20: mol_type = other DNA
ctgtttctcttgggtctcag

SEQ ID NO: 153 - DNA, 21 bp, synthetic construct
  source 1..21: mol_type = other DNA
gggacagaagtcaaaggttat

SEQ ID NO: 154 - DNA, 22 bp, synthetic construct
  source 1..22: mol_type = other DNA
aacagagaggttgagcaactgt

SEQ ID NO: 155 - DNA, 20 bp, synthetic construct
  source 1..20: mol_type = other DNA
tgaagtcatttaccccaggc

SEQ ID NO: 156 - DNA, 20 bp, synthetic construct
  source 1..20: mol_type = other DNA
ctcacaccatttctacccgg

SEQ ID NO: 157 - DNA, 24 bp, synthetic construct
  source 1..24: mol_type = other DNA
aataaacccaagtgtgaccaggcc

SEQ ID NO: 158 - DNA, 18 bp, synthetic construct
  source 1..18: mol_type = other DNA
gcgagactctgtctcaaa

SEQ ID NO: 159 - DNA, 18 bp, synthetic construct
  source 1..18: mol_type = other DNA
ggccatcactatccactt

SEQ ID NO: 160 - DNA, 17 bp, synthetic construct
  source 1..17: mol_type = other DNA
tccccagttgtgcaaag

SEQ ID NO: 161 - DNA, 17 bp, synthetic construct
  source 1..17: mol_type = other DNA
caggagcatcagcctat

SEQ ID NO: 162 - DNA, 18 bp, synthetic construct
  source 1..18: mol_type = other DNA
atggcaacagctagagag

SEQ ID NO: 163 - DNA, 24 bp, synthetic construct
  source 1..24: mol_type = other DNA
atccttgtgagtgttgggtgggaa

SEQ ID NO: 164 - DNA, 20 bp, synthetic construct
  source 1..20: mol_type = other DNA
ggcccgattcctggataatc

SEQ ID NO: 165 - DNA, 20 bp, synthetic construct
  source 1..20: mol_type = other DNA
gccagagagattaggtcagc

SEQ ID NO: 166 - DNA, 27 bp, synthetic construct
  source 1..27: mol_type = other DNA
ccagaatcctacatctaggtcctgcac

SEQ ID NO: 167 - DNA, 18 bp, synthetic construct
  source 1..18: mol_type = other DNA
ctccaaggccgtgcataa

SEQ ID NO: 168 - DNA, 20 bp, synthetic construct
  source 1..20: mol_type = other DNA
agacatgggtatggcctcta

SEQ ID NO: 169 - DNA, 20 bp, synthetic construct
  source 1..20: mol_type = other DNA
ctgaccatcgacgagaaagg

SEQ ID NO: 170 - DNA, 20 bp, synthetic construct
  source 1..20: mol_type = other DNA
ctgaccatcgacaagaaagg